SEQUENCE LISTING (18 sequences)

SEQ ID NO: 1 - DNA, 1458 bp, Artificial Sequence
  Anti-human-CD19-BBZ CAR
atggccttaccagtgaccgccttgctcctgccgctggccttgctgctccacgccgccaggccggacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggaccaagctggagatcacaggtggcggtggctcgggcggtggtgggtcgggtggcggcggatctgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggccaaggaacctcagtcaccgtctcctcaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 2 - DNA, 726 bp, Artificial Sequence
  Anti-human-CD19 scFv
gacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggaccaagctggagatcacaggtggcggtggctcgggcggtggtgggtcgggtggcggcggatctgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggccaaggaacctcagtcaccgtctcctca

SEQ ID NO: 3 - DNA, 63 bp, Artificial Sequence
  CD8 Leader
atggccttaccagtgaccgccttgctcctgccgctggccttgctgctccacgccgccaggccg

SEQ ID NO: 4 - DNA, 207 bp, Artificial Sequence
  CD8 Hinge and transmembrane domain
accacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgc

SEQ ID NO: 5 - DNA, 126 bp, Artificial Sequence
  4-1BB intracellular domain
aaacggggcagaaagaaactcctgtatatattcaaacaaccatttatgagaccagtacaaactactcaagaggaagatggctgtagctgccgatttccagaagaagaagaaggaggatgtgaactg

SEQ ID NO: 6 - DNA, 336 bp, Artificial Sequence
  CD3 zeta intracellular domain
agagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 7 - protein, 5 aa, Artificial Sequence
  linker
  REPEAT (1)..(5): repeat n times, where n represents an integer of 1
GSGGS

SEQ ID NO: 8 - protein, 4 aa, Artificial Sequence
  linker
  REPEAT (1)..(4): repeat n times, where n represents an integer of 1
GGGS

SEQ ID NO: 9 - protein, 5 aa, Artificial Sequence
  linker
  REPEAT (1)..(5): repeat n times, where n represents an integer of 1
GGGGS

SEQ ID NO: 10 - protein, 4 aa, Artificial Sequence
  linker
GGSG

SEQ ID NO: 11 - protein, 5 aa, Artificial Sequence
  linker
GGSGG

SEQ ID NO: 12 - protein, 5 aa, Artificial Sequence
  linker
GSGSG

SEQ ID NO: 13 - protein, 5 aa, Artificial Sequence
  linker
GSGGG

SEQ ID NO: 14 - protein, 5 aa, Artificial Sequence
  linekr
GGGSG

SEQ ID NO: 15 - protein, 5 aa, Artificial Sequence
  linker
GSSSG

SEQ ID NO: 16 - protein, 5 aa, Artificial Sequence
  linker
GGGGS

SEQ ID NO: 17 - protein, 15 aa, Artificial Sequence
  linker
GGGGSGGGGSGGGGS

SEQ ID NO: 18 - DNA, 45 bp, Artificial Sequence
  linker
ggtggcggtggctcgggcggtggtgggtcgggtggcggcggatct

(19) United States
(12) Patent Application Publication
(10) Pub. No.: US 2024/0197876 A1
(43) Pub. Date: Jun. 20, 2024

(54) METHODS TO DETERMINE TREATMENT EFFICACY WITH GAMMA-DELTA T CELLS

(71) Applicant: THE TRUSTEES OF THE UNIVERSITY OF PENNSYLVANIA, Philadelphia, PA (US)

(72) Inventors: Xiaowei XU, Monmouth Junction, NJ (US); Huaishan WANG, Philadelphia, PA (US); Hui CHEN, Beijing (CN); Lingling OU, Philadelphia, PA (US)

(21) Appl. No.: 18/555,214

(22) PCT Filed: Apr. 13, 2022
(86) PCT No.: PCT/US2022/024668

§ 371 Date: Oct. 12, 2023

Related U.S. Application Data
(60) Provisional application No. 63/174,879, filed on Apr. 14, 2021.
(60) Provisional application No. 63/285,361, filed on Dec. 2, 2021.

(51) Int. Cl. A61K 39/00; A61K 35/17; A61P 35/00; C07K 16/28; C12N 5/0783; G01N 33/50

(52) U.S. Cl. CPC A61K 39/4631; A61K 35/17; A61K 39/4611; A61K 39/464412; A61K 39/464449; A61P 35/00; C07K 16/2809; C07K 16/2818; C12N 5/0636; G01N 33/5094; A61K 2239/31; A61K 2239/38; A61K 2239/57; C12N 2501/2302; C12N 2510/00

(57) ABSTRACT

The present disclosure provides methods for selecting donors for γδT cell-based immunotherapy. The disclosure also provides methods for expanding and activating γδ T cells with improved cytotoxicity toward tumor cells and reduced T cell exhaustion. Pharmaceutical compositions comprising the expanded γδ T cells and methods of treatment are also provided.

### CROSS-REFERENCE TO RELATED APPLICATIONS

The present application is entitled to priority under 35 U.S.C. § 119(e) to U.S. Provisional Patent Application No. 63/174,879 filed Apr. 14, 2021, and U.S. Provisional Patent Application No. 63/285,361 filed Dec. 2, 2021, each of which is hereby incorporated by reference in its entirety herein.

### STATEMENT REGARDING FEDERALLY SPONSORED RESEARCH OR DEVELOPMENT

This invention was made with government support under contracts CA1 14046 and CA174523 awarded by National Institutes of Health. The government has certain rights in the invention.

### BACKGROUND OF THE INVENTION

Adoptive immunotherapy has shown unprecedented success. Adoptive T cell transfer has so far focused mainly on using αβT cells, which are effective in certain patient populations. Unlike αβT cells, γδ T cells manifest the features of both innate and adaptive immunity. γδ T cells are pre-programmed to locate and destroy cells that are ‘stressed’ by transformation, which makes them a good candidate for adoptive T cell transfer. γδ T cells differ from αβT cells by their TCR gene usage, tissue tropism, and MHC-independent antigen recognition. In addition, tumors may not always need to be immunogenic in the conventional sense of activating naïve αβT cells but may still be targets for γδ T cells. Downregulation or loss of HLA class I or β2 microglobulin which makes tumor cells undetectable to αβT cells is unlikely to affect γδ T cell recognition. An additional perquisite of using γδ T cells for immunotherapy lies in their ability to cross present processed tumor antigen to αβT cells. Meta-analysis of gene expression signatures from ˜18,000 human tumors across 39 malignancies indicated a tumor-associated γδT-cell profile as the best predicator of patient survival. Thus, γδ T cells may represent a unique cell source for immunotherapy.

Human peripheral blood contains both Vδ1 and Vδ2 γδ T cells, but the predominant γδT cells are the Vδ2 subset that co-expresses the Vγ9 chain, named Vγ9Vδ2 T cells. γδ T cells account for less than 5% of total T cells. Given that Vδ2 T cells are relatively abundant in peripheral blood, most γδ T cell clinical trials to date have focused on the Vδ2 T cell subset. For most clinical trials and preclinical studies, zoledronate (ZOL, an FDA-approved drug) and anti-γδTCR antibodies, have been used to selectively activate and expand γδ T cells both in vitro and in vivo. Since Vγ9Vδ2 T cells are not MHC-restricted, off-the-shelf allogeneic therapy using cells expanded from healthy donors is possible.

Adoptive immunotherapy with γδ T cells has been tested in hundreds of patients with various cancers and these clinic trials showed that γδ T cell immunotherapy was well tolerated and safe. However, despite complete remissions that were reported in cancer patients, for the majority of patients, the efficacy was limited. The expansion capacity of γδ T cells from cancer patients was lower than that in healthy donors, and cancer patient-derived γδ T cells had dysfunctional effector cytokine production and cytotoxicity.

The failure of current γδT-based therapies may be due to a highly variable capacity of the polyclonal γδT TCR repertoire to recognize tumors, functional instability, or dysfunction or exhaustion of chronically activated γδ T cells. The interindividual heterogeneity also manifests as a high degree of variation in γδ T cell expansion capacity, despite the fact that the circulating γδT cell profile in an individual is stable over time after birth.

There is a need in the art for a better understanding of the interindividual heterogeneity in γδ T cells, in order to choose donors for optimal γδ T cell-based immunotherapy efficacy. The present invention addresses this need.

### SUMMARY OF THE INVENTION

In one aspect, the disclosure provides a method of selecting a donor for γδ T cell-based immunotherapy. The method comprising assessing the expansion capacity of a population of γδT cells from a subject. When the expansion capacity is high, the subject is selected as a donor. When the expansion capacity is low, the subject is not selected as a donor.

In certain embodiments, the assessing comprises incubating the γδ T cells with an agent or agents that expand the γδ T cells. In certain embodiments, the agent or agents are selected from ZOL, an anti-γδTCR antibody, IPP plus a TLR agonist, and an anti-γδTCR antibody plus a TLR agonist.

In certain embodiments, assessing the expansion capacity comprises calculating an index score. In certain embodiments, the index score is calculated based on the baseline γδ T cell concentration in the subject's PMBC using the formula: Vδ2 Index Score=−8.23+10.25×Initial γδT %.

In another aspect, the disclosure provides a method of selecting a donor for γδ T cell-based immunotherapy comprising measuring the basal level of Vδ2 T cells in a subject. When the basal level of Vδ2 T cells is high, the subject is selected as a donor. When the basal level of Vδ2 T cells is low, the subject is not selected as a donor.

In certain embodiments, the method further comprises autologously transferring the γδT cell-based immunotherapy to the donor.

In certain embodiments, when the basal level of Vδ2 T cells is between 0.5-3% of the total PBMC population, the level is considered high. In certain embodiments, when the basal level of Vδ2 T cells is at least 0.82% of the total PBMC population, the level is considered high.

In certain embodiments, the method further comprising assesses the immune phenotypes of the γδ T cells. In certain embodiments, when the subject's γδ T cells display effector memory type and central memory phenotype the subject is selected as a donor.

In certain embodiments, the method further comprises assessing the levels of PD-1, CTLA-4, Eomes, IFN-γ, Granzyme B, and CD86 in the γδ T cells. In certain embodiments, when the levels of PD-1, CTLA-4 and Eomes are low, and the levels of IFN-γ, Granzyme B, and CD86 are high in the subject's γδ T cells compared to a reference sample, then the subject is selected as a donor. In certain embodiments, the reference sample comprises γδ T cells from a poor expansion group.

In another aspect, the disclosure provides a composition comprising a population of γδT cells for use in γδ T cell immunotherapy that is generated by any of the methods contemplated herein.

In another aspect, the disclosure provides a method of treating a disease or disorder in a subject in need thereof with a γδ T cell-based immunotherapy. The method comprises selecting a donor for the γδ T cell-based immunotherapy by assessing the expansion capacity of a population of γδ T cells from a healthy subject. When the expansion capacity is high, the healthy subject is selected as a donor, and a γδ T cell-based immunotherapy comprising the γδ T cells from the donor is administered to the subject in need thereof, thus treating the disease or disorder. When the expansion capacity is low, the healthy subject is not selected as a donor and an alternative treatment is administered or a different donor is selected.

In another aspect, the disclosure provides a method of treating a disease or disorder in a subject in need thereof with a γδ T cell-based immunotherapy. The method comprises assessing the expansion capacity of a population of γδ T cells from the subject. When the expansion capacity is high, the subject is administered a γδ T cell-based immunotherapy comprising the γδT cells from the subject, thus treating the disease or disorder. When the expansion capacity is low, the γδ T cell-based immunotherapy is not administered and an alternative treatment is administered and/or a new donor is selected.

In certain embodiments, the assessing comprises incubating the γδ T cells with an agent or agents that expand the γδ T cells. In certain embodiments, the agent or agents are selected from ZOL, an anti-γδTCR antibody, IPP plus a TLR agonist, and an anti-γδTCR antibody plus a TLR agonist.

In certain embodiments, assessing the expansion capacity comprises calculating an index score. In certain embodiments, the index score is calculated based on the baseline γδ T cell concentration in the subject's PMBC using the formula: Vδ2 Index Score=−8.23+10.25×Initial γδT %.

In another aspect, the disclosure provides a method of treating a disease or disorder in a subject in need thereof with a γδ T cell-based immunotherapy. The method comprises selecting a donor for the γδ T cell-based immunotherapy by measuring the basal level of Vδ2 T cells in a healthy subject. When the basal level of Vδ2 T cells is high, the healthy subject is selected as a donor, and a γδ T cell-based immunotherapy comprising the γδ T cells from the donor is administered to the subject in need thereof, thus treating the disease or disorder. When the basal level of Vδ2 T cells is low, the healthy subject is not selected as a donor, and an alternative treatment is administered or a different donor is selected.

In certain embodiments, when the basal level of Vδ2 T cells is between 0.5-3% of the total PBMC population, the level is considered high. In certain embodiments, when the basal level of Vδ2 T cells is at least 0.82% of the total PBMC population, the level is considered high. In certain embodiments, the method further comprising assessing the immune phenotypes of the γδ T cells. In certain embodiments, when the subject's γδ T cells display effector memory type and central memory phenotype the subject is selected as a donor.

In certain embodiments, the method further comprises assessing the levels of PD-1, CTLA-4, Eomes, IFN-γ, Granzyme B, and CD86 in the γδ T cells. In certain embodiments, when the levels of PD-1, CTLA-4 and Eomes are low, and the levels of IFN-γ, Granzyme B, and CD86 are high in the subject's γδ T cells compared to a reference sample, then the subject is selected as a donor. In certain embodiments, the reference sample comprises γδ T cells from a poor expansion group.

In certain embodiments, the disease or disorder is cancer. In certain embodiments, the cancer is selected from the group consisting of breast cancer and lung cancer.

In certain embodiments, the disease or disorder is an infection. In certain embodiments, the infection is selected from the group consisting of a viral infection, Hepatitis C, Hepatitis B, HIV, EBV, HPV, and a bacterial infection (e.g. tuberculosis).

In certain embodiments, the γδ T cell-based immunotherapy further comprises a chimeric antigen receptor.

In certain embodiments, the γδ T cell-based immunotherapy further comprises a modified T cell receptor (TCR).

In another aspect, the disclosure provides a method for expanding and activating a plurality of γδ T cells. The method comprises contacting a population of cells comprising a plurality of γδ T cells with: (a) at least one γδ T cell receptor (γδTCR) activator, wherein the γδTCR activator is not zoledronate and is a weaker γδTCR activator than zoledronate; and (b) a toll-like receptor (TLR) agonist.

In certain embodiments, the γδTCR activator comprises: (i) a phosphoantigen; and/or (ii) at least one anti-γδTCR antibody and/or anti-γδTCR antigen-binding domain.

In certain embodiments, the TLR agonist comprises an agonist of TLR7 and/or TLR8 (TLR7/8 agonist). In certain embodiments, the TLR7/8 agonist comprises an imidazoquinoline compound. In certain embodiments, the imidazoquinoline compound is selected from resiquimod, imiquimod, and gardiquimod. In certain embodiments, the imidazoquinoline compound is resiquimod.

In certain embodiments, the phosphoantigen is selected from the group consisting of isopentenyl pyrophosphate (IPP), bromohydrin pyrophosphate (BrHPP), and 2-methyl-3-butenyl-1-pyrophosphate (2M3B1PP).

In certain embodiments, the γδTCR activator is IPP and the TLR agonist is resiquimod.

In certain embodiments, the population of cells comprises peripheral blood mononuclear cells (PBMCs). In certain embodiments, the population of cells comprises human cells. In certain embodiments, the plurality of γδ T cells comprises human γδ T cells.

In certain embodiments, the method is performed ex vivo.

In certain embodiments, the contacting is performed in a cell culture medium. In certain embodiments, the contacting of step (a) and step (b) are performed on the same day.

In certain embodiments, the plurality of γδ T cells is expanded at least 10-fold, at least 100-fold, at least 1,000-fold, or at least 10,000-fold.

In certain embodiments, the expanded plurality of γδ T cells comprises Vδ2 T cells.

In certain embodiments, a plurality of the at least one anti-γδTCR antibody and/or anti-γδTCR antigen-binding domain is attached to a bead to generate an antibody-conjugated bead. In certain embodiments, the bead is further attached to a plurality of at least one anti-costimulatory receptor antibody and/or anti-costimulatory receptor antigen-binding domain. In certain embodiments, the costimulatory receptor is selected from CD27, CD28, CD137 (4-11BB), CD277 (BTN3A1), CD314 (NKG2D), and PD-1 (CD279). In certain embodiments, the bead has a diameter ranging from about 100 nm to about 10 μm. In certain embodiments, the bead is a magnetic bead.

In certain embodiments, the plurality of γδ T cells binds to a plurality of the antibody-conjugated beads, and wherein the method further comprises purifying the plurality of γδ T cells away from other cells within the population of cells. In certain embodiments, the method comprises applying a magnetic field, thereby separating the plurality of γδ T cells bound by the plurality of antibody-conjugated beads from other cells within the population of cells.

In certain embodiments, the expanded plurality of γδ T cells is characterized by one or more of the following phenotypes compared to a plurality of γδ T cells expanded in the absence of a TLR agonist and in the presence of the γδTCR activator or a bisphosphonate: (i) increased expression of cytotoxicity surface markers (e.g., granzyme B, CD107a, and/or CD86); (ii) increased expression of proinflammatory cytokines (e.g., IFN-γ, TNF-α, and/or IL-17A); (iii) decreased expression of immune checkpoint proteins (e.g., PD-1); (iv) increased cytotoxicity toward tumor cells; (v) enhanced tumor volume reduction in vivo; (vi) enhanced PI3K-Akt-mTOR pathway; and/or (vii) ability to suppress inhibitory functions of adherent antigen presenting cells (APCs) present in the population of cells.

In certain embodiments, the plurality of γδ T cells is genetically modified. In certain embodiments, the plurality of γδ T cells is genetically modified to express a receptor selected from a chimeric antigen receptor (CAR), a TCR, a dominant negative receptor, a switch receptor, or any combination thereof.

In another aspect, the disclosure provides a method for expanding and activating a plurality of γδ T cells. The method comprises contacting a population of cells comprising a plurality of γδ T cells with an antibody-conjugated bead, wherein the antibody-conjugated bead comprises a bead attached to a plurality of at least one anti-γδ T cell receptor (anti-γδTCR) antibody and/or anti-γδTCR antigen-binding domain; and wherein the method does not comprise contacting the population of cells with zoledronate.

In certain embodiments, the bead is further attached to a plurality of at least one anti-costimulatory receptor antibody and/or anti-costimulatory receptor antigen-binding domain. In certain embodiments, the costimulatory receptor is selected from CD27, CD28, CD137 (4-1BB), CD277 (BTN3A1), CD314 (NKG2D), and PD-1 (CD279). In certain embodiments, the bead is further attached to an anti-PD1 antibody.

In certain embodiments, the expanded plurality of γδ T cells is characterized by one or more of the following phenotypes compared to a plurality of γδ T cells expanded in the absence of an anti-costimulatory receptor antibody and/or anti-costimulatory receptor antigen binding domain and in the presence of the anti-γδTCR antibody or anti-γδTCR antigen binding domain: (i) increased expression of cytotoxicity surface markers (e.g., granzyme B, CD107a, and/or CD86); (ii) increased expression of proinflammatory cytokines (e.g., IFN-7, TNF-α, and/or IL-17A); (iii) decreased expression of immune checkpoint proteins (e.g., PD-1); (iv) increased cytotoxicity toward tumor cells; and/or (v) enhanced tumor volume reduction in vivo.

In certain embodiments, the method further comprising contacting the population of cells comprising a plurality of γδ T cells with a toll-like receptor (TLR) agonist. In certain embodiments, the TLR agonist comprises an agonist of TLR7 and/or TLR8 (TLR7/8 agonist). In certain embodiments, the TLR7/8 agonist comprises an imidazoquinoline compound. In certain embodiments, the imidazoquinoline compound is selected from resiquimod, imiquimod, and gardiquimod. In certain embodiments, the imidazoquinoline compound is resiquimod.

In certain embodiments, contacting the population of cells with the toll-like receptor (TLR) agonist is performed on the same day as contacting the population of cells with the antibody-conjugated bead.

In certain embodiments, the population of cells comprises peripheral blood mononuclear cells (PBMCs). In certain embodiments, the population of cells comprises human cells. In certain embodiments, the plurality of γδ T cells comprises human γδ T cells.

In certain embodiments, the method is performed ex vivo.

In certain embodiments, the contacting is performed in a cell culture medium.

In certain embodiments, the plurality of γδ T cells is expanded at least 10-fold, at least 100-fold, at least 1,000-fold, or at least 10,000-fold.

In certain embodiments, the expanded plurality of γδ T cells comprises Vδ2 T cells.

In certain embodiments, wherein the bead has a diameter ranging from about 100 nm to about 10 μm. In certain embodiments, 70, wherein the bead is a magnetic bead.

In certain embodiments, the plurality of γδ T cells binds to a plurality of the antibody-conjugated beads, and wherein the method further comprises purifying the plurality of γδ T cells away from other cells within the population of cells. In certain embodiments, the method comprises applying a magnetic field, thereby separating the plurality of γδ T cells bound by the plurality of antibody-conjugated beads from other cells within the population of cells.

In certain embodiments, the expanded plurality of γδ T cells is characterized by one or more of the following phenotypes compared to a plurality of γδ T cells expanded in the absence of a TLR agonist and in the presence of a γδTCR activator: (i) increased expression of cytotoxicity surface markers (e.g., granzyme B, CD107a, and/or CD86); (ii) increased expression of proinflammatory cytokines (e.g., IFN-γ, TNF-α, and/or IL-17A); (iii) decreased expression of immune checkpoint proteins (e.g., PD-1); (iv) increased cytotoxicity toward tumor cells; and/or (v) enhanced tumor volume reduction in vivo; (vi) enhanced PI3K-Akt-mTOR pathway; and/or (vii) ability to suppress inhibitory functions of adherent antigen presenting cells (APCs) present in the population of cells.

In certain embodiments, the plurality of γδ T cells is genetically modified. In certain embodiments, the plurality of γδ T cells is genetically modified to express a receptor selected from a chimeric antigen receptor (CAR), a TCR, a dominant negative receptor, a switch receptor, or any combination thereof.

In another aspect, the disclosure provides a pharmaceutical composition comprising the plurality of γδ T cells expanded and activated by any of the methods contemplated herein and a pharmaceutically-acceptable carrier.

### BRIEF DESCRIPTION OF THE DRAWINGS

The foregoing and other features and advantages of the present invention will be more fully understood from the following detailed description of illustrative embodiments taken in conjunction with the accompanying drawings.

FIGS. 1A-1G: Significant interindividual heterogeneity in γδ T cell expansion. FIG. 1A: Purity of expanded γδ T cells. 98 PBMC samples from 43 healthy donors were tested independently after 10 day expansion with ZOL (5 μM) and IL-2 (200 IU/ml). FIG. 1B: Purity of γδ T cells by different expansion methods. 32 PBMC samples from 13 healthy donors were tested independently after 10 day expansion by ZOL or anti-γδTCR Ab supplemented with IL-2. A paired t-test showed a significant difference between ZOL or anti-γδTCR Ab group, *p<0.05.

FIG. 1C: Cell number, expanded folds and viability of γδ T cells after expansion. 98 PBMC samples were tested independently after 10 day expansion with ZOL (5 μM). **p<0.01. FIG. 1D: Representative scatter plots of the time course of γδ T cells expansion. FIG. 1E: Time course of purity during γδ T cell expansion. n=5, **p<0.01. FIG. 1F: Time course of cell number during γδ T cell expansion. n=5, **p<0.01. FIG. 1G: Effect of age on γδ T cell expansion.

FIGS. 2A-2E: Interindividual γδ T cell immune phenotypic heterogeneity after expansion. FIG. 2A: Representative CD27/CD45RA contour plots of expanded γδ T cells. Effector memory (EM, CD45RA-CD27−), terminal-differentiated effector memory (TDEM, CD45RA+CD27−), naive (N, CD45RA+CD27+), and central memory (CM, CD45RA-CD27+) γδ T cells. FIG. 2B: Expanded γδ T cell phenotypic subset distribution. The data is presented as mean±SD. n=6, *p<0.05. FIG. 2C: Immune checkpoint protein expression in expanded γδ T cells. Flow cytometry analysis of PD-1, CTLA4, Eomes and T-bet expression by expanded γδ T cells at day 10. n=98, ns, no significant difference; *p<0.05; **p<0.01. FIG. 2D: Flow cytometry analysis of GranzB and IFN-7 expression by expanded γδ T cells at day 10. n=98, *p<0.05; **p<0.01. FIG. 2E: Flow cytometry analysis of CD80 and CD86 expression by expanded γδ T cells at day 10. n=98, ns, no significant difference; **p<0.01.

FIGS. 3A-3D: Interindividual functional heterogeneity of γδ T cells after expansion. FIG. 3A: γδ T cell migration. Expanded γδ T cells were tested in a transwell assay for their ability to migrate toward melanoma cells. n=6. *p<0.05; **p<0.01. FIG. 3B: Cytotoxic function of γδT cells. Expanded γδ T cells were incubated with different melanoma cell lines (A2058, A375 and UACC903). n=6. **p<0.01. FIG. 3C: Representative immunofluorescence images of tumor spheroid assay. Melanoma tumor spheroids were formed using A2058 or UACC903 cells and then incubated with expanded γδ T cells. The tumor cells were stained with CellTracker™ Orange CMRA Dye, and γδ T cells were stained with CFSE. More γδ T cells from good expanders were present in the center of the tumor spheroids and the tumor spheroids were smaller. Scale bar, 250 μm. FIG. 3D: Statistical analysis of size of tumor spheroids and number of infiltrated γδ T cells in spheroids. n=7. **p<0.01.

FIGS. 4A-4E: Initial Vδ2 T cell levels in PBMC predict their expansion capacity. FIG. 4A: Circulating γδ T cells. Initial percentage of Vδ2 T cells in 66 PBMC samples from 31 healthy donors was measured. FIG. 4B: Pearson correlation of the initial percentage of Vδ2 T cells in PBMC with purity of γδ T cells after expansion. n=66, **p<0.01. FIG. 4C: Initial γδ T cell proportion in good or poor expanders. n=66, **p<0.01. FIG. 4D: Representative scatter plots of the percentage of initial Vδ2 T cell and corresponding expanded Vδ2 T cells. FIG. 4E: Correlation of the initial Vδ2 T cell percentage and corresponding expanded Vδ2 T cells. Each line connects the results for an individual donor. *p<0.05.

FIGS. 5A-5E: γδ T cell intrinsic characters determine their expansion capacity. FIG. 5A: Representative contour plots of the circulating Vδ2 T cell phenotypic subsets. FIG. 5B: Circulating Vδ2 T cell phenotypic subset distribution. Effector memory (EM, CD45RA-CD27−), terminal-differentiated effector memory (TDEM, CD45RA+CD27−), naive (N, CD45RA+CD27+), and central memory (CM, CD45RA-CD27+) γδ T cells. The data is presented as mean±SD. n=7, *p<0.05. FIG. 5C: Immune checkpoint protein expression in circulating γδ T cells. Flow cytometry analysis of PD-1, CTLA4, Eomes and T-bet expression in the circulating γδT. n=66, *p<0.05; **p<0.01. FIG. 5D: GranzB and IFN-7 expression in the circulating γδ T cells. n=66, *p<0.05; **p<0.01. FIG. 5E: Proportion of Vδ2 T and Vδ1 T cells in circulation from 14 healthy individuals (D: Donor). Vδ1 T cells and the ratio of Vδ2/Vδ1 T cells were presented. ns, no significant difference; *p<0.05.

FIGS. 6A-6E: γδ T cell expansion capacity is independent of expansion methods. FIG. 6A: Representative scatter plots of γδ T cells expanded by ZOL or anti-γδTCR antibody. FIG. 6B: Purity of γδ T cells from good and poor expanders expanded by ZOL or anti-γδTCR antibody. n=6, *p<0.05. FIG. 6C: Immune checkpoint protein and Granzyme B expression in γδ T cells expanded by ZOL or anti-γδTCR antibody. PD-1, CTLA-4 and GranzB expression in γδ T cells from good and poor expanders expanded by ZOL or anti-γδTCR antibody for day 10. n=6, *p<0.05, **p<0.01. FIG. 6D: Melanoma spheroid assays with γδT expanded by ZOL or anti-γδ TCR antibody. Melanoma tumor spheroids were formed using UACC903 cells and then incubated with expanded γδ T cells using ZOL or anti-γδTCR antibody, respectively. More γδT cells from good expanders were present in the center of the tumor spheroids and the tumor spheroids were smaller. Melanoma sphere size and number of infiltrated γδ T cells in spheroids was analyzed. n=6. *p<0.05. FIG. 6E: Cytotoxicity of expanded γδ T cells using ZOL or anti-γδ TCR antibody against A2058 cells and UACC903 cells. n=6, **p<0.01.

FIGS. 7A-7G: γδ T cell prediction models and verification in patient derived melanoma organoids. FIG. 7A: Logistic regression analysis of proportion of circulating Vδ2 cells in individuals is to determine the optimal cut-off value of 0.82% for γδ T cell expansion with AUC=0.968. FIG. 7B: A plot showed that the cut-off point of 0.82% had significant difference with sensitivity of 97.8% and specificity of 90.5%. FIG. 7C: Vδ2 Index Score. The score was calculated in the based on Vδ2 cell concentration in the PBMC. *p<0.05. FIG. 7D: Representative live/dead staining image of organoids co-cultured with expanded γδ T cells from donors whose Vδ2 Index Score≥0 or <0. Scale bar, 100 μm. FIG. 7E: Cytotoxicity of expanded γδ T cells co-cultured with organoids and the expanded γδ T cells from donors whose Vδ2 Index Score ≥0 or <0. **p<0.01. FIG. 7F: Representative flow charts show apoptotic cells after coculturing organoids with expanded γδ T cells. FIG. 7G: Statistical analysis of the apoptotic cells (contained early apoptotic and late apoptotic) in organoids after culturing with γδ T cells. n=5, **p<0.01.

FIGS. 8A-8D: Repeated measure of γδT expansion capacity over time. FIG. 8A: PBMC samples from 17 healthy donors were collected and tested over a 2-year period. 3-4 samples were collected from each donor, respectively. γδ T cells were expanded using ZOL (5 μM) for 10 days. FIG. 8B: Pearson correlation of the percentage of ZOL expanded γδ T cells with the proportion of anti-γδTCR ab expanded γδ T cells. **p<0.01. FIG. 8C: The ratio of Vδ2/Vδ1 T cells after ZOL or anti-γδTCR Ab expansion. **p<0.01. FIG. 8D: Effect of gender on γδ T cell expansion. Fisher's exact test shows that gender does not affect γδ T cell expansion.

FIGS. 9A-9B: Gating strategy and representative scatter plots. FIG. 9A: Gating strategy for γδ T cells. FIG. 8B: Representative plots for PD-1, CTLA4, Eomes, T-bet, GranzB, IFN-7, CD80 and CD86.

FIG. 10: Marker expression in circulating γδ T cells. Flow cytometry analysis of CD80, CD86, CD69, Ki67, NKG2D, CD107a and CD277 expression in circulating γδ T cells. n=13-98, ns, no significant difference; *p<0.05; **p<0.01.

FIGS. 11A-11D: Gating strategy and other immune cells in circulation. FIG. 11A: Flow gating strategy for CD3, total γδT, Vδ1, Vδ2, CD4, CD14, CD11b and CD19. FIG. 11B: Flow cytometry analysis of CD3+, CD4+, CD14+, CD11b+ and CD19+ cells in the circulation in good expanders and poor expanders. ns, no significant difference; *p<0.05. FIG. 11C: Ratio of Vδ2/CD3, Vδ2/CD4, Vδ2/CD14, Vδ2/CD11b and Vδ2/CD19 in good expanders and poor expanders. *p<0.05; **p<0.01. FIG. 11D: Proportion of other immune cells in circulation. The proportion of CD3+, CD14+, CD19+, Vδ2 T, Vδ1 T cells and other cells in PBMC was presented.

FIG. 12: Melanoma spheroid assays with γδT expanded by ZOL or anti-γδTCR antibody. Melanoma tumor spheroids were formed using UACC903 cells and then incubated with expanded γδ T cells using ZOL or anti-γδTCR antibody, respectively. The tumor cells were stained with CellTracker™ Orange CMRA Dye, and γδ T cells were stained with CFSE. n=6. Scale bar, 250 μm.

FIG. 13: Effect of anti-PD1 and anti-CTLA4 antibody on γδT expansion. γδ T cells from poor expanders were collected and incubated with ZOL for 10 days with anti-PD1 or anti-CTLA4 antibody. Flow cytometry analysis of γδ T cell proportion, GranzB, CD107a and IFN-7 in expanded γδ T cells at day 10. n=2. No significant changes were found.

FIGS. 14A-14C: Circulating γδ T cells is a potential predictor for γδ T cell functions. FIG. 14A: Multivariate Logistic regression analysis is to determine the optimal cut-off value of 0.678 of the model fitted response for γδ T cell expansion with AUC=0.989. FIG. 14B: Vδ2 Index Score. The score was calculated in the based on comprehensive Vδ2 Index in PBMC. FIG. 14C: Tracking the basal γδ T cells and expanded γδ T cells may help predict γδ T cell function and determine whether the cell product will be successful or failure for the allogeneic adoptive γδT cell therapy.

FIGS. 15A-15E illustrate the finding that γδTCR and TLR costimulation promotes Vδ2 T cell expansion. FIG. 15A is a chart providing concentrations of human Vδ2 T cells in cultured PBMCs at day 5 or 7. Two million PBMCs were added in each well. 5 μg/ml IPP and different TLR agonists were added in each well. Concentrations of Vδ2 T cells were measured by FACS. n=5. FIG. 15B and FIG. 15C provide representative flow charts and statistical analysis, respectively, illustrating concentrations of human Vδ2 T cells in cultured PBMCs at day 7 after costimulation with IPP and different TLR7/8 agonists. Different concentrations of TLR7/8 agonists were used and n=5. Data are presented as the mean±S.E.M, *p<0.05, **p<0.01, ***p<0.001, NS means no significant difference. FIG. 15D provides flow cytometry data for purified naïve γδ T cells. Purified naïve γδ T cells from PBMCs were cultured with indicated stimulation for 4 and 6 days. 100K purified naïve γδT were labeled with CFSE and added to each well. Rapid dividing cells (daughter cells) are determined to be cells showing a decrease in mean fluorescence intensity (MFI) compared to the initial CFSE staining level. Flow was gated on fast dividing cells (CFSE diluted daughter cells). FIG. 15E provides brightfield photos of cultured purified naïve γδ T cells at day 4. 100 K purified naïve γδ T cells were added to each well and then cultured with indicated compounds. Scale bar indicates 1 mm.

FIGS. 16A-16F illustrate the finding that resiquimod promotes IPP induced Vδ2 T cell expansion in vitro. FIG. 16A is a chart providing concentrations of human Vδ2 T cells in cultured PBMCs at day 8. Two million PBMCs were added in each well. Different doses of IPP were added with resiquimod (10 μg/ml). The cells were expanded for 8 days. Vδ2 T cell concentration was measured by FACS. n=3. FIG. 16B provides flow cytometry data for stimulated PBMCs. Overlaid of IPP and IPP plus resiquimod stimulated PBMCs at day 8. PBMCs were labeled with CFSE dye and cultured with IPP or IPP plus resiquimod for 8 days. Representative flow charts gated on TCR Vδ2 positive (+) and negative (−) populations. FIG. 16C provides flow cytometry data illustrating that NK cells are a minority of cells after Vδ2 T cells expansion. 2 million PBMCs were cultured for 8 days with indicated stimulation. NK cell concentration was measured by FACS. FIG. 16D is a chart providing concentrations of Vδ2 T cells in cultured PBMCs expanded by IPP plus resiquimod for 8 days using full RPMI 1640 medium with or without 2-Me. N=3. Data are presented as mean S.E.M., *p<0.05, NS means no significant difference. FIGS. 16E-16F illustrate initial Vδ2 T cell levels in PBMCs predict their expansion capacity induced by IPP plus Resiquimod. FIG. 16E depicts initial Vδ2 T cell levels in PBMC of two donors. FIG. 16F illustrates the corresponding Vδ2 T cell absolute numbers expanded with IPP plus Resiquimod on day 7 of the two donors in FIG. 16E.

FIG. 17A provides representative flow cytometry data illustrating TLR agonists alone have a limited modest effect on purified Vδ2 T cell expansion. 100K purified γδ T cells were labeled with CFSE and cultured with indicted TLR agonists for 3 days. FIG. 17B provides representative flow cytometry data illustrating TLR7 and TLR8 expression in monocytes and γδ T cells freshly isolated from PBMCs.

FIGS. 18A-18E illustrate the finding that resiquimod promotes pan-anti-γδTCR antibodies induced Vδ2 T cell expansion. FIG. 18A and FIG. 18B provide typical flow charts and statistical analysis, respectively, showing concentrations of Vδ2 cells in cultured PBMCs at day 8 stimulated by pan-anti-γδTCR antibodies (5 μg/per well) with or without resiquimod (10 μg/ml), n=13. FIG. 18C provides statistical analysis showing absolute number of Vδ2 T cells expanded by pan-anti-γδTCR antibodies with or without resiquimod (10 μg/ml), n=3. FIG. 18D provides statistical analysis showing concentrations of Vδ2 T cells expanded from PBMCs by indicated stimulation on day 6, 8, 10 and 13. PBMCs were from the same donor. FIG. 18E provides brightfield photos showing the colony formation of γδ T cells in cultured PBMCs with pan-anti-γδTCR antibodies plus resiquimod on day 3. Data are presented as the mean±S.E.M. *p<0.05, ***p<0.001.

FIG. 19A and FIG. 19B provide typical flow charts and statistical analysis, respectively, showing concentrations of Vδ2 T cells in cultured PBMCs at day 10 after incubation with IPP and/or resiquimod. IPP or resiquimod was added at different time points. PBMCs were collected at day 0; IPP (days)+resiquimod: PBMCs were stimulated with IPP (5 μg/ml) starting on day 0 and resiquimod (10 μg/ml) was added on day 5 and cultured for another 5 days. Resiquimod (day 5)+IPP: PBMCs were stimulated with resiquimod starting on day 0 and IPP was added on day 5 and cultured for another 5 days. n=3. Data are presented as mean±S.E.M., *p<0.05, NS means no significant difference. FIG. 19C provides statistical analysis showing concentrations of Vδ2 T cells in cultured PBMCs. Resiquimod was added into cultured PBMCs at early stage (day 1-5) or late stage (day 6-12), and the concentrations of Vδ2 T cells were measured at day 13. Data are presented as the mean S.E.M. *p<0.05, ***p<0.001.

FIGS. 20A-20J illustrate the finding that TLR7/8 activation regulates functions of expanded Vδ2 T cells. FIGS. 20A-20C show PD-1 (FIG. 20A), CTLA-4 (FIG. 20B), and CD86 (FIG. 20C) expression in Vδ2 T cells. PBMCs from different donors were used in the experiments and each paired dot represent one donor. Two million PBMCs were added to each well and cultured with IPP or IPP plus resiquimod for 8 days. Protein expression on Vδ2 T cells was measured by FACS. FIGS. 20D-20F show IFN-7 (FIG. 20D), TNF-α (FIG. 20E), and IL-17A (FIG. 20F) concentrations in the medium. PBMCs were cultured with IPP or IPP plus resiquimod for 8 days. Cytokine levels in the culture supernatant were measured by ELISA.

FIGS. 20G-20I provide cytotoxicity associated marker expression on Vδ2 T cells. PBMCs were cultured with IPP or IPP plus resiquimod for 8 days. Granzyme B (FIG. 20G), CD107a (FIG. 20H), Fas-ligand (FasL) (FIG. 20I), NKG2D (FIG. 20J) expression in Vδ2 T cells was measured by FACS. *p<0.05, **p<0.01, ***p<0.001.

FIG. 21 illustrates the finding that resiquimod promotes cytotoxicity of γδ T cells expanded by pan-anti-γδTCR antibodies in different E:T ratio. LDH release assay result showing cytotoxicity of γδ T cells expanded by pan-anti-γδTCR antibody without or with resiquimod (10 μg/ml) to melanoma A375 cells in different E:T ratios. n=3, data shown as Mean±S.E.M., **p<0.01, ***p<0.001.

FIGS. 22A-22B illustrate the finding that resiquimod promotes cytotoxicity of γδ T cells expanded by pan-anti-γδTCR antibodies to a variety of tumor cells. FIG. 22A provides LDH release assay result showing cytotoxicity of γδ T cells expanded by pan-anti-γδTCR antibody (5 g per well) with or without resiquimod (10 μg/ml) at E:T ratio=5:1 to multiple melanoma cell lines. A2058CR and WM9CR melanoma cell lines are resistant to BRAF and MEK inhibitors. A2058 and WM9 are their parental cell lines. LDH release assay was performed after coculture Vδ2 T cells and tumor cells for 16 hours. n=5-7, data shown as Mean±S.E.M. FIG. 22B provides LDH release assay result showing cytotoxicity of γδ T cells expanded by pan-anti-γδTCR antibody without or with resiquimod (10 μg/ml) at E:T ratio=5:1 to epithelial cancer cell lines, n=5-6, data shown as Mean±S.E.M. **P<0.01, ***P<0.001.

FIGS. 23A-23G illustrate the finding that TLR7/8 activation promotes cytotoxicity of Vδ2 T cells. FIG. 23A shows cytotoxicity of expanded Vδ2 T cells to melanoma cells in 2D culture. Vδ2 T cells were expanded from PBMCs using IPP or IPP plus resiquimod for approximately 13 days. 2×10⁴ A375 and A2058 melanoma cells were used in each well. Effector:Target (E:T) ratio was 5:1 in the coculture. LDH release assay was performed after coculturing Vδ2 T cells and melanoma cells for 16 hours. FIG. 23B shows cytotoxicity of expanded Vδ2 T cells to melanoma cells in 3D culture. A2058 melanoma spheroids were coculturing with Vδ2 T cells expanded using IPP or IPP plus resiquimod for 24 hours. CFSE stained viable tumor cells, while PI stained dead cells. Representative confocal immunofluorescence images show cell death after treatment. Scale bar 1 mm. FIGS. 23C-23D provide representative flow charts after coculturing for 24 hours (FIG. 23C) and statistical analysis of early and late apoptosis cells (FIG. 23D) illustrating apoptosis and cell death in A2058 melanoma spheroids after coculturing with Vδ2 T cells. Each paired dots represent one healthy donor. Data are shown as Mean±S.E.M. FIG. 23E provides LDH release assay result showing cell death in A2058 melanoma spheroids after coculturing with Vδ2 T cells. Data are shown as Mean±S.E.M. FIG. 23F is the treatment schema of A375 melanoma xenograft model treated with Vδ2 T cells. 5×10⁶ melanoma cells were injected into the flanks of nude mice (n=6). Vδ2 T cells from 3 different donors were expanded from PBMCs using IPP or IPP plus resiquimod for 13 days. The last donor was a good expander and Vδ2 T cells from the last donor were used to treat all the mice twice. Expanded Vδ2 T cells from a donor were injected into all the mice to avoid donor-related bias. FIG. 23G provides data for melanoma tumor volume after treatment. Tumor volume was measured at least twice a week. All data shown as Mean±S.E.M. *p<0.05, **p<0.01, ***p<0.001.

FIGS. 24A-24C provide single-cell transcriptomics data which identifies distinct signaling pathway profiling during Vδ2 T cell fates. FIG. 24A provides a heat map showing the marker gene expression hits in relatively naive Vδ2 T cells and terminally differentiated effector memory Vδ2 T cells. FIG. 24B provides gene-ontology analysis of significantly different hits of phosphoprotein related to FIG. 25D. FIG. 24C provides UMAP and violin plots showing ssGSEA score of signaling pathway related genes of FIG. 25E.

FIGS. 25A-25N illustrate the finding that TLR7/8 agonist activates MyD88-dependent and PI3K-Akt-mTOR signaling pathways in Vδ2 T cells. FIG. 25A provides two-dimensional uniform manifold approximation and projection (UMAP) panel showing the cluster analysis of Vδ2 T cells with relative expression of 7 upregulated genes and 5 downregulated genes identified as unique to γδ T cells activation and differentiation. scRNA-seq data from three healthy donors were analyzed. FIG. 25B provides a heat map showing marker gene expression hits in naive Vδ2 T cells and terminally differentiated effector memory Vδ2 T cells. FIG. 25C provides UMAP sets showing clusters of selected naive Vδ2 T cells and terminally differentiated effector memory Vδ2 T cells for further analysis. FIG. 25D shows functional categories of significantly different hits between selected naive Vδ2 T cells and terminally differentiated effector memory Vδ2 T cells. FIG. 25E provides KEGG pathway analysis of phosphoprotein of significantly different hits shown in FIG. 25D. FIG. 25F is a heat map showing the single sample gene set enrichment analysis (ssGSEA) of single cell selected. FIG. 25G provides UMAP showing ssGSEA score of PI3K-Akt signaling pathway related genes. FIG. 25H provides violin plots showing the ssGSEA score of PI3K-Akt signaling pathway related genes. FIG. 25I is a heat map of cluster analysis of significantly different protein hits of RPPA assay analysis. FIG. 25J provides KEGG-pathway analysis of significantly different protein hits of RPPA assay analysis. FIG. 25K provides flow cytometry data illustrating inhibition of Vδ2 T cell expansion from PBMCs by mTOR inhibitors. PBMC was incubated with IPP or IPP plus resiquimod as well as mTOR inhibitors, rapamycin (Rapa) or torin1 (Torin). Representative flow cytometry results showing the concentrations of Vδ2 T cells. FIG. 25L provides statistical analysis of flow cytometry data shown in FIG. 25K, data shown as Mean±S.E.M. FIG. 25M provides flow cytometry data illustrating inhibition of Vδ2 T cell expansion from PBMCs by a MyD88 inhibitor ST2825. PBMCs were incubated with IPP or IPP plus resiquimod as well as ST2825 with indicated concentrations. Representative flow cytometry results showing the concentration of Vδ2 T cells. FIG. 25N provides statistical analysis of flow cytometry data shown in FIG. 25M, data are shown as Mean±S.E.M. *p<0.05, **p<0.01, ***p<0.001, NS means no significant difference.

FIG. 26 provides gene-ontology analysis of significantly different protein hits of RPPA analysis.

FIGS. 27A-27I provide data illustrating the advantage of Vδ2 T cell expansion by IPP plus resiquimod over ZOL. FIGS. 27A-27C show Vδ2 T cell expansion using IPP plus resiquimod (IR) or ZOL. Two million PBMCs were added in each well with indicated stimulation for 10 days. Representative flow charts (FIG. 27A) and statistical analysis results showing Vδ2 T cell concentration (FIG. 27B) and absolute number (FIG. 27C, independent experiments) of Vδ2 T cells expanded by IPP plus resiquimod or ZOL from PBMCs on day 10, n=4. Data are shown as the mean±S.E.M. p-value is labeled and NS means no significant difference. FIG. 27D shows functional marker expression on Vδ2 T cells expanded by IPP plus resiquimod or ZOL from PBMCs on day 8 and statistical analysis. Data are shown as the mean S.E.M. n≥7. FIG. 27E provides LDH assay results showing the cytotoxicity of Vδ2 T cells expanded and sorted by IPP plus resiquimod or ZOL from PBMCs to melanoma cells A375 in vitro using different E:T ratios, n=3, Data are presented as the mean S.E.M. FIGS. 27F-27G provide representative flow charts (FIG. 27F) and statistical analysis (FIG. 27G) results showing resiquimod does not promote ZOL stimulation of Vδ2 T cell proliferation from PBMCs in low concentration and inhibits its function in higher concentrations. Data are presented as the mean S.E.M. FIGS. 27H-27I provide CFSE proliferation assay data of purified naïve γδ T cells. Purified naïve γδ T cells were labeled with CFSE and stimulated with IPP plus resiquimod or ZOL for 3 days. PBMCs from the same-donor were not labeled with CFSE and used in the ZOL+PBMCs group. Representative flow charts are shown in FIG. 27H and statistical analysis in FIG. 27I. Data are presented as mean S.E.M. *p<0.05, **p<0.01, ***p<0.001, NS means no significant difference.

FIGS. 28A-28D provide data illustrating Vδ2 T cell expansion by IPP plus resiquimod vs ZOL. FIG. 28A provides independent experiment results showing expansion folds of Vδ2 T cells from PBMCs on day 8 stimulated by IPP plus resiquimod (10 μg/ml) or ZOL (5 μM), n=4. Data are presented as the mean±S.E.M. FIG. 28B illustrates cytotoxicity of expanded Vδ2 T cells to lymphoma cells. Vδ2 T cells were expanded from PBMCs using ZOL or IPP plus resiquimod for approximately 13 days. 2×10⁴ Daudi or K562 cells were added in each well. Effector:Target (E:T) ratio was 5:1, 2:1 and 1:1 in the coculture. LDH release assay was performed after coculturing Vδ2 T cells and lymphoma cells for 4 hours. Data shown as Mean±S.E.M. **p<0.01, ***p<0.001. FIG. 28C provides concentrations and absolute number of human Vδ2 T cells in cultured PBMCs at day 7 after co-stimulation with ZOL and Resiquimod. Data are presented as the mean S.E.M, ***p<0.001. FIG. 28D provides gating strategy of flow cytometry result of FIG. 28C group “ZOL+PBMCs” on day 3, purified naïve γδ T cells were labeled with CFSE, while PBMCs were not labeled.

FIGS. 29A-29G illustrate the finding that TLR7/8 activation suppresses formation and inhibitory function of adherent APCs. FIG. 29A provides representative bright-field images (phase-contrast) of adherent cells from PBMCs cultured with IPP or IPP plus resiquimod on day 3 showing adherent cells during Vδ2 T expansion from PBMCs. Scale bar, 100 μm. FIG. 29B provides representative flow charts to characterize adherent cells after culturing PBMCs with IPP for 3 days. Adherent CD14-CD80+ APC cells induced by IPP express both PD-L1 and CTLA-4. The adherent cells are mostly CD14−. A majority of these adherent cells are CD14-CD80+ APCs. FIG. 29C provides representative flow charts and FIG. 29D provides statistical analysis, respectively, illustrating inhibitory function of adherent APCs. Adherent APCs were induced by culturing PBMCs for 3 day. The adherent APCs were collected and then added to PBMCs that have been stimulated with IPP and IPP plus resiquimod for 3 days, concentrations of Vδ2 T cells were measured by FACS 3 days later. In a separate group (IPP day 0+resiquimod day 3), both adherent cells and resiquimod were added to PBMCs that have been stimulated with IPP for 3 days. Data are shown as Mean±S.E.M. n=5. (E-G) Flow cytometry and statistical analysis showing PD-L1 and CTLA-4 expression on CD80+CD86+ cells in cultured PBMCs with indicated stimulations. FIG. 29E provides representative flow charts of PD-L1 and CTLA-4 expression from a single donor. FIG. 29F and FIG. 29G provide aggregated PD-L1 and CTLA-4 data, respectively, from different donors. n=3, Data are shown as Mean±S.E.M. *p<0.05, **p<0.01, NS, no statistical significance.

FIGS. 30A-30C provide flow cytometry data. FIG. 30A shows APC phenotypes and checkpoint protein expression in PBMC monocytes. Four populations were shown based on the expression levels of CD14 (Monocyte's marker) and CD80 (APC marker), Monocyte-divided APCs showing higher forward and side scatter values, and checkpoint protein (PD-L1 and CTLA-4) expression in health donor PBMCs. FIG. 30B provides data for PBMCs cultured with IPP or IPP plus resiquimod for 48 h, and the viability and mortality of monocytes and lymphocytes were detected with Annexin V/7-AAD apoptosis kit and flow cytometry. FIG. 30C provides data for purified monocytes cultured with resiquimod for 24 h with indicated concentration, and the viability and mortality were detected with Annexin V/7-AAD apoptosis kit and flow cytometry.

FIGS. 31A-31B illustrate Vδ2 T cell expansion using IPP or IPP plus resiquimod (IR) in the presence of anti-PD-1 antibody (pembrolizumab, 4 μg/ml) for 10 days. FIG. 31A provides results showing Vδ2 T cell concentration and absolute number of Vδ2 T cells. Two million PBMCs were added to each well with indicated stimulation for 10 days. Data are shown as the mean±S.E.M, 3 replicates. FIG. 31B provides data showing functional marker expression on Vδ2 T cells.

FIGS. 32A-32C provide data for human γδ T cells in Hu-Mice. FIG. 32A provides flow cytometry results showing human T cell population (using anti-human-CD3-antibodies) and few Vδ2 T cells in Hu-Mice blood. Hu-Mice blood was collected and analyzed by FACS or cultured with IPP plus resiquimod or ZOL. Vδ2 T cells increased significantly after expansion for 10 days. FIG. 32B shows absolute number of Vδ2 T cells expanded by IPP plus resiquimod or ZOL, results shown as Mean±S.E.M. n=4. FIG. 32C provides flow cytometry results showing the percentage of human Vδ2 T cells in Hu-Mice spleen. Portion of mouse spleen was digested and cultured with IPP plus resiquimod or ZOL. Functional marker expression levels of Hu-Mice spleen Vδ2 T lymphocytes. I+R: IPP plus resiquimod. ***p<0.001, NS, no significance.

FIGS. 33A-33C provide schematic diagrams for various anti-γδTCR antibody formats. FIG. 33A illustrates γδTCR recognition of soluble anti-γδTCR antibodies. FIG. 33B illustrates γδTCR recognition of surface-coated anti-γδTCR antibodies. FIG. 33C illustrates γδTCR recognition of anti-γδTCR antibodies attached to a bead (referred to herein as GDBeads).

FIG. 34 provides a silver-stained SDS-PAGE gel of antibody-coated magnetic beads. Prepare eluents of antibody-coated magnetic beads were loaded with BSA standard with indicated amount and equivalent commercial magnetic beads.

FIG. 35 is a series of bright-field images (phase-contrast) of PBMCs cultured with indicated antibody-coated GDBeads on day 1, 2, 3, as well on day 5 after removal of the GDBeads. Scale bar, 1 mm.

FIG. 36 provides photographs of test tubes containing a mixture of PBMCs and GDBeads at 1:1 ratio cultured for 3 days (left), and PBMCs (center) and GDBeads (right) separated by magnetic field.

FIGS. 37A-37B show flow cytometry data for γδ T cell expansion by soluble anti-γδTCR antibodies and by GDBeads. Representative flow charts (FIG. 37A) and statistical analysis results showing Vδ1 T cells and Vδ2 T cell concentration (FIG. 37B) expanded by soluble antibody and by GDBeads from PBMCs on day 8, n=4. Data are shown as the mean±S.E.M., **P<0.01.

FIGS. 38A-38B show statistical analysis of flow cytometry data showing the purity of human γδ T cells on day 8 of culture with the indicated soluble antibodies (FIG. 38A) or GDBeads coated with the indicated antibodies (FIG. 38B). GDBeads stimulate γδ T cell expansion from PBMCs. Data are presented as the mean±S.E.M., Student's t test, *P<0.05, **P<0.01, ***P<0.001.

FIGS. 39A-39C show statistical analysis of flow cytometry data showing the purity of human γδ T cells on day 8 of culture with GDBeads coated with the indicated antibodies. GDBeads stimulate both Vδ1 (FIG. 39A) and Vδ2 (FIG. 39B) of two main subpopulations of γδ T cells expansion from PBMCs. FIG. 39C shows the purity and ratio of Vδ1 and Vδ2 T cells of human γδ T cells on day 8. Data are presented as the mean S.E.M., Student's t test, *P<0.05, **P<0.01, ***P<0.001.

FIGS. 40A-40E show statistical analysis of flow cytometry data showing expression of various markers on Vδ1 and Vδ2 T cells expanded by indicated GDBeads for 8 days from PBMCs, valued by Student's t test (*P<0.01, **P<0.01, ***P<0.001). FIG. 40A shows the percentage of cells expressing granzyme B. FIG. 40B shows the percentage of cells expressing IFN-7. FIG. 40C shows the percentage of cells expressing perforin. FIG. 40D shows the percentage of cells expressing TNF-α. FIG. 40E shows the percentage of cells expressing IL-17A.

FIGS. 41A-41E show statistical analysis of flow cytometry data showing expression of various markers on Vδ1 and Vδ2 T cells expanded by indicated GDBeads for 8 days from PBMCs, valued by Student's t test (*P<0.01, **P<0.01, ***P<0.001). FIG. 41A shows the percentage of cells expressing PD-1. FIG. 41B shows the percentage of cells expressing CTLA-4. FIG. 41C shows the percentage of cells expressing Tim3. FIG. 41D shows the percentage of cells expressing LAG-3. FIG. 41E shows the percentage of cells expressing T-Bet (in PD1+ cells).

FIG. 42 is a graph showing co-stimulatory receptor antibodies promote cytotoxicity of γδ T cells expanded by GDBeads targeting A375 melanoma cells at an Effector:Target=5:1 ratio. Both anti-TCRγδ antibodies and the indicated co-stimulatory receptor antibodies were coated on the GDBeads. Data are presented as the mean±S.E.M.

FIG. 43 is a graph showing co-stimulatory receptor antibodies promote cytotoxicity of γδ T cells expanded by GDBeads targeting A375 melanoma cells in different Effector:Target (E:T) ratios as indicated. Both anti-TCRγδ antibodies and the indicated co-stimulatory receptor antibodies were coated on the GDBeads. Data are presented as the mean±S.E.M.

FIGS. 44A-44D illustrate that adding anti-PD1 antibody during pan-anti-human-TCRγδ antibody induced γδ T cell expansion improves cellular product function. FIG. 44A is a graph of flow cytometry data showing the purity of human γδ T cells on day 13 of culture with GDBeads coated with pan-anti-human-TCRγδ antibody (TCRγδ) or pan-anti-human-TCRγδ antibody and anti-human-PD-1 antibody (TCRγδ+Anti-PD1). FIG. 44B shows the viability of expended γδT cells with GDBeads coated with indicated antibodies. FIG. 44C is a panel of flow cytometry data illustrating GranzymeB, Perforin, IFN-7 and TNF-α expression in γδ T cells expanded by GDBeads coated with indicated antibodies. FIG. 44D illustrates cytotoxicity of expanded γδT cells to A375 melanoma cells. γδ T cells were expanded from PBMC using GDBeads coated with indicated antibodies for approximately 13 days. 2×10⁴ A375 cells (Firefly Luciferase was stable transduced) were added in each well. Effector:Tarege (E:T) ratio was 5:1 and 2.5:1 in the coculture. Luciferase assay was performed after coculturing γδ T cells and A375 melanoma cells for 20 hours. Data shown as Mean±S.E.M. **p<0.01.

### DETAILED DESCRIPTION

Personalized adoptive T cell transfer (ACT) therapies with autologous tumor infiltrating lymphocytes (TIL), T cell receptor (TCR) T cells or chimeric antigen receptor (CAR) T cell have provided major breakthroughs in the treatment of a number of cancers including leukemia, lymphoma, and melanoma (June, et al., 2018, Science 359: 1361-1365; Rosenberg, et al., 2015, Science 348: 62-68). Current ACT research and clinical applications have focused primarily on alpha beta (αμ) T cells, which are human leukocyte antigen (HLA)-restricted in tumor cell recognition (Kalos, et al., 2013, Immunity 39: 49-60). During cellular immunotherapy, tumor cells may down-regulate or lose HLA class I or β2 microglobulin which allows tumor cells to evade detection from αβT cells, resulting in immune escape and treatment failure (Khong, et al., 2002, Nature immunology 3: 999-1005).

Gamma delta (γδ) T cells are an unconventional subset of T cells expressing heterodimeric TCRs composed of γ and δ chains, and they are HLA-unrestricted in tumor cell recognition (Kabelitz, et al., 2020, Cellular & molecular immunology 17: 925-939). The recognition of cancer antigens, such as phospho-antigens (pAgs), relies on the engagement of T-cell receptor (TCR)-γδ. Thus, tumors may still be targets for γδ T cells even if they are not efficiently recognized by αβ T cells. High circulating levels of γδ T cells have been associated with improved 5-year disease-free and overall survival after bone marrow transplantation in patients with acute leukemia (Godder, et al., 2007, Bone marrow transplantation 39: 751-757). In humans, two major subsets of γδ T cells have been identified by the variable (V) gene of γδTCR, which are Vδ1 T cells, the predominant population in peripheral tissues and intestine mucosa, and Vδ2 T cells, which represent a majority of γδ T cells in peripheral blood and co-express Vγ9 chain of TCR. VT9Vδ2 T cells (Vδ2 T cells) are unique in primates but they only account for less than 5% of total T cells (Vantourout, et al., 2013, Nat Rev Immunol 13: 88-100). Most of the γδ T cell clinical trials to date have focused on the Vδ2 T cell subset, given their relative abundance in peripheral blood (Hoeres, et al., Front Immunol 9: 800).

It is to be understood that the methods described in this disclosure are not limited to particular methods and experimental conditions disclosed herein as such methods and conditions may vary. It is also to be understood that the terminology used herein is for the purpose of describing particular embodiments only, and is not intended to be limiting.

Furthermore, the experiments described herein, unless otherwise indicated, use conventional molecular and cellular biological and immunological techniques within the skill of the art. Such techniques are well known to the skilled worker, and are explained fully in the literature. See, e.g., Ausubel, et al., ed., Current Protocols in Molecular Biology, John Wiley & Sons, Inc., NY, N.Y. (1987-2008), including all supplements, Molecular Cloning: A Laboratory Manual (Fourth Edition) by MR Green and J. Sambrook and Harlow et al., Antibodies: A Laboratory Manual, Chapter 14, Cold Spring Harbor Laboratory, Cold Spring Harbor (2013, 2nd edition).

Methods and techniques using T Cells with chimeric antigen receptors (CAR T cells) are described in e.g., Ruella, et al., J. Clin. Invest., 126(10):3814-3826 (2016) and Kalos, et al., 3 (95), 95ra73:1-11 (2011), the contents of which are hereby incorporated by reference in their entireties.

### A. Definitions

Unless otherwise defined, scientific and technical terms used herein have the meanings that are commonly understood by those of ordinary skill in the art. In the event of any latent ambiguity, definitions provided herein take precedent over any dictionary or extrinsic definition. Unless otherwise required by context, singular terms shall include pluralities and plural terms shall include the singular. The use of “or” means “and/or” unless stated otherwise. The use of the term “including,” as well as other forms, such as “includes” and “included,” is not limiting.

Generally, nomenclature used in connection with cell and tissue culture, molecular biology, immunology, microbiology, genetics and protein and nucleic acid chemistry and hybridization described herein is well-known and commonly used in the art. The methods and techniques provided herein are generally performed according to conventional methods well known in the art and as described in various general and more specific references that are cited and discussed throughout the present specification unless otherwise indicated. Enzymatic reactions and purification techniques are performed according to manufacturer's specifications, as commonly accomplished in the art or as described herein. The nomenclatures used in connection with, and the laboratory procedures and techniques of, analytical chemistry, synthetic organic chemistry, and medicinal and pharmaceutical chemistry described herein are those well-known and commonly used in the art. Standard techniques are used for chemical syntheses, chemical analyses, pharmaceutical preparation, formulation, and delivery, and treatment of patients.

That the disclosure may be more readily understood, select terms are defined below.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of 20% or ±10%, more preferably ±5%, even more preferably +1%, and still more preferably +0.1% from the specified value, as such variations are appropriate to perform the disclosed methods.

“Activation,” as used herein, refers to the state of a T cell that has been sufficiently stimulated to induce detectable cellular proliferation. Activation can also be associated with induced cytokine production, and detectable effector functions. The term “activated T cells” refers to, among other things, T cells that are undergoing cell division.

As used herein, to “alleviate” a disease means reducing the severity of one or more symptoms of the disease.

“Allogeneic” refers to a graft derived from a different animal of the same species.

“Xenogeneic” refers to a graft derived from an animal of a different species.

The term “antigen” as used herein is defined as a molecule that provokes an immune response. This immune response may involve either antibody production, or the activation of specific immunologically-competent cells, or both. The skilled artisan will understand that any macromolecule, including virtually all proteins or peptides, can serve as an antigen.

Furthermore, antigens can be derived from recombinant or genomic DNA. A skilled artisan will understand that any DNA, which comprises a nucleotide sequences or a partial nucleotide sequence encoding a protein that elicits an immune response therefore encodes an “antigen” as that term is used herein. Furthermore, one skilled in the art will understand that an antigen need not be encoded solely by a full length nucleotide sequence of a gene. It is readily apparent that the present invention includes, but is not limited to, the use of partial nucleotide sequences of more than one gene and that these nucleotide sequences are arranged in various combinations to elicit the desired immune response. Moreover, a skilled artisan will understand that an antigen need not be encoded by a “gene” at all. It is readily apparent that an antigen can be generated synthesized or can be derived from a biological sample. Such a biological sample can include, but is not limited to a tissue sample, a tumor sample, a cell or a biological fluid.

As used herein, the term “autologous” is meant to refer to any material derived from the same individual to which it is later to be re-introduced into the individual.

A “co-stimulatory molecule” refers to the cognate binding partner on a T cell that specifically binds with a co-stimulatory ligand, thereby mediating a co-stimulatory response by the T cell, such as, but not limited to, proliferation. Co-stimulatory molecules include, but are not limited to an MHC class I molecule, BTLA and a Toll ligand receptor.

A “co-stimulatory signal”, as used herein, refers to a signal, which in combination with a primary signal, such as TCR/CD3 ligation, leads to T cell proliferation and/or upregulation or downregulation of key molecules.

A “disease” is a state of health of an animal wherein the animal cannot maintain homeostasis, and wherein if the disease is not ameliorated then the animal's health continues to deteriorate. In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

The term “downregulation” as used herein refers to the decrease or elimination of gene expression of one or more genes.

“Effective amount” or “therapeutically effective amount” are used interchangeably herein, and refer to an amount of a compound, formulation, material, or composition, as described herein effective to achieve a particular biological result or provides a therapeutic or prophylactic benefit. Such results may include, but are not limited to an amount that when administered to a mammal, causes a detectable level of immune suppression or tolerance compared to the immune response detected in the absence of the composition of the invention.

The immune response can be readily assessed by a plethora of art-recognized methods. The skilled artisan would understand that the amount of the composition administered herein varies and can be readily determined based on a number of factors such as the disease or condition being treated, the age and health and physical condition of the mammal being treated, the severity of the disease, the particular compound being administered, and the like.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

As used herein “endogenous” refers to any material from or produced inside an organism, cell, tissue or system.

The term “epitope” as used herein is defined as a small chemical molecule on an antigen that can elicit an immune response, inducing B and/or T cell responses. An antigen can have one or more epitopes. Most antigens have many epitopes; i.e., they are multivalent. In general, an epitope is roughly about 10 amino acids and/or sugars in size. Preferably, the epitope is about 4-18 amino acids, more preferably about 5-16 amino acids, and even more most preferably 6-14 amino acids, more preferably about 7-12, and most preferably about 8-10 amino acids. One skilled in the art understands that generally the overall three-dimensional structure, rather than the specific linear sequence of the molecule, is the main criterion of antigenic specificity and therefore distinguishes one epitope from another. Based on the present disclosure, a peptide used in the present invention can be an epitope.

As used herein, the term “exogenous” refers to any material introduced from or produced outside an organism, cell, tissue or system.

The term “expand” as used herein refers to increasing in number, as in an increase in the number of T cells. In one embodiment, the T cells that are expanded ex vivo increase in number relative to the number originally present in the culture. In another embodiment, the T cells that are expanded ex vivo increase in number relative to other cell types in the culture. The term “ex vivo,” as used herein, refers to cells that have been removed from a living organism, (e.g., a human) and propagated outside the organism (e.g., in a culture dish, test tube, or bioreactor).

The term “expression” as used herein is defined as the transcription and/or translation of a particular nucleotide sequence driven by its promoter.

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system.

Expression vectors include all those known in the art, such as cosmids, plasmids (e.g., naked or contained in liposomes) and viruses (e.g., Sendai viruses, lentiviruses, retroviruses, adenoviruses, and adeno-associated viruses) that incorporate the recombinant polynucleotide.

“Identity” as used herein refers to the subunit sequence identity between two polymeric molecules particularly between two amino acid molecules, such as, between two polypeptide molecules. When two amino acid sequences have the same residues at the same positions; e.g., if a position in each of two polypeptide molecules is occupied by an arginine, then they are identical at that position. The identity or extent to which two amino acid sequences have the same residues at the same positions in an alignment is often expressed as a percentage. The identity between two amino acid sequences is a direct function of the number of matching or identical positions; e.g., if half (e.g., five positions in a polymer ten amino acids in length) of the positions in two sequences are identical, the two sequences are 50% identical; if 90% of the positions (e.g., 9 of 10), are matched or identical, the two amino acids sequences are 90% identical.

The term “immune response” as used herein is defined as a cellular response to an antigen that occurs when lymphocytes identify antigenic molecules as foreign and induce the formation of antibodies and/or activate lymphocytes to remove the antigen.

The term “immunosuppressive” is used herein to refer to reducing overall immune response.

“Isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

A “lentivirus” as used herein refers to a genus of the Retroviridae family. Lentiviruses are unique among the retroviruses in being able to infect non-dividing cells; they can deliver a significant amount of genetic information into the DNA of the host cell, so they are one of the most efficient methods of a gene delivery vector. HIV, SIV, and FIV are all examples of lentiviruses. Vectors derived from lentiviruses offer the means to achieve significant levels of gene transfer in vivo.

By the term “modified” as used herein, is meant a changed state or structure of a molecule or cell of the invention. Molecules may be modified in many ways, including chemically, structurally, and functionally. Cells may be modified through the introduction of nucleic acids.

By the term “modulating,” as used herein, is meant mediating a detectable increase or decrease in the level of a response in a subject compared with the level of a response in the subject in the absence of a treatment or compound, and/or compared with the level of a response in an otherwise identical but untreated subject. The term encompasses perturbing and/or affecting a native signal or response thereby mediating a beneficial therapeutic response in a subject, preferably, a human.

In the context of the present invention, the following abbreviations for the commonly occurring nucleic acid bases are used. “A” refers to adenosine, “C” refers to cytosine, “G” refers to guanosine, “T” refers to thymidine, and “U” refers to uridine.

The term “oligonucleotide” typically refers to short polynucleotides. It will be understood that when a nucleotide sequence is represented by a DNA sequence (i.e., A, T, C, G), this also includes an RNA sequence (i.e., A, U, C, G) in which “U” replaces “T.”

Unless otherwise specified, a “nucleotide sequence encoding an amino acid sequence” includes all nucleotide sequences that are degenerate versions of each other and that encode the same amino acid sequence. The phrase nucleotide sequence that encodes a protein or an RNA may also include introns to the extent that the nucleotide sequence encoding the protein may in some version contain an intron(s).

“Parenteral” administration of an immunogenic composition includes, e.g., subcutaneous (s.c.), intravenous (i.v.), intramuscular (i.m.), or intrasternal injection, or infusion techniques.

The term “polynucleotide” as used herein is defined as a chain of nucleotides. Furthermore, nucleic acids are polymers of nucleotides. Thus, nucleic acids and polynucleotides as used herein are interchangeable. One skilled in the art has the general knowledge that nucleic acids are polynucleotides, which can be hydrolyzed into the monomeric “nucleotides.” The monomeric nucleotides can be hydrolyzed into nucleosides. As used herein polynucleotides include, but are not limited to, all nucleic acid sequences which are obtained by any means available in the art, including, without limitation, recombinant means, i.e., the cloning of nucleic acid sequences from a recombinant library or a cell genome, using ordinary cloning technology and PCR, and the like, and by synthetic means.

As used herein, the terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

By the term “specifically binds,” as used herein with respect to an antibody, is meant an antibody which recognizes a specific antigen, but does not substantially recognize or bind other molecules in a sample. For example, an antibody that specifically binds to an antigen from one species may also bind to that antigen from one or more species. But, such cross-species reactivity does not itself alter the classification of an antibody as specific. In another example, an antibody that specifically binds to an antigen may also bind to different allelic forms of the antigen. However, such cross reactivity does not itself alter the classification of an antibody as specific. In some instances, the terms “specific binding” or “specifically binding,” can be used in reference to the interaction of an antibody, a protein, or a peptide with a second chemical species, to mean that the interaction is dependent upon the presence of a particular structure (e.g., an antigenic determinant or epitope) on the chemical species; for example, an antibody recognizes and binds to a specific protein structure rather than to proteins generally. If an antibody is specific for epitope “A”, the presence of a molecule containing epitope A (or free, unlabeled A), in a reaction containing labeled “A” and the antibody, will reduce the amount of labeled A bound to the antibody.

By the term “stimulation,” is meant a primary response induced by binding of a stimulatory molecule (e.g., a TCR/CD3 complex) with its cognate ligand thereby mediating a signal transduction event, such as, but not limited to, signal transduction via the TCR/CD3 complex. Stimulation can mediate altered expression of certain molecules, such as downregulation of TGF-beta, and/or reorganization of cytoskeletal structures, and the like.

A “stimulatory molecule,” as the term is used herein, means a molecule on a T cell that specifically binds with a cognate stimulatory ligand present on an antigen presenting cell.

A “stimulatory ligand,” as used herein, means a ligand that when present on an antigen presenting cell (e.g., an aAPC, a dendritic cell, a B-cell, and the like) can specifically bind with a cognate binding partner (referred to herein as a “stimulatory molecule”) on a T cell, thereby mediating a primary response by the T cell, including, but not limited to, activation, initiation of an immune response, proliferation, and the like. Stimulatory ligands are well-known in the art and encompass, inter alia, an MHC Class I molecule loaded with a peptide, an anti-CD3 antibody, a superagonist anti-CD28 antibody, and a superagonist anti-CD2 antibody.

The term “subject” is intended to include living organisms in which an immune response can be elicited (e.g., mammals). A “subject” or “patient,” as used therein, may be a human or non-human mammal. Non-human mammals include, for example, livestock and pets, such as ovine, bovine, porcine, canine, feline and murine mammals. Preferably, the subject is human.

A “target site” or “target sequence” refers to a nucleic acid sequence that defines a portion of a nucleic acid to which a binding molecule may specifically bind under conditions sufficient for binding to occur. In some embodiments, a target sequence refers to a genomic nucleic acid sequence that defines a portion of a nucleic acid to which a binding molecule may specifically bind under conditions sufficient for binding to occur.

As used herein, the term “T cell receptor” or “TCR” refers to a complex of membrane proteins that participate in the activation of T cells in response to the presentation of antigen. The TCR is responsible for recognizing antigens bound to major histocompatibility complex molecules. TCR is composed of a heterodimer of an alpha (a) and beta (p) chain, or gamma and delta (γ/δ) chains. TCRs may exist in alpha/beta and gamma/delta forms, which are structurally similar but have distinct anatomical locations and functions. Each chain is composed of two extracellular domains, a variable and constant domain. In some embodiments, the TCR may be modified on any cell comprising a TCR, including, for example, a helper T cell, a cytotoxic T cell, a memory T cell, regulatory T cell, natural killer T cell, and gamma delta T cell.

The term “therapeutic” as used herein means a treatment and/or prophylaxis. A therapeutic effect is obtained by suppression, remission, or eradication of a disease state.

The term “γδTCR activator” refers to any compound or substance having the ability to activate a γδ T cell receptor resulting in activation of γδ T cells and stimulation of γδ T cell proliferation. Indicators of γδ T cell activation and proliferation include final cell purity of γδT cells in the cell population, the γδ T cell expansion fold, number of expanded γδ T cells, and γδT cell functions. Commonly used γδTCR activators include aminobisphosphonates, such as zoledronate, and phosphoantigens. As used herein, a “weaker γδTCR activator than zoledronate” refers to any compound or substance which, on its own and at an optimized concentration, results in lower γδ T cell activation and proliferation than does zoledronate on its own and at an optimized concentration. For example, a weaker γδTCR activator than zoledronate will result in a γδ T cell expansion fold which is at least 2-fold lower than a γδ T cell expansion fold achieved using zoledronate under otherwise identical conditions.

As used herein, the term “TLR7/8 agonist(s)” refers to agonist(s) of TLR7, agonist(s) of TLR8, or agonist(s) of both TLR7 and TLR8.

“Transplant” refers to a biocompatible lattice or a donor tissue, organ or cell, to be transplanted. An example of a transplant may include but is not limited to skin cells or tissue, bone marrow, and solid organs such as heart, pancreas, kidney, lung and liver. A transplant can also refer to any material that is to be administered to a host. For example, a transplant can refer to a nucleic acid or a protein.

The term “transfected” or “transformed” or “transduced” as used herein refers to a process by which exogenous nucleic acid is transferred or introduced into the host cell. A “transfected” or “transformed” or “transduced” cell is one which has been transfected, transformed or transduced with exogenous nucleic acid. The cell includes the primary subject cell and its progeny.

To “treat” a disease as the term is used herein, means to reduce the frequency or severity of at least one sign or symptom of a disease or disorder experienced by a subject.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, and the like. Examples of viral vectors include, but are not limited to, Sendai viral vectors, adenoviral vectors, adeno-associated virus vectors, retroviral vectors, lentiviral vectors, and the like.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### B. Methods of Selecting γδT Cell-Based Immunotherapy Donors

In one aspect, the present invention provides methods for selecting γδ T cell-based immunotherapy donors. γδ T cell-based immunotherapies are known in the art, and are described, for example, in de Weerdt et al., Blood. 2018; 132(21):2260-72; Xu et al., Cell Mol Immunol. 2020; Nicol et al., Br J Cancer. 2011; 105(6):778-86; Kobayashi et al., Anticancer Res. 2010; 30(2):575-9; Kakimi et al., Translational lung cancer research. 2014; 3(1):23-33; Kunzmann et. al., J Immunother. 2012; 35(2):205-13); Sebestyen et al., Nature reviews Drug discovery. 2020; 19(3):169-8436; and Xu et al., Cell Mol Immunol. 2020; Chauvin et al., Clin Cancer Res. 2019; 25(23):7218-28. This list is not intended to be limiting, and the invention should be construed to include any γδ T cell-based immunotherapy.

In one aspect, the invention includes a method of selecting a donor for γδ T cell-based immunotherapy comprising assessing the expansion capacity of a population of γδ T cells from a subject. When the expansion capacity is high, the subject is selected as a donor, and when the expansion capacity is low, the subject is not selected as a donor.

In certain embodiments, assessing the expansion capacity of a population of γδ T cells comprises incubating the γδ T cells with an agent/composition that expands the γδ T cells. For example, zoledronate (ZOL) or an anti-γδTCR antibody can be used to expand the γδ T cells, but the invention should be construed to include any agent known in the art to expand γδ T cells.

In certain embodiments, assessing an index score is calculated to assess the expansion capacity. In certain embodiments the index score is calculated based on the baseline γδ T cell concentration in the subject's PMBC using the formula: Vδ2 Index Score=−8.23+10.25×Initial γδT %. In certain embodiments, the index score is calculated using the formula:

$$Comprehensive V δ 2 Index Score = ln ( \frac{P}{1 - P} ) = - 20.57 + 24.05 \times Initial\_Vδ2T \% + 0.04 \times PD 1 - 0.08 \times CTLA 4 - 0.07 \times Emoes + 0.04 \times T - bet - 0.25 \times INF - γ + 0.02 \times GranzB + 0.48 \times CD 80 - 0.27 \times CD 86$$

The probability P that an individual's γδ T cells can be expanded ≥50% was calculated as:

$$P = \frac{{e}^{S c o r e}}{1 + {e}^{S c o r e}}$$

In another aspect, the invention includes a method of selecting a donor for γδ T cell-based immunotherapy comprising measuring the basal level of Vδ2 T cells in a subject. When the basal level of Vδ2 T cells is high, the subject is selected as a donor, and wherein when the basal level of Vδ2 T cells is low, the subject is not selected as a donor. In certain embodiments, a basal level of Vδ2 T cells of at least 0.5% of the total PBMC population is considered high. In certain embodiments, a basal level of Vδ2 T cells between 0.5%-3% of the total PBMC population is considered high. In certain embodiments, a basal level of Vδ2 T cells of at least 0.82% of the total PBMC population is considered high. In certain embodiments, this method is used for autologous transfer of γδ T cells. For example, when a subject is selected as a donor, his/her own γδ T cell are used for the γδ T cell-therapy.

In certain embodiments of the methods, the immune phenotypes of the γδ T cells are assessed. In certain embodiments, when the subject's γδ T cells display effector memory type and central memory phenotype the subject is selected as a donor.

In certain embodiments the levels of PD-1, CTLA-4, Eomes, IFN-γ, Granzyme B, and CD86 are assessed in the γδ T cells. In certain embodiments, when the levels of PD-1, CTLA-4 and Eomes are low, and the levels of IFN-γ, Granzyme B, and CD86 are high in the subject's γδ T cells compared to a reference sample, then the subject is selected as a donor. In certain embodiments, the reference sample comprises γδ T cells from a poor expansion group.

### C. Methods for Expansion and Activation of γδT Cells

In one aspect, the present invention provides a method for expanding and activating a plurality of γδ T cells, the method comprising contacting a population of cells comprising a plurality of γδ T cells with: (a) at least one γδ T cell receptor (γδTCR) activator, wherein the γδTCR activator is not zoledronate and is a weaker γδTCR activator than zoledronate; and (b) a toll-like receptor (TLR) agonist. In some embodiments, the γδTCR activator comprises a phosphoantigen. In some embodiments, the γδTCR activator comprises at least one anti-γδTCR antibody and/or anti-γδTCR antigen-binding domain. In some embodiments, the γδTCR activator comprises a phosphoantigen and at least one anti-γδTCR antibody and/or anti-γδTCR antigen-binding domain.

In some embodiments, the γδTCR activator comprises a phosphoantigen. In some embodiments, the phosphoantigen is selected from the group consisting of isopentenyl pyrophosphate (IPP), bromohydrin pyrophosphate (BrHPP), and 2-methyl-3-butenyl-1-pyrophosphate (2M3B1PP). In some embodiments, the phosphoantigen is IPP.

In some embodiments, the γδTCR activator comprises at least one anti-γδTCR antibody and/or anti-γδTCR antigen-binding domain. Anti-γδTCR antibodies and antigen-binding domains are known and are commercially available. Any anti-γδTCR antibody or anti-γδTCR antigen-binding domain may be used in the present invention, including a monoclonal antibody (mAb), a polyclonal antibody, a synthetic antibody, a human antibody, a humanized antibody, a non-human antibody, an aglycosylated antibody, a single-domain antibody, a full length antibody or any antigen-binding fragment thereof, a Fab, and/or a single-chain variable fragment (scFv).

Toll-like receptors (TLRs) are pattern-recognition receptors that recognize pathogen-associated molecular patterns (PAMPs) and/or damage-associated molecular patterns (DAMPs). They are essential receptors in host defense against various pathogens and cancer cells (Wesch, et al., 2011, Cell Mol Life Sci 68: 2357-2370). There are ten functional TLRs in humans (TLR1-10) and twelve in mice (TLR1-9, and TLR11-13). TLRs can be found as homo or heterodimers (such as TLR1/2 and TLR2/6 heterodimers). Upon recognition of a PAMP, the TLR changes its conformation and recruits adapter proteins such as TRIF, TRAM, MyD88 and TIRAP. Human γδ T cells are known to express multiple TLRs, including TLR2, TLR3, TLR4, TLR7, TLR8, and TLR9 (Dar, et al., 2014, Frontiers in immunology 5: 366; Pietschmann, et al., 2009, Scand J Immunol 70: 245-255). TLRs interact with γδTCR to modulate γδ T cell functions (Pietschmann, et al., 2009, Scand J Immunol 70: 245-255).

TLR agonists come from a variety of natural sources and synthetic molecules. Agonists of TLR1/2 include, but are not necessarily limited to, triacylated lipoproteins, lipoteichoic acid, peptidoglycans, zymosan, CU-T12-9, and Pam3CSK4. Agonists of TLR2/6 include, but are not necessarily limited to, diacylated lipopeptides, HSPs, HMGB1, uric acid, fibronectin, ECM proteins, and Pam3CSK4. Agonists of TLR3 include, but are not necessarily limited to, dsRNA, poly I:C, poly A:U. Agonists of TLR4 include, but are not necessarily limited to, LPS, lipid A (endotoxin), lipoteichoic acid, β-defensin 2, fibronectin EDA, HMGB1, snapin, and tenascin C. Exemplary TLR5 agonists include, but are not necessarily limited to, natural or recombinant flagellin and analogs thereof. Exemplary TLR9 agonists include, but are not necessarily limited to, unmethylated CpG DNA. Exemplary TLR13 agonists include, but are not necessarily limited to, 23S ribosomal RNA.

TLR7 and TLR8 are phylogenetically similar. Agonists of TLR7, TLR8, or TLR7 and TLR8, collectively referred to herein as “TLR7/8 agonists,” include, but are not necessarily limited to, ssRNA, short dsRNA, poly(dT), nucleoside analogs (e.g., CL264, CL307, and loxoribine), imidazoquinolone compounds (e.g., resiquimod (R848), imiquimod, and gardiquimod), motolimod, thiazoquinolone CL075, and benzoazepine analogs TL8-506 and VTX-2337. In some embodiments, the TLR agonist comprises an agonist of TLR7 and/or TLR8 (TLR7/8 agonist). In some embodiments, the TLR7/8 agonist comprises an imidazoquinoline compound. In some embodiments, the imidazoquinoline compound is selected from resiquimod, imiquimod, and gardiquimod. In some embodiments, the imidazoquinoline compound is resiquimod. In some embodiments, the at least one γδTCR activator is IPP and the TLR agonist is resiquimod.

Immune cells can be obtained from a number of sources, including blood, peripheral blood mononuclear cells (PBMCs), bone marrow, lymph node tissue, spleen tissue, umbilical cord, lymph, or lymphoid organs. Gamma delta T cells are a small subset of T cells making up about 2% of the total T cells in humans and mice, and are found predominately in the gut mucosa, within the intraepithelial lymphocytes. In certain embodiments, the methods described herein expand γδ T cells from peripheral blood. That is, in certain embodiments, the population of cells comprises peripheral blood mononuclear cells (PBMCs). The PBMCs may be from any human or non-human mammalian species. Non-human mammals include, for example, livestock and pets, such as ovine, bovine, porcine, canine, feline and murine mammals, as well as simian and non-human primate mammals. In some embodiments, the population of cells comprises human cells, e.g., human PBMCs). In some embodiments, the plurality of γδ T cells comprises human γδ T cells. The methods described here are generally performed after isolating or removing a population of cells (e.g., PBMCs) from a mammal. That is, in some embodiments, the method is performed ex vivo.

In some embodiments, contacting the population of cells with at least one γδTCR activator and with a TLR agonist is performed in a cell culture medium. In certain embodiments, contacting the population of cells with a γδTCR activator and contacting the population of cells with a TLR agonist are performed on the same day.

Appropriate methods for culturing immune cells are well known in the art. In some embodiments, the method is performed in a cell culture medium in a culture apparatus for a set period of time. In some embodiments, the population of cells is cultured until the cells reach confluency or high cell density for optimal passage before passing the cells to another culture apparatus. The culturing apparatus can be of any culture apparatus commonly used for culturing cells in vitro. Preferably, the level of confluence is 50% or greater before passing the cells to another culture apparatus. More preferably, the level of confluence is 90% or greater. A set period of time can be any time suitable for the culture of cells in vitro. The cell culture medium may be replaced during the culture of the cells at any time. Preferably, the cell culture medium is replaced about every 2 to 3 days. The cells are then harvested from the culture apparatus whereupon the cells can be used immediately or cryopreserved to be stored for use at a later time. In one embodiment, the invention includes cryopreserving the expanded γδ T cells.

The culturing step as described herein (contact with agents as described herein or after electroporation) can be very short, for example less than 24 hours such as 1, 2, 3, 4, 5, 6, 7, 8, 9, 10, 11, 12, 13, 14, 15, 16, 17, 18, 19, 20, 21, 22, or 23 hours. The culturing step as described further herein (contact with agents as described herein) can be longer, for example 1, 2, 3, 4, 5, 6, 7, 8, 9, 10, 11, 12, 13, 14, or more days.

Various terms are used to describe cells in culture. Cell culture refers generally to cells taken from a living organism and grown under controlled condition. A primary cell culture is a culture of cells, tissues or organs taken directly from an organism and before the first subculture. Cells are expanded in culture when they are placed in a growth medium under conditions that facilitate cell growth and/or division, resulting in a larger population of the cells. When cells are expanded in culture, the rate of cell proliferation is typically measured by the amount of time required for the cells to double in number, otherwise known as the doubling time. Each round of subculturing is referred to as a passage. When cells are subcultured, they are referred to as having been passaged. A specific population of cells, or a cell line, is sometimes referred to or characterized by the number of times it has been passaged. For example, a cultured cell population that has been passaged ten times may be referred to as a P10 culture. The primary culture, i.e., the first culture following the isolation of cells from tissue, is designated P0. Following the first subculture, the cells are described as a secondary culture (P1 or passage 1). After the second subculture, the cells become a tertiary culture (P2 or passage 2), and so on. It will be understood by those of skill in the art that there may be many population doublings during the period of passaging; therefore the number of population doublings of a culture is greater than the passage number. The expansion of cells (i.e., the number of population doublings) during the period between passaging depends on many factors, including but is not limited to the seeding density, substrate, medium, and time between passaging.

In some embodiments, the cells may be cultured for several hours (about 3 hours) to about 31 days or any hourly integer value in between. Conditions appropriate for T cell culture include an appropriate media (e.g., Minimal Essential Media or RPMI Media 1640 or, X-vivo 15, (Lonza), CTS™ OpTmizer™ T Cell Expansion SFM (ThermoFisher Scientific), LymphoONE T-Cell Expansion Xeno-Free Medium (Takara)) that may contain additional factors necessary for proliferation and viability, including serum (e.g., fetal bovine or human serum), interleukin-2 (IL-2), insulin, IFN-gamma, IL-4, IL-7, GM-CSF, IL-10, IL-12, IL-15, TGF-beta, and TNF-α or any other additives for the growth of cells known to the skilled artisan. Examples of other additives for the growth of cells include, but are not limited to, surfactant, plasmanate, and reducing agents such as N-acetyl-cysteine and 2-mercaptoethanol. Media can include RPMI 1640, AIM-V, DMEM, MEM, α-MEM, F-12, X-Vivo 15, and X-Vivo 20, Optimizer, with added amino acids, sodium pyruvate, and vitamins, either serum-free or supplemented with an appropriate amount of serum (or plasma) or a defined set of hormones, and/or an amount of cytokine(s) sufficient for the growth and expansion of γδ T cells. Antibiotics, e.g., penicillin and streptomycin, are included only in experimental cultures, not in cultures of cells that are to be infused into a subject. The target cells are maintained under conditions necessary to support growth, for example, an appropriate temperature (e.g., 37° C.) and atmosphere (e.g., air plus 5% CO₂).

In some embodiments, the method of expanding the δγT cells further comprises isolating the expanded γδ T cells for further applications. In some embodiments, the method of expanding further comprises a subsequent electroporation of the expanded T cells followed by culturing. The subsequent electroporation may include introducing a nucleic acid encoding an agent, such as transducing the expanded T cells, transfecting the expanded T cells, or electroporating the expanded T cells with a nucleic acid, into the expanded population of T cells, wherein the agent further stimulates the T cell. The agent may stimulate the T cells, such as by stimulating further expansion, effector function, or another T cell function.

The γδ T cell expansion methods disclosed herein provides γδ T cells to be multiplied by about 10 fold, 20 fold, 30 fold, 40 fold, 50 fold, 60 fold, 70 fold, 80 fold, 90 fold, 100 fold, 200 fold, 300 fold, 400 fold, 500 fold, 600 fold, 700 fold, 800 fold, 900 fold, 1,000 fold, 2,000 fold, 3,000 fold, 4,000 fold, 5,000 fold, 6,000 fold, 7,000 fold, 8,000 fold, 9,000 fold, 10,000 fold, 100,000 fold, 1,000,000 fold, 10,000,000 fold, or greater, and any and all whole or partial integers therebetween. In some embodiments, the γδ T cells are expanded by at least 10-fold, at least 100-fold, at least 1,000-fold, or at least 10,000-fold.

Human γδ T cells are usually sub-divided based on two variable regions of TCR6: Vδ1 and Vδ2. Vδ1 cells are the predominant subset found at mucosal surfaces, whereas Vδ2 cells (which are almost exclusively Vγ9 cells) mostly dominate the peripheral blood. Vδ2 T cells (e.g., Vγ9/Vδ2 T cells) are a subset of γδ T cells which are unique to humans and primates. In some embodiments of the method described herein, the expanded plurality of γδ T cells comprises Vδ2 T cells.

In some embodiments of the method described herein, the γδTCR activator comprises at least one anti-γδTCR antibody and/or anti-γδTCR antigen-binding domain. In some embodiments, a plurality of the at least one anti-γδTCR antibody and/or anti-γδTCR antigen-binding domain is attached to a bead to generate an antibody-conjugated bead.

In some embodiments, the bead is further attached to a plurality of at least one anti-costimulatory receptor antibody and/or anti-costimulatory receptor antigen-binding domain. In some embodiments, the costimulatory receptor is selected from CD27, CD28, CD137 (4-1BB), CD277 (BTN3A1), CD314 (NKG2D), and PD-1 (CD279). In some embodiments, the bead is further attached to an anti-PD1 antibody. Numerous examples of monoclonal anti-costimulatory receptor antibodies are known in the art and many are commercially available, e.g., from Invitrogen™ A variety of beads for attaching or conjugating proteins, including antibodies and antigen binding domains, are commercially available, and a skilled artisan will be able to select an appropriate bead. In some embodiments, the bead has a diameter ranging from about 100 nm to about 10 μm. In some embodiments, the bead has a diameter of about 0.45 μm. In some embodiments, the bead is a magnetic bead, such as a Dynabead. In some embodiments, the plurality of γδ T cells binds to a plurality of the antibody-conjugated beads, and the method further comprises purifying the plurality of γδ T cells away from other cells within the population of cells. The purification is performed by applying a magnetic field, thereby separating the plurality of γδ T cells bound by the plurality of antibody-conjugated beads from other cells within the population of cells.

The expanded plurality of γδ T cells have improved cytotoxicity toward tumor cells and reduced T cell exhaustion. In certain embodiments, the expanded plurality of γδ T cells are characterized by one or more of the following phenotypes compared to a plurality of γδ T cells expanded in the absence of a TLR agonist and in the presence of the γδTCR activator or a bisphosphonate:
(i) increased expression of cytotoxicity surface markers (e.g., granzyme B, CD107a, and/or CD86);
(ii) increased expression of proinflammatory cytokines (e.g., IFN-γ, TNF-α, and/or IL-17A);
(iii) decreased expression of immune checkpoint proteins (e.g., PD-1);
(iv) increased cytotoxicity toward tumor cells;
(v) enhanced tumor volume reduction in vivo;
(vi) enhanced PI3K-Akt-mTOR pathway; and/or
(vii) ability to suppress inhibitory functions of adherent antigen presenting cells (APCs) present in the population of cells.

In some embodiments, the plurality of γδ T cells is genetically modified, such as by transfecting, transforming, or transducing the γδ T cells (and/or the population of cells comprising the γδ T cells) with a nucleic acid, such as a vector. In certain embodiments, the vector is a retroviral vector or a lentiviral vector. In some embodiments, the plurality of γδT cells is genetically modified to express a receptor selected from a chimeric antigen receptor (CAR), a TCR, a switch receptor, a dominant negative receptor, or any combination thereof.

CARs are well-known in the art (see, e.g., WO2014153270A1, WO2014130657A1, and WO2012079000A1). CARs comprise an extracellular antigen binding domain, a transmembrane domain, and an intracellular domain. The antigen binding domain of a CAR can include any domain that binds to the antigen (e.g., a tumor antigen) and may include, but is not limited to, a monoclonal antibody (mAb), a polyclonal antibody, a synthetic antibody, a human antibody, a humanized antibody, a non-human antibody, an aglycosylated antibody, a single-domain antibody, a full length antibody or any antigen-binding fragment thereof, a Fab, and a single-chain variable fragment (scFv). The transmembrane domain of the CAR is a region that is capable of spanning the plasma membrane of a cell and connects the antigen binding domain of the CAR to the intracellular domain of the CAR. A CAR may further comprise a hinge domain positioned N-terminal to the transmembrane domain. The intracellular domain (or “intracellular signaling domain”) of the CAR is responsible for activation of at least one of the effector functions of the cell in which the CAR is expressed (e.g., immune cell). The intracellular signaling domain transduces the effector function signal and directs the cell (e.g., immune cell) to perform its specialized function, e.g., harming and/or destroying a target cell. The intracellular domain of a CAR typically includes, but is not limited to, the cytoplasmic portion of a surface receptor, co-stimulatory molecule, and any molecule that acts in concert to initiate signal transduction in the immune cell (i.e., the T cell), as well as any derivative or variant of these elements and any synthetic sequence that has the same functional capability. Examples include, e.g., a CD3 zeta signaling domain or any other immunoreceptor tyrosine-based activation motif (ITAM) bearing cytoplasmic domain. The intracellular domain of a CAR typically further comprises at least one costimulatory domain from a costimulatory protein such as, e.g., CD28 and/or 4-1BB.

In some embodiments, the γδ T cells are engineered to express an engineered TCR, such as a tumor neoantigen-specific TCRs. Methods and compositions for ACT using recombinant TCRs are described (see, e.g., Blankenstein, et al., 2015, Curr Opin Immunol., 33:112-9; de Witte, et al., 2015, Cancer Immunol Immunother., 64(7):893-902; Schmitt, et al., 2016, Clin. Cancer Res., 21(23):5191-7; Debets, et al., 2016, Semin Immunol., 28(1):10-21; Ye et al., 2017, Immunol Res., 2017:5210459; Tendeiro Rego, et al., 2019, Cytotherapy, 21(3):341-357; Ghaffari et al., 2021, J Exp Clin Cancer Res., 40(1):269; and references cited in any of the foregoing).

Switch receptors comprise an extracellular domain of a first immune receptor and an intracellular domain of a second immune receptor, linked by a transmembrane domain. The transmembrane domain of a switch receptor is typically derived from the first receptor or from the second receptor, but may be any suitable transmembrane domain. The swich receptor switches the signal from the signal normally transduced by the first immune receptor to the signal transduced by the second immune receptor upon binding of a ligand to the extracellular domain of the first immune receptor. See, e.g., WO2013019615A2.

Dominant-negative (DN) mutants represent an important class of mutation in which a DN mutant receptor interferes with the function of the wild-type (WT) version of the receptor. In some embodiments, the dominant negative receptor is a dominant negative TGF-β Receptor (dnTGFβR). See, e.g., Brand, et al., 1993, J Biol Chem., 268(16):11500-3.

In another embodiment, the method comprises isolating the cell population (e.g., PBMCs) prior to expanding the γδ T cells. In another embodiment, the invention further comprises cryopreserving the cell population prior to expansion. In yet another embodiment, the cryopreserved cells are thawed, and may be transformed, transfected, or transduced with a nucleic acid, such as a vector, encoding a membrane receptor (a CAR, a TCR, a switch receptor, or a dominant negative receptor) prior to expansion of the γδ T cells. In certain embodiments, the vector is a retroviral vector or a lentiviral vector. In other embodiments, the γδ T cells are isolated (purified) after being expanded. In some embodiments, the isolated γδ T cells are transformed, transfected, or transduced with a nucleic acid, such as a vector, encoding a membrane receptor. In certain embodiments, the vector is a retroviral vector or a lentiviral vector.

In another aspect, the invention provides a method for expanding and activating a plurality of γδ T cells, the method comprising contacting a population of cells comprising a plurality of γδ T cells with an antibody-conjugated bead, wherein the antibody-conjugated bead comprises a bead attached to a plurality of at least one anti-γδ T cell receptor (anti-γδTCR) antibody and/or anti-γδTCR antigen-binding domain; and wherein the method does not comprise contacting the population of cells with zoledronate. In certain embodiments, the bead is further attached to a plurality of at least one anti-costimulatory receptor antibody and/or anti-costimulatory receptor antigen-binding domain. In some embodiments, the costimulatory receptor is selected from CD27, CD28, CD137 (4-1BB), CD277 (BTN3A1), CD314 (NKG2D) and PD-1 (CD279). Numerous examples of monoclonal anti-costimulatory receptor antibodies are known in the art and many are commercially available, e.g., from Invitrogen™.

As described elsewhere herein, a variety of beads for attaching or conjugating proteins, including antibodies and antigen binding domains, are commercially available, and a skilled artisan will be able to select an appropriate bead for use in the methods described herein. In certain embodiments, the bead has a diameter ranging from about 100 nm to about 10 μm. In some embodiments, the bead has a diameter of about 0.45 μm. In some embodiments, the bead is a magnetic bead, such as a Dynabead. In certain embodiments, the plurality of γδ T cells binds to a plurality of the antibody-conjugated beads, and the method further comprises purifying the plurality of γδ T cells away from other cells within the population of cells. The purification is performed by applying a magnetic field, thereby separating the plurality of γδ T cells bound by the plurality of antibody-conjugated beads from other cells within the population of cells.

The expanded plurality of γδ T cells have improved cytotoxicity toward tumor cells and reduced T cell exhaustion. In certain embodiments, the expanded plurality of γδ T cells are characterized by one or more of the following phenotypes compared to a plurality of γδ T cells expanded in the absence of an anti-costimulatory receptor antibody and/or anti-costimulatory receptor antigen binding domain and in the presence of the anti-γδTCR antibody or anti-γδTCR antigen binding domain:
(i) increased expression of cytotoxicity surface markers (e.g., granzyme B, CD107a, and/or CD86);
(ii) increased expression of proinflammatory cytokines (e.g., IFN-γ, TNF-α, and/or IL-17A);
(iii) decreased expression of immune checkpoint proteins (e.g., PD-1);
(iv) increased cytotoxicity toward tumor cells; and/or
(v) enhanced tumor volume reduction in vivo.

In certain embodiments, the method further comprises contacting the population of cells comprising a plurality of γδ T cells with a toll-like receptor (TLR) agonist as described elsewhere herein. In some embodiments, the TLR agonist comprises an agonist of TLR7 and/or TLR8 (TLR7/8 agonist). In some embodiments, the TLR7/8 agonist comprises an imidazoquinoline compound. In some embodiments, the imidazoquinoline compound is selected from resiquimod, imiquimod, and gardiquimod. In some embodiments, the imidazoquinoline compound is resiquimod. In certain embodiments, contacting the population of cells with the toll-like receptor (TLR) agonist is performed on the same day as contacting the population of cells with the antibody-conjugated bead.

As described elsewhere herein, the methods described herein expand γδ T cells from peripheral blood. That is, in some embodiments, the population of cells comprises peripheral blood mononuclear cells (PBMCs). The PBMCs may be from any human or non-human mammalian species. Non-human mammals include, for example, livestock and pets, such as ovine, bovine, porcine, canine, feline and murine mammals, as well as simian and non-human primate mammals. In some embodiments, the population of cells comprises human cells, e.g., human PBMCs). In some embodiments, the plurality of γδ T cells comprises human γδ T cells. The methods described here are generally performed after isolating or removing a population of cells (e.g., PBMCs) from a mammal. That is, in some embodiments, the method is performed ex vivo.

In some embodiments, contacting the population of cells with at least one γδTCR activator and with a TLR agonist is performed in a cell culture medium. Appropriate methods for culturing immune cells are well known in the art and described elsewhere herein. In certain embodiments, the plurality of γδ T cells is expanded at least 10-fold, at least 100-fold, at least 1,000-fold, or at least 10,000-fold. In some embodiments, the expanded plurality of γδ T cells comprises Vδ2 T cells.

The expanded plurality of γδ T cells have improved cytotoxicity toward tumor cells and reduced T cell exhaustion. In certain embodiments, the expanded plurality of γδ T cells are characterized by one or more of the following phenotypes compared to a plurality of γδ T cells expanded in the absence of a TLR agonist and in the presence of a γδTCR activator:
(i) increased expression of cytotoxicity surface markers (e.g., granzyme B, CD107a, and/or CD86);
(ii) increased expression of proinflammatory cytokines (e.g., IFN-γ, TNF-α, and/or IL-17A);
(iii) decreased expression of immune checkpoint proteins (e.g., PD-1);
(iv) increased cytotoxicity toward tumor cells; and/or
(v) enhanced tumor volume reduction in vivo;
(vi) enhanced PI3K-Akt-mTOR pathway; and/or
(vii) ability to suppress inhibitory functions of adherent antigen presenting cells (APCs) present in the population of cells.

As described elsewhere herein, in certain embodiments, the plurality of γδ T cells is genetically modified. In some embodiments, the plurality of γδ T cells is genetically modified to express a receptor selected from a chimeric antigen receptor (CAR), a TCR, a dominant negative receptor, a switch receptor, or any combination thereof as described elsewhere herein.

### D. Methods of Treatment

Also provided herein are methods of treating a disease or disorder in a subject in need thereof with a γδ T cell-based immunotherapy. The methods should be construed to include any γδ T cell-based immunotherapy.

In one aspect, the method comprises selecting a donor for the γδ T cell-based immunotherapy by assessing the expansion capacity of a population of γδ T cells from a healthy subject. When the expansion capacity is high, the healthy subject is selected as a donor, and a γδ T cell-based immunotherapy comprising the γδ T cells from the donor is administered to the subject in need thereof, thus treating the disease or disorder. When the expansion capacity is low, the healthy subject is not selected as a donor and an alternative treatment is administered or a different donor is selected.

In one aspect, the method comprises selecting an individual for the γδ T cell-based immunotherapy by assessing the expansion capacity of a population of γδ T cells from this subject. When the expansion capacity is high, the individual subject is selected for a γδ T cell-based immunotherapy comprising the γδ T cells from the same subject is administered to the subject in need thereof, thus treating the disease or disorder. When the expansion capacity is low, the subject is not selected for γδ T cell-based immunotherapy and an alternative treatment is administered or a donor is selected.

In certain embodiments, the assessing comprises incubating the γδ T cells with an agent/composition that expands the γδ T cells. In certain embodiments, the agent is ZOL or an anti-γδTCR antibody. In certain embodiments, the expansion capacity comprises calculating an index score. In certain embodiments, the index score is calculated based on the baseline γδ T cell concentration in the subject's PMBC using the formula: Vδ2 Index Score=−8.23+10.25×Initial γδT %.

In certain embodiments, assessing an index score is calculated to assess the expansion capacity. In certain embodiments the index score is calculated based on the baseline γδ T cell concentration in the subject's PMBC using the formula: Vδ2 Index Score=−8.23+10.25×Initial γδT %. In certain embodiments, the index score is calculated using the formula:

$$Comprehensive V δ 2 Index Score = ln ( \frac{P}{1 - P} ) = - 20.57 + 24.05 \times Initial\_Vδ2T \% + 0.04 \times PD 1 - 0.08 \times CTLA 4 - 0.07 \times Emoes + 0.04 \times T - bet - 0.25 \times INF - γ + 0.02 \times GranzB + 0.48 \times CD 80 - 0.27 \times CD 86$$

The probability P that an individual's γδ T cells can be expanded ≥50% was calculated as:

$$P = \frac{{e}^{S c o r e}}{1 + {e}^{S c o r e}}$$

In another aspect, the invention includes a method of treating a disease or disorder in a subject in need thereof with a γδ T cell-based immunotherapy comprising selecting a donor for the γδ T cell-based immunotherapy by measuring the basal level of Vδ2 T cells in a healthy subject. When the basal level of Vδ2 T cells is high, the healthy subject is selected as a donor and a γδ T cell-based immunotherapy comprising the γδ T cells from the donor is administered to the subject in need thereof, thus treating the disease or disorder. When the basal level of Vδ2 T cells is low, the healthy subject is not selected as a donor, and an alternative treatment is administered or a different donor is selected.

In certain embodiments, a basal level of Vδ2 T cells of at least 0.5% of the total PBMC population is considered high. In certain embodiments, a basal level of Vδ2 T cells between 0.5%-3% of the total PBMC population is considered high. In certain embodiments, a basal level of Vδ2 T cells of at least 0.82% of the total PBMC population is considered high.

In certain embodiments, the method comprises assessing the immune phenotypes of the γδ T cells. In certain embodiments, when the subject's γδ T cells display effector memory type and central memory phenotype the subject is selected as a donor.

In certain embodiments, the method comprises assessing the levels of PD-1, CTLA-4, Eomes, IFN-γ, Granzyme B, and CD86 in the γδ T cells. In certain embodiments, when the levels of PD-1, CTLA-4 and Eomes are low, and the levels of IFN-γ, Granzyme B, and CD86 are high in the subject's γδ T cells compared to a reference sample, then the subject is selected as a donor. In certain embodiments, the reference sample comprises γδ T cells from a poor expansion group.

In certain embodiments, the disease or disorder is cancer. In certain embodiments, the cancer is breast cancer, In certain embodiments, the cancer is lung cancer.

In certain embodiments, the disease or disorder is an infection. In certain embodiments, the disease is a viral infection. In certain embodiments, the disease is Hepatitis C. In certain embodiments, the disease is Hepatitis B. In certain embodiments, the disease is HIV. In certain embodiments, the disease is EBV, including EBV induced lymphoma. In certain embodiments, the disease is a bacterial infection, such as Mycobacterium tuberculosis, salmonellosis, or brucellosis.

In certain embodiments, the γδ T cell-based immunotherapy comprises a modified cell (e.g., γδ T cell) comprising a chimeric antigen receptor (CAR). In certain embodiments, the γδT cell-based immunotherapy comprises a modified cell (e.g., γδ T cell) comprising a modified or exogeneous T cell receptor (TCR).

The modified cells (e.g., γδ T cells) described herein may be included in a composition for immunotherapy. The composition may include a pharmaceutical composition and further include a pharmaceutically acceptable carrier. A therapeutically effective amount of the pharmaceutical composition comprising the modified T cells may be administered.

The expanded plurality of γδ T cells described herein may be included in a composition for immunotherapy. In one aspect, the invention provides a composition comprising a plurality of γδ T cells expanded by any one of the methods described herein and a pharmaceutically-acceptable carrier. A therapeutically effective amount of the pharmaceutical composition comprising the expanded plurality of γδ T cells may be administered to a subject in a method for adoptive cell therapy, e.g., to treat a subject having a disease such as cancer. In some embodiments, the expanded plurality of γδ T cells are engineered to express a CAR, a TCR, a switch receptor, a dominant negative receptor, or any combination thereof, prior to being administered to a subject.

In some embodiments, the expanded plurality of γδ T cells comprises about 1×10⁶ to about 1×10¹² cells. Upon administration to a subject, such as a mammal or a human, the γδT cells exhibit expansion in peripheral blood of the subject. In certain embodiments, the in vivo expansion is at least 10-fold, at least 100-fold, or at least 1000-fold. In certain embodiments, the γδ T cells are detectable for at least 24 months after the cells are administered.

Methods for administration of immune cells for adoptive cell therapy are known and may be used in connection with the provided methods and compositions. For example, adoptive T cell therapy methods are described, e.g., in US Patent Application Publication No. 2003/0170238 to Gruenberg et al; U.S. Pat. No. 4,690,915 to Rosenberg; Rosenberg (2011) Nat Rev Clin Oncol. 8(10):577-85). See, e.g., Themeli et al. (2013) Nat Biotechnol. 31(10): 928-933; Tsukahara et al. (2013) Biochem Biophys Res Commun 438(1): 84-9; Davila et al. (2013) PLoS ONE 8(4): e61338. In some embodiments, the cell therapy, e.g., adoptive T cell therapy is carried out by autologous transfer, in which the cells are isolated and/or otherwise prepared from the subject who is to receive the cell therapy, or from a sample derived from such a subject. Thus, in some aspects, the cells are derived from a subject, e.g., patient, in need of a treatment and the cells, following isolation and processing are administered to the same subject.

In some embodiments, the cell therapy, e.g., adoptive T cell therapy, is carried out by allogeneic transfer, in which the cells are isolated and/or otherwise prepared from a subject other than a subject who is to receive or who ultimately receives the cell therapy, e.g., a first subject. In such embodiments, the cells then are administered to a different subject, e.g., a second subject, of the same species. In some embodiments, the first and second subjects are genetically identical. In some embodiments, the first and second subjects are genetically similar. In some embodiments, the second subject expresses the same HLA class or supertype as the first subject.

In some embodiments, the subject has been treated with a therapeutic agent targeting the disease or condition, e.g. the tumor, prior to administration of the cells or composition containing the cells. In some aspects, the subject is refractory or non-responsive to the other therapeutic agent. In some embodiments, the subject has persistent or relapsed disease, e.g., following treatment with another therapeutic intervention, including chemotherapy, radiation, and/or hematopoietic stem cell transplantation (HSCT), e.g., allogenic HSCT. In some embodiments, the administration effectively treats the subject despite the subject having become resistant to another therapy.

In some embodiments, the subject is responsive to the other therapeutic agent, and treatment with the therapeutic agent reduces disease burden. In some aspects, the subject is initially responsive to the therapeutic agent, but exhibits a relapse of the disease or condition over time. In some embodiments, the subject has not relapsed. In some such embodiments, the subject is determined to be at risk for relapse, such as at a high risk of relapse, and thus the cells are administered prophylactically, e.g., to reduce the likelihood of or prevent relapse. In some aspects, the subject has not received prior treatment with another therapeutic agent.

In some embodiments, the subject has persistent or relapsed disease, e.g., following treatment with another therapeutic intervention, including chemotherapy, radiation, and/or hematopoietic stem cell transplantation (HSCT), e.g., allogenic HSCT. In some embodiments, the administration effectively treats the subject despite the subject having become resistant to another therapy.

The γδ T cells of the present invention can be administered to an animal, preferably a mammal, even more preferably a human, to treat a cancer. In addition, the cells of the present invention can be used for the treatment of any condition related to a cancer, especially a cell-mediated immune response against a tumor cell(s), where it is desirable to treat or alleviate the disease. The types of cancers to be treated with the modified cells or pharmaceutical compositions of the invention include, carcinoma, blastoma, and sarcoma, and certain leukemia or lymphoid malignancies, benign and malignant tumors, and malignancies e.g., sarcomas, carcinomas, and melanomas. Other exemplary cancers include but are not limited breast cancer, prostate cancer, ovarian cancer, cervical cancer, skin cancer, pancreatic cancer, colorectal cancer, renal cancer, liver cancer, brain cancer, lymphoma, leukemia, lung cancer, thyroid cancer, and the like. The cancers may be non-solid tumors (such as hematological tumors) or solid tumors. Adult tumors/cancers and pediatric tumors/cancers are also included. In one embodiment, the cancer is a solid tumor or a hematological tumor. In one embodiment, the cancer is a carcinoma. In one embodiment, the cancer is a sarcoma. In one embodiment, the cancer is a leukemia. In one embodiment the cancer is a solid tumor.

Solid tumors are abnormal masses of tissue that usually do not contain cysts or liquid areas. Solid tumors can be benign or malignant. Different types of solid tumors are named for the type of cells that form them (such as sarcomas, carcinomas, and lymphomas). Examples of solid tumors, such as sarcomas and carcinomas, include fibrosarcoma, myxosarcoma, liposarcoma, chondrosarcoma, osteosarcoma, and other sarcomas, synovioma, mesothelioma, Ewing's tumor, leiomyosarcoma, rhabdomyosarcoma, colon carcinoma, lymphoid malignancy, pancreatic cancer, breast cancer, lung cancers, ovarian cancer, prostate cancer, hepatocellular carcinoma, squamous cell carcinoma, basal cell carcinoma, adenocarcinoma, sweat gland carcinoma, medullary thyroid carcinoma, papillary thyroid carcinoma, pheochromocytomas sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, Wilms' tumor, cervical cancer, testicular tumor, seminoma, bladder carcinoma, melanoma, and CNS tumors (such as a glioma (such as brainstem glioma and mixed gliomas), glioblastoma (also known as glioblastoma multiforme) astrocytoma, CNS lymphoma, germinoma, medulloblastoma, Schwannoma craniopharyogioma, ependymoma, pinealoma, hemangioblastoma, acoustic neuroma, oligodendroglioma, menangioma, neuroblastoma, retinoblastoma and brain metastases).

Carcinomas that can be amenable to therapy by a method disclosed herein include, but are not limited to, esophageal carcinoma, hepatocellular carcinoma, basal cell carcinoma (a form of skin cancer), squamous cell carcinoma (various tissues), bladder carcinoma, including transitional cell carcinoma (a malignant neoplasm of the bladder), bronchogenic carcinoma, colon carcinoma, colorectal carcinoma, gastric carcinoma, lung carcinoma, including small cell carcinoma and non-small cell carcinoma of the lung, adrenocortical carcinoma, thyroid carcinoma, pancreatic carcinoma, breast carcinoma, ovarian carcinoma, prostate carcinoma, adenocarcinoma, sweat gland carcinoma, sebaceous gland carcinoma, papillary carcinoma, papillary adenocarcinoma, cystadenocarcinoma, medullary carcinoma, renal cell carcinoma, ductal carcinoma in situ or bile duct carcinoma, choriocarcinoma, seminoma, embryonal carcinoma, Wilm's tumor, cervical carcinoma, uterine carcinoma, testicular carcinoma, osteogenic carcinoma, epithelial carcinoma, and nasopharyngeal carcinoma.

Sarcomas that can be amenable to therapy by a method disclosed herein include, but are not limited to, fibrosarcoma, myxosarcoma, liposarcoma, chondrosarcoma, chordoma, osteogenic sarcoma, osteosarcoma, angiosarcoma, endotheliosarcoma, lymphangiosarcoma, lymphangioendotheliosarcoma, synovioma, mesothelioma, Ewing's sarcoma, leiomyosarcoma, rhabdomyosarcoma, and other soft tissue sarcomas.

In certain exemplary embodiments, the modified immune cells of the invention are used to treat a myeloma, or a condition related to myeloma. Examples of myeloma or conditions related thereto include, without limitation, light chain myeloma, non-secretory myeloma, monoclonal gamopathy of undertermined significance (MGUS), plasmacytoma (e.g., solitary, multiple solitary, extramedullary plasmacytoma), amyloidosis, and multiple myeloma. In one embodiment, a method of the present disclosure is used to treat multiple myeloma. In one embodiment, a method of the present disclosure is used to treat refractory myeloma. In one embodiment, a method of the present disclosure is used to treat relapsed myeloma.

In certain exemplary embodiments, the modified immune cells of the invention are used to treat a melanoma, or a condition related to melanoma. Examples of melanoma or conditions related thereto include, without limitation, superficial spreading melanoma, nodular melanoma, lentigo maligna melanoma, acral lentiginous melanoma, amelanotic melanoma, or melanoma of the skin (e.g., cutaneous, eye, vulva, vagina, rectum melanoma). In one embodiment, a method of the present disclosure is used to treat cutaneous melanoma. In one embodiment, a method of the present disclosure is used to treat refractory melanoma. In one embodiment, a method of the present disclosure is used to treat relapsed melanoma.

In yet other exemplary embodiments, the modified immune cells of the invention are used to treat a sarcoma, or a condition related to sarcoma. Examples of sarcoma or conditions related thereto include, without limitation, angiosarcoma, chondrosarcoma, Ewing's sarcoma, fibrosarcoma, gastrointestinal stromal tumor, leiomyosarcoma, liposarcoma, malignant peripheral nerve sheath tumor, osteosarcoma, pleomorphic sarcoma, rhabdomyosarcoma, and synovial sarcoma. In one embodiment, a method of the present disclosure is used to treat liposarcoma such as myxoid/round cell liposarcoma, differentiated/dedifferentiated liposarcoma, and pleomorphic liposarcoma. In one embodiment, a method of the present disclosure is used to treat myxoid/round cell liposarcoma. In one embodiment, a method of the present disclosure is used to treat a refractory sarcoma. In one embodiment, a method of the present disclosure is used to treat a relapsed sarcoma.

The cells of the invention to be administered may be autologous, with respect to the subject undergoing therapy.

The administration of the cells of the invention may be carried out in any convenient manner known to those of skill in the art. The cells of the present invention may be administered to a subject by aerosol inhalation, injection, ingestion, transfusion, implantation or transplantation. The compositions described herein may be administered to a patient transarterially, subcutaneously, intradermally, intratumorally, intranodally, intramedullary, intramuscularly, by intravenous (i.v.) injection, or intraperitoneally. In other instances, the cells of the invention are injected directly into a site of inflammation in the subject, a local disease site in the subject, a lymph node, an organ, a tumor, and the like.

In certain embodiments, the cells, or individual populations of sub-types of cells, are administered to the subject at a range of about one million to about 100 billion cells, such as, e.g., 1 million to about 50 billion cells (e.g., about 5 million cells, about 25 million cells, about 500 million cells, about 1 billion cells, about 5 billion cells, about 20 billion cells, about 30 billion cells, about 40 billion cells, or a range defined by any two of the foregoing values), such as about 10 million to about 100 billion cells (e.g., about 20 million cells, about 30 million cells, about 40 million cells, about 60 million cells, about 70 million cells, about 80 million cells, about 90 million cells, about 10 billion cells, about 25 billion cells, about 50 billion cells, about 75 billion cells, about 90 billion cells, or a range defined by any two of the foregoing values), and in some cases about 100 million cells to about 50 billion cells (e.g., about 120 million cells, about 250 million cells, about 350 million cells, about 450 million cells, about 650 million cells, about 800 million cells, about 900 million cells, about 3 billion cells, about 30 billion cells, about 45 billion cells) or any value in between these ranges.

In some embodiments, the dose of total cells and/or dose of individual sub-populations of cells is within a range of between at or about 1×10⁵ cells/kg to about 1×10¹¹ cells/kg 10⁴ and at or about 10¹¹ cells/kilograms (kg) body weight, such as between 10⁵ and 10⁶ cells/kg body weight, for example, at or about 1×10⁵ cells/kg, 1.5×10⁵ cells/kg, 2×10⁵ cells/kg, or 1×10⁶ cells/kg body weight. For example, in some embodiments, the cells are administered at, or within a certain range of error of, between at or about 10⁴ and at or about 10⁹ T cells/kilograms (kg) body weight, such as between 10⁵ and 10⁶ T cells/kg body weight, for example, at or about 1×10⁵ T cells/kg, 1.5×10⁵ T cells/kg, 2×10⁵ T cells/kg, or 1×10⁶ T cells/kg body weight. In other exemplary embodiments, a suitable dosage range of modified cells for use in a method of the present disclosure includes, without limitation, from about 1×10⁵ cells/kg to about 1×10⁶ cells/kg, from about 1×10⁶ cells/kg to about 1×10⁷ cells/kg, from about 1×10⁷ cells/kg about 1×10⁸ cells/kg, from about 1×10⁸ cells/kg about 1×10⁹ cells/kg, from about 1×10⁹ cells/kg about 1×10¹⁰ cells/kg, from about 1×10¹⁰ cells/kg about 1×10¹¹ cells/kg. In an exemplary embodiment, a suitable dosage for use in a method of the present disclosure is about 1×10⁸ cells/kg. In an exemplary embodiment, a suitable dosage for use in a method of the present disclosure is about 1×10⁷ cells/kg. In other embodiments, a suitable dosage is from about 1×10⁷ total cells to about 5×10⁷ total cells. In some embodiments, a suitable dosage is from about 1×10⁸ total cells to about 5×10⁸ total cells. In some embodiments, a suitable dosage is from about 1.4×10⁷ total cells to about 1.1×10⁹ total cells. In an exemplary embodiment, a suitable dosage for use in a method of the present disclosure is about 7×10⁹ total cells.

In some embodiments, a dose of modified cells is administered to a subject in need thereof, in a single dose or multiple doses. In some embodiments, a dose of modified cells is administered in multiple doses, e.g., once a week or every 7 days, once every 2 weeks or every 14 days, once every 3 weeks or every 21 days, once every 4 weeks or every 28 days. In an exemplary embodiment, a single dose of modified cells is administered to a subject in need thereof. In an exemplary embodiment, a single dose of modified cells is administered to a subject in need thereof by rapid intravenous infusion.

For the prevention or treatment of disease, the appropriate dosage may depend on the type of disease to be treated, the type of cells or recombinant receptors, the severity and course of the disease, whether the cells are administered for preventive or therapeutic purposes, previous therapy, the subject's clinical history and response to the cells, and the discretion of the attending physician. The compositions and cells are in some embodiments suitably administered to the subject at one time or over a series of treatments.

In some embodiments, the cells are administered as part of a combination treatment, such as simultaneously with or sequentially with, in any order, another therapeutic intervention, such as an antibody or engineered cell or receptor or agent, such as a cytotoxic or therapeutic agent. The cells in some embodiments are co-administered with one or more additional therapeutic agents or in connection with another therapeutic intervention, either simultaneously or sequentially in any order. In some contexts, the cells are co-administered with another therapy sufficiently close in time such that the cell populations enhance the effect of one or more additional therapeutic agents, or vice versa. In some embodiments, the cells are administered prior to the one or more additional therapeutic agents. In some embodiments, the cells are administered after the one or more additional therapeutic agents. In some embodiments, the one or more additional agents includes a cytokine, such as IL-2, for example, to enhance persistence. In some embodiments, the methods comprise administration of a chemotherapeutic agent.

Following administration of the cells, the biological activity of the engineered cell populations in some embodiments is measured, e.g., by any of a number of known methods. Parameters to assess include specific binding of an engineered or natural T cell or other immune cell to antigen, in vivo, e.g., by imaging, or ex vivo, e.g., by ELISA or flow cytometry. In certain embodiments, the ability of the engineered cells to destroy target cells can be measured using any suitable method known in the art, such as cytotoxicity assays described in, for example, Kochenderfer et al., J. Immunotherapy, 32(7): 689-702 (2009), and Herman et al. J. Immunological Methods, 285(1): 25-40 (2004). In certain embodiments, the biological activity of the cells is measured by assaying expression and/or secretion of one or more cytokines, such as CD107a, IFNγ, IL-2, and TNF. In some aspects the biological activity is measured by assessing clinical outcome, such as reduction in tumor burden or load.

In certain embodiments, the subject is provided a secondary or alternative treatment. Secondary/alternative treatments include but are not limited to chemotherapy, radiation, surgery, and medications.

In some embodiments, the subject can be administered a conditioning therapy, such as a lymphodepletion step, prior to adoptive cell therapy. In some embodiments, the conditioning therapy comprises administering an effective amount of cyclophosphamide to the subject. In some embodiments, the conditioning therapy comprises administering an effective amount of fludarabine to the subject. In preferred embodiments, the conditioning therapy comprises administering an effective amount of a combination of cyclophosphamide and fludarabine to the subject. Administration of a conditioning therapy prior to CAR T cell therapy may increase the efficacy of the CAR T cell therapy. Methods of conditioning patients for T cell therapy are described in U.S. Pat. No. 9,855,298, which is incorporated herein by reference in its entirety.

### E. T Cell Receptors

The present invention provides compositions and methods for γδ T cell-based immunotherapies. In certain embodiments, the γδ T cell-based immunotherapy comprises modified immune cells or precursors thereof (e.g., modified γδ T cells) comprising an exogenous or modified T cell receptor (TCR). Thus, in some embodiments, the cell has been altered to contain specific T cell receptor (TCR) genes (e.g., a nucleic acid encoding a gamma/delta TCR). TCRs or antigen-binding portions thereof include those that recognize a peptide epitope or T cell epitope of a target polypeptide, such as an antigen of a tumor, viral or autoimmune protein. In certain embodiments, the TCR has binding specificity for a tumor associated antigen, e.g., human NY-ESO-1.

A TCR is a disulfide-linked heterodimeric protein comprised of six different membrane bound chains that participate in the activation of T cells in response to an antigen. There exists alpha/beta TCRs and gamma/delta TCRs. An alpha/beta TCR comprises a TCR alpha chain and a TCR beta chain. T cells expressing a TCR comprising a TCR alpha chain and a TCR beta chain are commonly referred to as alpha/beta T cells. Gamma/delta TCRs comprise a TCR gamma chain and a TCR delta chain. T cells expressing a TCR comprising a TCR gamma chain and a TCR delta chain are commonly referred to as gamma/delta T cells. A TCR of the present disclosure is a TCR comprising a TCR gamma chain and a TCR delta chain.

The TCR gamma chain and the TCR delta chain are each comprised of two extracellular domains, a variable region and a constant region. The TCR gamma chain variable region and the TCR delta chain variable region are required for the affinity of a TCR to a target antigen. Each variable region comprises three hypervariable or complementarity-determining regions (CDRs) which provide for binding to a target antigen. The constant region of the TCR gamma chain and the constant region of the TCR delta chain are proximal to the cell membrane. A TCR further comprises a transmembrane region and a short cytoplasmic tail. CD3 molecules are assembled together with the TCR heterodimer. CD3 molecules comprise a characteristic sequence motif for tyrosine phosphorylation, known as immunoreceptor tyrosine-based activation motifs (ITAMs).

Proximal signaling events are mediated through the CD3 molecules, and accordingly, TCR-CD3 complex interaction plays an important role in mediating cell recognition events. Engagement of the TCR initiates both positive and negative signaling cascades that result in cellular proliferation, cytokine production, and/or activation-induced cell death.

A TCR of the present invention can be a wild-type TCR, a high affinity TCR, and/or a chimeric TCR. A high affinity TCR may be the result of modifications to a wild-type TCR that confers a higher affinity for a target antigen compared to the wild-type TCR. A high affinity TCR may be an affinity-matured TCR. Methods for modifying TCRs and/or the affinity-maturation of TCRs are known to those of skill in the art. Techniques for engineering and expressing TCRs include, but are not limited to, the production of TCR heterodimers which include the native disulphide bridge which connects the respective subunits (Garboczi, et al., (1996), Nature 384(6605): 134-41; Garboczi, et al., (1996), J Immunol 157(12): 5403-10; Chang et al., (1994), PNAS USA 91: 11408-11412; Davodeau et al., (1993), J. Biol. Chem. 268(21): 15455-15460; Golden et al., (1997), J. Imm. Meth. 206: 163-169; U.S. Pat. No. 6,080,840).

In some embodiments, the exogenous TCR is a full TCR or an antigen-binding portion or antigen-binding fragment thereof. In some embodiments, the TCR is an intact or full-length TCR, including TCRs in the αβ form or γδ form. In some embodiments, the TCR is an antigen-binding portion that is less than a full-length TCR but that binds to a specific peptide. In some cases, an antigen-binding portion or fragment of a TCR can contain only a portion of the structural domains of a full-length or intact TCR, but yet is able to bind the peptide epitope, to which the full TCR binds. In some cases, an antigen-binding portion contains the variable domains of a TCR, such as variable γ chain and variable 6 chain of a TCR, sufficient to form a binding site. Generally, the variable chains of a TCR contain complementarity determining regions (CDRs) involved in recognition of the peptide.

In some embodiments, the variable domains of the TCR contain hypervariable loops, or CDRs, which generally are the primary contributors to antigen recognition and binding capabilities and specificity. In some embodiments, a CDR of a TCR or combination thereof forms all or substantially all of the antigen-binding site of a given TCR molecule. The various CDRs within a variable region of a TCR chain generally are separated by framework regions (FRs), which generally display less variability among TCR molecules as compared to the CDRs (see, e.g., Jores et al, Proc. Nat'l Acad. Sci. U.S.A. 87:9138, 1990; Chothia et al., EMBO J. 7:3745, 1988; see also Lefranc et al., Dev. Comp. Immunol. 27:55, 2003).

It is within the level of a skilled artisan to determine or identify the various domains or regions of a TCR. In some aspects, residues of a TCR are known or can be identified according to the International Immunogenetics Information System (IMGT) numbering system (see e.g. www.imgt.org; see also, Lefranc et al. (2003) Developmental and Comparative Immunology, 2&; 55-77; and The T Cell Factsbook 2nd Edition, Lefranc and LeFranc Academic Press 2001). Using this system, the CDR1 sequences within a TCR Vα chain and/or Vβ chain correspond to the amino acids present between residue numbers 27-38, inclusive, the CDR2 sequences within a TCR Vα chain and/or Vβ chain correspond to the amino acids present between residue numbers 56-65, inclusive, and the CDR3 sequences within a TCR Vα chain and/or Vβ chain correspond to the amino acids present between residue numbers 105-117, inclusive. The IMGT numbering system should not be construed as limiting in any way, as there are other numbering systems known to those of skill in the art, and it is within the level of the skilled artisan to use any of the numbering systems available to identify the various domains or regions of a TCR.

In some embodiments, the TCR may be a heterodimer of two chains γ and δ (or optionally α and β) that are linked, such as by a disulfide bond or disulfide bonds. In some embodiments, the constant domain of the TCR may contain short connecting sequences in which a cysteine residue forms a disulfide bond, thereby linking the two chains of the TCR. In some embodiments, a TCR may have an additional cysteine residue in each of the γ and δ chains, such that the TCR contains two disulfide bonds in the constant domains. In some embodiments, each of the constant and variable domains contain disulfide bonds formed by cysteine residues.

In some embodiments, the TCR for engineering cells as described is one generated from a known TCR sequence(s), such as sequences of Vγ δ chains, for which a substantially full-length coding sequence is readily available. Methods for obtaining full-length TCR sequences, including V chain sequences, from cell sources are well known. In some embodiments, nucleic acids encoding the TCR can be obtained from a variety of sources, such as by polymerase chain reaction (PCR) amplification of TCR-encoding nucleic acids within or isolated from a given cell or cells, or synthesis of publicly available TCR DNA sequences. In some embodiments, the TCR is obtained from a biological source, such as from cells such as from a T cell (e.g. cytotoxic T cell), T cell hybridomas or other publicly available source. In some embodiments, the T cells can be obtained from in vivo isolated cells. In some embodiments, the T-cells can be a cultured T cell hybridoma or clone. In some embodiments, the TCR or antigen-binding portion thereof can be synthetically generated from knowledge of the sequence of the TCR. In some embodiments, a high-affinity T cell clone for a target antigen (e.g., a cancer antigen) is identified, isolated from a patient, and introduced into the cells. In some embodiments, the TCR clone for a target antigen has been generated in transgenic mice engineered with human immune system genes (e.g., the human leukocyte antigen system, or HLA). See, e.g., tumor antigens (see, e.g., Parkhurst et al. (2009) Clin Cancer Res. 15: 169-180 and Cohen et al. (2005) J Immunol. 175:5799-5808. In some embodiments, phage display is used to isolate TCRs against a target antigen (see, e.g., Varela-Rohena et al. (2008) Nat Med. 14: 1390-1395 and Li (2005) Nat Biotechnol. 23:349-354.

In some embodiments, the TCR or antigen-binding portion thereof is one that has been modified or engineered. In some embodiments, directed evolution methods are used to generate TCRs with altered properties, such as with higher affinity for a specific target. In some embodiments, directed evolution is achieved by display methods including, but not limited to, yeast display (Holler et al. (2003) Nat Immunol, 4, 55-62; Holler et al. (2000) Proc Natl Acad Sci USA, 97, 5387-92), phage display (Li et al. (2005) Nat Biotechnol, 23, 349-54), or T cell display (Chervin et al. (2008) J Immunol Methods, 339, 175-84). In some embodiments, display approaches involve engineering, or modifying, a known, parent or reference TCR. For example, in some cases, a wild-type TCR can be used as a template for producing mutagenized TCRs in which in one or more residues of the CDRs are mutated, and mutants with an desired altered property, such as higher affinity for a desired target antigen, are selected.

In some embodiments as described, the TCR can contain an introduced disulfide bond or bonds. In some embodiments, the native disulfide bonds are not present. In some embodiments, the one or more of the native cysteines (e.g. in the constant domain of the α chain and β chain) that form a native interchain disulfide bond are substituted with another residue, such as with a serine or alanine. In some embodiments, an introduced disulfide bond can be formed by mutating non-cysteine residues on the gamma and delta chains, such as in the constant domain of the gamma chain and delta chain, to cysteine. Exemplary non-native disulfide bonds of a TCR are described in published International PCT No. WO2006/000830 and WO2006/037960. In some embodiments, the presence of non-native cysteine residues (e.g. resulting in one or more non-native disulfide bonds) in a recombinant TCR can favor production of the desired recombinant TCR in a cell in which it is introduced over expression of a mismatched TCR pair containing a native TCR chain.

In some embodiments, the TCβ chains contain a transmembrane domain. In some embodiments, the transmembrane domain is positively charged. In some cases, the TCR chain contains a cytoplasmic tail. In some aspects, each chain (e.g. gamma or delta) of the TCR can possess one N-terminal immunoglobulin variable domain, one immunoglobulin constant domain, a transmembrane region, and a short cytoplasmic tail at the C-terminal end. In some embodiments, a TCR, for example via the cytoplasmic tail, is associated with invariant proteins of the CD3 complex involved in mediating signal transduction. In some cases, the structure allows the TCR to associate with other molecules like CD3 and subunits thereof. For example, a TCR containing constant domains with a transmembrane region may anchor the protein in the cell membrane and associate with invariant subunits of the CD3 signaling apparatus or complex. The intracellular tails of CD3 signaling subunits (e.g. CD3y, CD35, CD3s and CD3ζ chains) contain one or more immunoreceptor tyrosine-based activation motif or ITAM that are involved in the signaling capacity of the TCR complex.

In some embodiments, the TCR is a full-length TCR. In some embodiments, the TCR is an antigen-binding portion. In some embodiments, the TCR is a dimeric TCR (dTCR). In some embodiments, the TCR is a single-chain TCR (sc-TCR). A TCR may be cell-bound or in soluble form. In some embodiments, for purposes of the provided methods, the TCR is in cell-bound form expressed on the surface of a cell. In some embodiments a dTCR contains a first polypeptide wherein a sequence corresponding to a TCR γ chain variable region sequence is fused to the N terminus of a sequence corresponding to a TCR δ chain constant region extracellular sequence, and a second polypeptide wherein a sequence corresponding to a TCR γ chain variable region sequence is fused to the N terminus a sequence corresponding to a TCR δ chain constant region extracellular sequence, the first and second polypeptides being linked by a disulfide bond. In some embodiments, the bond can correspond to the native interchain disulfide bond present in native dimeric γδTCRs. In some embodiments, the interchain disulfide bonds are not present in a native TCR. For example, in some embodiments, one or more cysteines can be incorporated into the constant region extracellular sequences of dTCR polypeptide pair. In some cases, both a native and a non-native disulfide bond may be desirable. In some embodiments, the TCR contains a transmembrane sequence to anchor to the membrane. In some embodiments, a dTCR contains a TCR γ chain containing a variable γ domain, a constant γ domain and a first dimerization motif attached to the C-terminus of the constant γ domain, and a TCR δ chain comprising a variable S domain, a constant S domain and a first dimerization motif attached to the C-terminus of the constant S domain, wherein the first and second dimerization motifs easily interact to form a covalent bond between an amino acid in the first dimerization motif and an amino acid in the second dimerization motif linking the TCR γ chain and TCR δ chain together.

In some embodiments, any of the TCRs, including a dTCR, can be linked to signaling domains that yield an active TCR on the surface of a T cell. In some embodiments, the TCR is expressed on the surface of cells. In some embodiments, the TCR does contain a sequence corresponding to a transmembrane sequence. In some embodiments, the transmembrane domain can be a Ca or CP transmembrane domain. In some embodiments, the transmembrane domain can be from a non-TCR origin, for example, a transmembrane region from CD3z, CD28 or B7.1. In some embodiments, the TCR does contain a sequence corresponding to cytoplasmic sequences. In some embodiments, the TCR contains a CD3z signaling domain. In some embodiments, the TCR is capable of forming a TCR complex with CD3. In some embodiments, the TCR or antigen binding portion thereof may be a recombinantly produced natural protein or mutated form thereof in which one or more property, such as binding characteristic, has been altered. In some embodiments, a TCR may be derived from one of various animal species, such as human, mouse, rat, or other mammal.

In some embodiments, the TCR comprises affinity to a target antigen on a target cell. The target antigen may include any type of protein, or epitope thereof, associated with the target cell. For example, the TCR may comprise affinity to a target antigen on a target cell that indicates a particular disease state (e.g. cancer) of the target cell.

### F. Chimeric Antigen Receptors

The present invention provides compositions and methods for γδ T cell-based immunotherapies. In certain embodiments, the γδ T cell-based immunotherapy comprises a modified immune cell or precursor thereof, e.g., modified γδ T cell, comprising a chimeric antigen receptor (CAR). Thus, in some embodiments, the immune cell has been genetically modified to express the CAR. CARs of the present invention comprise an antigen binding domain, a transmembrane domain, and an intracellular domain.

The antigen binding domain may be operably linked to another domain of the CAR, such as the transmembrane domain or the intracellular domain, both described elsewhere herein, for expression in the cell. In one embodiment, a first nucleic acid sequence encoding the antigen binding domain is operably linked to a second nucleic acid encoding a transmembrane domain, and further operably linked to a third a nucleic acid sequence encoding an intracellular domain.

The antigen binding domains described herein can be combined with any of the transmembrane domains described herein, any of the intracellular domains or cytoplasmic domains described herein, or any of the other domains described herein that may be included in a CAR of the present invention. A subject CAR of the present invention may also include a hinge domain as described herein. A subject CAR of the present invention may also include a spacer domain as described herein. In some embodiments, each of the antigen binding domain, transmembrane domain, and intracellular domain is separated by a linker.

### Antigen Binding Domain

The antigen binding domain of a CAR is an extracellular region of the CAR for binding to a specific target antigen including proteins, carbohydrates, and glycolipids. In some embodiments, the CAR comprises affinity to a target antigen on a target cell. The target antigen may include any type of protein, or epitope thereof, associated with the target cell. For example, the CAR may comprise affinity to a target antigen on a target cell that indicates a particular disease state of the target cell.

In one embodiment, the target cell antigen is a tumor associated antigen (TAA). Examples of tumor associated antigens (TAAs), include but are not limited to, differentiation antigens such as MART-1/MelanA (MART-I), gp100 (Pmel 17), tyrosinase, TRP-1, TRP-2 and tumor-specific multilineage antigens such as MAGE-1, MAGE-3, BAGE, GAGE-1, GAGE-2, p15; overexpressed embryonic antigens such as CEA; overexpressed oncogenes and mutated tumor-suppressor genes such as p53, Ras, HER-2/neu; unique tumor antigens resulting from chromosomal translocations; such as BCR-ABL, E2A-PRL, H4-RET, IGH-IGK, MYL-RAR; and viral antigens, such as the Epstein Barr virus antigens EBVA and the human papillomavirus (HPV) antigens E6 and E7. Other large, protein-based antigens include TSP-180, MAGE-4, MAGE-5, MAGE-6, RAGE, NY-ESO, p185erbB2, p180erbB-3, c-met, nm-23H1, PSA, TAG-72, CA 19-9, CA 72-4, CAM 17.1, NuMa, K-ras, beta-Catenin, CDK4, Mum-1, p15, p16, 43-9F, 5T4, 791Tgp72, alpha-fetoprotein, beta-HCG, BCA225, BTAA, CA 125, CA 15-3CA 27.29BCAA, CA 195, CA 242, CA-50, CAM43, CD68P1, CO-029, FGF-5, G250, Ga733EpCAM, HTgp-175, M344, MA-50, MG7-Ag, MOV18, NB/70K, NY-CO-1, RCAS1, SDCCAGi6, TA-90Mac-2 binding proteincyclophilin C-associated protein, TAAL6, TAG72, TLP, and TPS. In a preferred embodiment, the antigen binding domain of the CAR targets an antigen that includes but is not limited to CD19, CD20, CD22, ROR1, Mesothelin, CD33/IL3Ra, c-Met, PSMA, PSCA, Glycolipid F77, EGFRvIII, GD-2, MY-ESO-1 TCR, MAGE A3 TCR, and the like.

Depending on the desired antigen to be targeted, the CAR of the invention can be engineered to include the appropriate antigen binding domain that is specific to the desired antigen target. For example, if CD19 is the desired antigen that is to be targeted, an antibody for CD19 can be used as the antigen bind moiety for incorporation into the CAR of the invention. This should not be construed as limiting in any way, as a CAR having affinity for any target antigen is suitable for use in a composition or method of the present invention.

As described herein, a CAR of the present disclosure having affinity for a specific target antigen on a target cell may comprise a target-specific binding domain. In some embodiments, the target-specific binding domain is a murine target-specific binding domain, e.g., the target-specific binding domain is of murine origin. In some embodiments, the target-specific binding domain is a human target-specific binding domain, e.g., the target-specific binding domain is of human origin. For example, a CAR of the present disclosure having affinity for CD19 on a target cell may comprise a CD19 binding domain.

In some embodiments, a CAR of the present disclosure may have affinity for one or more target antigens on one or more target cells. In some embodiments, a CAR may have affinity for one or more target antigens on a target cell. In such embodiments, the CAR is a bispecific CAR, or a multispecific CAR. In some embodiments, the CAR comprises one or more target-specific binding domains that confer affinity for one or more target antigens. In some embodiments, the CAR comprises one or more target-specific binding domains that confer affinity for the same target antigen. For example, a CAR comprising one or more target-specific binding domains having affinity for the same target antigen could bind distinct epitopes of the target antigen. When a plurality of target-specific binding domains is present in a CAR, the binding domains may be arranged in tandem and may be separated by linker peptides. For example, in a CAR comprising two target-specific binding domains, the binding domains are connected to each other covalently on a single polypeptide chain, through an oligo- or polypeptide linker, an Fc hinge region, or a membrane hinge region.

In some embodiments, the antigen binding domain is selected from the group consisting of an antibody, an antigen binding fragment (Fab), and a single-chain variable fragment (scFv). For example, a CD19 binding domain of the present invention can be selected from the group consisting of a CD19-specific antibody, a CD19-specific Fab, and a CD19-specific scFv. In one embodiment, a CD19 binding domain is a CD19-specific antibody. In one embodiment, a CD19 binding domain is a CD19-specific Fab. In one embodiment, a CD19 binding domain is a CD19-specific scFv (e.g. SEQ ID NO: 2).

The antigen binding domain can include any domain that binds to the antigen and may include, but is not limited to, a monoclonal antibody, a polyclonal antibody, a synthetic antibody, a human antibody, a humanized antibody, a non-human antibody, and any fragment thereof. In some embodiments, the antigen binding domain portion comprises a mammalian antibody or a fragment thereof. The choice of antigen binding domain may depend upon the type and number of antigens that are present on the surface of a target cell.

As used herein, the term “single-chain variable fragment” or “scFv” is a fusion protein of the variable regions of the heavy (VH) and light chains (VL) of an immunoglobulin (e.g., mouse or human) covalently linked to form a VH:VL heterodimer. The heavy (VH) and light chains (VL) are either joined directly or joined by a peptide-encoding linker, which connects the N-terminus of the VH with the C-terminus of the VL, or the C-terminus of the VH with the N-terminus of the VL. In some embodiments, the antigen binding domain (e.g., CD19 binding domain) comprises an scFv having the configuration from N-terminus to C-terminus, VH-linker-VL. In some embodiments, the antigen binding domain comprises an scFv having the configuration from N-terminus to C-terminus, VL-linker-VH. Those of skill in the art would be able to select the appropriate configuration for use in the present invention.

The linker is usually rich in glycine for flexibility, as well as serine or threonine for solubility. The linker can link the heavy chain variable region and the light chain variable region of the extracellular antigen-binding domain. Non-limiting examples of linkers are disclosed in Shen et al., Anal. Chem. 80(6):1910-1917 (2008) and WO 2014/087010, the contents of which are hereby incorporated by reference in their entireties. Various linker sequences are known in the art, including, without limitation, glycine serine (GS) linkers such as (GS)ₙ, (GSGGS)ₙ (SEQ ID NO:7), (GGGS)ₙ (SEQ ID NO:8), and (GGGGS)ₙ (SEQ ID NO:9), where n represents an integer of at least 1. Exemplary linker sequences can comprise amino acid sequences including, without limitation, GGSG (SEQ ID NO:10), GGSGG (SEQ ID NO:11), GSGSG (SEQ ID NO:12), GSGGG (SEQ ID NO:13), GGGSG (SEQ ID NO:14), GSSSG (SEQ ID NO:15), GGGGS (SEQ ID NO:16), GGGGSGGGGSGGGGS (SEQ ID NO:17) and the like. Those of skill in the art would be able to select the appropriate linker sequence for use in the present invention. In one embodiment, an antigen binding domain of the present invention comprises a heavy chain variable region (VH) and a light chain variable region (VL), wherein the VH and VL is separated by the linker sequence having the amino acid sequence GGGGSGGGGSGGGGS (SEQ ID NO:17), which may be encoded by the nucleic acid sequence GGTGGCGGTGGCTCGGGCGGTGGTGGGTCGGGTGGCGGCGGATCT (SEQ ID NO:18).

Despite removal of the constant regions and the introduction of a linker, scFv proteins retain the specificity of the original immunoglobulin. Single chain Fv polypeptide antibodies can be expressed from a nucleic acid comprising VH- and VL-encoding sequences as described by Huston, et al. (Proc. Nat. Acad. Sci. USA, 85:5879-5883, 1988). See, also, U.S. Pat. Nos. 5,091,513, 5,132,405 and 4,956,778; and U.S. Patent Publication Nos. 20050196754 and 20050196754. Antagonistic scFvs having inhibitory activity have been described (see, e.g., Zhao et al., Hyrbidoma (Larchmt) 2008 27(6):455-51; Peter et al., J Cachexia Sarcopenia Muscle 2012 Aug. 12; Shieh et al., J Imunol 2009 183(4):2277-85; Giomarelli et al., Thromb Haemost 2007 97(6):955-63; Fife eta., J Clin Invst 2006 116(8):2252-61; Brocks et al., Immunotechnology 1997 3(3):173-84; Moosmayer et al., Ther Immunol 1995 2 (10:31-40). Agonistic scFvs having stimulatory activity have been described (see, e.g., Peter et al., J Bioi Chem 2003 25278(38):36740-7; Xie et al., Nat Biotech 1997 15(8):768-71; Ledbetter et al., Crit Rev Immunol 1997 17 (5-6):427-55; Ho et al., Bio Chim Biophys Acta 2003 1638(3):257-66).

As used herein, “Fab” refers to a fragment of an antibody structure that binds to an antigen but is monovalent and does not have a Fc portion, for example, an antibody digested by the enzyme papain yields two Fab fragments and an Fc fragment (e.g., a heavy (H) chain constant region; Fc region that does not bind to an antigen).

As used herein, “F(ab′)2” refers to an antibody fragment generated by pepsin digestion of whole IgG antibodies, wherein this fragment has two antigen binding (ab′) (bivalent) regions, wherein each (ab′) region comprises two separate amino acid chains, a part of a H chain and a light (L) chain linked by an S—S bond for binding an antigen and where the remaining H chain portions are linked together. A “F(ab′)2” fragment can be split into two individual Fab′ fragments.

In some embodiments, the antigen binding domain may be derived from the same species in which the CAR will ultimately be used. For example, for use in humans, the antigen binding domain of the CAR may comprise a human antibody or a fragment thereof. In some embodiments, the antigen binding domain may be derived from a different species in which the CAR will ultimately be used. For example, for use in humans, the antigen binding domain of the CAR may comprise a murine antibody or a fragment thereof.

### Transmembrane Domain

CARs of the present invention may comprise a transmembrane domain that connects the antigen binding domain of the CAR to the intracellular domain of the CAR. The transmembrane domain of a subject CAR is a region that is capable of spanning the plasma membrane of a cell (e.g., an immune cell or precursor thereof). The transmembrane domain is for insertion into a cell membrane, e.g., a eukaryotic cell membrane. In some embodiments, the transmembrane domain is interposed between the antigen binding domain and the intracellular domain of a CAR.

In some embodiments, the transmembrane domain is naturally associated with one or more of the domains in the CAR. In some embodiments, the transmembrane domain can be selected or modified by one or more amino acid substitutions to avoid binding of such domains to the transmembrane domains of the same or different surface membrane proteins, to minimize interactions with other members of the receptor complex.

The transmembrane domain may be derived either from a natural or a synthetic source. Where the source is natural, the domain may be derived from any membrane-bound or transmembrane protein, e.g., a Type I transmembrane protein. Where the source is synthetic, the transmembrane domain may be any artificial sequence that facilitates insertion of the CAR into a cell membrane, e.g., an artificial hydrophobic sequence. Examples of the transmembrane domain of particular use in this invention include, without limitation, transmembrane domains derived from (i.e. comprise at least the transmembrane region(s) of) the alpha, beta or zeta chain of the T cell receptor, CD28, CD3 epsilon, CD45, CD4, CD5, CD7, CD8, CD9, CD16, CD22, CD33, CD37, CD64, CD80, CD86, CD134 (OX-40), CD137 (4-1BB), CD154 (CD40L), Toll-like receptor 1 (TLR1), TLR2, TLR3, TLR4, TLR5, TLR6, TLR7, TLR8, and TLR9. In some embodiments, the transmembrane domain may be synthetic, in which case it will comprise predominantly hydrophobic residues such as leucine and valine. Preferably a triplet of phenylalanine, tryptophan and valine will be found at each end of a synthetic transmembrane domain.

The transmembrane domains described herein can be combined with any of the antigen binding domains described herein, any of the intracellular domains described herein, or any of the other domains described herein that may be included in a subject CAR.

In some embodiments, the transmembrane domain further comprises a hinge region. A subject CAR of the present invention may also include a hinge region. The hinge region of the CAR is a hydrophilic region which is located between the antigen binding domain and the transmembrane domain. In some embodiments, this domain facilitates proper protein folding for the CAR. The hinge region is an optional component for the CAR. The hinge region may include a domain selected from Fc fragments of antibodies, hinge regions of antibodies, CH2 regions of antibodies, CH3 regions of antibodies, artificial hinge sequences or combinations thereof. Examples of hinge regions include, without limitation, a CD8a hinge, artificial hinges made of polypeptides which may be as small as, three glycines (Gly), as well as CH1 and CH3 domains of IgGs (such as human IgG4).

In some embodiments, a subject CAR of the present disclosure includes a hinge region that connects the antigen binding domain with the transmembrane domain, which, in turn, connects to the intracellular domain. The hinge region is preferably capable of supporting the antigen binding domain to recognize and bind to the target antigen on the target cells (see, e.g., Hudecek et al., Cancer Immunol. Res. (2015) 3(2): 125-135). In some embodiments, the hinge region is a flexible domain, thus allowing the antigen binding domain to have a structure to optimally recognize the specific structure and density of the target antigens on a cell such as tumor cell (Hudecek et al., supra). The flexibility of the hinge region permits the hinge region to adopt many different conformations.

In some embodiments, the hinge region is an immunoglobulin heavy chain hinge region. In some embodiments, the hinge region is a hinge region polypeptide derived from a receptor (e.g., a CD8-derived hinge region).

The hinge region can have a length of from about 4 amino acids to about 50 amino acids, e.g., from about 4 aa to about 10 aa, from about 10 aa to about 15 aa, from about 15 aa to about aa, from about 20 aa to about 25 aa, from about 25 aa to about 30 aa, from about 30 aa to about 40 aa, or from about 40 aa to about 50 aa. In some embodiments, the hinge region can have a length of greater than 5 aa, greater than 10 aa, greater than 15 aa, greater than 20 aa, greater than 25 aa, greater than 30 aa, greater than 35 aa, greater than 40 aa, greater than 45 aa, greater than 50 aa, greater than 55 aa, or more.

Suitable hinge regions can be readily selected and can be of any of a number of suitable lengths, such as from 1 amino acid (e.g., Gly) to 20 amino acids, from 2 amino acids to 15 amino acids, from 3 amino acids to 12 amino acids, including 4 amino acids to 10 amino acids, 5 amino acids to 9 amino acids, 6 amino acids to 8 amino acids, or 7 amino acids to 8 amino acids, and can be 1, 2, 3, 4, 5, 6, or 7 amino acids. Suitable hinge regions can have a length of greater than amino acids (e.g., 30, 40, 50, 60 or more amino acids).

For example, hinge regions include glycine polymers (G)ₙ, glycine-serine polymers (including, for example, (GS)ₙ, (GSGGS)ₙ (SEQ ID NO:7) and (GGGS)ₙ (SEQ ID NO:8), where n is an integer of at least one), glycine-alanine polymers, alanine-serine polymers, and other flexible linkers known in the art. Glycine and glycine-serine polymers can be used; both Gly and Ser are relatively unstructured, and therefore can serve as a neutral tether between components. Glycine polymers can be used; glycine accesses significantly more phi-psi space than even alanine, and is much less restricted than residues with longer side chains (see, e.g., Scheraga, Rev. Computational. Chem. (1992) 2: 73-142). Exemplary hinge regions can comprise amino acid sequences including, but not limited to, GGSG (SEQ ID NO:10), GGSGG (SEQ ID NO:11), GSGSG (SEQ ID NO:12), GSGGG (SEQ ID NO:13), GGGSG (SEQ ID NO:14), GSSSG (SEQ ID NO:15), and the like.

In some embodiments, the hinge region is an immunoglobulin heavy chain hinge region. Immunoglobulin hinge region amino acid sequences are known in the art; see, e.g., Tan et al., Proc. Natd. Acad. Sci. USA (1990) 87(1):162-166; and Huck et al., Nucleic Acids Res. (1986) 14(4): 1779-1789. The hinge region can comprise an amino acid sequence of a human IgG1, IgG2, IgG3, or IgG4, hinge region. In one embodiment, the hinge region can include one or more amino acid substitutions and/or insertions and/or deletions compared to a wild-type (naturally-occurring) hinge region. For example, His229 of human IgG1 hinge can be substituted with Tyr; see, e.g., Yan et al., J. Biol. Chem. (2012) 287: 5891-5897. In one embodiment, the hinge region can comprise an amino acid sequence derived from human CD8, or a variant thereof.

### Intracellular Signaling Domain

A subject CAR of the present invention also includes an intracellular signaling domain. The terms “intracellular signaling domain” and “intracellular domain” are used interchangeably herein. The intracellular signaling domain of the CAR is responsible for activation of at least one of the effector functions of the cell in which the CAR is expressed (e.g., immune cell). The intracellular signaling domain transduces the effector function signal and directs the cell (e.g., immune cell) to perform its specialized function, e.g., harming and/or destroying a target cell. Examples of an intracellular domain for use in the invention include, but are not limited to, the cytoplasmic portion of a surface receptor, co-stimulatory molecule, and any molecule that acts in concert to initiate signal transduction in the T cell, as well as any derivative or variant of these elements and any synthetic sequence that has the same functional capability.

Examples of the intracellular signaling domain include, without limitation, the ζ chain of the T cell receptor complex or any of its homologs, e.g., η chain, FcsRIγ and β chains, MB 1 (Iga) chain, B29 (Ig) chain, etc., human CD3 zeta chain, CD3 polypeptides (Δ, δ and ε), syk family tyrosine kinases (Syk, ZAP 70, etc.), src family tyrosine kinases (Lck, Fyn, Lyn, etc.), and other molecules involved in T cell transduction, such as CD2, CD5 and CD28. In one embodiment, the intracellular signaling domain may be human CD3 zeta chain (e.g. SEQ ID NO: 6), FcγRIII, FcsRI, cytoplasmic tails of Fc receptors, an immunoreceptor tyrosine-based activation motif (ITAM) bearing cytoplasmic receptors, and combinations thereof.

In one embodiment, the intracellular signaling domain of the CAR includes any portion of one or more co-stimulatory molecules, such as at least one signaling domain from CD2, CD3 (e.g. SEQ ID NO: 6), CD8, CD27, CD28, ICOS, 4-1BB (e.g. SEQ ID NO: 5), PD-1, any derivative or variant thereof, any synthetic sequence thereof that has the same functional capability, and any combination thereof.

Other examples of the intracellular domain include a fragment or domain from one or more molecules or receptors including, but not limited to, TCR, CD3 zeta, CD3 gamma, CD3 delta, CD3 epsilon, CD86, common FcR gamma, FcR beta (Fc Epsilon RIb), CD79a, CD79b, Fcgamma RIIa, DAP10, DAP12, T cell receptor (TCR), CD8, CD27, CD28, 4-1BB (CD137), OX9, OX40, CD30, CD40, PD-1, ICOS, a KIR family protein, lymphocyte function-associated antigen-1 (LFA-1), CD2, CD7, LIGHT, NKG2C, B7-H3, a ligand that specifically binds with CD83, CDS, ICAM-1, GITR, BAFFR, HVEM (LIGHTR), SLAMF7, NKp80 (KLRF1), CD127, CD160, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CDlib, ITGAX, CD11c, ITGB1, CD29, ITGB2, CD18, LFA-1, ITGB7, TNFR2, TRANCE/RANKL, DNAM1 (CD226), SLAMF4 (CD244, 2B4), CD84, CD96 (Tactile), CEACAMI, CRT AM, Ly9 (CD229), CD160 (BY55), PSGL1, CD100 (SEMA4D), CD69, SLAMF6 (NTB-A, Ly108), SLAM (SLAMFI, CD150, IPO-3), BLAME (SLAMF8), SELPLG (CD162), LTBR, LAT, GADS, SLP-76, PAG/Cbp, NKp44, NKp30, NKp46, NKG2D, Toll-like receptor 1 (TLR1), TLR2, TLR3, TLR4, TLR5, TLR6, TLR7, TLR8, TLR9, other co-stimulatory molecules described herein, any derivative, variant, or fragment thereof, any synthetic sequence of a co-stimulatory molecule that has the same functional capability, and any combination thereof.

Additional examples of intracellular domains include, without limitation, intracellular signaling domains of several types of various other immune signaling receptors, including, but not limited to, first, second, and third generation T cell signaling proteins including CD3, B7 family costimulatory, and Tumor Necrosis Factor Receptor (TNFR) superfamily receptors (see, e.g., Park and Brentjens, J. Clin. Oncol. (2015) 33(6): 651-653). Additionally, intracellular signaling domains may include signaling domains used by NK and NKT cells (see, e.g., Hermanson and Kaufman, Front. Immunol. (2015) 6: 195) such as signaling domains of NKp30 (B7-H6) (see, e.g., Zhang et al., J. Immunol. (2012) 189(5): 2290-2299), and DAP 12 (see, e.g., Topfer et al., J. Immunol. (2015) 194(7): 3201-3212), NKG2D, NKp44, NKp46, DAP10, and CD3z.

Intracellular signaling domains suitable for use in a subject CAR of the present invention include any desired signaling domain that provides a distinct and detectable signal (e.g., increased production of one or more cytokines by the cell; change in transcription of a target gene; change in activity of a protein; change in cell behavior, e.g., cell death; cellular proliferation; cellular differentiation; cell survival; modulation of cellular signaling responses; etc.) in response to activation of the CAR (i.e., activated by antigen and dimerizing agent). In some embodiments, the intracellular signaling domain includes at least one (e.g., one, two, three, four, five, six, etc.) ITAM motifs as described below. In some embodiments, the intracellular signaling domain includes DAP10/CD28 type signaling chains. In some embodiments, the intracellular signaling domain is not covalently attached to the membrane bound CAR, but is instead diffused in the cytoplasm.

Intracellular signaling domains suitable for use in a subject CAR of the present invention include immunoreceptor tyrosine-based activation motif (ITAM)-containing intracellular signaling polypeptides. In some embodiments, an ITAM motif is repeated twice in an intracellular signaling domain, where the first and second instances of the ITAM motif are separated from one another by 6 to 8 amino acids. In one embodiment, the intracellular signaling domain of a subject CAR comprises 3 ITAM motifs.

In some embodiments, intracellular signaling domains includes the signaling domains of human immunoglobulin receptors that contain immunoreceptor tyrosine based activation motifs (ITAMs) such as, but not limited to, FcgammaRI, FcgammaRIIA, FcgammaRIIC, FcgammaRIIIA, FcRL5 (see, e.g., Gillis et al., Front. Immunol. (2014) 5:254).

A suitable intracellular signaling domain can be an ITAM motif-containing portion that is derived from a polypeptide that contains an ITAM motif. For example, a suitable intracellular signaling domain can be an ITAM motif-containing domain from any ITAM motif-containing protein. Thus, a suitable intracellular signaling domain need not contain the entire sequence of the entire protein from which it is derived. Examples of suitable ITAM motif-containing polypeptides include, but are not limited to: DAP12, FCER1G (Fc epsilon receptor I gamma chain), CD3D (CD3 delta), CD3E (CD3 epsilon), CD3G (CD3 gamma), CD3Z (CD3 zeta), and CD79A (antigen receptor complex-associated protein alpha chain).

In one embodiment, the intracellular signaling domain is derived from DAP12 (also known as TYROBP; TYRO protein tyrosine kinase binding protein; KARAP; PLOSL; DNAX-activation protein 12; KAR-associated protein; TYRO protein tyrosine kinase-binding protein; killer activating receptor associated protein; killer-activating receptor-associated protein; etc.). In one embodiment, the intracellular signaling domain is derived from FCER1G (also known as FCRG; Fc epsilon receptor I gamma chain; Fc receptor gamma-chain; fc-epsilon RI-gamma; fcRgamma; fceR1 gamma; high affinity immunoglobulin epsilon receptor subunit gamma; immunoglobulin E receptor, high affinity, gamma chain; etc.). In one embodiment, the intracellular signaling domain is derived from T-cell surface glycoprotein CD3 delta chain (also known as CD3D; CD3-DELTA; T3D; CD3 antigen, delta subunit; CD3 delta; CD3d antigen, delta polypeptide (TiT3 complex); OKT3, delta chain; T-cell receptor T3 delta chain; T-cell surface glycoprotein CD3 delta chain; etc.). In one embodiment, the intracellular signaling domain is derived from T-cell surface glycoprotein CD3 epsilon chain (also known as CD3e, T-cell surface antigen T3/Leu-4 epsilon chain, T-cell surface glycoprotein CD3 epsilon chain, AI504783, CD3, CD3epsilon, T3e, etc.). In one embodiment, the intracellular signaling domain is derived from T-cell surface glycoprotein CD3 gamma chain (also known as CD3G, T-cell receptor T3 gamma chain, CD3-GAMMA, T3G, gamma polypeptide (TiT3 complex), etc.). In one embodiment, the intracellular signaling domain is derived from T-cell surface glycoprotein CD3 zeta chain (also known as CD3Z, T-cell receptor T3 zeta chain, CD247, CD3-ZETA, CD3H, CD3Q, T3Z, TCRZ, etc.). In one embodiment, the intracellular signaling domain is derived from CD79A (also known as B-cell antigen receptor complex-associated protein alpha chain; CD79a antigen (immunoglobulin-associated alpha); MB-1 membrane glycoprotein; ig-alpha; membrane-bound immunoglobulin-associated protein; surface IgM-associated protein; etc.). In one embodiment, an intracellular signaling domain suitable for use in an FN3 CAR of the present disclosure includes a DAP10/CD28 type signaling chain. In one embodiment, an intracellular signaling domain suitable for use in an FN3 CAR of the present disclosure includes a ZAP70 polypeptide. In some embodiments, the intracellular signaling domain includes a cytoplasmic signaling domain of TCR zeta, FcR gamma, FcR beta, CD3 gamma, CD3 delta, CD3 epsilon, CD5, CD22, CD79a, CD79b, or CD66d. In one embodiment, the intracellular signaling domain in the CAR includes a cytoplasmic signaling domain of human CD3 zeta.

While usually the entire intracellular signaling domain can be employed, in many cases it is not necessary to use the entire chain. To the extent that a truncated portion of the intracellular signaling domain is used, such truncated portion may be used in place of the intact chain as long as it transduces the effector function signal. The intracellular signaling domain includes any truncated portion of the intracellular signaling domain sufficient to transduce the effector function signal.

The intracellular signaling domains described herein can be combined with any of the antigen binding domains described herein, any of the transmembrane domains described herein, or any of the other domains described herein that may be included in the CAR.

The invention should be construed to include any CAR known in the art and/or disclosed herein. Exemplary CARs include, but are not limited to, those disclosed herein, those disclosed in U.S. Ser. No. 10/357,514B2, U.S. Ser. No. 10/221,245B2, U.S. Ser. No. 10/603,378B2, U.S. Pat. No. 8,916,381B1, U.S. Pat. No. 9,394,368B2, US20140050708A1, U.S. Pat. No. 9,598,489B2, U.S. Pat. No. 9,365,641B2, US20210079059A1, U.S. Pat. No. 9,783,591B2, WO2016028896A1, U.S. Pat. No. 9,446,105B2, WO2016014576A1, US20210284752A1, WO2016014565A2, WO2016014535A1, and U.S. Pat. No. 9,272,002B2, and any other CAR generally disclosed in the art.

Exemplary CAR sequences include, but are not limited to, the following:

Anti-human-CD19-BBZ CAR sequence (Full Length):
(SEQ ID NO: 1)
atggccttaccagtgaccgccttgctcctgccgctggccttgctgctccacgccgccaggccggacatccagatgacacagactacatcct
ccctgtctgcctctctgggagacagagtcaccatcagttgcagggcaagtcaggacattagtaaatatttaaattggtatcagcagaaaccag
atggaactgttaaactcctgatctaccatacatcaagattacactcaggagtcccatcaaggttcagtggcagtgggtctggaacagattattct
ctcaccattagcaacctggagcaagaagatattgccacttacttttgccaacagggtaatacgcttccgtacacgttcggaggggggaccaa
gctggagatcacaggtggcggtggctcgggcggtggtgggtcgggtggcggcggatctgaggtgaaactgcaggagtcaggacctggc
ctggtggcgccctcacagagcctgtccgtcacatgcactgtctcaggggtctcattacccgactatggtgtaagctggattcgccagcctcca
cgaaagggtctggagtggctgggagtaatatggggtagtgaaaccacatactataattcagctctcaaatccagactgaccatcatcaagga
caactccaagagccaagttttcttaaaaatgaacagtctgcaaactgatgacacagccatttactactgtgccaaacattattactacggtggta
gctatgctatggactactggggccaaggaacctcagtcaccgtctcctcaaccacgacgccagcgccgcgaccaccaacaccggcgccc
accatcgcgtcgcagcccctgtccctgcgcccagaggcgtgccggccagcggcggggggcgcagtgcacacgagggggctggacttc
gcctgtgatatctacatctgggcgcccttggccgggacttgtggggtccttctcctgtcactggttatcaccctttactgcaaacggggcagaa
agaaactcctgtatatattcaaacaaccatttatgagaccagtacaaactactcaagaggaagatggctgtagctgccgatttccagaagaag
aagaaggaggatgtgaactgagagtgaagttcagcaggagcgcagacgcccccgcgtacaagcagggccagaaccagctctataacga
gctcaatctaggacgaagagaggagtacgatgttttggacaagagacgtggccgggaccctgagatggggggaaagccgagaaggaa
gaaccctcaggaaggcctgtacaatgaactgcagaaagataagatggcggaggcctacagtgagattgggatgaaaggcgagcgccgg
aggggcaaggggcacgatggcctttaccagggtctcagtacagccaccaaggacacctacgacgcccttcacatgcaggccctgccccc
tcgc
Anti-human-CD19-BBZ CAR sequence (scFv):
(SEQ ID NO: 2)
gacatccagatgacacagactacatcctccctgtctgcctctctgggagacagagtcaccatcagttgcagggcaagtcaggacattagtaa
atatttaaattggtatcagcagaaaccagatggaactgttaaactcctgatctaccatacatcaagattacactcaggagtcccatcaaggttca
gtggcagtgggtctggaacagattattctctcaccattagcaacctggagcaagaagatattgccacttacttttgccaacagggtaatacgct
tccgtacacgttcggaggggggaccaagctggagatcacaggtggcggtggctcgggcggtggtgggtcgggtggcggcggatctgag
gtgaaactgcaggagtcaggacctggcctggtggcgccctcacagagcctgtccgtcacatgcactgtctcaggggtctcattacccgact
atggtgtaagctggattcgccagcctccacgaaagggtctggagtggctgggagtaatatggggtagtgaaaccacatactataattcagct
ctcaaatccagactgaccatcatcaaggacaactccaagagccaagttttcttaaaaatgaacagtctgcaaactgatgacacagccatttact
actgtgccaaacattattactacggtggtagctatgctatggactactggggccaaggaacctcagtcaccgtctcctca
Anti-human-CD19-BBZ CAR sequence (CD8 Leader):
(SEQ ID NO: 3)
atggccttaccagtgaccgccttgctcctgccgctggccttgctgctccacgccgccaggccg
Anti-human-CD19-BBZ CAR sequence (CD8 Hinge and transmembrane domain):
(SEQ ID NO: 4)
Accacgacgccagcgccgcgaccaccaacaccggcgcccaccatcgcgtcgcagcccctgtccctgcgcccagaggcgtgccggcc
agcggggggggcgcagtgcacacgagggggctggacttcgcctgtgatatctacatctgggcgcccttggccgggacttgtggggtcc
ttctcctgtcactggttatcaccctttactgc
Anti-human-CD19-BBZ CAR sequence (4-1BB intracellular domain):
(SEQ ID NO: 5)
aaacggggcagaaagaaactcctgtatatattcaaacaaccatttatgagaccagtacaaactactcaagaggaagatggctgtagctgccg
atttccagaagaagaagaaggaggatgtgaactg
Anti-human-CD19-BBZ CAR sequence (CD3ζ intracellular domain):
(SEQ ID NO: 6)
agagtgaagttcagcaggagcgcagacgcccccgcgtacaagcagggccagaaccagctctataacgagctcaatctaggacgaagag
aggagtacgatgttttggacaagagacgtggccgggaccctgagatggggggaaagccgagaaggaagaaccctcaggaaggcctgta
caatgaactgcagaaagataagatggcggaggcctacagtgagattgggatgaaaggcgagcgccggaggggcaaggggcacgatgg
cctttaccagggtctcagtacagccaccaaggacacctacgacgcccttcacatgcaggccctgccccctgc

### G. Pharmaceutical Compositions

Also provided are composition comprising populations of γδ T cells for use in γδ T cell immunotherapy. The population of cells can be generated by any of the methods contemplated herein. The cells can include further modification, such as inclusion of a chimeric antigen receptor (CAR), a modified or exogeneous T cell receptor (TCR). Among the compositions are pharmaceutical compositions and formulations for administration, such as for adoptive cell therapy. Also provided are therapeutic methods for administering the cells and compositions to subjects, e.g., patients.

Also provided are compositions including the cells for administration, including pharmaceutical compositions and formulations, such as unit dose form compositions including the number of cells for administration in a given dose or fraction thereof. The pharmaceutical compositions and formulations generally include one or more optional pharmaceutically acceptable carrier or excipient. In some embodiments, the composition includes at least one additional therapeutic agent.

The term “pharmaceutical formulation” refers to a preparation which is in such form as to permit the biological activity of an active ingredient contained therein to be effective, and which contains no additional components which are unacceptably toxic to a subject to which the formulation would be administered. A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative. In some aspects, the choice of carrier is determined in part by the particular cell and/or by the method of administration. Accordingly, there are a variety of suitable formulations. For example, the pharmaceutical composition can contain preservatives. Suitable preservatives may include, for example, methylparaben, propylparaben, sodium benzoate, and benzalkonium chloride. In some aspects, a mixture of two or more preservatives is used. The preservative or mixtures thereof are typically present in an amount of about 0.0001% to about 2% by weight of the total composition. Carriers are described, e.g., by Remington's Pharmaceutical Sciences 16th edition, Osol, A. Ed. (1980). Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG).

Buffering agents in some aspects are included in the compositions. Suitable buffering agents include, for example, citric acid, sodium citrate, phosphoric acid, potassium phosphate, and various other acids and salts. In some aspects, a mixture of two or more buffering agents is used. The buffering agent or mixtures thereof are typically present in an amount of about 0.001% to about 4% by weight of the total composition. Methods for preparing administrable pharmaceutical compositions are known. Exemplary methods are described in more detail in, for example, Remington: The Science and Practice of Pharmacy, Lippincott Williams & Wilkins; 21st ed. (May 1, 2005).

The formulations can include aqueous solutions. The formulation or composition may also contain more than one active ingredient useful for the particular indication, disease, or condition being treated with the cells, preferably those with activities complementary to the cells, where the respective activities do not adversely affect one another. Such active ingredients are suitably present in combination in amounts that are effective for the purpose intended. Thus, in some embodiments, the pharmaceutical composition further includes other pharmaceutically active agents or drugs, such as chemotherapeutic agents, e.g., asparaginase, busulfan, carboplatin, cisplatin, daunorubicin, doxorubicin, fluorouracil, gemcitabine, hydroxyurea, methotrexate, paclitaxel, rituximab, vinblastine, and/or vincristine. The pharmaceutical composition in some embodiments contains the cells in amounts effective to treat or prevent the disease or condition, such as a therapeutically effective or prophylactically effective amount. Therapeutic or prophylactic efficacy in some embodiments is monitored by periodic assessment of treated subjects. The desired dosage can be delivered by a single bolus administration of the cells, by multiple bolus administrations of the cells, or by continuous infusion administration of the cells.

Formulations include those for oral, intravenous, intraperitoneal, subcutaneous, pulmonary, transdermal, intramuscular, intranasal, buccal, sublingual, or suppository administration. In some embodiments, the cell populations are administered parenterally. The term “parenteral,” as used herein, includes intravenous, intramuscular, subcutaneous, rectal, vaginal, and intraperitoneal administration. In some embodiments, the cells are administered to the subject using peripheral systemic delivery by intravenous, intraperitoneal, or subcutaneous injection. Compositions in some embodiments are provided as sterile liquid preparations, e.g., isotonic aqueous solutions, suspensions, emulsions, dispersions, or viscous compositions, which may in some aspects be buffered to a selected pH. Liquid preparations are normally easier to prepare than gels, other viscous compositions, and solid compositions. Additionally, liquid compositions are somewhat more convenient to administer, especially by injection. Viscous compositions, on the other hand, can be formulated within the appropriate viscosity range to provide longer contact periods with specific tissues. Liquid or viscous compositions can comprise carriers, which can be a solvent or dispersing medium containing, for example, water, saline, phosphate buffered saline, polyoi (for example, glycerol, propylene glycol, liquid polyethylene glycol) and suitable mixtures thereof.

Sterile injectable solutions can be prepared by incorporating the cells in a solvent, such as in admixture with a suitable carrier, diluent, or excipient such as sterile water, physiological saline, glucose, dextrose, or the like. The compositions can contain auxiliary substances such as wetting, dispersing, or emulsifying agents (e.g., methylcellulose), pH buffering agents, gelling or viscosity enhancing additives, preservatives, flavoring agents, and/or colors, depending upon the route of administration and the preparation desired. Standard texts may in some aspects be consulted to prepare suitable preparations.

Various additives which enhance the stability and sterility of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, and sorbic acid. Prolonged absorption of the injectable pharmaceutical form can be brought about by the use of agents delaying absorption, for example, aluminum monostearate and gelatin.

The formulations to be used for in vivo administration are generally sterile. Sterility may be readily accomplished, e.g., by filtration through sterile filtration membranes.

The contents of the articles, patents, and patent applications, and all other documents and electronically available information mentioned or cited herein, are hereby incorporated by reference in their entirety to the same extent as if each individual publication was specifically and individually indicated to be incorporated by reference. Applicants reserve the right to physically incorporate into this application any and all materials and information from any such articles, patents, patent applications, or other physical and electronic documents.

While the present invention has been described with reference to the specific embodiments thereof, it should be understood by those skilled in the art that various changes may be made and equivalents may be substituted without departing from the true spirit and scope of the invention. It will be readily apparent to those skilled in the art that other suitable modifications and adaptations of the methods described herein may be made using suitable equivalents without departing from the scope of the embodiments disclosed herein. In addition, many modifications may be made to adapt a particular situation, material, composition of matter, process, process step or steps, to the objective, spirit and scope of the present invention. All such modifications are intended to be within the scope of the claims appended hereto. Having now described certain embodiments in detail, the same will be more clearly understood by reference to the following examples, which are included for purposes of illustration only and are not intended to be limiting.

### EXPERIMENTAL EXAMPLES

The invention is now described with reference to the following Examples. These Examples are provided for the purpose of illustration only, and the invention is not limited to these Examples, but rather encompasses all variations that are evident as a result of the teachings provided herein.

### Materials and Methods Corresponding to Examples 1-9

Isolation of PBMCs. Peripheral blood samples were collected from apheresis circuit of 43 healthy blood donors (23 male, 20 female, age range 21-55 year) through Human Immunology Core at the University of Pennsylvania with informed consent and IRB approval (Protocol number 707906).

Expansion of γδ T cells ex vivo. PBMC were plated at 2×10⁶ cells per well in 24-well flat-bottomed plates at 37° C. in a humidified atmosphere of 5% CO₂. The standard culture medium contains RPMI 1640 (Gibco), containing 10% FBS (Gibco), 2 mM L-glutamine (Gibco), 100 U/ml penicillin (Gibco), streptomycin (Gibco) and 200 IU/ml IL-2 (PeproTech, Rocky Hill, NJ) (Castella et al., Nature communications. 2017; 8:15663.) 5 μM zoledronic acid (Sigma-Aldrich, St. Louis, MO USA) was added into the culture medium at the first day and the medium containing IL-2 changed every two days. In some cases, 24-well plates were pre-coated with 0.5 μg/ml anti-γδTCR Ab (Immunotech, Beckman Coulter, Fullerton, CA) and then PBMCs were added to the anti-γδTCR Ab-coated wells and cultured in the standard culture medium mentioned above. The viability and cell number of γδ T cells were evaluated by the trypan blue staining assay, and the percentage of γδ T cells counted by flow cytometry (Ryan et al., Proc NatlAcadSci USA. 2016; 113(50):14378-83).

Antibodies and flow cytometry analyses. Fluorochrome-conjugated antibodies were from eBioscience or BioLegend: CD3 (OKT3), CD4 (OKT4), CD14 (63D3), CD11b (ICRF44), CD19 (HIB19), CD27 (LG.3A10), CD45RA (HI100), CD107a (H4A3), TCR γ/6 (B1), Vδ2 (B6), Vδ1 (TS8.2), Granzyme B (QA16A02), IFN-γ (B27), PD-1 (EH12.1), CTLA4 (BNI3), T-bet (4B10), Eomes (WD1928), CD80 (2D10) and CD86 (IT2.2). For surface markers, expanded Vγ9Vδ2 T cells or PBMCs were stained with diluted fluorochrome-conjugated antibodies on ice and washed with FACS buffer; and for intracellular biomarkers, cells were permeabilized and stained with antibodies diluted in permeabilization buffer (BioLegend, San Diego, CA). Samples were acquired using a LSR-A flow cytometer (BD Biosciences, Baltimore, MD) and analyzed using FlowJo software (Tree Star Inc., Ashland, OR).

Cytotoxicity assays. The CytoTox 96® Non-Radioactive Cytotoxicity Assay (Promega, Madison, WI) was applied to measure the cytotoxicity of expanded Vγ9Vδ2 T cells. In this respect, A375 (2×10⁴ cells/well), A2058 (2×10⁴ cells/well), and 903 (2×10⁴ cells/well) melanoma cells were used as target cells and seeded into a 96-round well plate, and expanded Vγ9Vδ2 T cells (1×10⁵ cells/well) served as effector cells were directly added to individual wells incubated overnight. The supernatants were collected and the lactate dehydrogenase activity was detected. Controls for spontaneous lactate dehydrogenase release from effector and target cells, the target maximum release, as well as the culture medium background were measured simultaneously (Zhou et al., Cell Mol Immunol. 2012; 9(1):34-44).

Chemotaxis assays. Chemotaxis assays of expanded Vγ9Vδ2 T cells were performed using 24-well transwell plates with 5-μm pore polycarbonate membrane inserts (Costar; Corning, NY). Briefly, expanded Vγ9Vδ2 T cells (2×10⁵ cell/well) suspended in 200 μl RPMI 1640 containing 0.5% FBS were placed in the upper chamber, and RPMI 1640 (600 l) containing 10% FBS was added to the lower chamber of the transwell. The cells that migrated into the lower chambers and those left in the upper chambers were collected and counted separately after 8 h incubation at 37° C., 5% CO2.

3-D Spheroid Assays. The 96-well plate was pre-coated with 50 μL 1.5% Agarose (LONZA, Rockland, ME). Melanoma cells (A2058 and UACC 903 cells) were labeled with CellTracker™ Orange CMRA Dye (Thermo Fisher Scientific, Waltham, MA) and seeded at 2×10⁴ cells/well and allowed to form spheroids over 48 hours. Before starting the co-culture, PBMCs were stained with CFSE (Thermo Fisher Scientific) according to manufacturer procedure. Infiltrated spheroids were analyzed at 48 h using confocal microscope (Leica, Wetzlar, Germany). Spheroids were divided into single cells and CFSE+ cells infiltrating the spheroids was counted by flow cytometry.

Patient-derived melanoma organoid assays. Human melanoma samples were received from the Hospital of the University of Pennsylvania. This study was approved by the Institution Review Board at the University of Pennsylvania. Fresh tumor specimens were washed with PBS (containing 2% penicillin/streptomycin (P/S)) and minced into pieces of 1 mm. Fragments were enzymatically digested in DMEM (4.5 mmol/L glucose, 2% FBS, 100 U/mL collagenase type IV (200 U/ml, Sigma-Aldrich), DNase I (50 U/ml, Sigma-Aldrich) for 20 min at 37° C. Samples were diluted in 25 ml media and pelleted, and resuspended in fresh DMEM and filtered over 100-μm strainers. Red blood cells were removed using RBC lysis buffer (Biolegend). Matrigel (Corning) and complete DMEM/F12 medium mixed at 1:2 ratio and pre-seeded on 24 well plates to solidification. The cell mixture seeded on the Matrigel layer and cultured for up to 10 days, and the medium changed every 3 days. The culture medium was ADMEM/F12 contained of 1% P/S, 50% Wnt3a, RSPO1, Noggin-conditioned media (L-WRN, ATCC), Nicotinamide (10 mM, Sigma), N-Acetylcysteine (1 mM, Sigma), B-27 without vitamin A (1×, Invitrogen), A83-01 (0.5 mM, Tocris), EGF (50 ng/mL, Invitrogen) FGF (50 ng/ml, Peprotech) and Forskolin (10 ng/ml, Tocris).

Live/Dead Staining Dual labeling was performed using ViaStain AOPI Staining Solution (Nexcelom, CS2-0106) following γδ T cells co-cultured with organoids for 48 h. The images were captured on Zeiss LSM 710 (Carl Zeiss, Thornwood, New York) after cells incubation with the dyes for 20 minutes at room temperature in the dark. Images were then analyzed using the ZEN software.

Apoptosis of cells in the organoids was measured after γδ T cells and organoids co-culture for 48 h. Organoids were washed with cold PBS to remove the Matrigel and dissociated to single cells by Trypsin (Gibco). Cells were washed twice with cold PBS, and resuspended in 100 l Annexin V Binding Buffer (FITC Annexin V Apoptosis Detection Kit, Biolegend). 5 μl of AV-FITC and 5 μl of 7-AAD solution were added and incubated for 15 min at room temperature in the dark. FACS was performed to measure AV and 7-AAD, yielding AV+/7-AAD− early apoptotic cells and AV+/7-AAD+ late apoptotic/necrotic cells.

The cytotoxicity assays of γδ T cells against organoids were also performed by the CytoTox 96® Non-Radioactive Cytotoxicity Assay. The lactate dehydrogenase activity released by organoids was detected and analyzed.

Statistics. Data are analyzed by Prism 8.0 (GraphPad Software, San Diego, CA) and R software. The significant differences between two groups were analyzed by Mann-Whitney non-parametric test for unpaired samples and several groups were assessed by one-way ANOVA with multiple comparisons. Correlation analyses were performed with the non-parametric Spearman Rank Order test. P values<0.05 were considered significant difference (*P<0.05; **P<0.01). Logistic regression models were applied to explore if initial γδ T cell proportion, each surface marker expression of γδ T cell or their combination would be useful to predict whether γδ T cells can be well expanded ≥50% in an individual blood sample. Receiver Operating Characteristic (ROC) curves were generated based on the value of initial γδ T cell proportion or Index obtained from multivariate models. Area under the curve (AUC) of each model's ROC with the optimal sensitivity and specificity were calculated. Delong's test was applied to determine the difference on AUC between regression models. A total of 66 samples have complete data. Due to such small sample size, complex multivariate predictive models were not considered for the data analysis.

### Example 1: Significant Interindividual Heterogeneity in γδT Cell Expansion

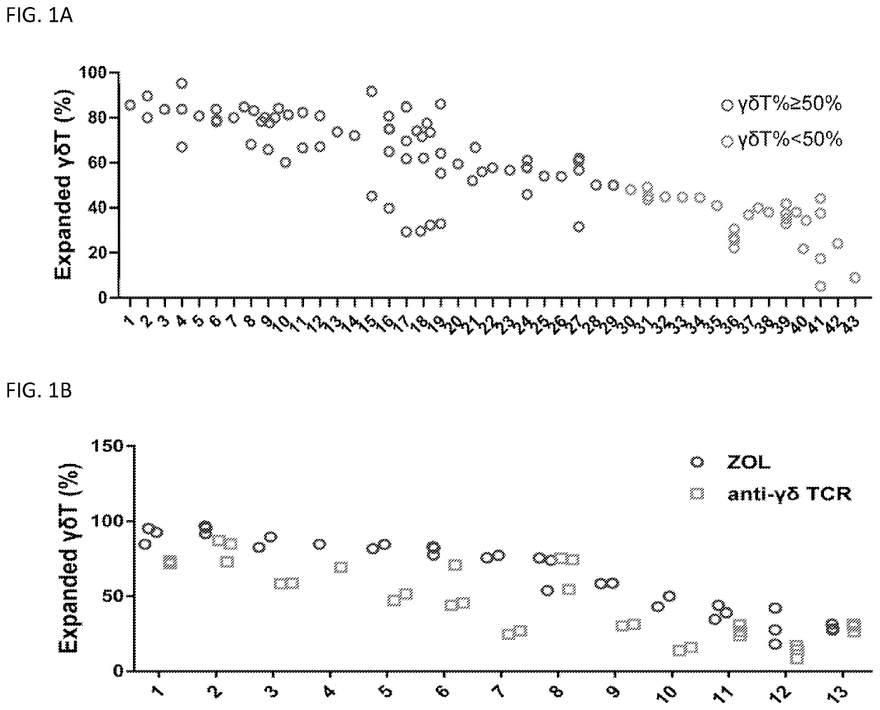

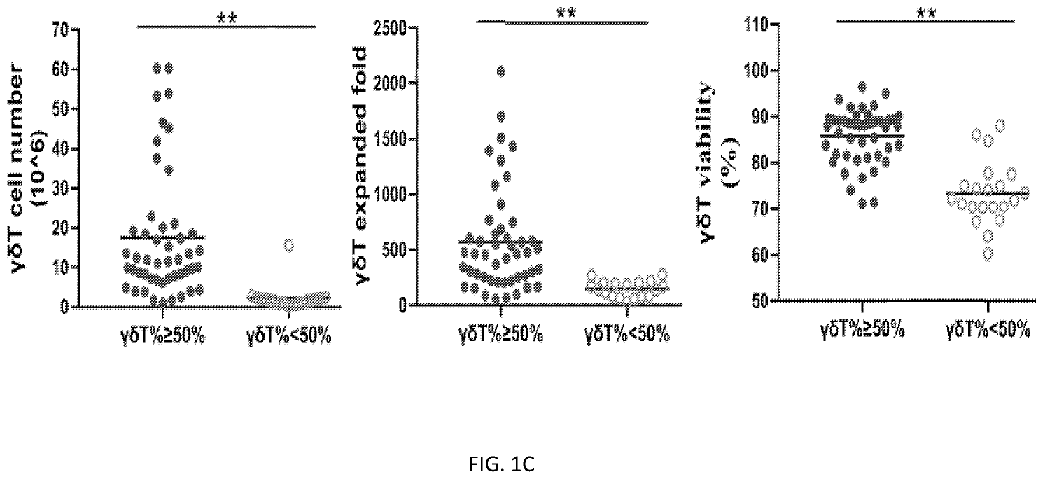

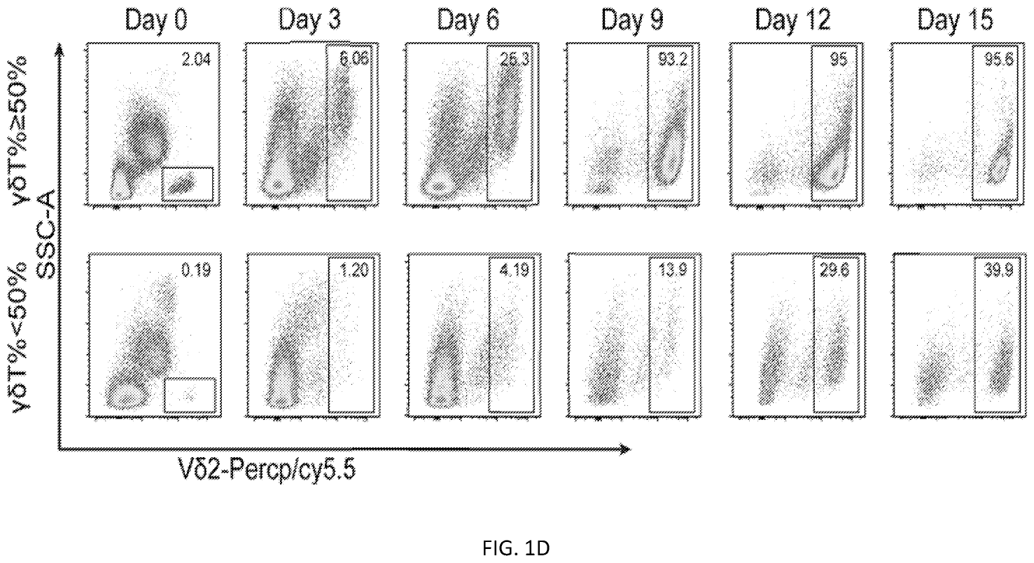

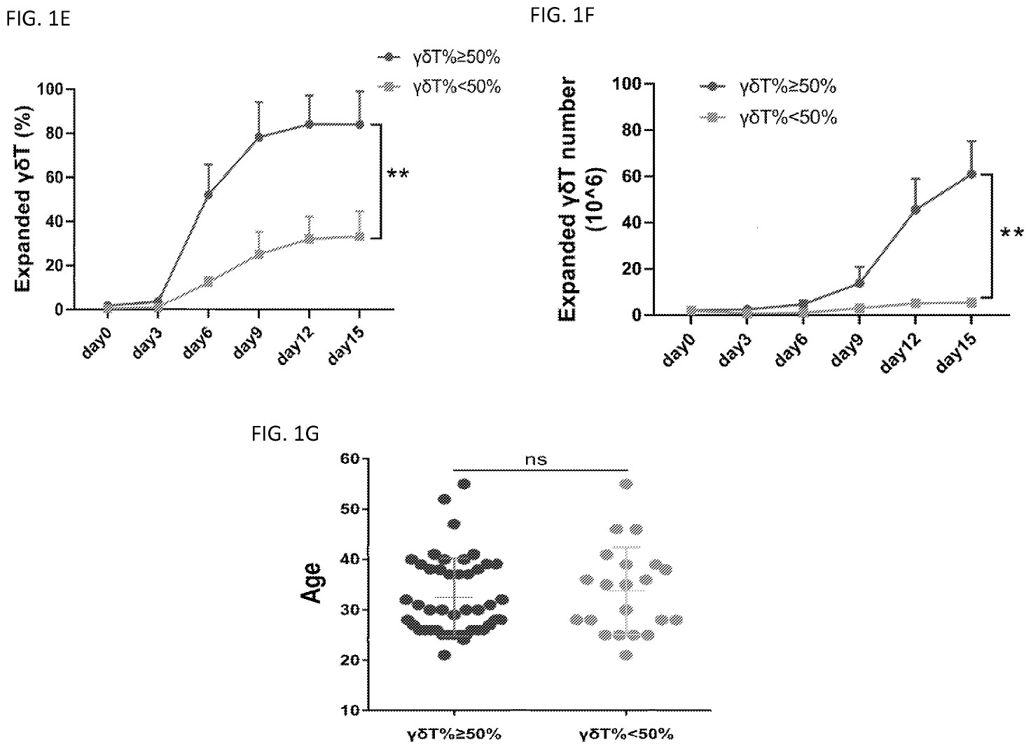

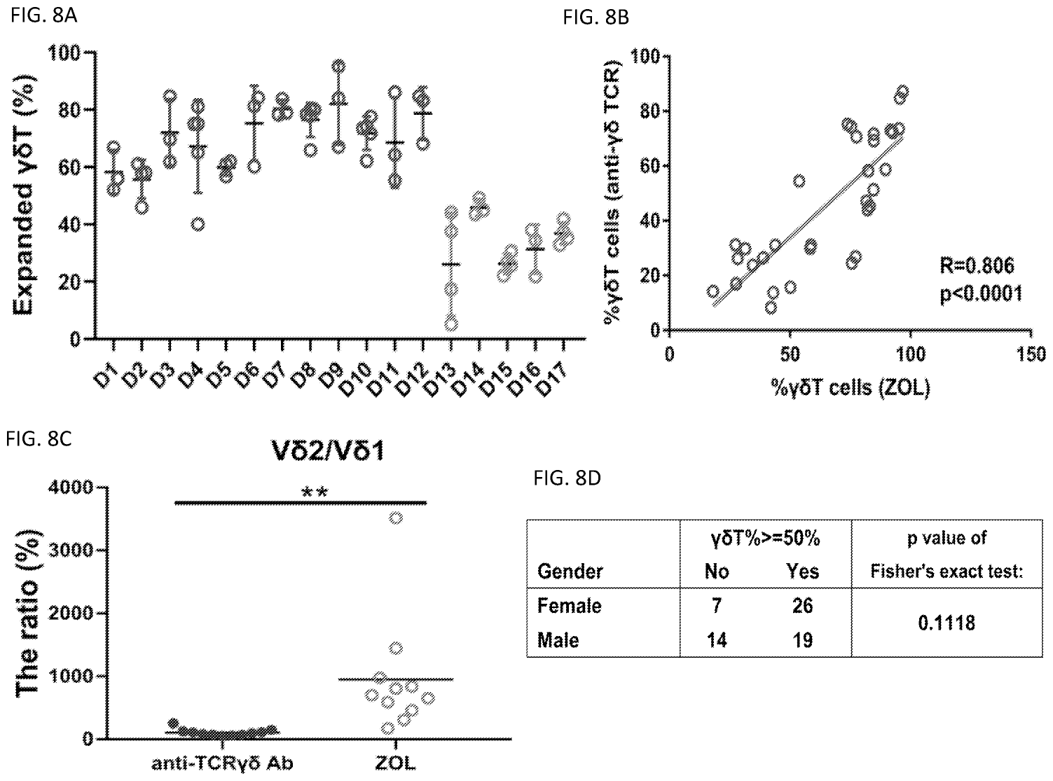

Not all PBMC samples from healthy donors can be used to effectively expand γδ T cells using zoledronate (ZOL, an FDA-approved drug). In the current study, 98 PBMC samples from 43 healthy donors were tested independently. Some of the donors had multiple samples collected at different time points during a period of 2 years. Consistent with published data, the purity of expanded γδ T cells was shown to be one of the important indicators for γδ T cell function (Nakajima et al., Eur J Cardiothorac Surg. 2010; 37(5):1191-7; Noguchi et al., Cytotherapy. 2011; 13(1):92-7). The purity of the expanded γδ T cells by ZOL in all of the 43 donors is displayed in FIG. 1A. PBMCs from 17 donors were tested three times or more independently at different time points and the purity of expanded γδ T cells from the same donor yielded similar results (FIG. 8A). PBMCs from 13 donors were expanded by both ZOL and anti-γδT TCR antibody. ZOL expanded γδ T cells significantly better than that of anti-γδT TCR antibody (FIG. 1), however, both expansion methods showed a similar trend (FIG. 8B). Anti-TCRγδ Ab expanded both Vδ2 and Vδ1 cells and the ratio of Vδ2/Vδ1 was 50%-250%, while ZOL preferentially expanded Vδ2 cells and the ratio of Vδ2/Vδ1 was 173%-3500% (FIG. 8C).

These donors were separated into two groups: good expansion group (γδ T cell purity ≥50%) and poor expansion group (γδ T cell purity <50%), as previously reported (Van Acker et al., J Hematol Oncol. 2016; 9(1):101). Absolute cell number, the fold change of expansion, and the viability of expanded γδ T cells in the two groups are illustrated in FIG. 1C. γδ T cell expansion fold ranged from 200 to 5000-folds in the good expanders, while 60 to 600-folds in the poor expanders. Purer γδ T cells were accompanied by higher absolute γδ T cell number and cell viability at day 10 after expansion (FIG. 1C). A more detailed analysis of γδ T cell expansion showed that the difference between good and poor expanders appeared 6 days after expansion (FIGS. 1D-1F). Age (FIG. 1G), gender (FIG. 8D) and HLA type (Table 1) of the donors was not significantly associated with γδ T cell expansion capacity.

TABLE 1
Summary of donor characteristics

| donor | age | Gender | HLA-A | HLA-B | HLA-DR | DPA1 | DPB1 | DQA1 | DQB1 |
|---|---|---|---|---|---|---|---|---|---|
| ND224 | 40 | Male | A 02:01, A | B 51:01, B | DRB1 | DPA1 | DPB1 | DQA1 | DQB1 |
| | | | 68:01 | 44:02 C | 03:01, | 01:03, | 01:01, | 03:03, | 02:01, |
| | | | | 05:01, B | 04:01 | DRB302:01 | 04:01 | 05:01 | 03:01 |
| | | | | 14:02 | 01:01 | DRB4 | | | |
| | | | | | 01:03 | | | | |
| ND317 | 40 | Male | A 30:02, | B 18:01, | DRB1 | DPA1 | DPB1 | DQA1 | DQB1 |
| | | | 66:01 | 41:02 C | 03:01, | 01:03, | 03:01, | 01:02, | 02:01, |
| | | | | 05:01, | 13:02 | DRB302:01 | 11:01 | 05:01 | 06:04 |
| | | | | 17:03 | 02:02, | | | | |
| | | | | | 03:01 | | | | |
| ND365 | 41 | Male | A24:02, | B08:01, | DRB1 | DPA1 | DPB1 | DQA1 | DQB1 |
| | | | A32:01 | B15:17 | 03:01, | 01:03, | 01:01, | 01:02, | 02:01, |
| | | | | C07:01 | 13:02 | DRB302:01 | 04:01 | 05:01 | 06:04 |
| | | | | | 01:01, | | | | |
| | | | | | 03:01 | | | | |
| ND410 | 55 | Female | A 01:01, | B 37:01, | DRB1 | DPA1 | 01:03DPB1 | 04:01DQA1 | DQB1 |
| | | | 24:02 | 51:01 C | 15:01, | | | 01:02, | 05:03, |
| | | | | 02:02, | 14:01 DRB3 | | | 01:04 | 06:02 |
| | | | | 06:02 | 02:24 DRB5 | | | | |
| | | | | | 01:01 | | | | |
| ND429 | 30 | Male | A 03:01, | B 07:02, | DRB1 | DPA1 | DPB1 | DQA1 | DQB1 |
| | | | 30:02 | 14:01 C | 04:04, | 01:03, | 01:01, | 02:01, | 02:02, |
| | | | | 07:02, | 07:01 | DRB402:01 | 04:01 | 03:01 | 03:02 |
| | | | | 08:02 | 01:03 | | | | |
| ND436 | 25 | Male | A25, A2 | B18, B8 | DR17, DR16 | | | | |
| ND451 | 31 | Female | A 29:01, | B 15:17, | DRB1 | DPA1 | 01:03DPB1 | 04:01 DQA1 | DQB1 |
| | | | 68:01 | 39:01 C | 16:01, | | | 01:02 | 05:02, |
| | | | | 07:01, | 13:02 DRB3 | | | | 06:04 |
| | | | | 12:03 | 03:01 DRB5 | | | | |
| | | | | | 02:02 | | | | |
| ND485 | 28 | Female | A2, A68 | B62, B57 | DR4, DR7 | | | | |
| ND492 | 37 | Male | A 01:01, | B 07:02, | DRB1 | DPA1 | DPB1 | DQA1 | DQB1 |
| | | | 03:01 | 37:01 C | 01:01, | 01:03, | 04:01, | 01:01, | 02:02, |
| | | | | 06:02, | 07:01 | DRB402:01 | 11:01 | 02:01 | 05:01 |
| | | | | 07:02 | 01:01 | | | | |
| ND499 | 31 | Female | A 02:01 | B 37:01, | DRB1 | DPA1 | DPB1 | DQA1 | DQB1 |
| | | | | 51:01 C | 01:01, | 01:03, | 02:01, | 01:01, | 05:01, |
| | | | | 06:02, | 14:54 | DRB302:01 | 17:01 | 01:04 | 05:03 |
| | | | | 15:02 | 02:02 | | | | |
| ND500 | 25 | Female | A 02:06, | B 15:02, | DRB3 | 03:01DPA1 | DPB1 | DQA1 | DQB1 |
| | | | 11:01 | 40:01 C | DRB5 | 01:0101:02, | 05:01, | 01:02, | 03:01, |
| | | | | 04:03, | | 06:01 | 21:01 | 06:01 | 05:02 |
| | | | | 08:01 | | | | | |
| ND502 | 52 | Female | A29, A30 | B35, B53 | DR7, DR13 | — | — | — | — |
| ND503 | 25 | Female | A 02:01, | B 44:03, | DRB1 | DPA1 | 01:03DPB1 | 04:01 DQA1 | DQB1 |
| | | | 29:02 | 50:01 C | 07:01, | | | 01:02, | 02:02, |
| | | | | 06:02, | 15:01 | | | 02:01 | 06:02 |
| | | | | 16:01 | DRB4: | | | | |
| | | | | | 01:01 | | | | |
| | | | | | DRB5: | | | | |
| | | | | | 01:01 | | | | |
| ND510 | 38 | Male | A1, A33 | B57, B81 | DR12, DR | — | — | — | — |
| | | | | | 13 | | | | |
| ND512 | 38 | Male | A 01:01, | B 08:01, | DRB1 | DPA1 | DPB1 | DQA1 | DQB1 |
| | | | 23:01 | 18:01 C | 03:01, | 01:03, | 01:01, | 05:01, | 02:01, |
| | | | | 07:01, | 11:04 | 02:01 | 04,02 | 05:05 | 03:01 |
| | | | | 06:02 | DRB3: | | | | |
| | | | | | 01:01, | | | | |
| | | | | | 02:02 | | | | |
| ND515 | 37 | Male | A11, A31 | B7, B44 | DR4, DR7 | | | | |
| ND517 | 27 | Female | A 11:01, A | B 18:01, B | DRB1 | DPA1 | 01:03DPB1 | DQA1 | DQB1 |
| | | | 30:02 | 51:01 C | 03:01, | | 02:02, | 05:01, | 02:01, |
| | | | | 05:01, B | 11:01 DRB3 | | 04:01 | 05:05 | 03:01 |
| | | | | 15:02 | 02:02 | | | | |
| ND519 | 39 | Female | A 11:01, | B 41:02, | DRB1 | DPA1 | 01:03 DPB1 | 04:01 DQA1 | DQB1 |
| | | | 66:01 | 52:01 C | 13:03, | | | 01:03, | 03:01, |
| | | | | 12:02, | 15:02 DRB3 | | | 05:05 | 06:01 |
| | | | | 17:03 | 01:01 DRB5 | | | | |
| | | | | | 01:02 | | | | |
| ND520 | 47 | Male | 02:01, A | B | DRB1 | 01:03 | 04:01 | 01:02, 03:03 | 03:02, 06:02 |
| | | | 23:01 | 49:0107:01, | 15:01, 04:05 | | | | |
| | | | 07:02, | 07:02 | DRB4 01:03 | | | | |
| | | | | | DRB5 01:01 | | | | |
| ND523 | 26 | Female | A 01:01, A | B 08:01, B | DRB1 | DPA1 | DPB1 | DQA1 | DQB1 |
| | | | 02:01 | 44:02 C | 03:01, | 01:03, | 04:01, | 04:02, | 02:01, |
| | | | | 05:01, C | 08:01 | DRB302:01 | 10:01 | 05:01 | 04:02 |
| | | | | 07:01 | 01:01 | | | | |
| ND527 | 40 | Male | A 02:01, | B 35:01, | DRB1 | DPA1 | DPB1 | DQA1 | DQB1 |
| | | | 03:01 | 44:03 C | 01:01, | 01:03, | 04:01, | 01:01, | 02:02, |
| | | | | 04:01, | 14:01 | DRB402:01 | 14:01 | 02:01 | 05:01 |
| | | | | 16:01 | 01:01 | | | | |
| ND528 | 28 | Male | A 01:01, A | B 35:12, B | DRB1 | 01:03 | 04:01, | 04:01 | 04:02 |
| | | | 68:03 | 55:01 | 08:01, 08:02 | | 04:02 | | |
| | | | | 03:03, 04:01 | | | | | |
| ND534 | 26 | Male | A 01:01, A | B 15:17, B | DRB1 | DPA1 | DPB1 | DQA1 | DQB1 |
| | | | 25:01 | 44:02 C | 01:01, | 01:03, | 04:01, | 01:01, | 05:01, |
| | | | | 05:01, C | 13:02 | DRB302:01 | 10:01 | 01:02 | 06:04 |
| | | | | 07:01 | 03:01 | | | | |
| ND535 | 32 | Male | A 02:01, | B 15:220, | DRB1 | DPA1 | DPB1 | DQA1 | DQB1 |
| | | | 29:02 | 49:01 C | 11:01, | 01:03, | 03:01, | 01:01, | 05:01, |
| | | | | 04:01, | 13:02 | DRB302:07 | 04:01 | 01:02 | 06:09 |
| | | | | 07:01 | 02:02, | | | | |
| | | | | | 03:01 | | | | |
| ND538 | 46 | Female | A 02:01 | B 18:01, | DRB1 | DPA1 | 01:03DPB1 | DQA1 | DQB1 |
| | | | | 57:01 C | 15:01, | | 03:01, | 01:02, | 03:02, |
| | | | | 06:02, | 04:03 DRB4 | | 04:02 | 03:01 | 06:02 |
| | | | | 07:01 | 01:03 DRB5 | | | | |
| | | | | | 01:01 | | | | |
| ND539 | 37 | Male | A 02:02, | B 44:03, | DRB1 | 15:03DPA1 | DPB1 | DQA1 | DQB1 |
| | | | 34:02 | 53:01 C | DRB5: | 02:02, | 01:01, | 01:02 | 06:02 |
| | | | | 04:01 | 01:01 | 03:01 | 105:01 | | |
| ND543 | 24 | Male | A 34:02, A | B 07:02, B | DRB1 | DPA1 | 02:02DPB1 | 01:01 DQA1 | DQB1 |
| | | | 68:02 | 57:03 C | 03:02, | | | 03:03, | 02:02, |
| | | | | 07:01, C | DRB1 09:01 | | | 04:01 | 04:02 |
| | | | | 07:02 | DRB3 03:01 | | | | |
| | | | | | DRB4 01:01 | | | | |
| ND544 | 29 | Female | A 02:01, | B 07:02, | DRB1 | DPA1 | DPB1 | DQA1 | DQB1 |
| | | | 11:01 | 51:01 C | 13:01, | 01:03, | 15:01, | 01:03, | 02:02, 06:0 |
| | | | | 02:02 | 07:01 | DRB302:07 | 19:01 | 02:01 | |
| | | | | 07:02 | 02:02 DRB4 | | | | |
| | | | | | 01:03 | | | | |
| ND552 | 26 | Female | A 01:01, | B 07:02, | DRB1 | DPA1 | 01:03DPB1 | DQA1 | DQB1 |
| | | | 02:01 | 08:01 C | 03:01, | | 02:01, | 05:01, | 02:01, |
| | | | | 07:01, | 11:01 DRB3 | | 04:01 | 05:05 | 03:01 |
| | | | | 07:02 | 01:01, | | | | |
| | | | | | 02:02 | | | | |
| ND560 | 21 | Female | A 02:06, | B 15:25, | DRB1 | DPA1 | DPB1 | DQA1 | DQB1 |
| | | | 24:02 | 40:02 C | 15:01, | 01:03, | 03:01, | 01:02, | 03:01, 06:0 |
| | | | | 03:04, | 12:02 | DRB302:02 | 05:01 | 06:01 | |
| | | | | 04:03 | 03:01 | DRB5 | | | |
| | | | | | 01:01 | | | | |
| ND150 | 38 | Male | A 02:01, | B 07:02, | — | DPA1 | DPB1 | DQA1 | DQB1 |
| | | | 23:01 | 44:03 C | | 01:03, | 04:01, | 01:02, | 02:02, |
| | | | | 04:01, | | 02:01 | 17:01 | 02:01 | 06:02 |
| | | | | 07:02 | | | | | |
| ND307 | 46 | Male | A 30:02, | B 18:01, | DRB1 | DPA1 | DPB1 | DQA1 | DQB1 |
| | | | 66:01 | 41:02 C | 03:01, | 01:03, | 03:01, | 01:02, | 01:03, |
| | | | | 05:01, | 13:02 | DRB302:01 | 11:01 | 05:01 | 02:01 |
| | | | | 17:03 | 02:02, | | | | |
| | | | | | 03:01 | | | | |
| ND388 | 51 | Male | A 02:01, | B 18:01, | DRB1: | DPA1: | DPB1: | DQA1: | DQB1: |
| | | | 68:01 | 44:02 C | 11:01, | 01:03 | 02:01, | 05:05 | 03:01 |
| | | | | 07:01, | 11:04 | | 04:01 | | |
| | | | | 07:04 | DRB3: | | | | |
| | | | | | 02:02 | | | | |
| ND463 | 54 | Male | A 02:01, | B 07:02, | | DPA1 | 01:03DPB1 | DQA1 | DQB1 |
| | | | 11:01 | 15:01 C | | | 01:03, | 01:02, | 03:02, |
| | | | | 07:02, | | | 04:02 | 03:01 | 06:02 |
| | | | | 03:04 | | | | | |
| ND495 | | Female | A 01:01, | B 08:01, | DRB1 | DPA1 | 01:03DPB1 | 04:02DQA1 | DQB1 |
| | | | 02:01 | 14:02 C | 01:02, | | | 01:01, | 02:01, |
| | | | | 07:01, | 03:01 DRB3 | | | 05:01 | 05:01 |
| | | | | 08:02 | 01:01 | | | | |
| ND516 | 35 | Female | A 02:01, | B 07:02, | DRB1 | DPA1 | 01:03DPB1 | 04:01 DQA1 | DQB1 |
| | | | 03:01 | 51:01 C | 15:01, | | | 01:02, | 03:01, |
| | | | | 07:02, | 04:07 DRB4 | | | 03:03 | 06:02 |
| | | | | 14:02 | 01:03 DRB5 | | | | |
| | | | | | 01:01 | | | | |
| ND518 | 36 | Female | A 02:01, A | B 14:02, B | DRB1 | DPA1 | 01:03 DPB1 | 04:01 DQA1 | DQB1 |
| | | | 03:01 | 39:01 C | 01:02, | | | 01:01, | 03:03, |
| | | | | 07:02, C | 07:01 DRB4 | | | 02:01 | 05:01 |
| | | | | 08:02 | 01:03N | | | | |
| ND525 | 25 | Male | 01:01, 03:01 | 08:01, 49:01 | DRB1 | 01:03, 03:01 | 04:01, 105:01 | 01:01, 05:01 | 02:01,05:01 |
| | | | | 07:01 | 01:01, 03:01 | | | | |
| | | | | | DRB3 02:02 | | | | |
| ND526 | | Female | A2, A24 | B27, B62 | DR8 | | | | |
| ND528 | 28 | Male | A 01:01, A | B 35:12, B | DRB1 | 01:03 | 04:01, | 04:01 | 04:02 |
| | | | 68:03 | 55:01 | 08:01, 08:02 | | 04:02 | | |
| | | | | 03:03, 04:01 | | | | | |
| ND531 | | Female | A 01:02, A | B 49:01, B | DRB1 | DPA1 | 01:03DPB1 | DQA1 | DQB1 |
| | | | 24:02 | 15:17 C | 04:05, | | 02:01, | 01:02, | 03:02, |
| | | | | 07:01 | 13:02 DRB3 | | 04:01 | 03:03 | 06:04 |
| | | | | | 03:01 DRB4 | | | | |
| | | | | | 01:03 | | | | |
| ND541 | 28 | Female | A 02:01, A | B 44:03, B | DRB1 | DPA1 | DPB1 | DQA1 | DQB1 |
| | | | 25:01 | 51:01 | 08:03, | 01:03, | 04:01, | 05:01, | 02:01, |
| | | | | 07:06, 15:02 | 03:01 | DRB302:01 | 90:01 | 06:01 | 03:01 |
| | | | | | 01:01 | | | | |
| ND542 | 39 | Male | A 01:01, | B 08:01, | DRB1 | DPA1 | 01:03DPB1 | DQA1 | DQB1 |
| | | | 24:02 | 15:01 C | 03:01, | | 04:01, | 05:01, | 02:01, |
| | | | | 03:03, | 11:03 DRB3 | | 04:02 | 05:05 | 03:01 |
| | | | | 07:01 | 01:01, | | | | |
| | | | | | 02:02 | | | | |
| ND561 | 25 | Male | A 23:01, | B 13:02, | DRB1 | DPA1 | DPB1 | DQA1 | DQB1 |
| | | | 30:01 | 49:01 C | 04:03, | 01:03, | 04:01, | 02:01, | 02:02, |
| | | | | 06:02, | 07:01 | DRB402:01 | 17:01 | 03:01 | 03:02 |
| | | | | 07:01 | 01:03 | | | | |

### Example 2: Interindividual γδT Cell Immune Phenotypic Heterogeneity after Expansion

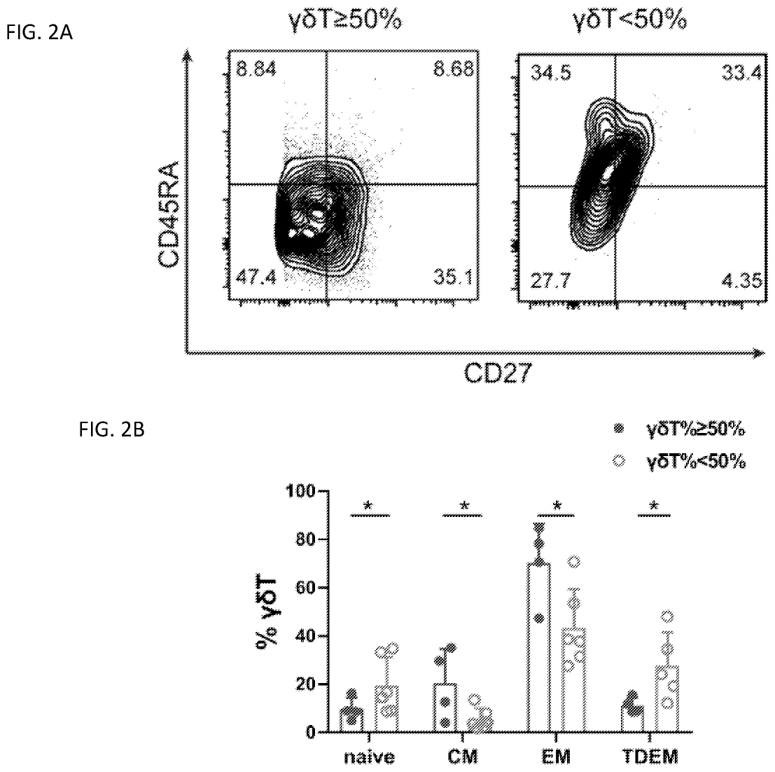

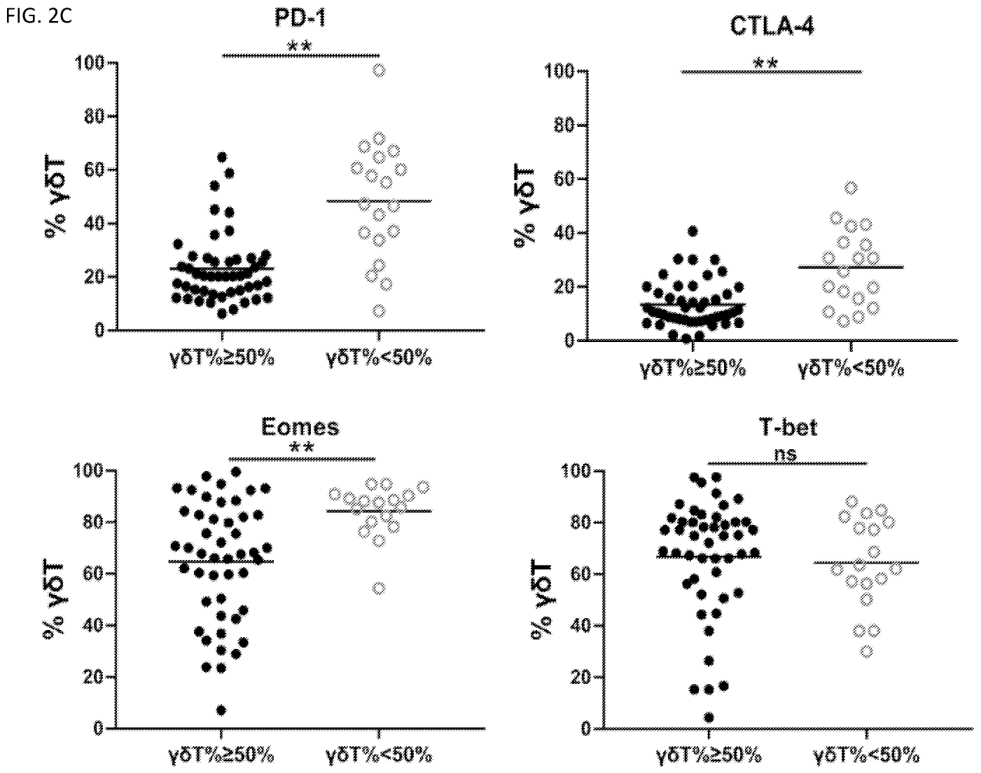

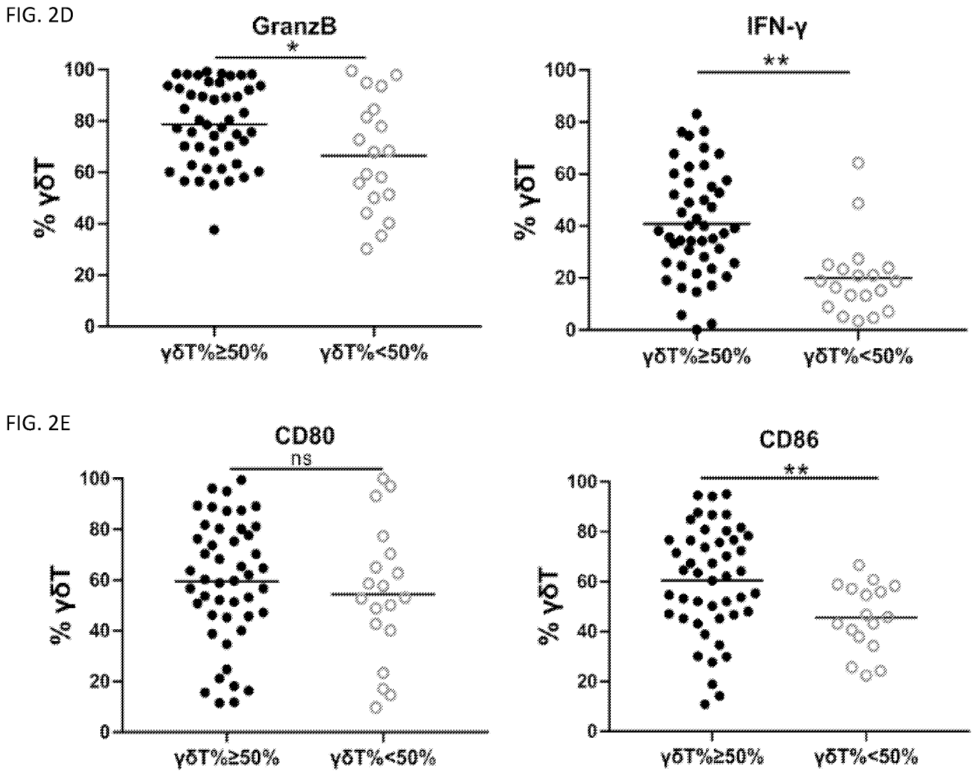

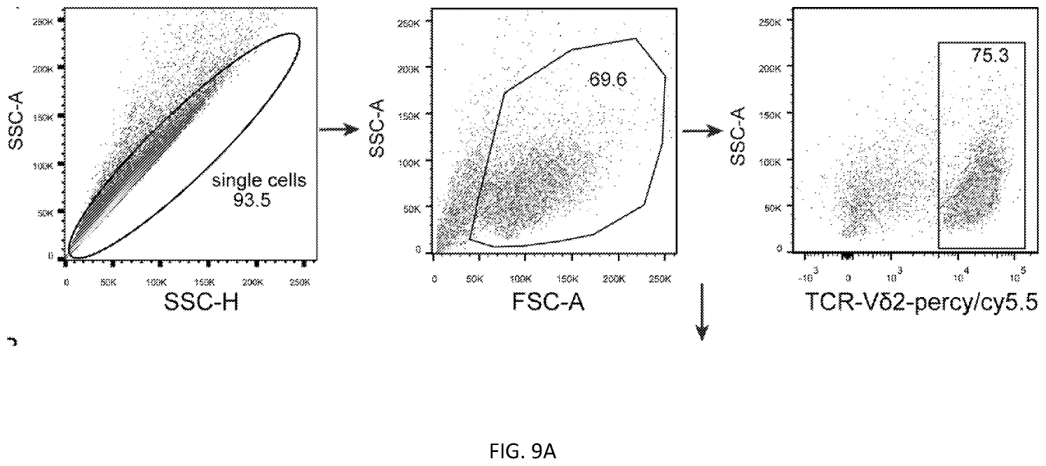

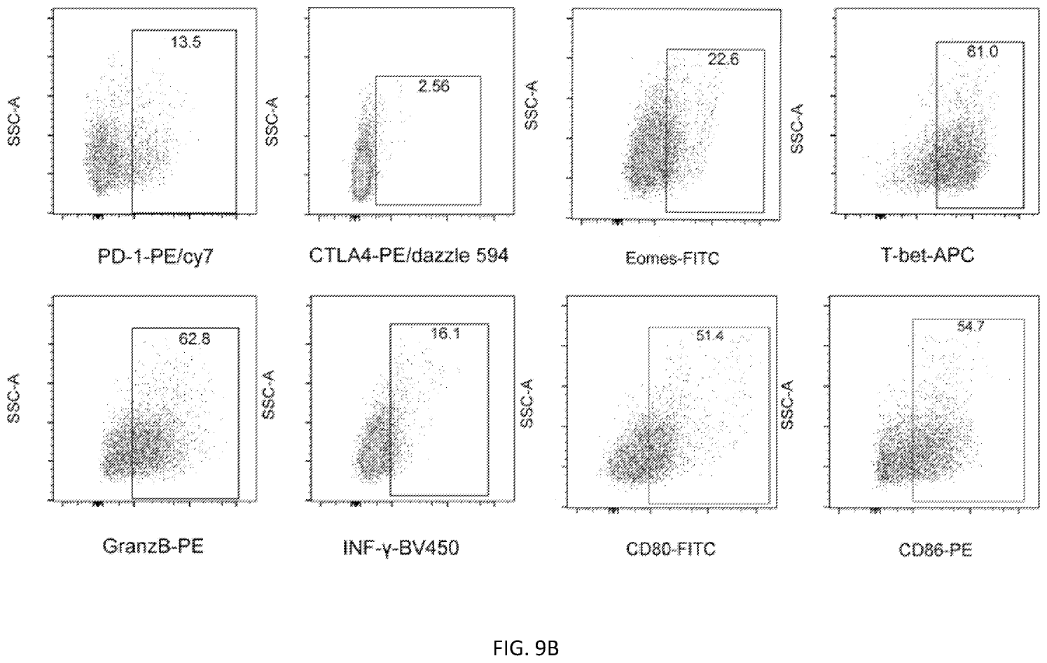

It has been shown that the immune phenotype of γδ T cells may predict their function. To study the phenotypic difference in γδ T cell in healthy donors, CD27 and CD45RA expression was examined on γδ T cells after 10 days of expansion. These receptors distinguish effector memory (EM, CD45RA-CD27−) cells from naive (N, CD45RA+CD27+), central memory (CM, CD45RA-CD27+), or terminal-differentiated effector memory (TDEM, CD45RA+CD27−) cells (FIG. 2A). The proportion of the effector memory and central memory γδ T cells were significantly higher in the good expanders compared to that in the poor expanders (FIG. 2B). The expression of immune checkpoint proteins in the expanded γδ T cells was then examined (FIG. 9). γδ T cells in the good expanders showed significantly lower expression of exhaustion markers, PD-1, CTLA-4 and Eomes (FIG. 2C), but higher expression of effector cytokine, IFN-γ, Granzyme B (FIG. 2D), and costimulatory molecule of CD86 (FIG. 2E), compared to those in the poor expanders. There were no significant changes in T-bet and CD80 expression.

### Example 3: Interindividual γδT Cells Display Heterogeneous Migratory and Cytotoxic Effector Function after Expansion

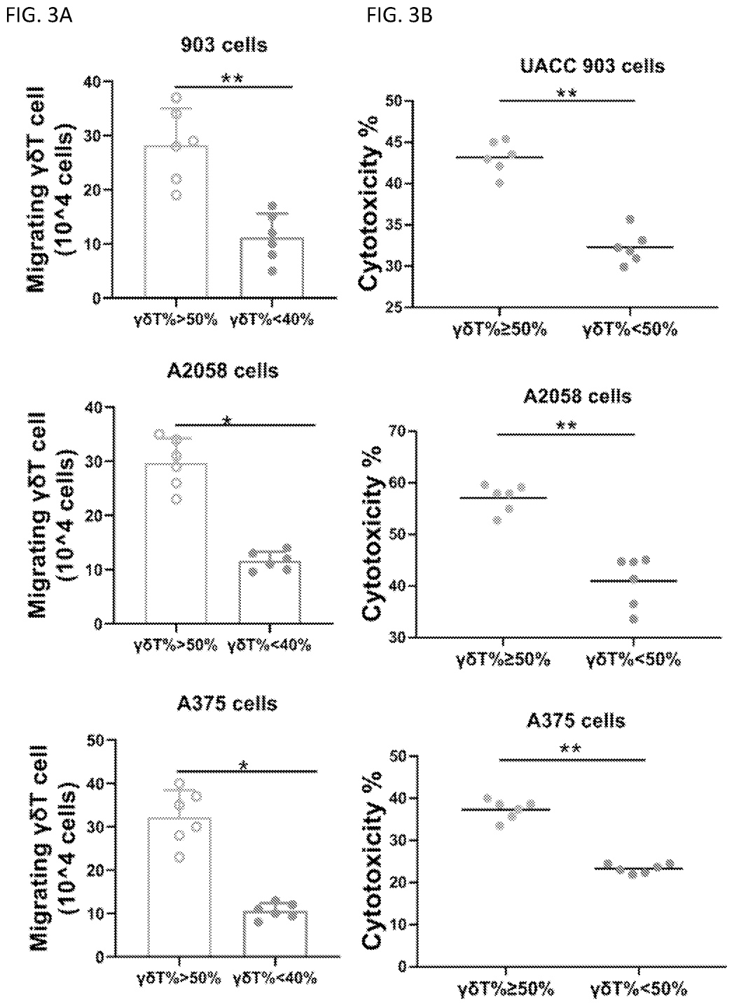

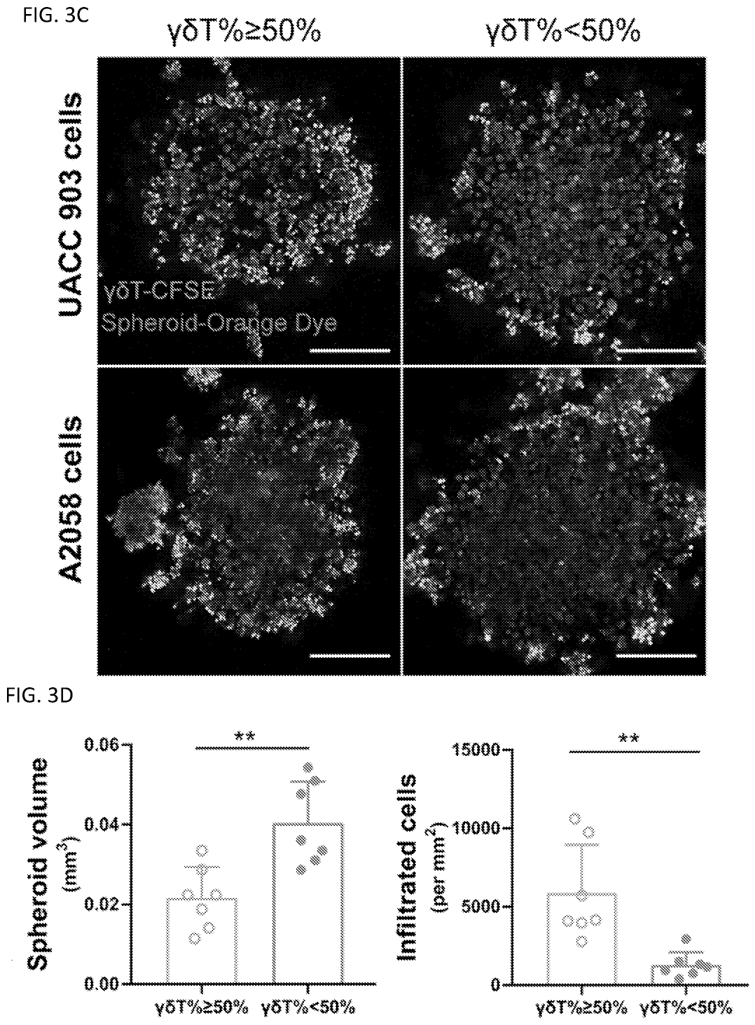

To study the migratory ability of the expanded γδ T cells to tumor cells, transwell migration assays were performed. Significantly enhanced migration of γδ T cells towards A2058, A375 and UACC903 melanoma cells was observed in the good expansion group compared to that in the poor expansion group after 8 hours incubation (FIG. 3A). Expanded γδ T cells were mixed with tumor cells at the effector:target (E:T) ratio of 5:1. γδ T cells in the good expansion group more effectively killed tumor cells than those in the poor expansion group (FIG. 3B). The migration and cytotoxic effector function of the expanded γδ T cells was further explored using 3D melanoma spheroid assays. Spheroid integrity was examined by monitoring spheroid size and γδ T cell infiltration by flow cytometry. A2058 and UACC903 melanoma spheroids were co-cultured with γδ T cells for 48 h. Melanoma spheroids had greater γδ T cell infiltration and were significantly smaller when cultured with expanded γδ T cells from good expanders than those from poor expanders (FIGS. 3C and 3D). These data suggest that γδ T cell expansion capacity is correlated with their anti-tumor capacity.

### Example 4: Basal γδT Cell Levels in PBMC Predict their Expansion Capacity

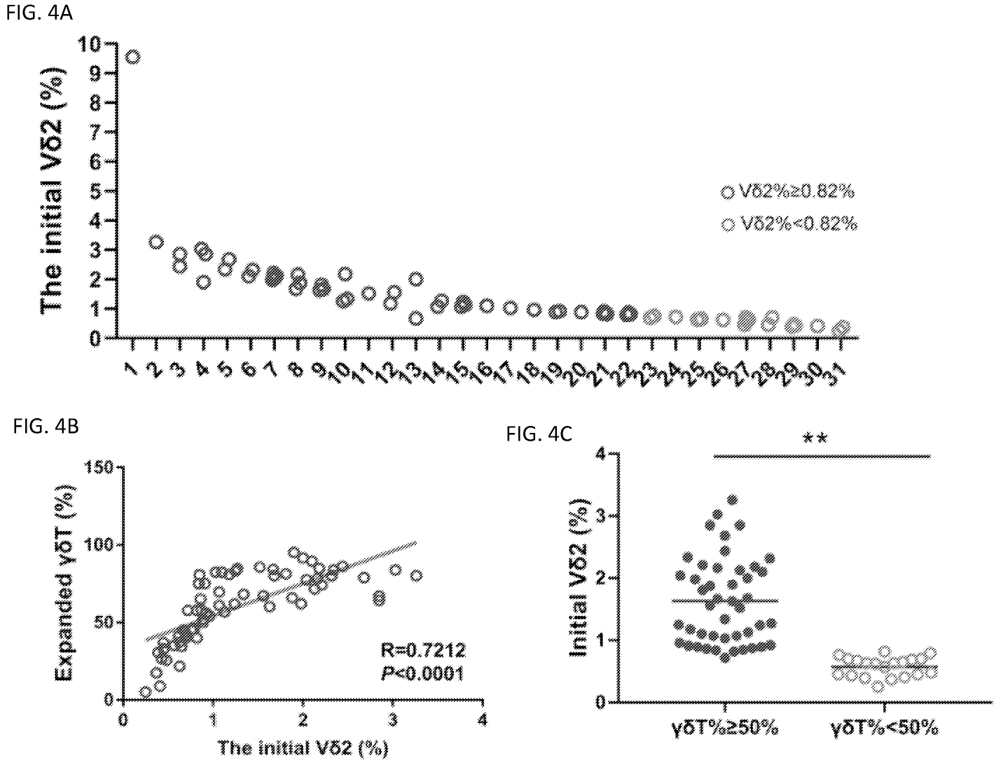

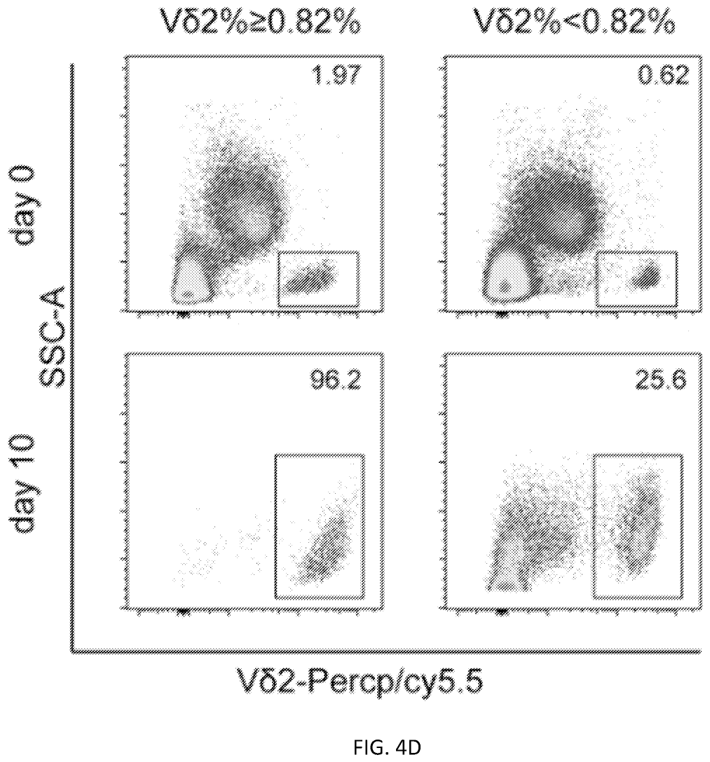

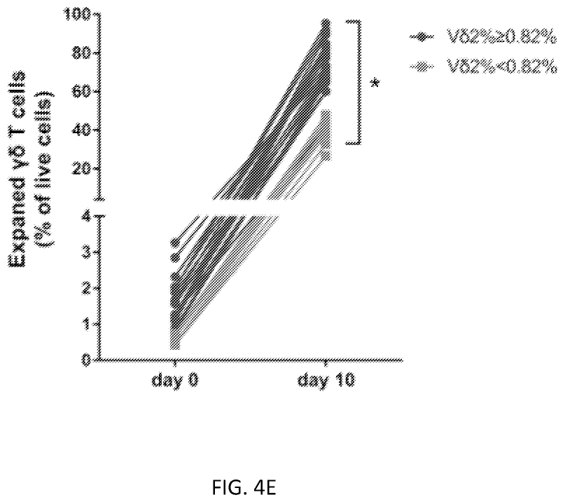

Basal levels of γδ T cells in PBMC were measured in 66 samples from 31 donors using flow cytometry (FIG. 4A). There was a significant correlation between basal Vδ2 T cells and γδT expansion capacity (FIG. 4B). Better expanders had a significantly higher basal Vδ2 T cells in the PBMCs (FIG. 4C). Using logistic regression and LASSO models, a baseline Vδ2 T cell cut-off of 0.82% was calculated to separate good expanders from poor expanders (FIGS. 4D-4E).

### Example 5: γδT Cell Intrinsic Characters Determine their Expansion Capacity

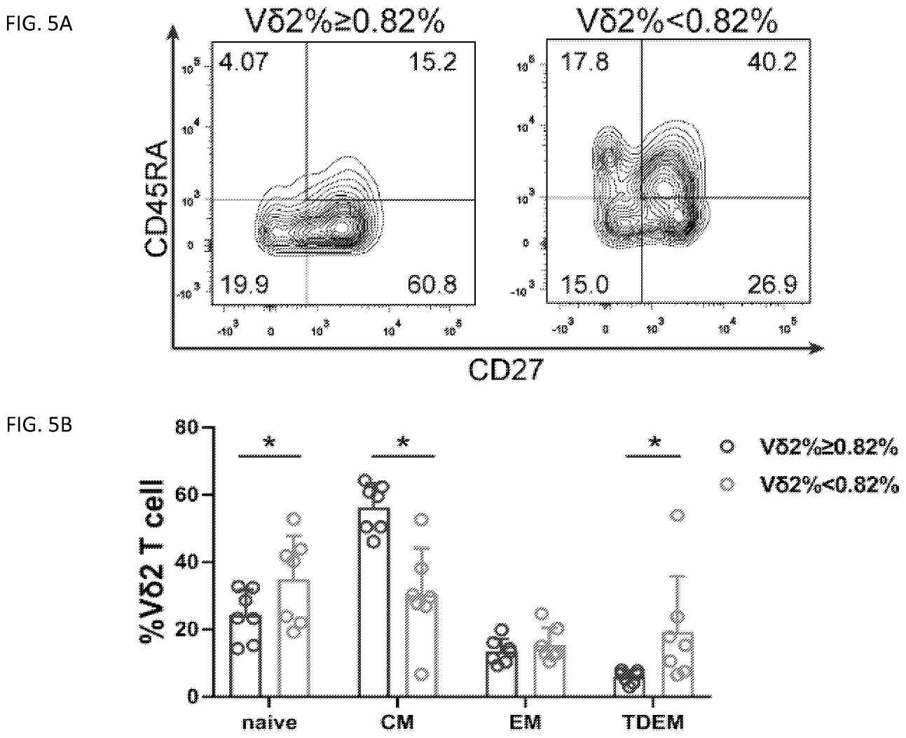

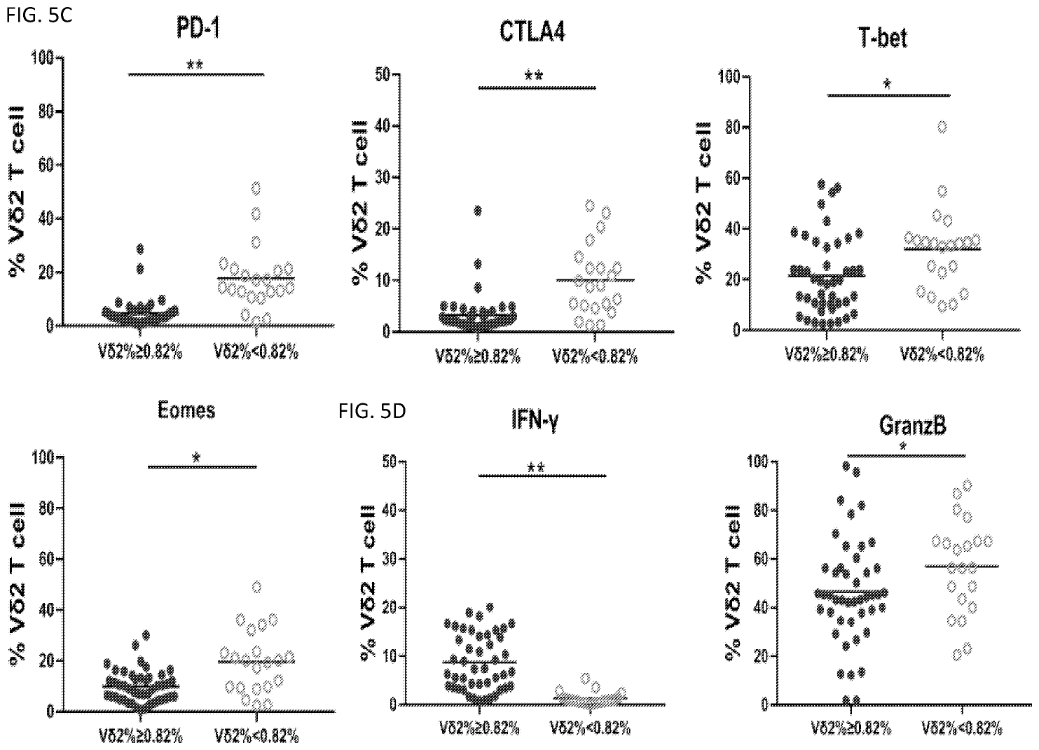

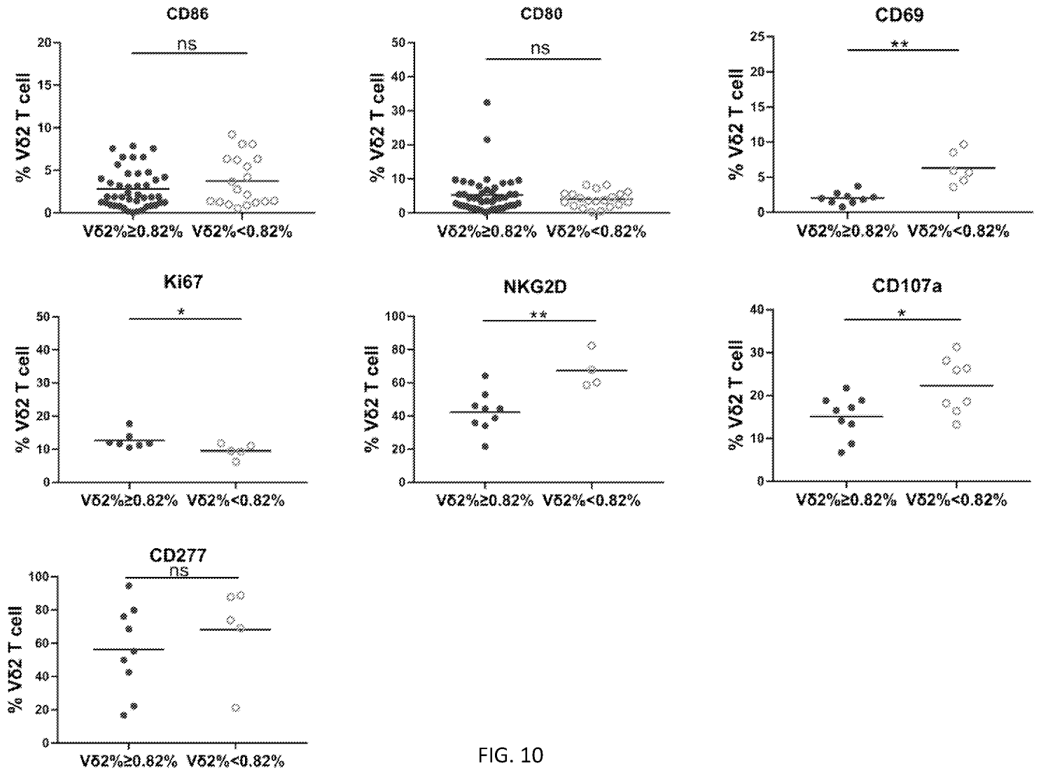

It was then investigated whether immune phenotypes of basal γδ T cells in PBMC correlate with their expansion capacity. The results demonstrated that there were more central memory (CD27+CD45RA−) and fewer terminally differentiated γδ T cells (CD27-CD45RA+) in the Vδ2 T cells ≥0.82% group than in the <0.82% group (FIGS. 5A-5B). Central memory cells have better proliferation capacity, while terminally differentiated cells display immediate effector functions with poorer proliferation capacity. In addition, PD-1, CTLA-4, Eomes and T-bet levels were significantly higher in the Vδ2 T cells <0.82% group than those in the ≥0.82% group (FIG. 5C). Vδ2 T cells with high PD-1 and CTLA-4 expression suppress Vδ2 T cell proliferative responses and cytotoxic potential. Interestingly, γδ T cells expressed less IFN-7 but more Granzyme B in the <0.82% group (FIG. 5D). Moreover, the activation marker CD69 and the cytotoxicity markers NKG2D and CD107a were also significantly higher in the <0.82% group. Inversely, the proliferation marker Ki67 was significantly lower in the <0.82% group, while other activation markers, such as CD80, CD86 and CD277 showed no significant difference (FIG. 10). Altogether, the γδ T cells in <0.82% group expressed markers of exhaustion and cytotoxicity, while the ≥0.82% group showed memory-like phenotypes.

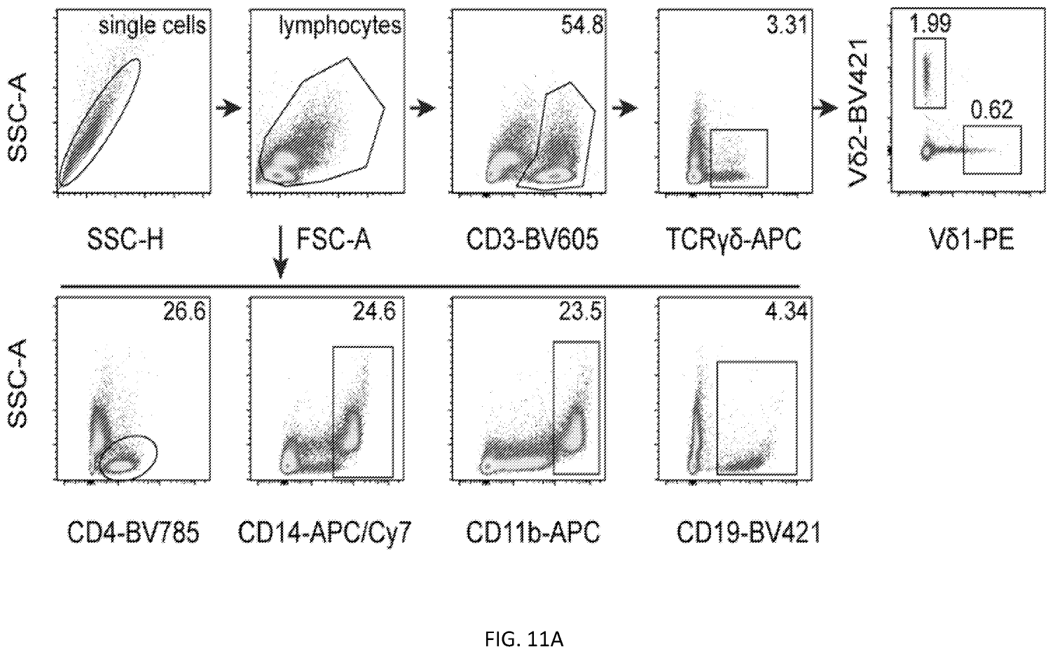

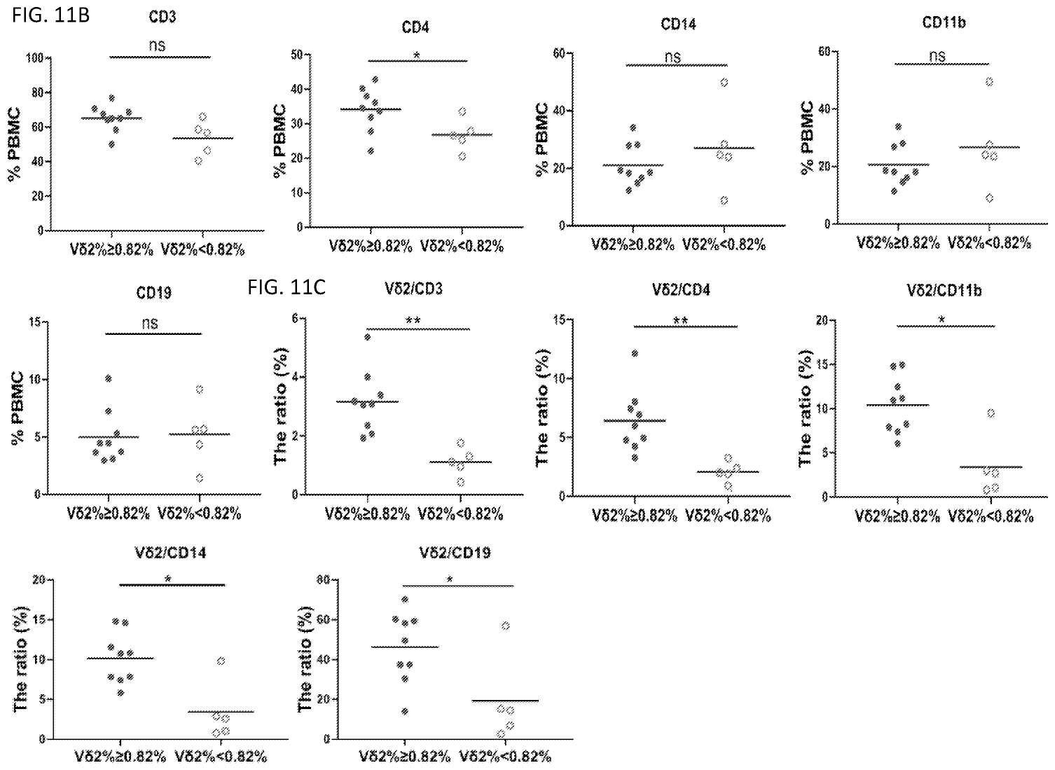

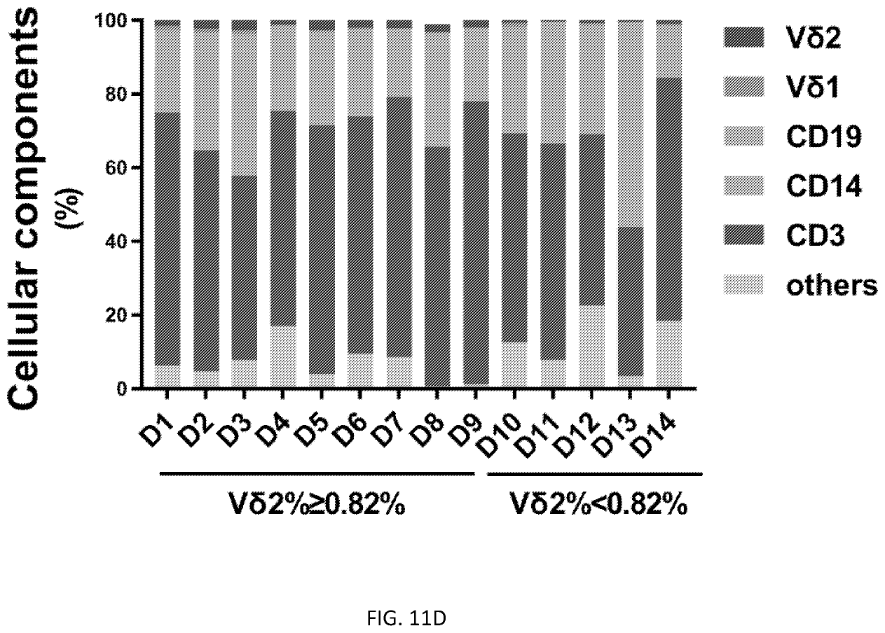

To study whether other cell components in PBMC impacted γδ T cell expansion, other immune cells in PBMC from 25 healthy individuals were examined. In the good expansion group, Vδ2 T cells comprised a larger percentage of all γδ T cells. The Vδ1 T cells in different individuals varied but no significant difference was found in these two groups, however, the ratio of Vδ2/Vδ1 T cells was significantly different in these two groups (FIG. 5E). CD3+, CD4+, CD14+, CD19+ and CD11b+ cell population was measured in the PBMC (FIG. 11A). CD4+ cell proportion in the ≥0.82% group was higher. However, CD3+, CD14+, CD11b+ and CD19+ cells showed no significant difference in these two groups (FIGS. 11B-11D).

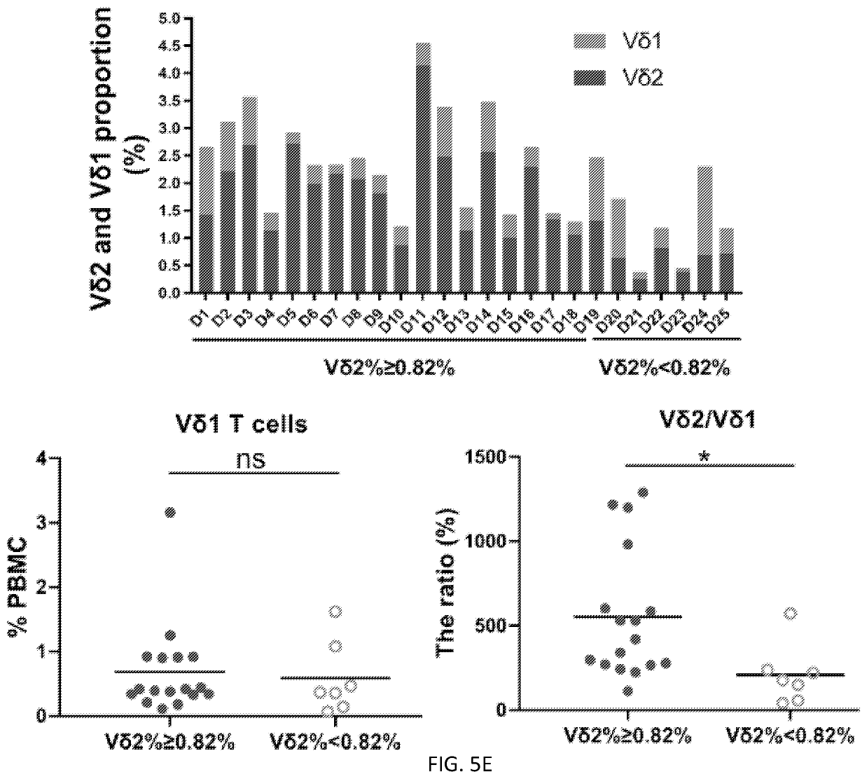

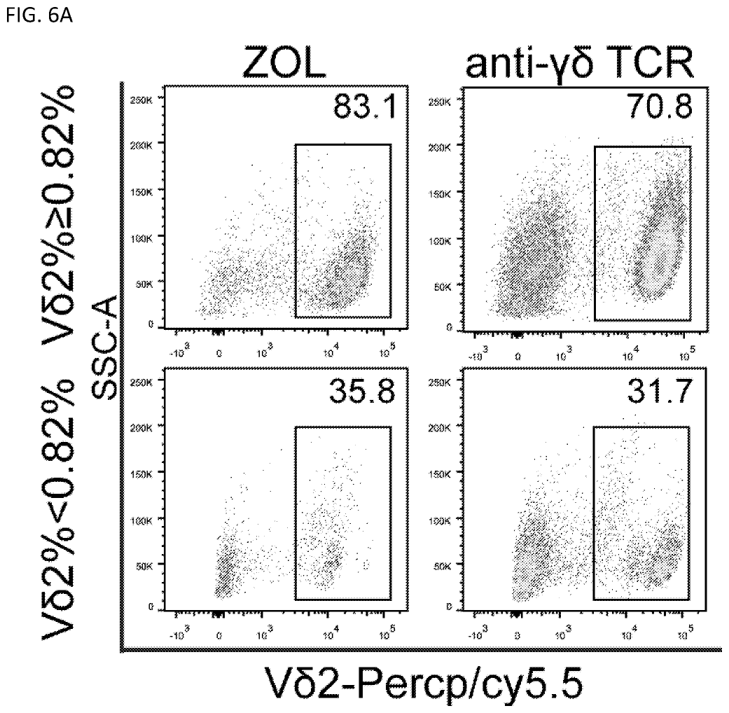

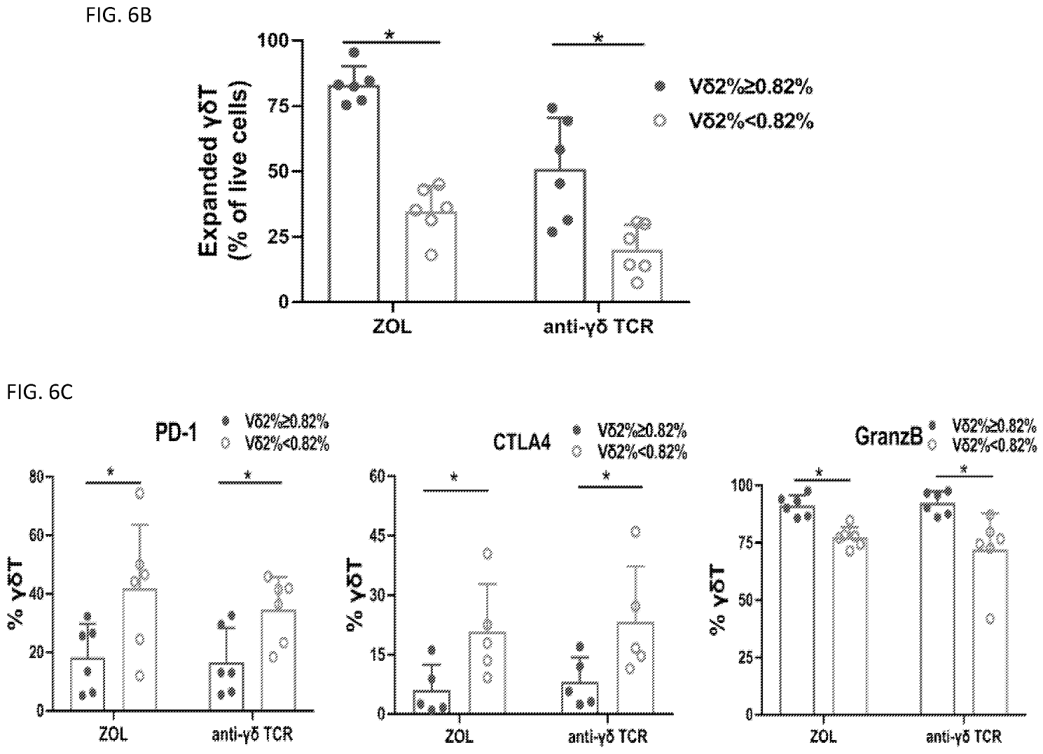

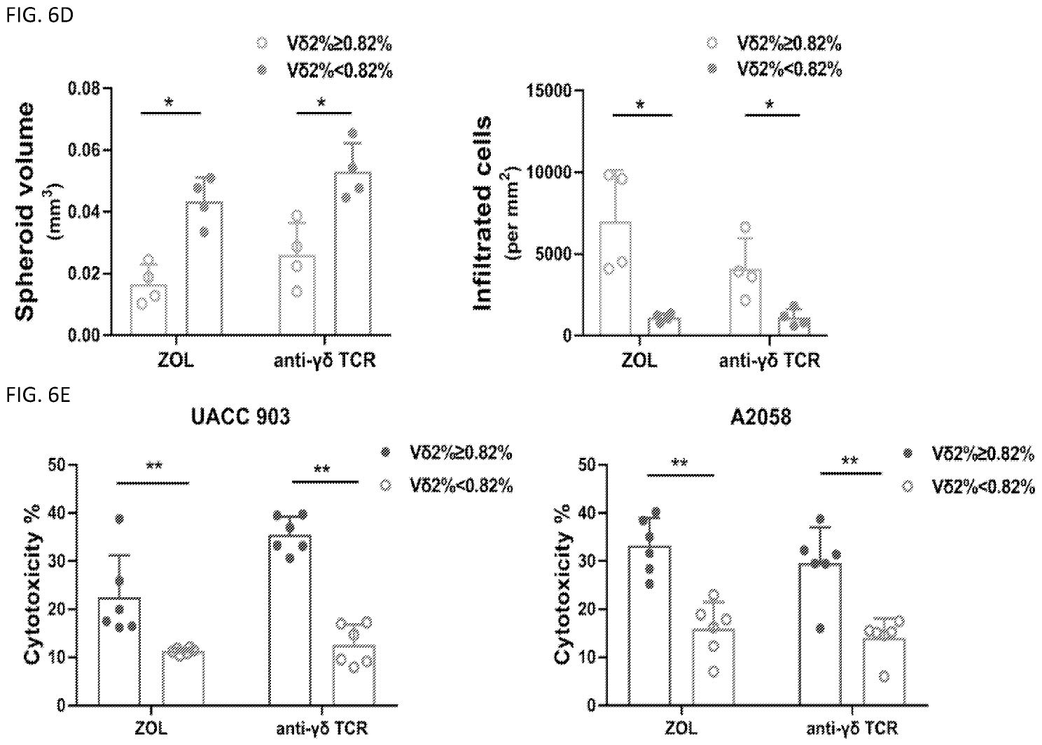

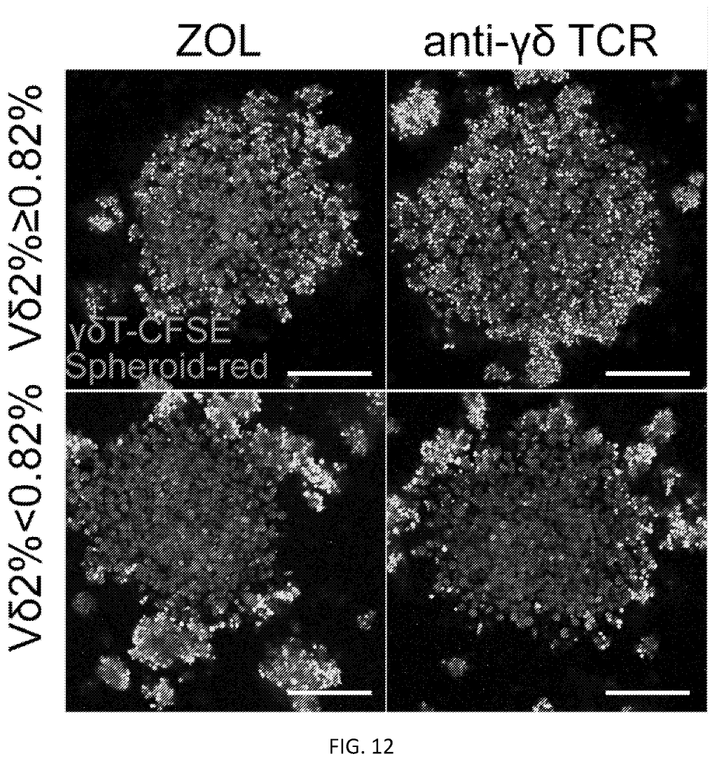

To study whether different culture methods influenced γδ T cell expansion, ZOL or anti-γδTCR antibody were used to expand γδ T cells from PBMC (n=6). The data demonstrated that ZOL induced better expansion of γδ T cells than anti-γδTCR antibody. Nevertheless, similar to ZOL induced γδ T cell expansion, anti-γδTCR antibody induced significantly better γδ T cell expansion in the basal Vδ2 T cells ≥0.82% group than that in the <0.82% group (FIGS. 6A-6B). Immune checkpoint protein expression was also measured on the anti-γδTCR antibody-expanded γδ T cells. The Vδ2 T cells in the ≥0.82% group expressed lower PD-1 and CTLA-4 and higher Granzyme B than those in the ≤0.82% group (FIG. 6C), consistent with ZOL expanded cells. The cytotoxicity of tumor spheroid assays in UACC903 and A2058 melanoma cells showed comparable results, regardless of whether ZOL or anti-γδTCR antibody expansion were used (FIG. 6D, 6E and FIG. 12). These data suggest that interindividual γδT expansion capacity is not related to expansion methods.

### Example 6: Anti-PD-1 or Anti-CTLA-4 Antibodies Cannot Rescue ZOL Induced TST Cell Expansion

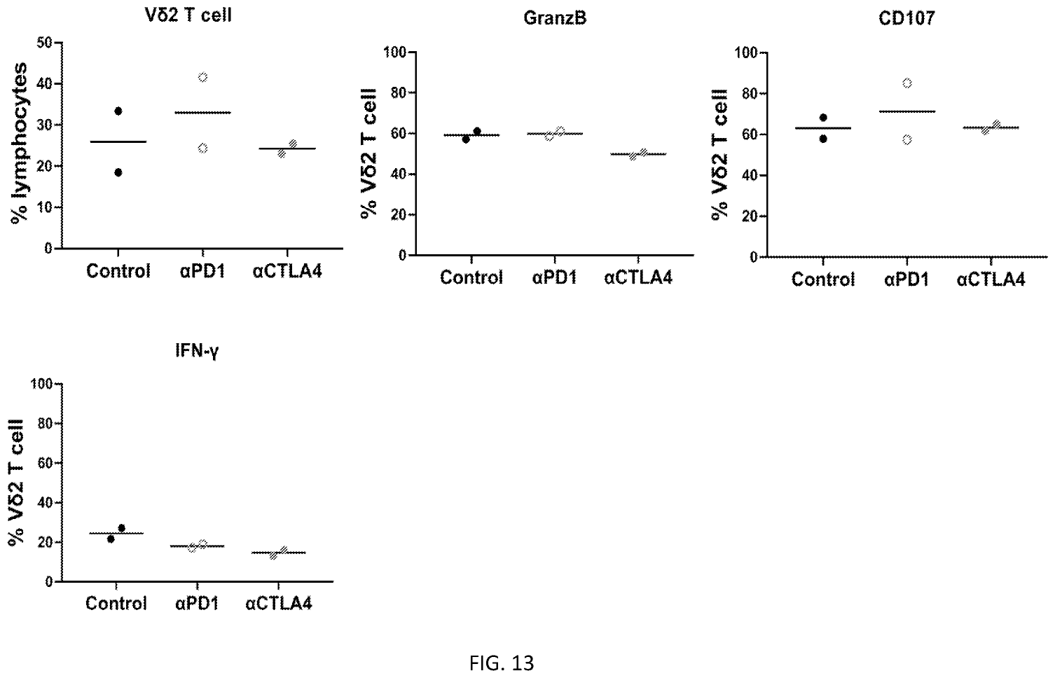

Since PD-1 and CTLA-4 proteins are highly expressed in the γδ T cells in the poor expanders, anti-PD-1 (50 μg/ml) or anti-CTLA-4 (50 μg/ml) antibody was added in the culture medium during the ZOL induced expansion period in an attempt to rescue the cells. γδ T cell number, cell purity, CD107a, Granzyme B and IFN-7 expression was examined in the γδ T cells after expansion. However, there was no increase of γδ T cell expansion nor improved cytokine expression after the treatment (FIG. 13).

### Example 7: Logistic Regression Models Predict γδT Cell Expansion Capacity: Vδ2 Index Score

To predict expansion capacity of γδ T cells ex vivo, two logistic regression models were developed. The first predictive model is based solely on the Baseline γδ T cell concentration in PMBC, from which each individual sample has a score calculated as:

Vδ2Index Score=−8.23+10.25×Initial_γδT %

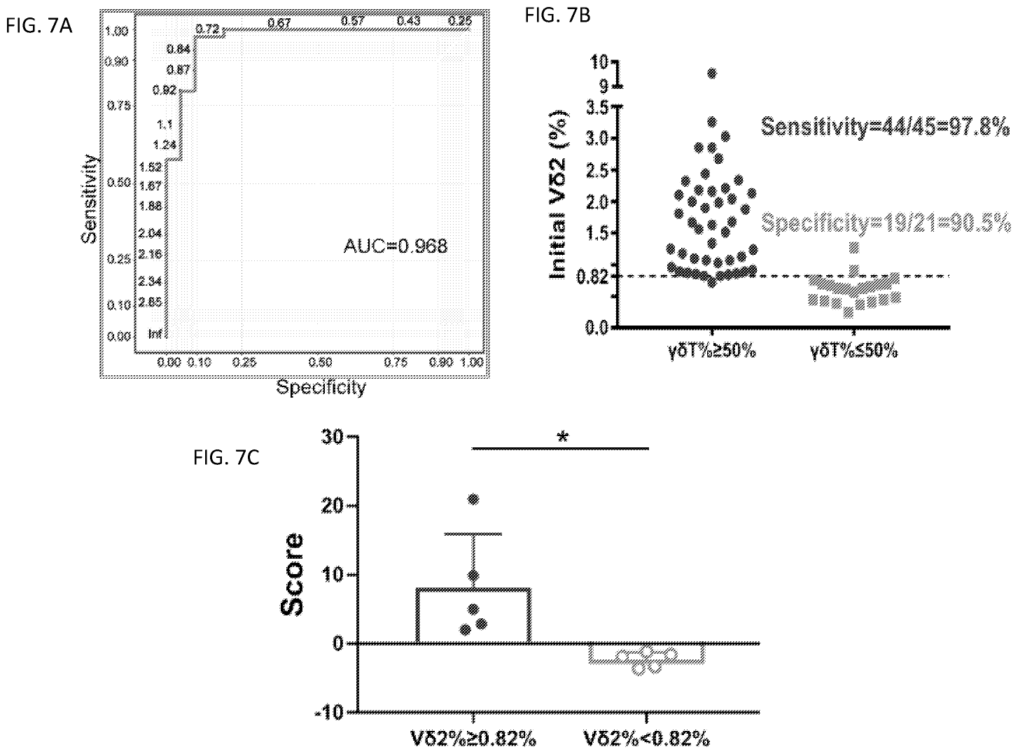

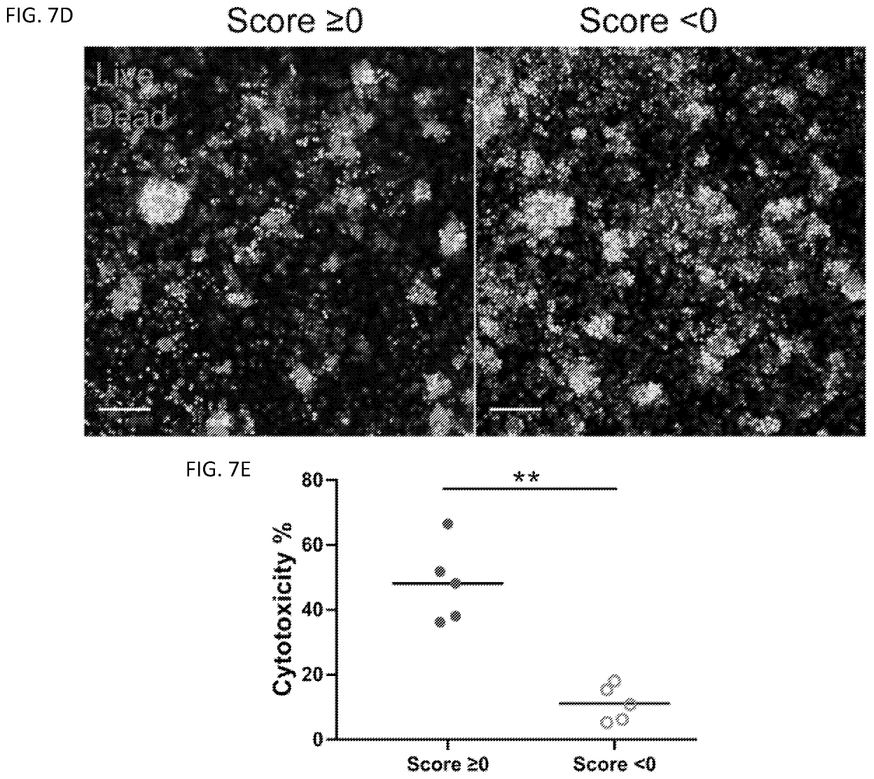

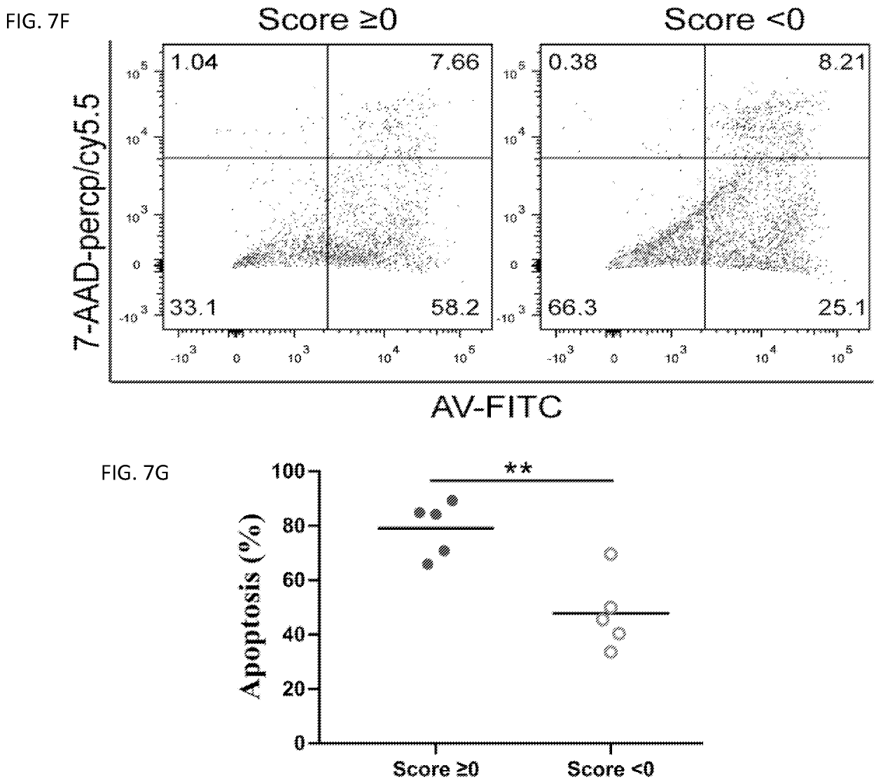

Using this model, a sensitivity of 97.78% and specificity of 90.48% is reached at cut point of 0.82% of the initial γδT %, with AUC=0.968 (FIGS. 7A-7B). In the second comprehensive predictive model, the initial γδ T cell level and surface marker expression of γδT cells were included. Each individual sample has a score from the established logistic regression model:

$$Comprehensive V δ 2 Index Score = ln ( \frac{P}{1 - P} ) = - 20.57 + 24.05 \times Initial\_Vδ2T \% + 0.04 \times PD 1 - 0.08 \times CTLA 4 - 0.07 \times Emoes + 0.04 \times T - bet - 0.25 \times INF - γ + 0.02 \times GranzB + 0.48 \times CD 80 - 0.27 \times CD 86$$

The probability P that an individual's γδ T cells can be expanded ≥50% was calculated as:

$$P = \frac{{e}^{S c o r e}}{1 + {e}^{S c o r e}}$$

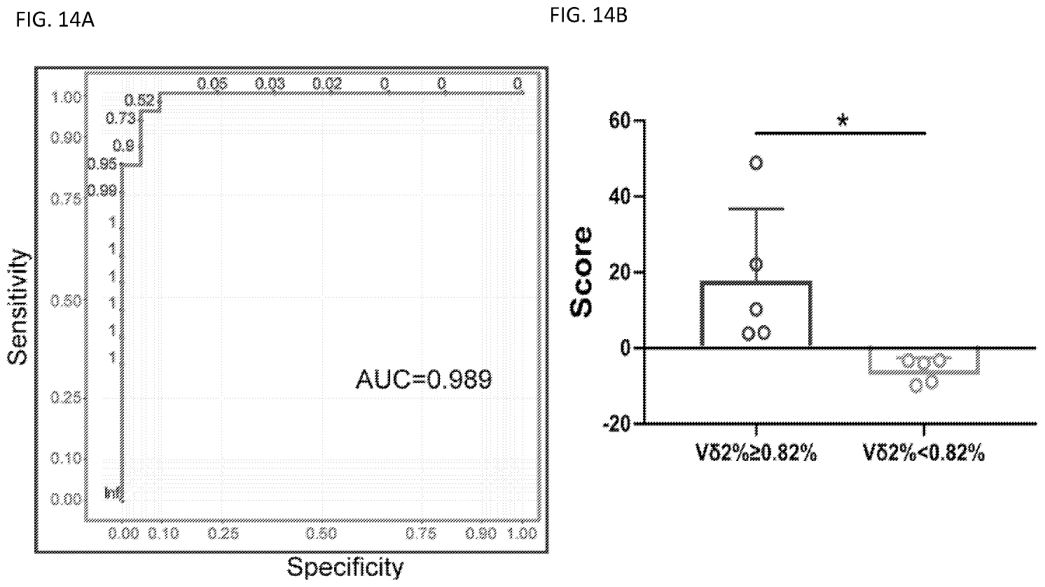

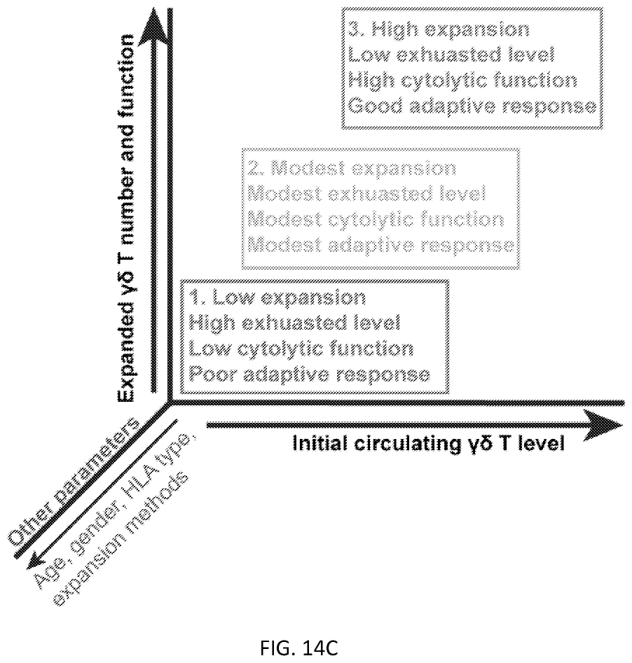

The optimal sensitivity of 95.56% and specificity of 95.24% is reached at cut point 0.74 of the Score, corresponding to P=0.678, with AUC=0.989 (FIG. 14A). There is no significant difference of AUC between these two models (Delong's test p value=0.3047).

### Example 8: Confirmation of Interindividual γδT Cell Functions Using Patient-Derived Melanoma Organoids

Patient-derived cancer organoids retain the heterogeneity of original tumors and provide an opportunity for in vitro testing of human γδ T cell therapy, since good in vivo models are not available. To study whether these findings may predict effects of γδ T cells on patient-derived melanoma organoids, PBMC samples were screened and the 10 most recent samples were used. Vδ2 Index Score was calculated and shown in FIG. 7C and FIG. 14B. Fresh human melanoma tissues were obtained and processed to establish patient-derived melanoma organoids as previously described (Ohlund et al., J Exp Med. 2017; 214(3):579-96). Melanoma organoids grew well over time and few cells were positive for propidium iodide (PI, dead cells) staining, which stains dead cells. Following γδ T cell expansion, the expanded γδT were co-cultured with the organoids for 24 h, and then cells were stained with acridine orange (AO; live cells) and propidium iodide (PI; dead cells). Vδ2 Index Score ≥0 group exhibited significant more cell death than the Vδ2 Index Score <0 group (FIG. 7D). The cytotoxicity assay showed similar results (FIG. 7E). FACS analysis of Annexin V (AV) and 7-amino actinomycin D (7-AAD) stained cells showed a greater number of apoptotic cells in the Vδ2 Index Score ≥0 group than that in the Vδ2 Index Score <0 group (FIGS. 7F-7G). These data support that the Vδ2 Index Score can be used to predicted γδT anti-tumor function.

### Example 9: Discussion

Adoptive immunotherapy with Vδ2 T cells has been used in a variety of cancer treatments without severe toxicities (de Weerdt et al., Blood. 2018; 132(21):2260-72; Xu et al., Cell Mol Immunol. 2020), but the treatment outcomes were mixed (Nicol et al., Br J Cancer. 2011; 105(6):778-86; Kobayashi et al., Anticancer Res. 2010; 30(2):575-9; Kakimi et al., Translational lung cancer research. 2014; 3(1):23-33), and clinical benefit rate was approximately 40% (Kunzmann et. al., J Immunother. 2012; 35(2):205-13). γδ T cells from patients face obstacles, such as exhaustion and immunosuppression, induced by the tumor microenvironment. Thus, researchers and companies have studied allogeneic γδ T cell therapy, in order to bypass these obstacles (Sebestyen et al., Nature reviews Drug discovery. 2020; 19(3):169-8436). However, the therapeutic efficacy of γδ T cells derived from healthy individuals is still unsatisfactory (Xu et al., Cell Mol Immunol. 2020; Chauvin et al., Clin Cancer Res. 2019; 25(23):7218-28).

One of the underlying factors that determines the observed clinical responses in patients may be the interindividual heterogeneity in γδ T cell levels. Indeed, studies herein confirm that there is significant γδ T cell interindividual heterogeneity in healthy donors and that basal γδT cell levels in the blood may be used to stratify their antitumor function. These findings make it possible to screen and select healthy donors for γδ T cell-based therapies, which is crucial to fully realize the therapeutic potential of γδ T cells.

It has been shown that the purity and absolute cell number of γδ T cells are important for adoptive cell therapy. This study shows similar results and finds that poorly expanded γδ T cells demonstrate exhausted T cell phenotypes with significantly elevated expression of PD-1, CTLA-4, Eomes and T-bet. These cells demonstrated inferior migratory and cytolytic activity to tumor cells in 2D culture and 3D spheroid assays. It was discovered that basal γδT levels and their immune phenotypes correlate with their expansion and cytotoxic effector potentials. High percentage of circulating γδ T cells with effector memory type and central memory phenotype may predict better expansion and cytotoxic effector function, independent of expansion methods, age, gender or HLA type. Furthermore, circulating γδ T cells in the good expansion group show significantly lower expression of exhaustion markers, PD-1, CTLA-4 and Eomes, and higher expression of IFN-γ and Granzyme B as well as costimulatory molecule of CD86 compared to those in the poor expansion group. It is unknown why circulating γδ T cells in some healthy donors express higher levels of checkpoint proteins. This may be determined genetically or related to donors' underlying medical conditions. Nevertheless, results herein support that expression of higher levels of checkpoint proteins reflects impairment of γδ T cell function. It is interesting that this defect cannot be overcome by addition of anti-PD1 or anti-CTLA-4 antibody in the culture medium.

In the present study, it was discovered that basal Vδ2 T cells with a larger proportion of central memory and effector memory types expand better. Similar to the results seen in adoptive OT cell transfer, a balanced distribution in Vδ2 T cell phenotype between central and effector memory versus terminally differentiated effector Vδ2 T cells may ensure both immediate effector functions, while maintaining proliferative potential to improve long-term protection. Increased effector memory γδ T cells are reported to correlate with objective clinical outcomes in patients treated with ZOL and IL-2. In contrast, the terminally differentiated effector Vδ2 T cells lose the proliferative potential and might tend to be functionally exhausted. Data herein suggest that circulating γδ T cells in healthy donors have intrinsic characteristics that impact their response to proliferation stimuli.

It has been shown that interindividual γδ T cell phenotypic heterogeneity develops after birth without correlation with gender, age, country of birth, or chronic stimulation in vivo and that γδ T cell concentrations in the PBMC were constant over time. Results herein are consistent with these findings. Furthermore, it was shown that γδ T cell expansion capacity is independent of HLA type and expansion methods. Importantly, it was discovered that the basal Vδ2 levels in PBMC correlate well with γδ T cell purity after expansion, expansion ratio, and absolute cell number. We develop a mathematical model to calculate a Vδ2 Index Score based on basal Vδ2 cell concentration in PBMC. Using Vδ2 Index Score of ≥0, it has a sensitivity of 97.78% and specificity of 90.48% with AUC of 0.968 to determine whether γδ T cell can expand well in a healthy donor. By combining the initial Vδ2 cell concentration, exhaustion markers such as PD-1, CTLA4, Eomes and T-bet, activation markers such as IFN-γ and Granzyme B and other costimulatory molecules such as CD80 and CD86 on the γδ T cells, we develop a comprehensive Vδ2 Index Score with a sensitivity of 95.56% and a specificity of 95.24% with AUC of 0.989. Surprisingly, addition of other immune markers does not significantly further enhance the sensitivity and specificity of the initial Vδ2 Index Score. The utility of this Vδ2 Index Score was tested in patient-derived melanoma organoids. Since γδ T cell function is independent of MHC recognition, patient-derived melanoma organoids are well suited to test anti-tumor functions of γδ T cells ex vivo. γδ T cells with a Vδ2 Index Score ≥0 have significantly better anti-tumor function than those with Vδ2 Index Score <0.

In summary, this study demonstrates that higher basal level of circulating γδ T cells predicts better expansion and functions (FIG. 14C). Checkpoint protein expression on γδ T cells correlates with their function and may be used as a quality control method before γδ T cell infusion in adoptive immunotherapy. These findings can be used to stratify healthy donors for γδ T cell-based therapy and enhance the efficacy of this promising treatment modality.

### Materials and Methods Corresponding to Examples 10-18

Study design: The study presented here was designed to develop a new method to expand Vδ2 T cells from PBMC ex vivo for adoptive T cell therapy (ACT). For in vitro and in vivo biomarker tests and anti-tumor assays, a minimum of three independent cytotoxicity experiments were performed as detailed below unless otherwise noted. Investigators were not blinded during the in vivo studies. In reverse phase protein array (RPPA) analysis, each group included five healthy donor PBMC samples and the data was analyzed by independent and blinded investigators.

Reagents: TLR1/2 agonist Pam3CSK4 (tlrl-pms), TLR2/NOD2 agonist CL429 (tlrl-C429), TLR2/4 agonist LPS-EB (LPS from E. coli O111:B4, tlrl-eblps), TLR4 agonist MPLA Synthetic (tlrl-mpls), TLR5 agonist FLA-ST (Flagellin from S. typhimurium, tlrl-stfla), TLR7 agonists Imiquimod (R837, tlrl-imqs) and Gardiquimod (tlrl-gdqs) were purchased from InvivoGen (CA, USA). TLR7/8 agonist Resiquimod (R848, SML0196), IPP triammonium salt solution (I0503), and Zoledronic acid (1724827) (also known as zoledronate, abbreviated herein as “ZOL”) were purchased from Sigma-Aldrich (MO, USA). Anti-human PD-1 antibody, pembrolizumab (Keytruda®, Merck & Co, New Jersey, USA. R014267), was stored at −80° C. at 25 mg/ml before use. MyD88 inhibitor ST-2825 was purchased from MedChemExpress (NJ. USA). mTOR inhibitors Torin1 (S2827, Selleck Chemicals, TX, USA) and Rapamycin (NC9362949, LC Laboratories, MA, USA) were described previously (Liu, et al., 2018, Nat Commun 9: 5426).

Primary cells and cell lines: PBMCs and monocytes were obtained from Human Immunology Core at the University of Pennsylvania, which were obtained from healthy donors-Using informed consent and approved by Institutional Review Board. For γδ T cell expansion, PBMCs were cultured with RPMI1640 media supplemented with 10% FBS (HyClone, GE Healthcare, UT, USA), 100 U/ml Penicillin-Streptomycin, 2 mM L-Glutamine, 1/1000 2-Mercaptoethanol (2-Me) (Gibco, ThermoFisher Scientific, MA, USA) (or without 2-Me as indicated) and 200 units/ml of Recombinant Human IL-2 (PeproTech, NJ, USA) in 24-well microplates, new media added and supplemented every other day. TCR-γδ T cells were expanded in 24-well tissue culture treated microplates that were coated with mouse-monoclonal Anti-Pan-TCR γδ antibody (1.0 ug/ml in PBS; IMMU510, IM1349, Beckman). For naive γδT cell isolation, PBMCs were purified using a commercial Human TCR γ/δ T Cell Isolation Kit (Miltenyi Biotec, Germany). Purified naive γδ T cells routinely exceeded >95% concentration by flow cytometry. Human melanoma cell lines A375, A2058, WM9, 903 were obtained from Meenhard Herlyn's laboratory (The Wistar Institute, Philadelphia, PA, USA) and they were routinely tested for mycoplasma and DNA fingerprinted (Liu, et al., 2018, Nat Commun 9: 5426). Lung cancer cell line H1975, Colon cancer cell line HT-29, Gastric cancer cell line NCI-N87 were purchased from ATCC. Daudi cells were obtained from Andrei Thomas-Tikhonenko Lab at the University of Pennsylvania and Children's Hospital of Philadelphia, K562 cells were obtained from Michael Milone Lab at the University of Pennsylvania. BRAFi and MEKi combination therapy resistant (CR) cell lines WM9-CR and A2058-CR were generated as described before (Lu, et al., 2017, Nature 550: 133-136).

Nude mice andxenograft: All animal procedures were approved by the Institutional Animal Care and Use Committees (IACUC) at the University of Pennsylvania and Wistar Institute. For xenograft, single A375 cell suspensions (2 million in 200 μl PBS) were injected into the left flank of each nude mouse (Jackson Laboratories) subcutaneously to obtain melanoma xenografts. Six mice per group for human IL-2 (i.p.) only as negative control group, IL-2 (i.p.)+γδ T cells (intratumorally, i.t.) expanded by IPP, and IL-2 (i.p.)+γδ T cells (i.t.) expanded by IPP plus Resiquimod. The first treatment was given when palpable tumor could be detected and tumor size reached approximately 50 mm³ and treated four times in 12 days.

Tumors were measured with a ruler and volumes were calculated in micrometer (mm) as length×width×height/2.

Humanized Mice (Hu-Mice): Humanized mice were generated at the Wistar Institute as described (Somasundaram, et al., 2021, Nat Commun 12: 346). Briefly, the humanized mouse model is developed by reconstituting immunodeficient NSG mice with fetal liver-derived CD34+ human hematopoietic stem cells, grafted intravenously, and autologous fetal thymus chunks, grafted under the renal capsule, to promote rapid human T-cell differentiation.

Flow cytometry: PBMCs and γδ T cell phenotypes were analyzed by flow cytometry. Briefly, single-cell suspensions were surface-stained for 30 min at 4° C. in dark, and intercellular staining was performed for 60 min after fixation and permeabilization using a True-Nuclear™ Transcription Factor Buffer Set (BioLegend, CA, USA). CFSE Cell Proliferation Kit for flow cytometry was purchased from Invitrogen (ThermoFisher Scientific, MA, USA). Fluorescein-conjugated antibodies were used. Data were acquired on LSRA/B or Fortessa B flow cytometers (BD Biosciences, NJ, USA) at Flow Cytometry and Cell Sorting Resource Laboratory at the University of Pennsylvania and analyzed with FlowJo software (Tree Star, Ashland, OR).

ELISA: IFN-7, IL-17A, and TNF-α levels of supernatant were determined by using commercial human ELISA kits from BioLegend (CA, USA) (430104, 433914 and 430204, respectively). PBMCs were cultured in 24-well microplate, supernatants were harvested and centrifuged at 5000 rpm for 5 min to remove lymphocytes without further purification. Samples were aliquot and stored at −80° C. until use and detected.

Cytotoxicity: Cytotoxicity of γδ T cells against melanoma cells was measured by lactate dehydrogenase (LDH) release using the CytoTox 96® Non-Radioactive Cytotoxicity Assay kit (G1780, Promega, WI, USA) as described (Zhu, et al., 2016, Molecular Medicine 22: 737-746). Briefly, Melanoma cells were seeded at 4×10⁴ cells/well in 50 μl standard growth medium in 96-well plates, while γδ T cells were seeded at effective target ratio E:T=5:1 in the same volume of PBMC culture media at the same time. After the indicated time, plates were spin down briefly and 50 μl supernatants were then harvested for further analysis. Absorbance at 490 nm was read using a BioTek Synergy HT reader (BioTek Instruments, VT, USA) after LDH activity detection, the percentage of cytotoxicity was calculated as (Experimental-Effector Spontaneous-Target Spontaneous)/(Target Maximum-Target Spontaneous)×100.

Melanoma spheroid: Multicellular melanoma spheroids were cultured as described (Ou, et al., 2021, Journalfor Immunotherapy of Cancer 0: e002274). Briefly, the 96-well plate was pre-coated with 50 μL 1.5% agarose before seeding CFSE (Thermo Fisher) labeled A2058 melanoma cells at 2×10⁴ cells/well and allowed to form spheroids over 48 hours. After coculturing with γδ T cells, an Annexin V/PI or PI (Biolegend, CA, USA) staining was performed for further analysis by Flow Cytometry or confocal microscope, respectively. The supernatant was harvested for cytotoxicity by LDH releasing assay (Promega, WI, USA) as described above

Single-cell RNA sequencing (scRNA-seq) dataset: scRNA-seq data of sorted γδ (Vδ2) T cells from three healthy human donor PBMCs were published by Gabriele Pizzolato et al. (Pizzolato, et al., 2019, Proc Natl Acad Sci USA 116: 11906-11915) and obtained from NCBI GEO data set repository (GSE128223). Three preprocessed mRNA datasets of GSM3667468 (763 cells), GSM3667470 (1277 cells), and GSM3667472 (1720 cells) were downloaded and analyzed with pipelines provided by Seurat Package (Stuart, et al., 2019, Cell 177: 1888-1902. e1821).

Identification of markers of yo T cellfates and cluster analysis of signaling pathways: To characterize the stage of maturation of γδ T cells, seven upregulated genes (NKG7, GZMA, IFNG, GZMB, FCGR3A, CST7 and KLRF1) and downregulated genes (CCR7, LEF1, LTB, SELL and IL7R) during γδ T cell activation and differentiation were employed as makers for cluster analysis. All three donor γδ T cells were combined for the UMAP representation. Cutoff setting was performed on each gene within each cell as 25% of all 12 genes to identify naive cells and terminally differentiated effector memory T cells. KEGG (Kyoto Encyclopedia of Genes and Genomes) pathway and Genontology (GO) analysis of significantly different expression genes of naive γδ T cells and terminally differentiated effector memory T cells were performed using DAVID bioinformatics resources (38, 39). Phosphoproteins in Functional_Categories analysis were specifically analyzed for signaling pathways. The single sample gene set enrichment analysis (ssGSEA) of single cell selected were performed as described (40).

Reverse Phase Protein Array (RPPA) assay: RPPA assay was performed as described previously (35). PBMCs from 5 healthy donors were cultured with IPP (5 μg/ml) or IPP (5 μg/ml) plus Resiquimod (10 μg/ml) for three or four days, then harvested and lysed for RPPA analysis. The array was probed with 297 antibodies and profiled by the RPPA platform at the MD Anderson Functional Proteomics Core facility by a standard operating practice (SOP) as described previously (41). KEGG pathway and GO analysis were performed as described above (38, 39). Heat maps were generated using Multiple Experiment Viewer (WebMeV) software as described (39).

Statistical analyses: Statistical analyses were performed with GraphPad 6.0 (Prism software package version) and Microsoft Excel Software. Data are presented as Mean S.E.M. and significant differences were examined with paired Student's t test. P<0.05 is considered statistically significant.

### Example 10: TLR Agonists Enhance Vδ2 T Cell Expansion In Vitro

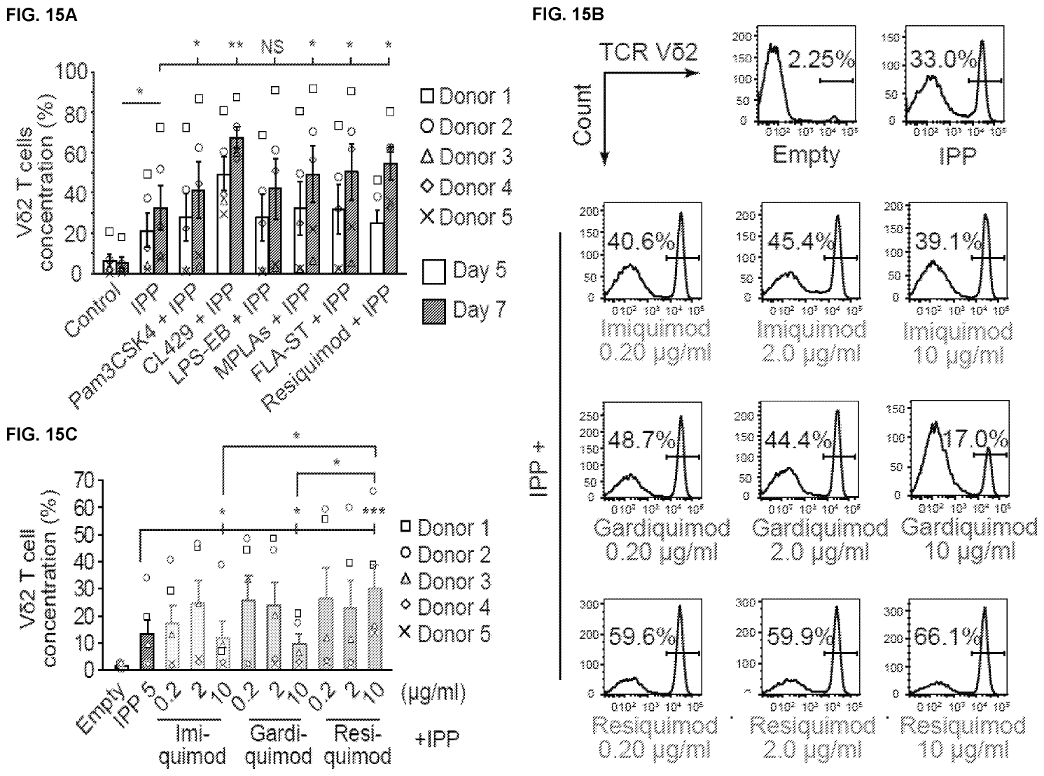

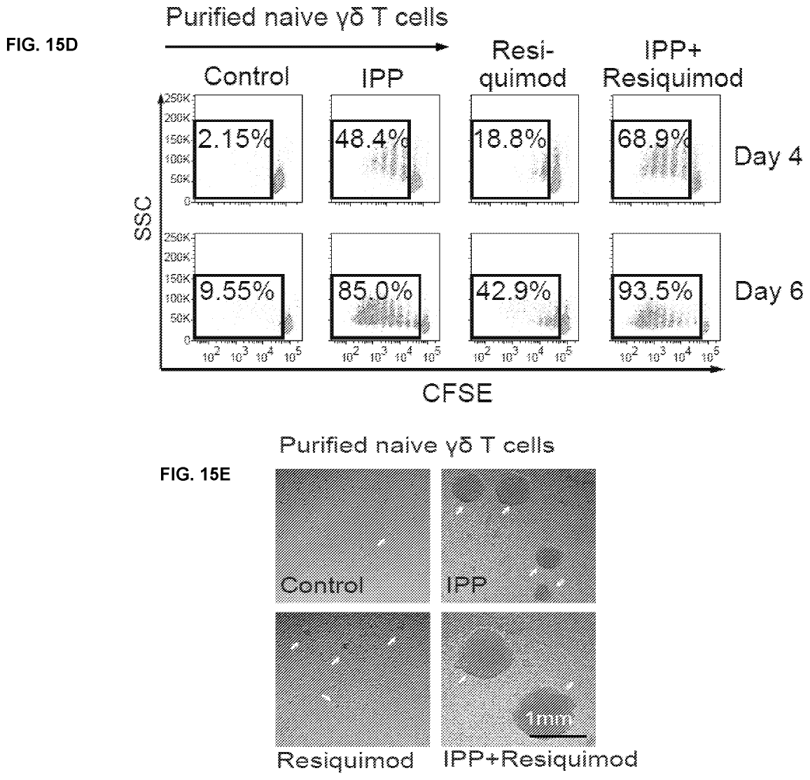

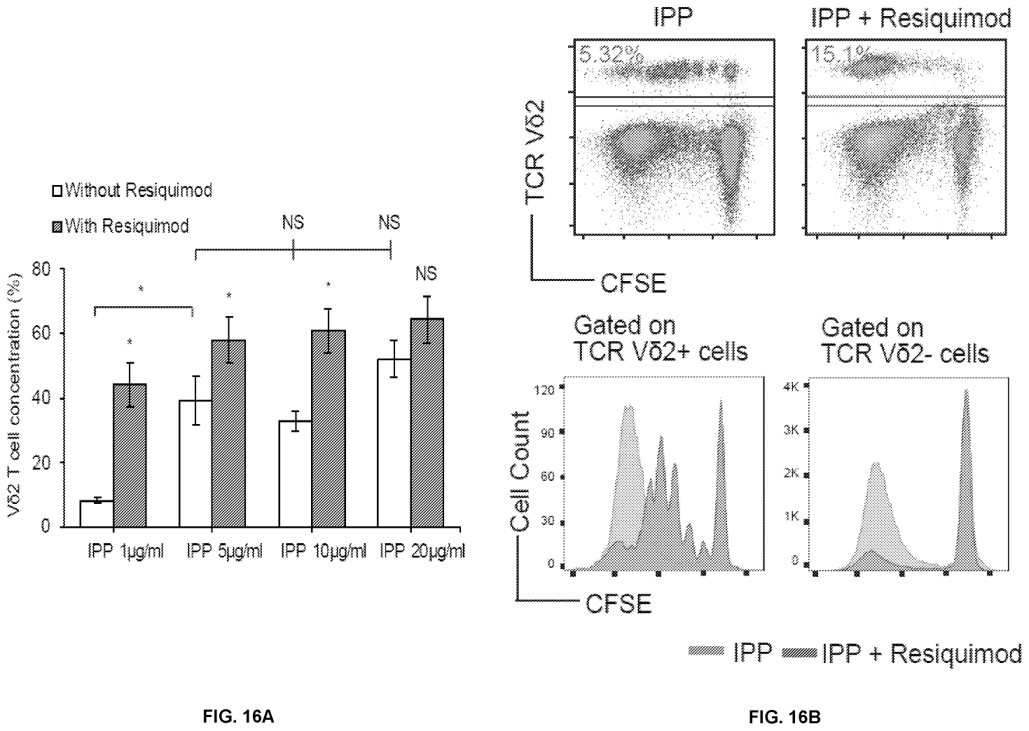

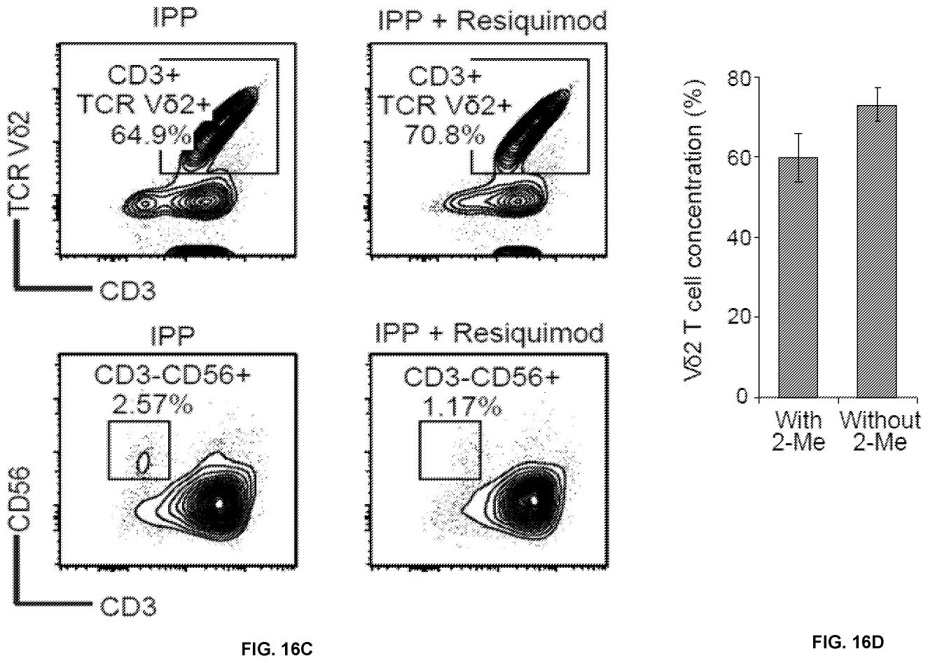

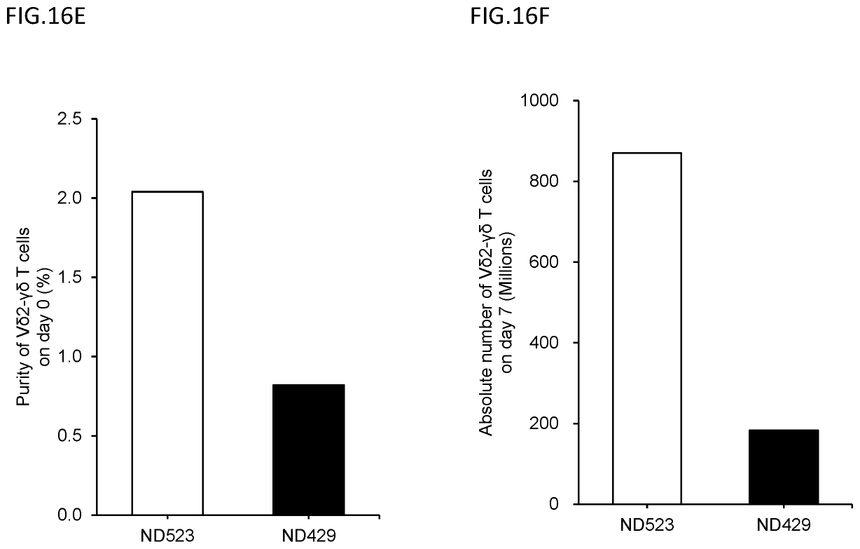

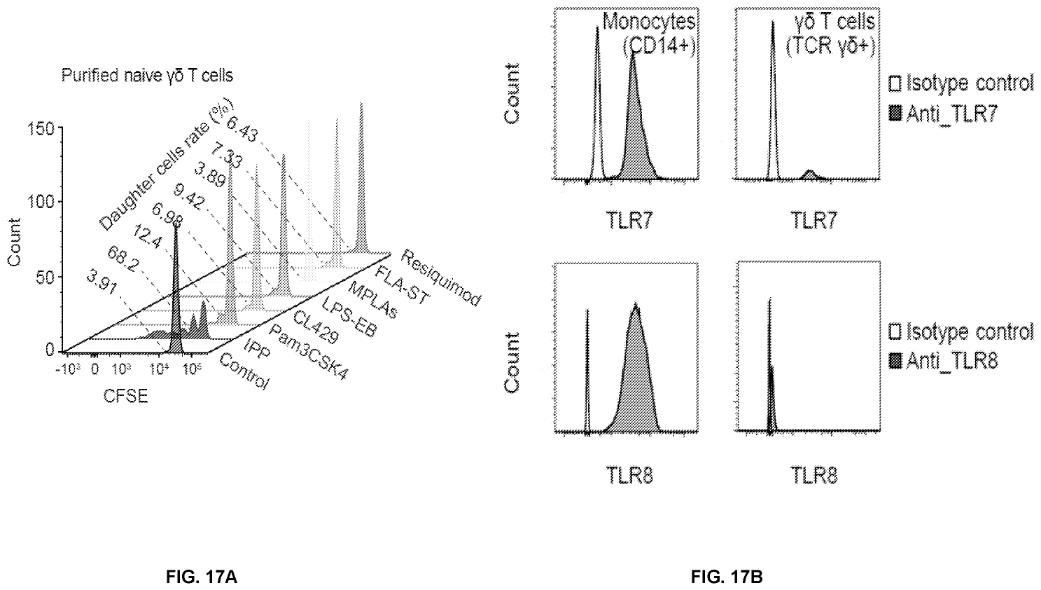

To identify TLR agonists that are costimulators for Vδ2 T cell expansion in vitro, several TLR agonists that were reported as potential adjuvants (Wesch, et al., 2011, Cell Mol Life Sci 68: 2357-2370), including Pam3CSK4 (TLR1/2 agonist, 0.3 μg/ml), CL429 (TLR1 & NOD2 agonist, 10 μg/ml), LPS-EB (TLR4 agonist, 10 μg/ml), MPLAs (TLR4 agonists, 10 μg/ml), FLA-ST (TLR3 agonist, 10 μg/ml), and Resiquimod (TLR7/8 agonist, 10 μg/ml), were screened with the phosphoantigen isopentenyl pyrophosphate (IPP) (5 μg/ml) (FIG. 15A) In vitro Vδ2 T cell expansion was additionally tested using various IPP concentrations with or without resiquimod (FIG. 16A). Although Vδ2 T cells in PBMCs from different donors showed different degrees of responses to IPP, nearly all TLR agonists demonstrated co-stimulatory effect (FIG. 15A). Next, the study was focused on TLR7/8 agonists which are small molecule stimulators that are clinically available or have been tested in clinical trials and therefore have the potential to be used to expand clinical grade γδ T cells. The co-stimulatory effect of three imidazoquinoline TLR7/8 agonists (imiquimod, gardiquimod, and resiquimod) on Vδ2 T cell expansion from PBMCs was compared (FIGS. 15B and 15C). The result showed that all three TLR7/8 agonists promoted Vδ2 T cell expansion, but resiquimod showed the best efficacy with the least cytotoxicity. IPP plus resiquimod induced proliferation of Vδ2 T cells specifically, but not other cell populations, including CD56+NK cells, in PBMCs (FIGS. 16B and 16C). An antioxidant used in the culture medium, 2-mercaptoethanol (2-ME), did not affect Vδ2 T cell expansion (FIG. 16D). Resiquimod also enhanced IPP induced proliferation of purified γδ T cells from PBMC. Costimulation of purified γδ T cells with IPP plus resiquimod induced more cell proliferation (CFSE dye dilution) and bigger colonies than IPP alone (FIGS. 15D-15E). TLR agonists alone showed only a modest effect on purified γδ T cell expansion (FIG. 17A). The naïve γδ T cells express a similar level of TLR7 as monocytes, but a lower level of TLR8 than monocytes (FIG. 17B).

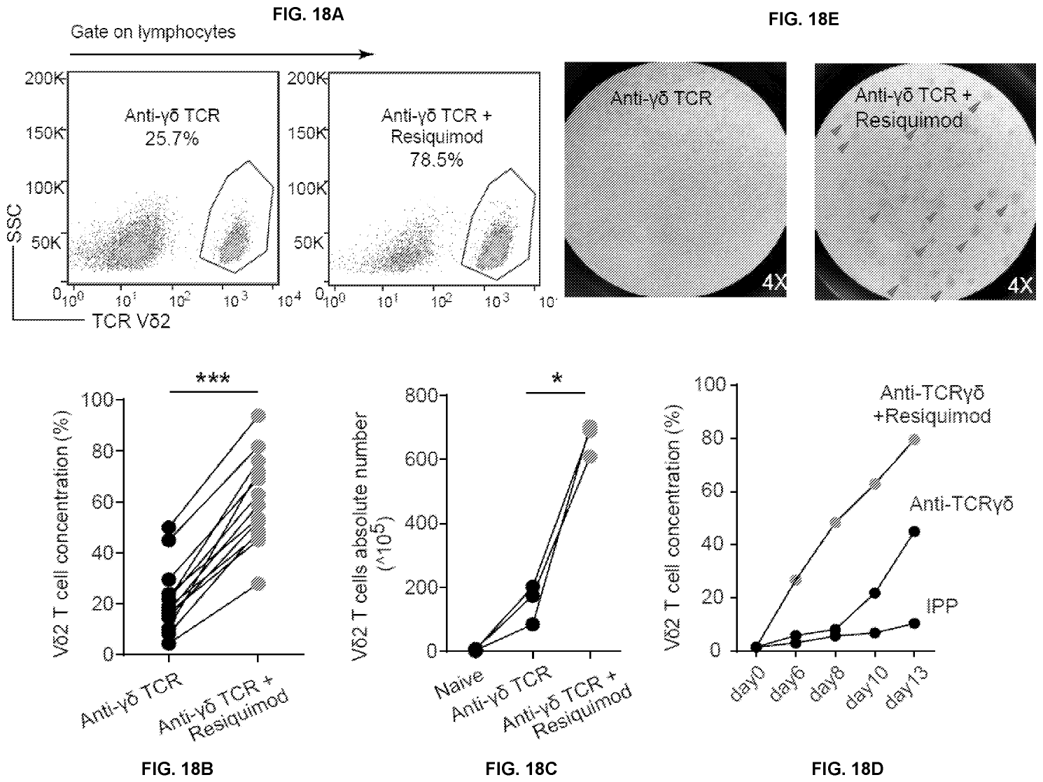

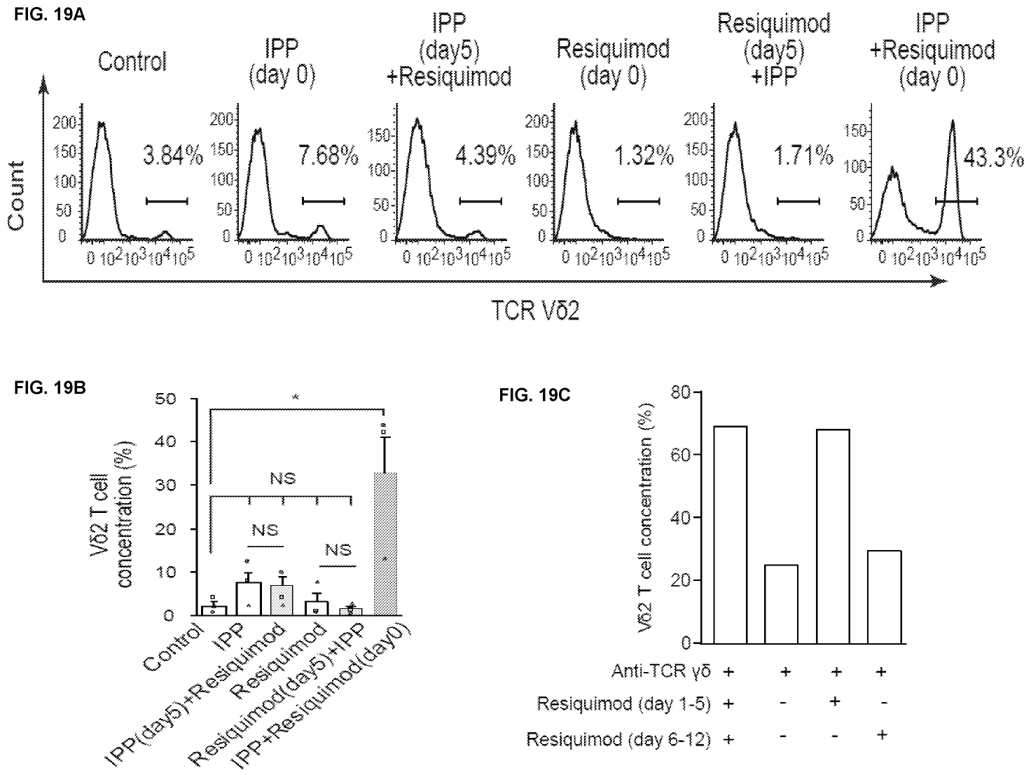

The costimulatory effect of resiquimod was not only seen in IPP-induced Vδ2 cell expansion but also in pan-anti-γδTCR antibody-mediated Vδ2 T cell expansion (FIGS. 18A-18E). In addition, the costimulatory effect of resiquimod was found to be dependent on the simultaneous activation of γδTCR and TLR7/8 during the early stage of Vδ2 T cell expansion. The addition of resiquimod to IPP containing Vδ2 T culture at day 5 did not increase the yield of Vδ2 T cells (FIGS. 19A-19C). Previously, it had been shown that there is large interindividual heterogeneity of Vδ2 cell expansion capacity that is independent of expansion methods (Ou, et al., 2021, Journal for Immunotherapy of Cancer 0: e002274). Vδ2 T cell expansion capacity correlated well with basal Vδ2 cell concentration in the PBMCs (Ou, et al., 2021, Journal for Immunotherapy of Cancer 0: e002274). Since different donors were used in the present experiments, this interindividual heterogeneity contributes to the variability of Vδ2 T cell expansion efficiency seen in the data.

### Example 11: TLR7/8 Activation Regulates Vδ2 T Cell Functional Marker Expression and Cytokine Release

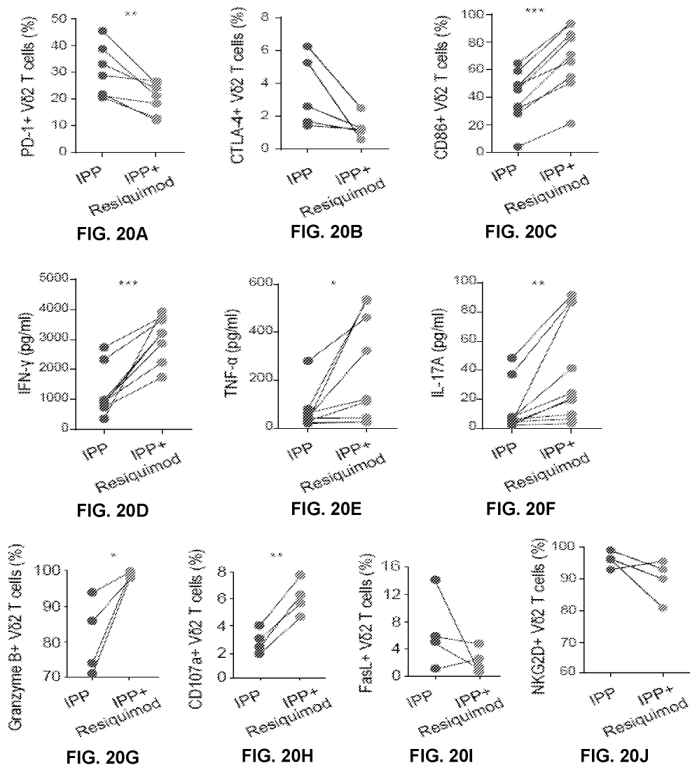

To gain insight into TLR7/8 activation induced functional changes in Vδ2 T cells, the expression of functional markers and secretion of cytokines after expansion were determined. Compared to expansion by IPP only, Vδ2 T cells expanded by IPP plus resiquimod showed more activation potential by expressing lower level of PD-1 (FIG. 20A) and higher level of CD86 (FIG. 20C) after 8 days of culture, while there was no significant change in CTLA-4 level (FIG. 20B). Meanwhile, IFN-7, TNF-α and IL-17A protein levels in the medium (FIGS. 20D-20F) and expression levels of cytotoxicity markers, Granzyme B and CD107a, (FIGS. 20G-20H) were significantly more elevated in Vδ2 T cells expanded by IPP plus resiquimod than by IPP alone. No significant change of Fas-ligand (FasL) or NKG2D was detected (FIGS. 20I-20J). These results support that resiquimod increases anti-tumor function and decreases the potential exhaustion of Vδ2 T cells in the culture.

### Example 12: TLR7/8 Activation Enhances Cytotoxicity of Vδ2 T Cells Both In Vitro and In Vivo

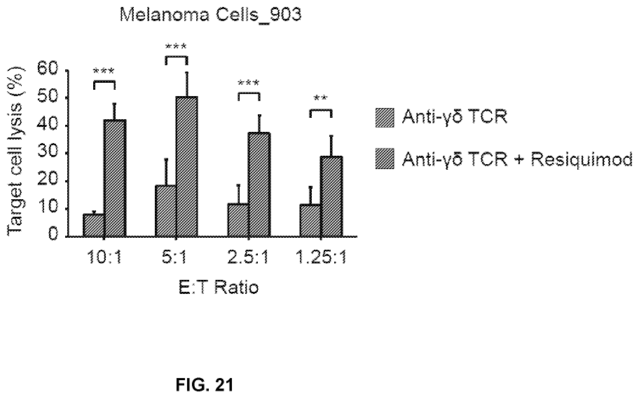

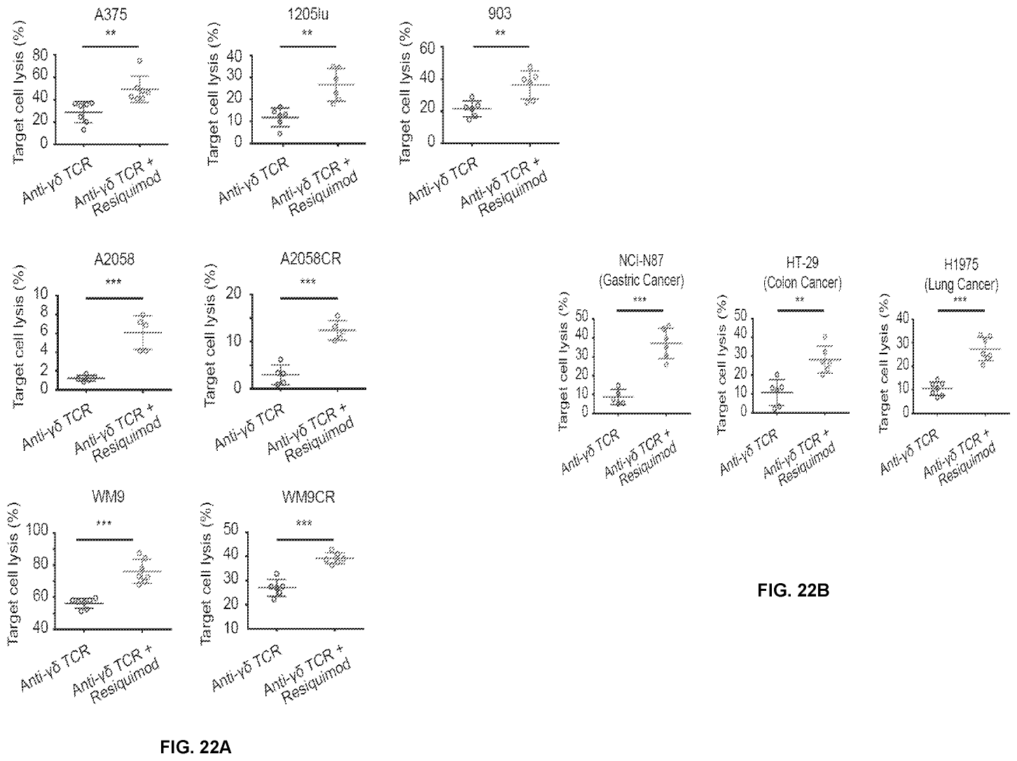

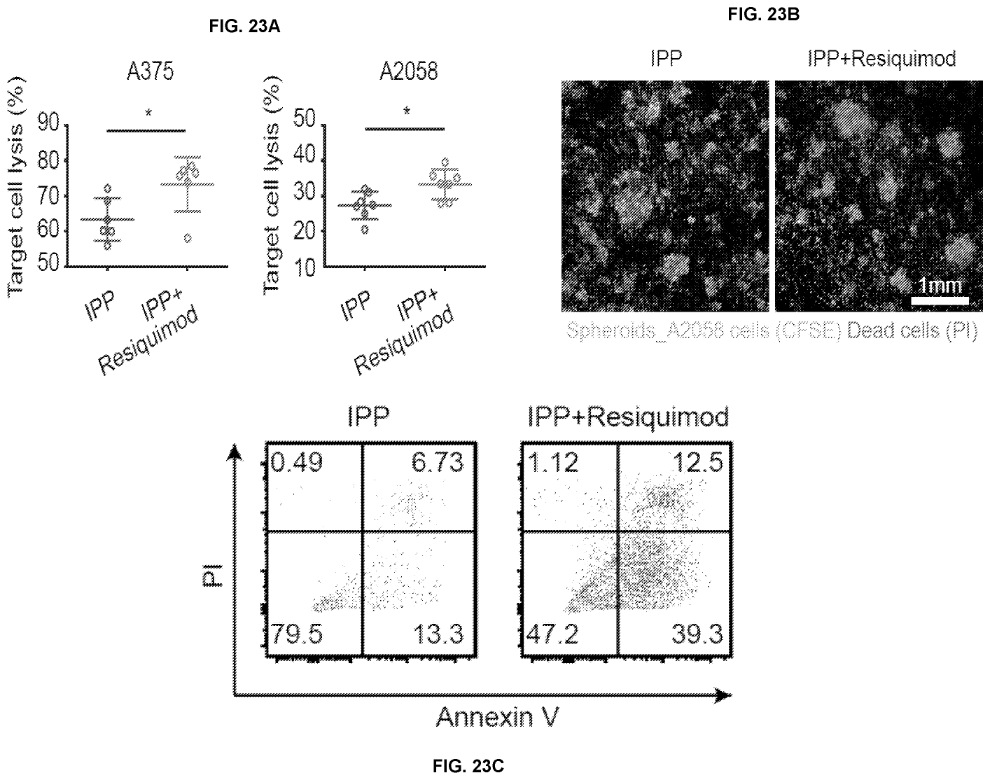

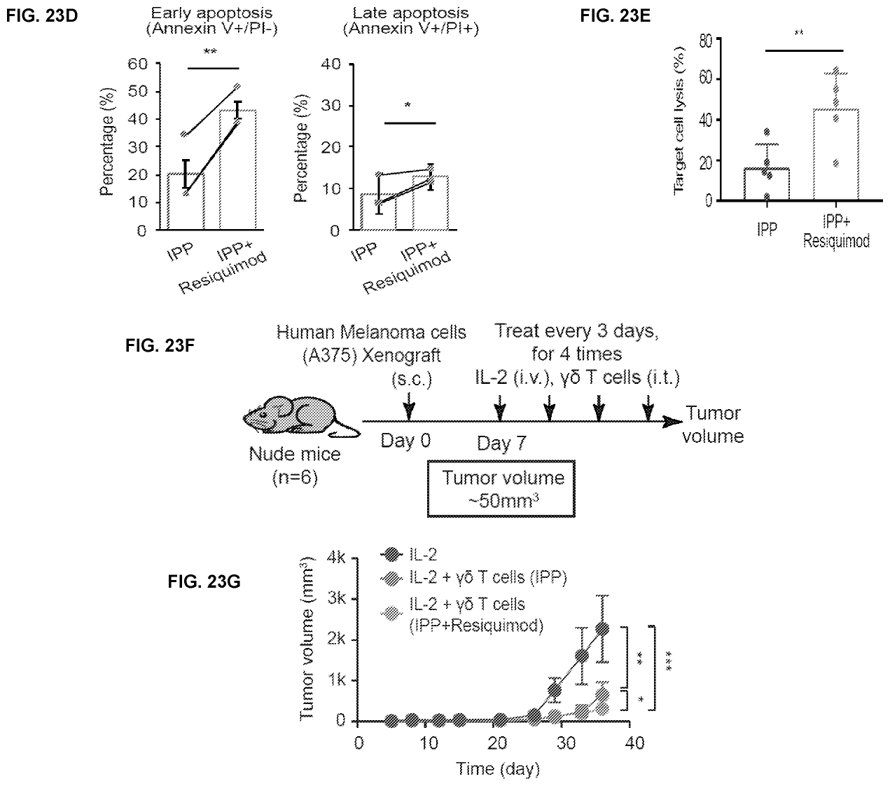

γδ T cells were expanded by IPP or pan-anti-γδTCR antibody with or without resiquimod for approximately 13 days. Expanded γδ T cells were incubated with melanoma cells (903 cells) at different Effector:Target (E:T) ratios of 10:1, 5:1, 2.5:1 and 1.25:1. An E:T ratio=5:1 showed the best tumor killing and less spontaneous LDH releasing (FIG. 21). Accordingly, E:T ratio=5:1 was used for other cytotoxicity assays in vitro. Different types of tumor cells, such as melanoma, lung cancer, gastric cancer, and colon cancer cells were tested. Resiquimod significantly enhanced the cytotoxic effect of γδ T cells expanded using IPP to two melanoma cell lines, A375 and A2058 (FIG. 23A). Additionally, resiquimod significantly enhanced the cytotoxic effect of γδ T cells expanded using pan-anti-γδTCR antibody to A375, 12051u, 903, A2058, and WM9 melanoma cells lines, as well as A2058CR and WM9CR, two melanoma cell lines that are resistant to BRAF inhibitor and MEK inhibitor combination therapy (FIG. 22A), and to gastric, colon and lung cancer cells (FIG. 22B). 3D melanoma spheroids (A2058) were cultured with Vδ2 T cells expanded by IPP or IPP plus resiquimod and stained with CFSE and propidium iodide (PI). Vδ2 T cells expanded by IPP and resiquimod induced significantly more PI stained dead tumor cells (FIG. 23B), more early (Annexin V+PI−) and late (Annexin V+PI+) apoptotic cells by FACS (FIGS. 23C-23D), and significantly more cell death by LDH cytotoxicity assay (FIG. 23E).

Next, the anti-tumor effect of the γδ T cells was tested in a nude mouse xenograft model (FIG. 23F). A375 melanoma cells were injected into the flank of nude mice. When the tumor became palpable (50-100 mm³), expanded Vδ2 T cells were injected intratumorally (i.t.). IL-2 was given intraperitoneally to sustain Vδ2 cells in vivo. Vδ2 T cells expanded by either IPP or IPP plus resiquimod significantly reduced tumor volume compared to the control group that was treated with IL-2 alone (FIG. 23G). The tumor volume reduction by cells expanded by IPP plus resiquimod was also significantly enhanced compared to that shown for cells expanded by IPP without resiquimod (FIG. 23G). These results support that co-stimulation of γδTCR and TLR7/8 promotes γδ T cell cytotoxic functions and enhances tumor control.

### Example 13: TLR7/8 Activation Enhances PI3K-Akt-mTOR Signaling Pathway in Vδ2 T Cells

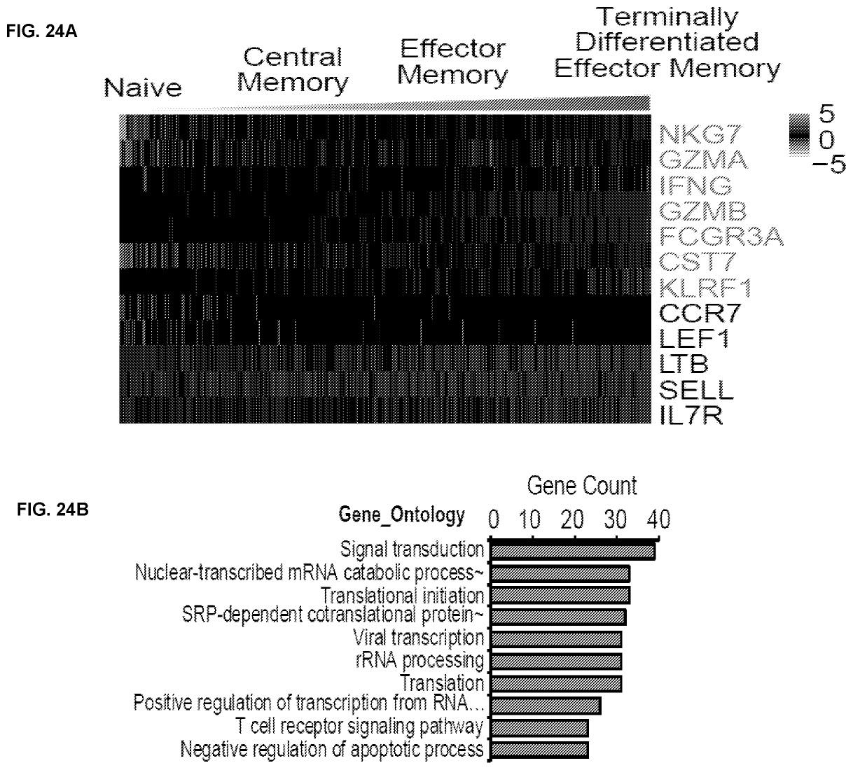

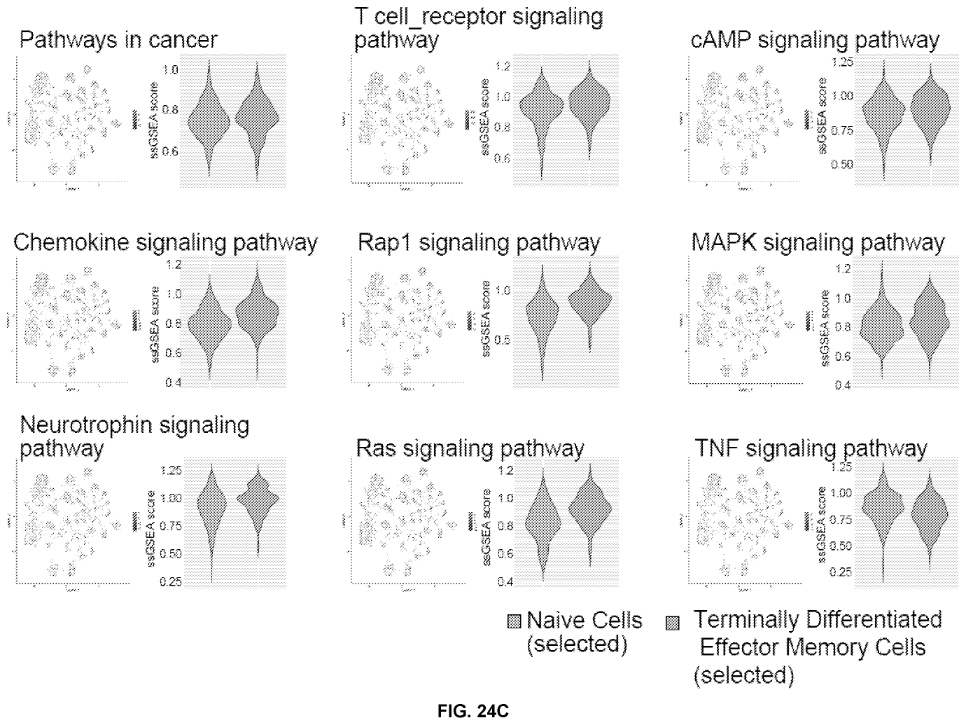

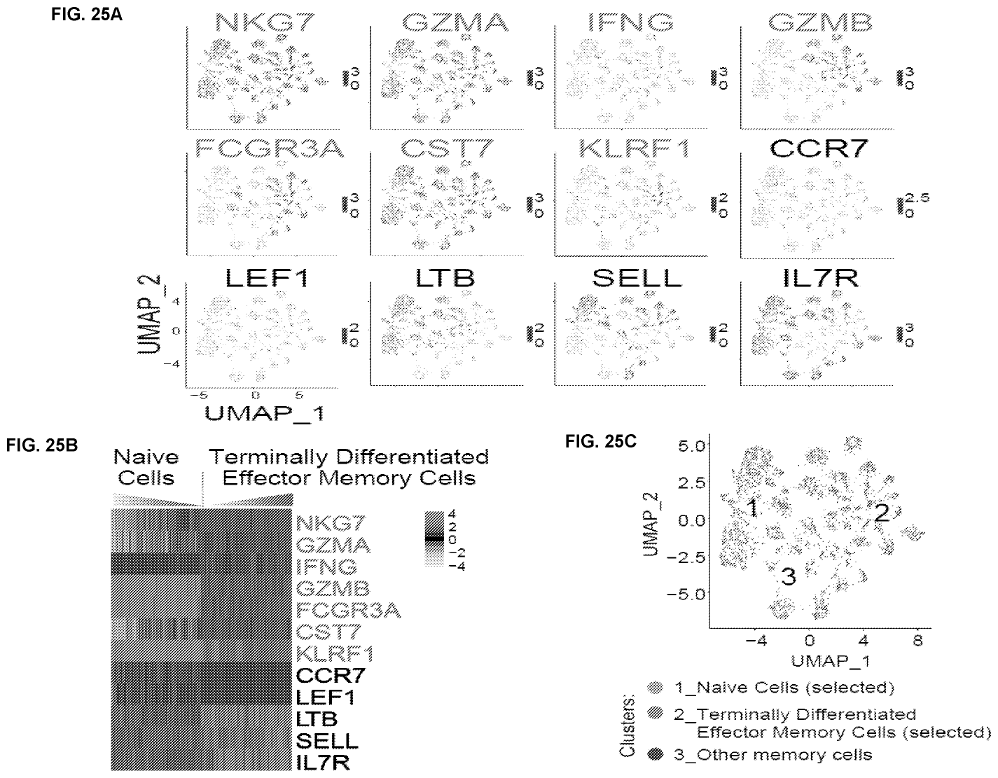

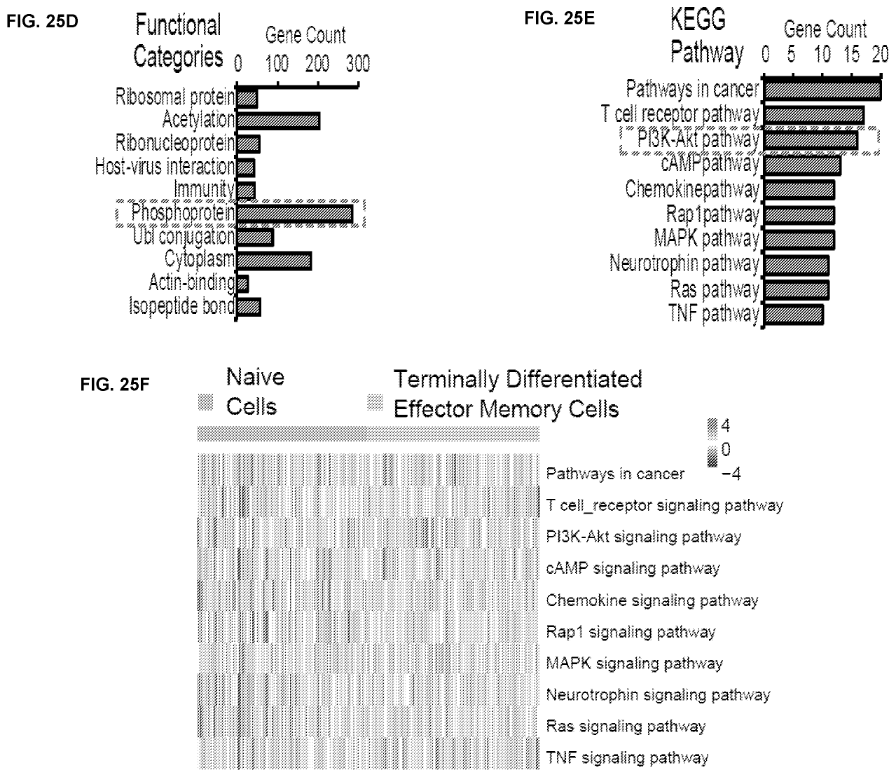

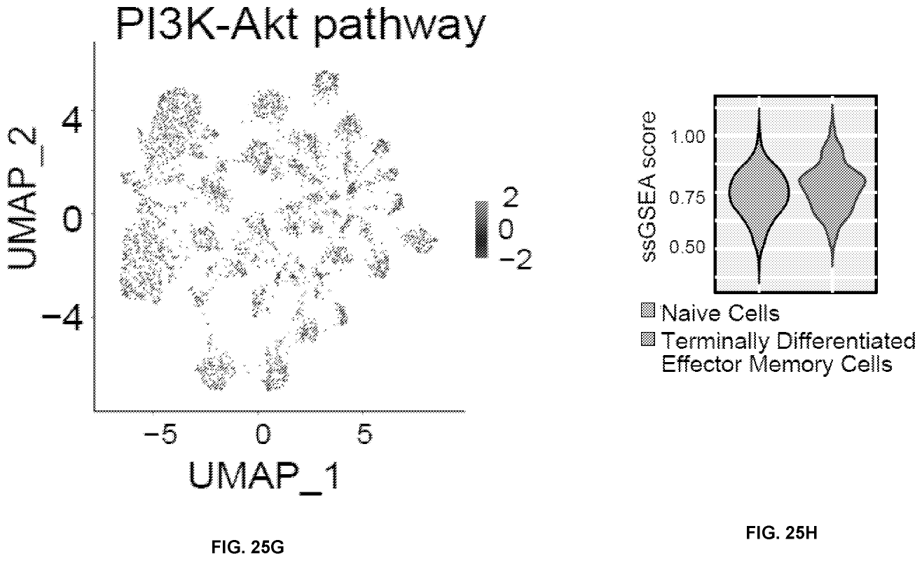

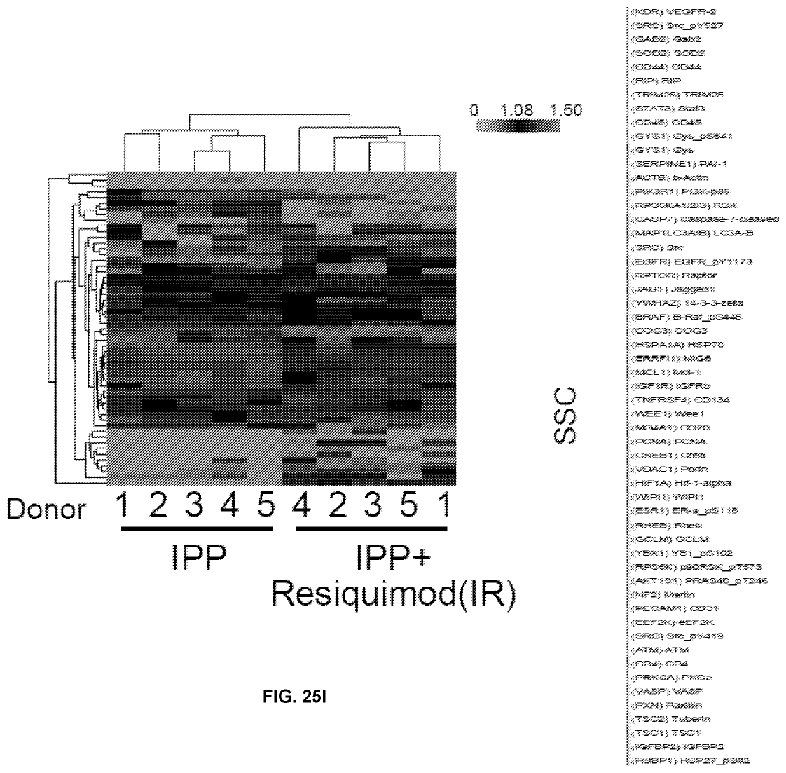

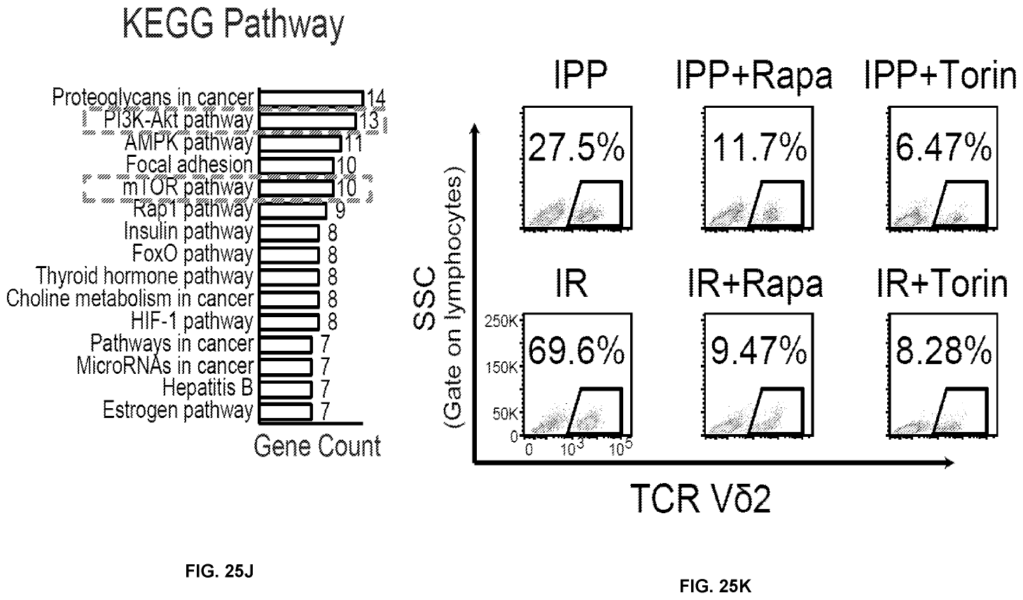

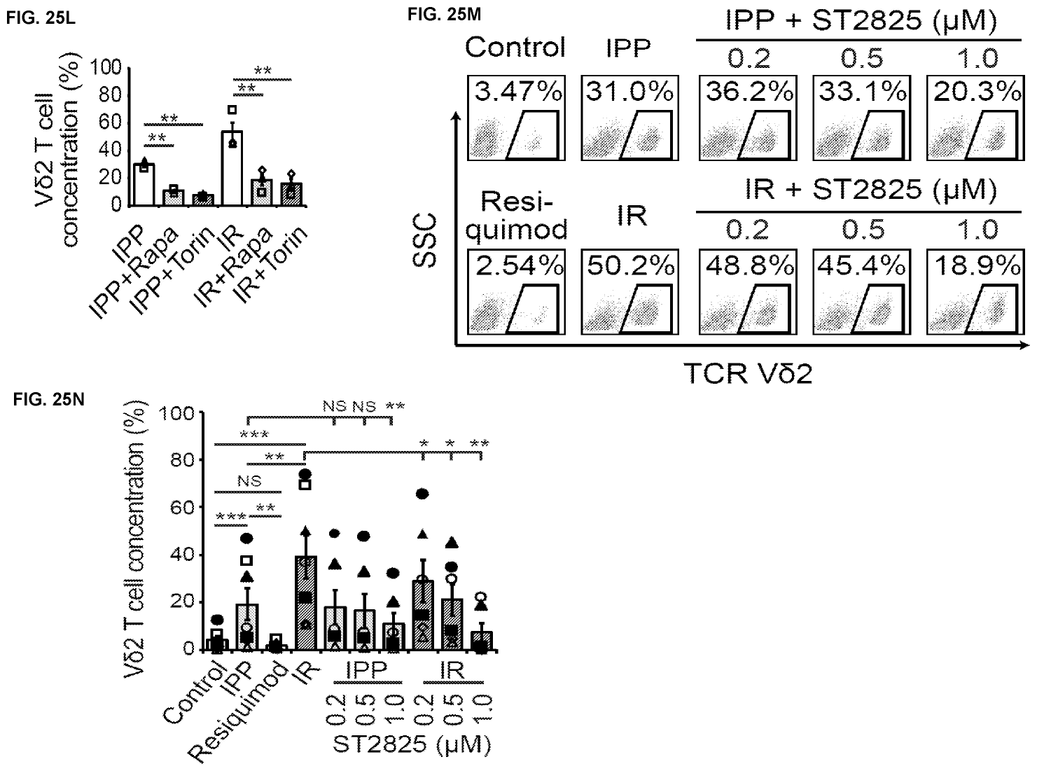

To explore the underlying mechanisms of Vδ2 T cell proliferative capacity and co-stimulation of γδTCR and TLR7/8 in Vδ2 T cells, published Vδ2 T cell scRNA-seq data from three healthy donors (19) was first analyzed by assessing the maturation status of Vδ2 T cells using 12 genes that were selected based on genes associated with Vδ2 T cell activation and maturation. These genes included genes that were upregulated in Vδ2 T cell activation and maturation, including NKG7, GZMA, IFNG, GZMB, FCGR3A, CST7, KLRF1 and downregulated genes including, CCR7, LEF1, LTB, SELL, IL7R (FIG. 24A). Clustering by Uniform Manifold Approximation and Projection (UMAP) identified 3 clusters of Vδ2 T cells that represented naive, terminally differentiated effector memory, and other memory cells, respectively, demonstrating the distinct maturation and fate stages of Vδ2 T cells (FIGS. 25A-25C). Signaling pathway analysis (FIGS. 25D-25E, FIG. 24B) and Single Sample Gene Set Enrichment Analysis (ssGSEA) (FIGS. 25F-25H, FIG. 24C) of selected naive cell cluster and terminally differentiated effector memory cell cluster were performed and, in particular, indicated that the PI3K-Akt pathway is one of the most relevant pathways in the process.

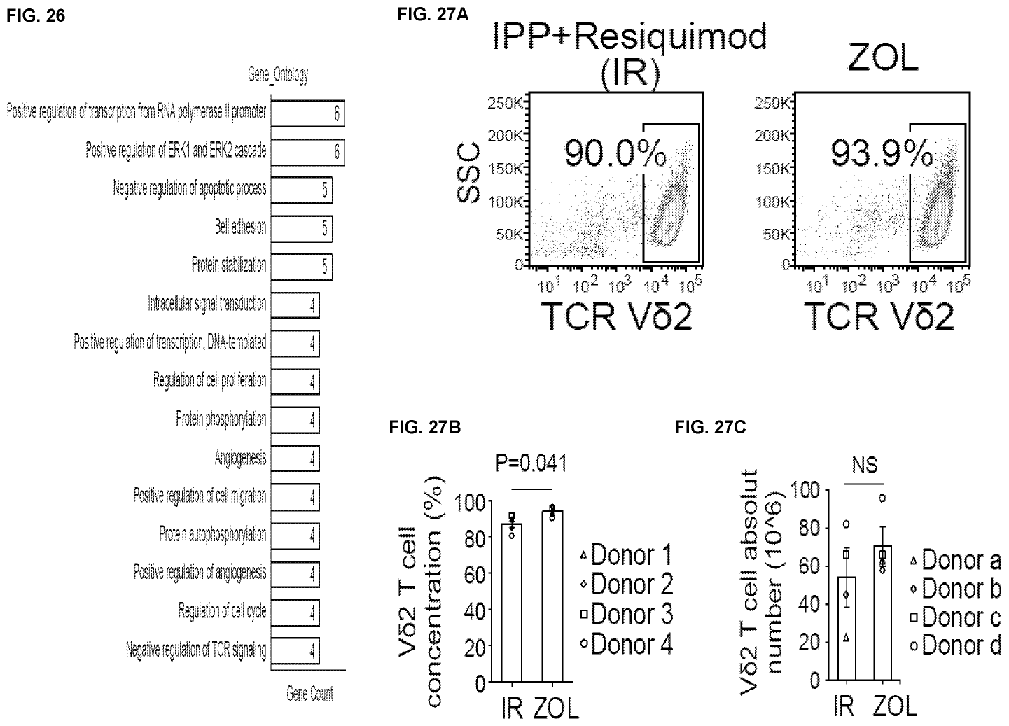

Next, reverse phase protein array (RPPA) assay was performed. RPPA data also showed that multiple pathways were activated, particularly, the PI3K-Akt-mTOR pathway (FIGS. 25I-25J, FIG. 26). To confirm the findings, two mTOR inhibitors, rapamycin and torin1, were used to inhibit the mTOR signaling in Vδ2 T cells. The results indicated that expansion of Vδ2 T cells induced by IPP was significantly reduced and the inhibition was more prominent in Vδ2 T cells expanded by IPP plus resiquimod (FIGS. 25K-25L). In addition, TLR7/8-MyD88 pathway was inhibitede by a MyD88 dimerization inhibitor, ST2825, which showed that inhibition of TLR7/8-MyD88 pathway eliminated the co-stimulatory function of resiquimod (FIGS. 25M-25N). These results support the idea that resiquimod promotes Vδ2 T cell proliferation through the TLR7/8-MyD88 and PI3K-Akt-mTOR signaling pathways.

### Example 14: Advantages of Vδ2 T Cell Expansion by IPP Plus Resiquimod Over Bisphosphonates

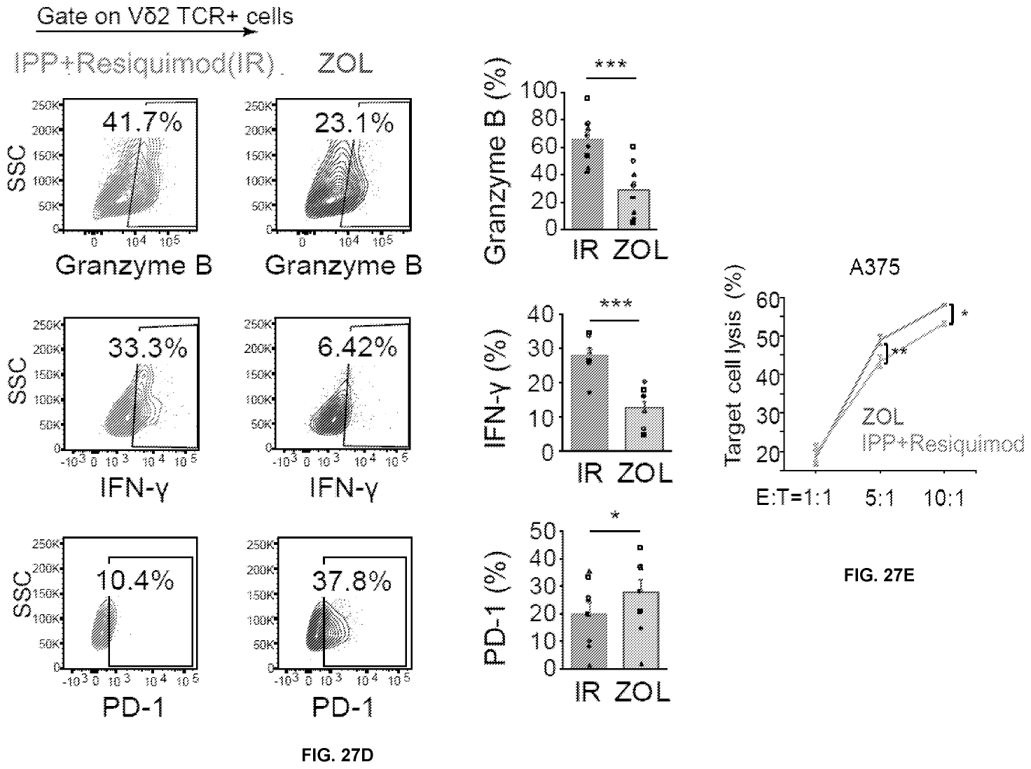

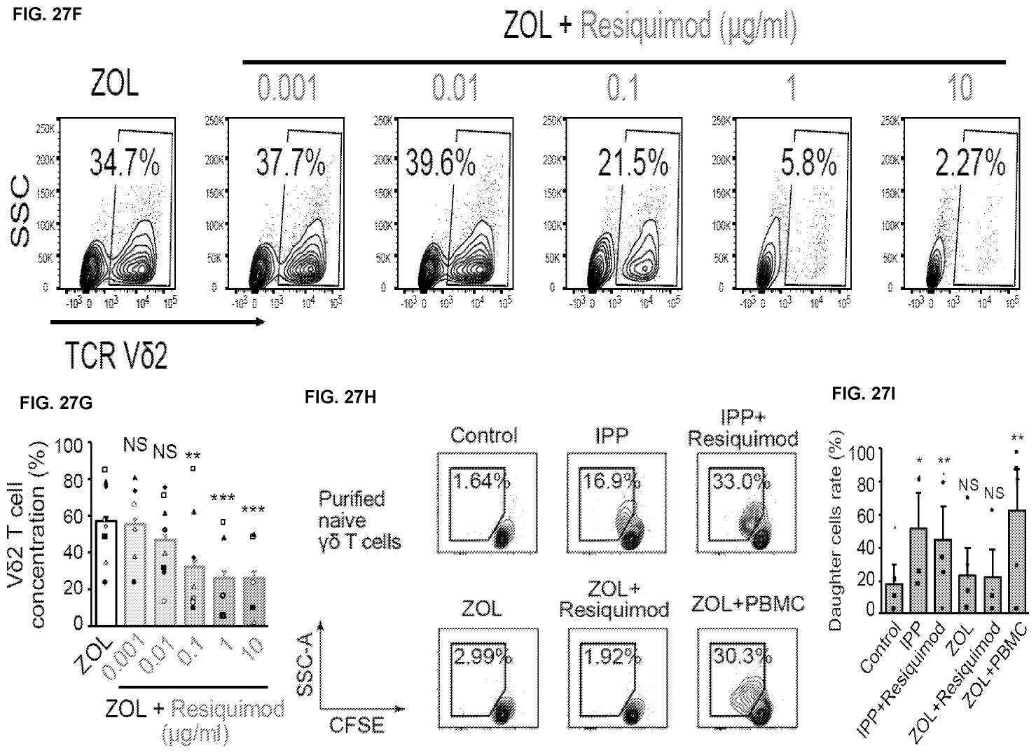

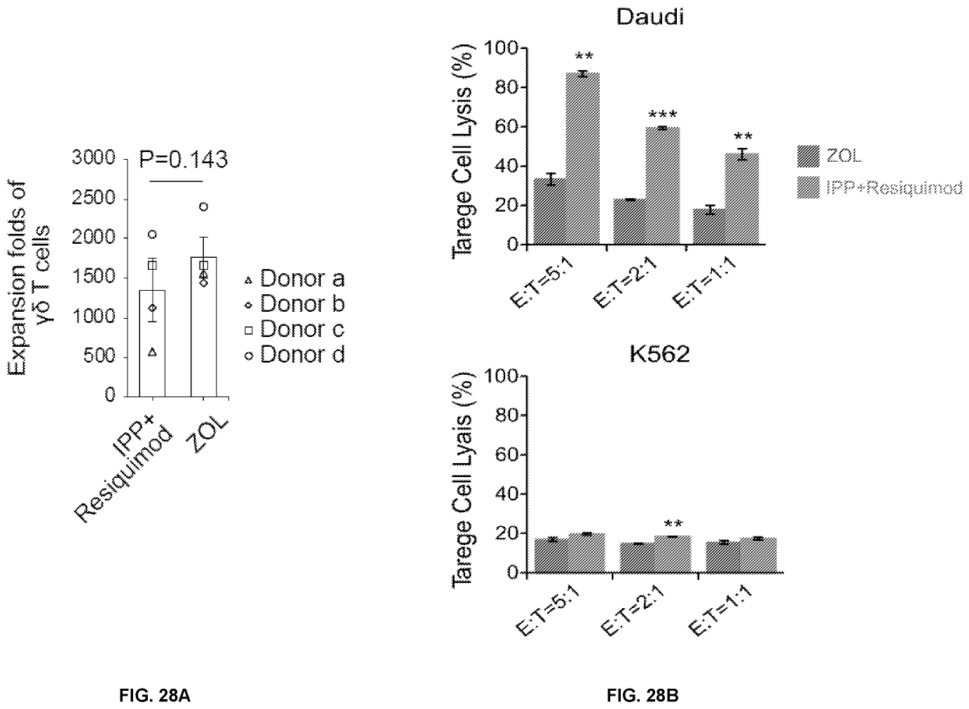

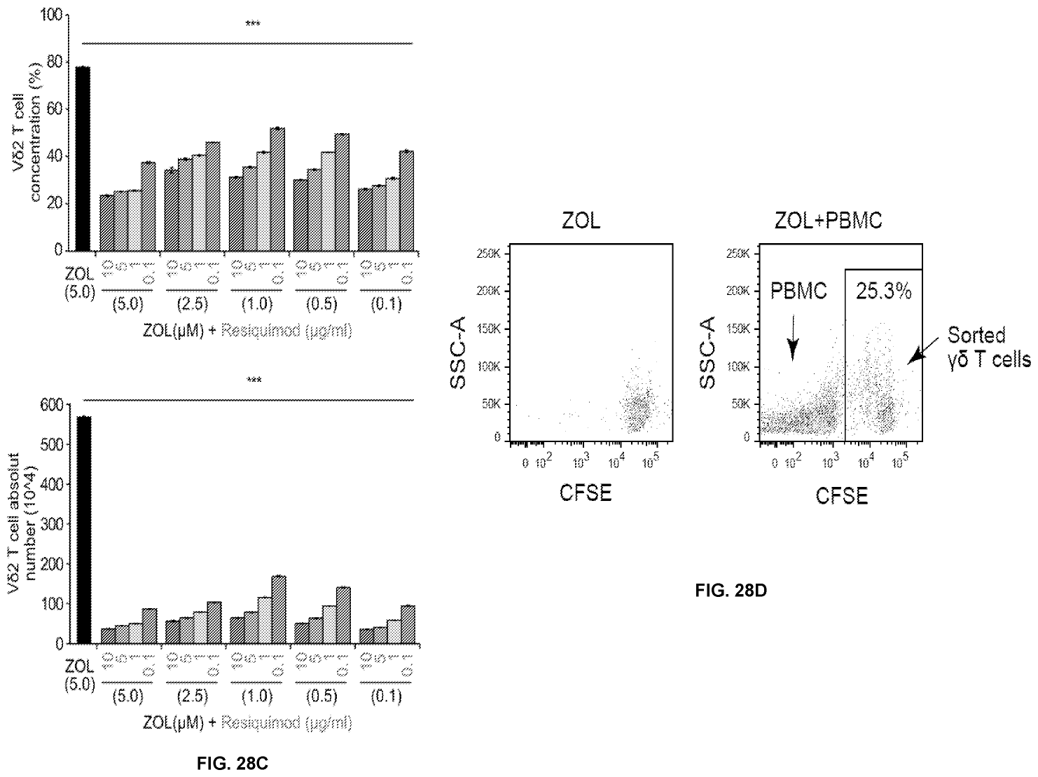

ZOL is the most commonly used agent to expand Vδ2 T cells from PBMCs as it is more efficient than IPP to induce Vδ2 T cell expansion. We compared the efficiency of Vδ2 T cell expansion using ZOL vs IPP plus resiquimod. Our data showed that the expansion rate of γδT cells using IPP plus resiquimod was comparable with the expansion using ZOL alone (FIGS. 27A-13C, FIG. 28A). Surprisingly, Vδ2 T cells expanded by IPP plus resiquimod expressed higher levels of Granzyme B and IFN-7, and a lower level of PD-1 compared to ZOL induced expansion (FIG. 27D). Furthermore, IPP plus resiquimod expanded Vδ2 T cells showed more cytotoxicity against A375 melanoma cells, Daudi and K562 lymphoma cells than Vδ2 T cells expanded by ZOL (FIG. 27E, FIG. 28B). These results are particularly unexpected, given that expansion of PBMCs with ZOL plus resiquimod was shown to not increase Vδ2 T cell expansion, but rather to decreased the yield of Vδ2 T cells (Serrano, et al., 2020, Cells 9), a result which was confirmed herein, particularly at higher concentrations of resiquimod (i.e., >0.1 μg/ml) (FIGS. 27F-27G, FIG. 28C),

APCs are essential for Vδ2 T cell expansion induced by ZOL. APCs release IPP to the surrounding microenvironment and present pAgs to γδTCR by BTN3A1 and BTN2A1 (Kabelitz, et al, 2020, Cellular & Molecular Immunology 17: 925-939). Using purified naive γδ T cells from PBMC and CFSE staining, TLR7/8 activation with resiquimod was shown herein to promote IPP-induced Vδ2 T cell proliferation, but not ZOL-induced Vδ2 T cell proliferation (FIG. 27H-27I, FIG. 28D). However, when PBMCs (CFSE—population in FIG. 28D) which contain monocyte-derived dendritic cells, were added in the culture system, ZOL regained the capability of inducing Vδ2 T cells (CFSE+) proliferation (FIGS. 27H-27I, FIG. 28D, ZOL+PBMCs). These results demonstrate that although the expansion efficiency of IPP plus resiquimod is equivalent to ZOL, these two expansion methods are mechanistically different. IPP plus resiquimod induced Vδ2 T cell expansion in a manner that is independent of APCs.

### Example 15: TLR7/8 Agonist Suppresses Inhibitory Function of Adherent APCs

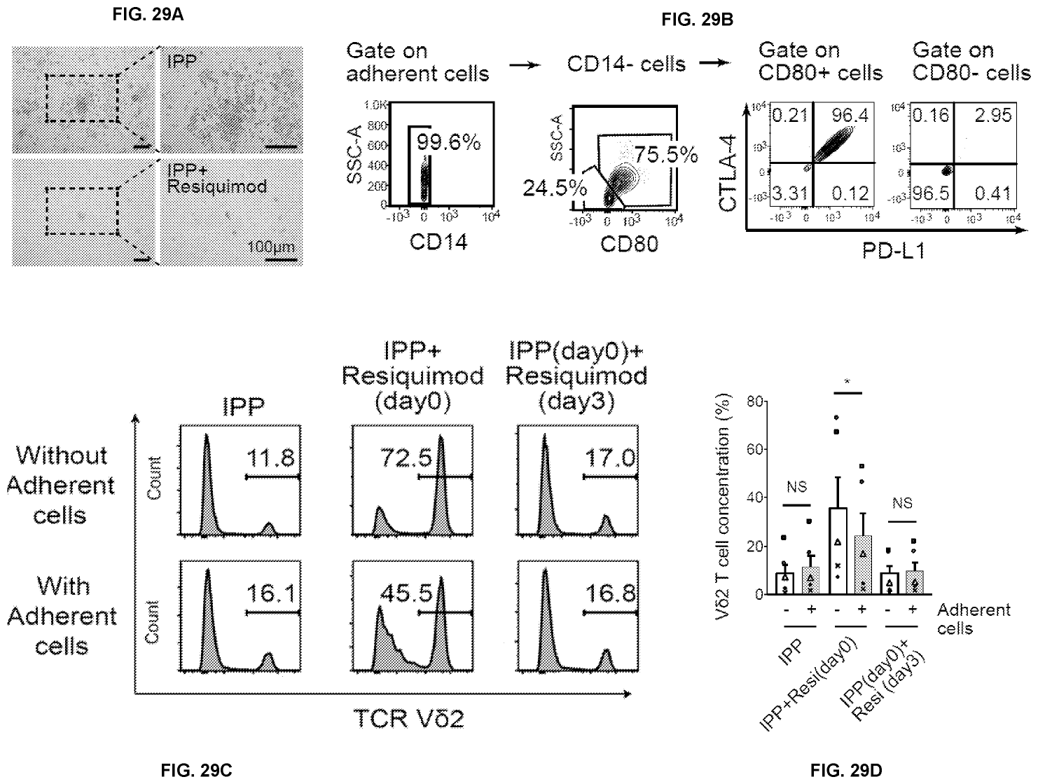

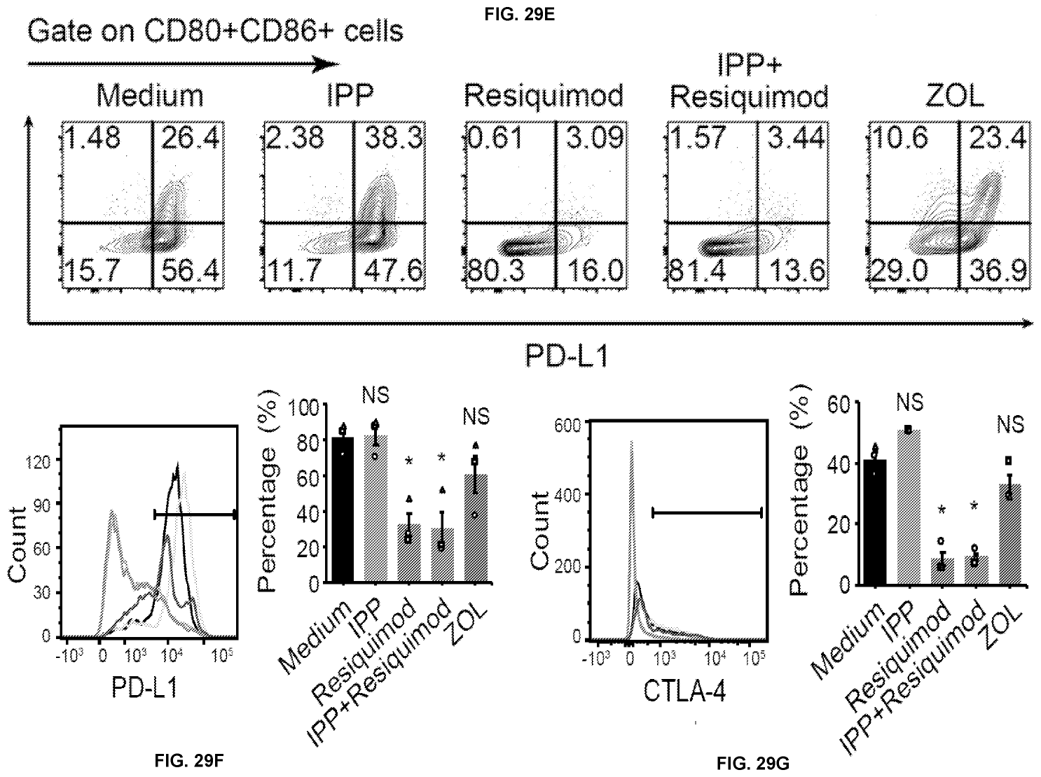

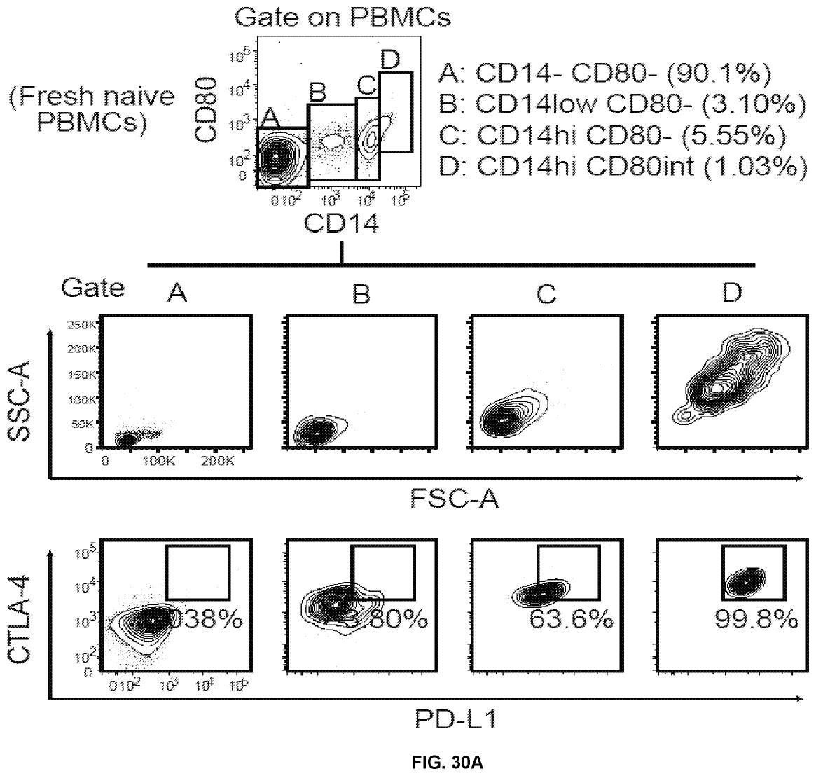

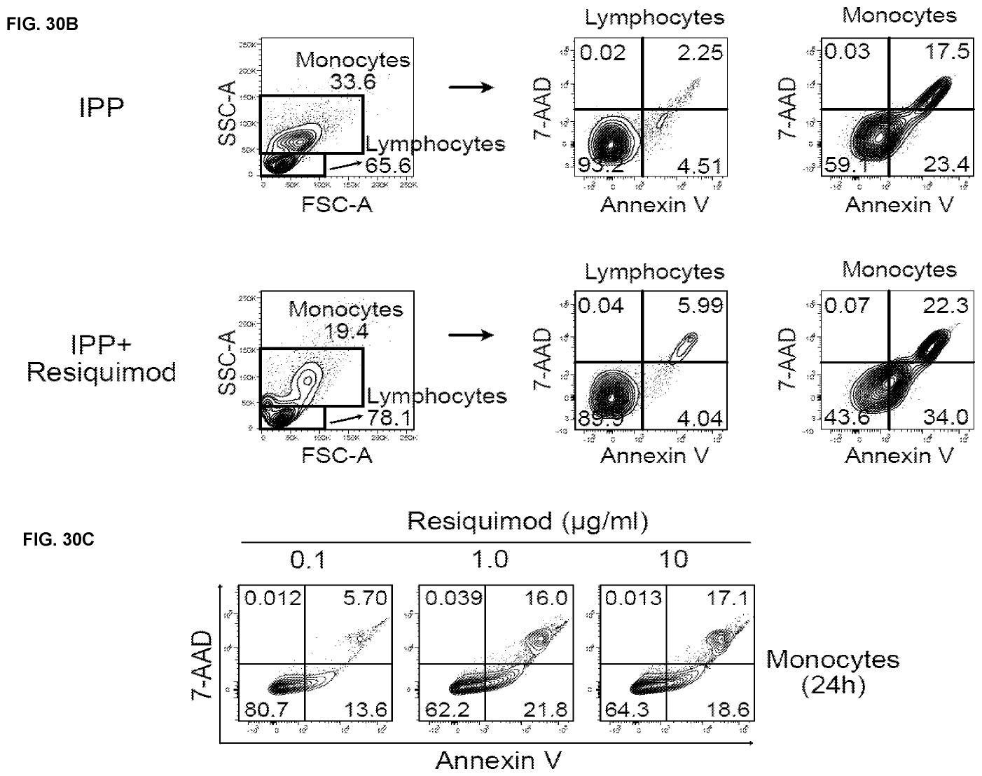

APCs are derived from CD14⁺ monocytes in the PBMCs during in vitro culture, which are adherent and highly express CD80 that binds to CD28 and CTLA-4 (Wang, et al., 2011, BMC Immunol 12: 21). Adherent APCs from PBMCs cultured with IPP or IPP plus resiquimod were distinctively different. Abundant adherent cells were present in the PBMCs cultured with IPP, but few with IPP plus resiquimod (FIG. 29A). Further analysis demonstrated that the adherent cells were CD14⁻CD80⁺, and they highly expressed PD-L1 and CTLA-4 (FIG. 29B). CD80⁺ cells were rare in fresh isolated PBMCs but all CD80⁺ cells highly expressed CD14, PD-L1, and CTLA-4 (FIG. 30A). Resiquimod induced apoptosis and cell death in some monocytes (FIGS. 30B-30C). These results suggest that CD14+ monocytes in PBMCs give rise to the adherent CD80+ APCs that express high levels of PD-L1 and CTLA-4.

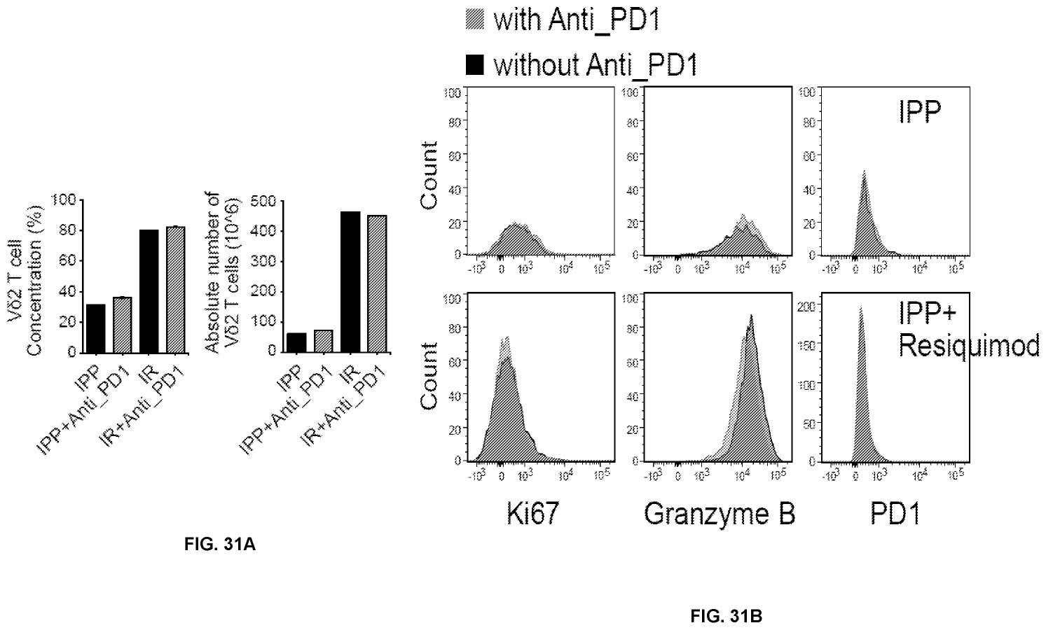

To further decipher the function of adherent APCs during γδ T cell expansion, the adherent APCs were isolated after culturing PBMCs with IPP for 3 days. These adherent cells were then added to PBMCs from the same donor that has been stimulated with IPP, IPP plus resiquimod, or ZOL for 3 days. Cells were harvested for Vδ2 T cell FACS analysis 3 additional days later. In a separate group, adherent cells and resiquimod were added to PBMCs that had been stimulated with IPP for 3 days to test whether the timing of TLR7/8 activation affected Vδ2 T cell proliferation. The results showed that adherent APCs significantly inhibited the enrichment of Vδ2 T cells induced by IPP plus resiquimod and ZOL (FIGS. 29C-29D). The effect of TLR7/8 activation occurred early during Vδ2 T cell expansion as the addition of resiquimod at day 3 did not affect Vδ2 T cell expansion (FIGS. 29C-29D). To further evaluate the inhibitory role of APCs, checkpoint protein expression was analyzed on CD80+CD86+ APCs in the cultured PBMCs (both adherent and non-adherent cells) after incubation with IPP, resiquimod, IPP plus resiquimod, or ZOL, respectively. The presence of resiquimod, with or without IPP, inhibited the expression of PD-L1 and CTLA-4 on CD80+CD86+ APCs (FIGS. 29E-29G). However, anti-PD1 antibodies failed to reverse the inhibitory effect of APCs in Vδ2 cell expansion induced by IPP and functional marker expression (FIGS. 31A-31B). These results support that activation of TLR7/8 suppresses formation of adherent APCs and their inhibitory functions.

### Example 16: Vδ2 T Cell Expansion in Humanized Mice (Hu-Mice)

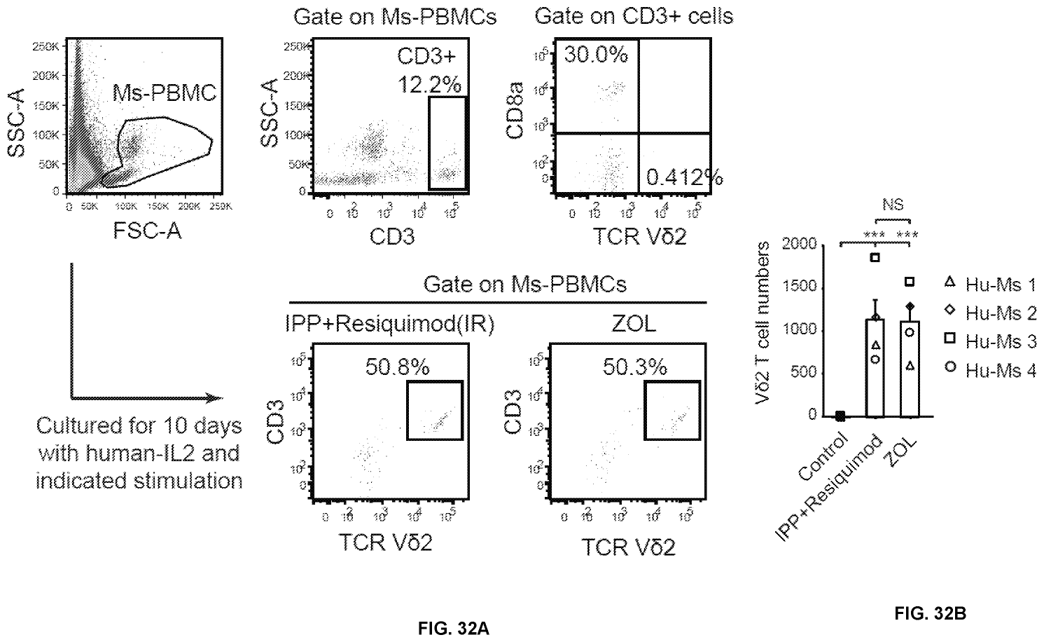

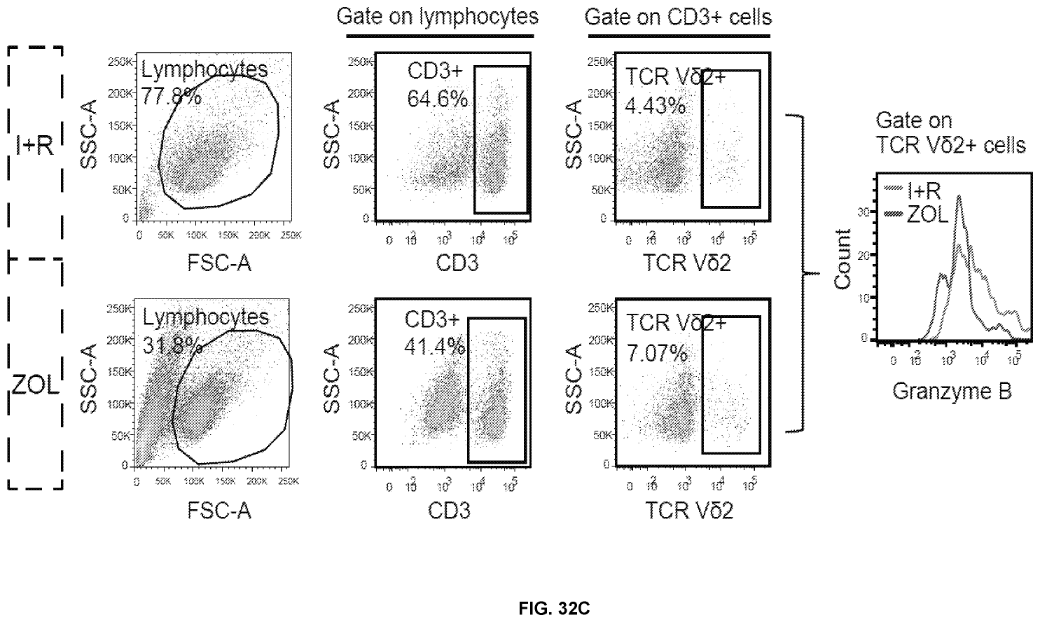

NSG mice engrafted with human hematopoietic stem (CD34+) cells or peripheral blood leukocytes (Hu-Mice) are a useful tool to study immunotherapy (Somasundaram, et al., 2021, Nat Commun 12: 346). However, it was previously unclear whether these mice have functional human γδ T cells. As shown herein, CD3⁺VS2⁺ T cells are present in the PBMCs of Hu-Mice (FIG. 32A). PBMCs from four Hu-Mice were cultured using human PBMCs culture media and were stimulated with IPP plus resiquimod, or ZOL, for 10 days. Peripheral Vδ2 T cells in Hu-Mice responded to stimulation and showed approximately a thousand-fold expansion by either IPP plus resiquimod or by ZOL (FIG. 32B). However, Vδ2 T from Hu-Mice expanded by IPP plus resiquimod showed more Granzyme B expression than those expanded by ZOL (FIG. 32C). These data show that Hu-Mice engrafted human peripheral Vδ2 T cells, and as such, that such Hu-Mice may be a useful preclinical model for γδ T cell functional studies.

### Example 17: Anti-γδTCR Antibody-Coated Magnetic Beads

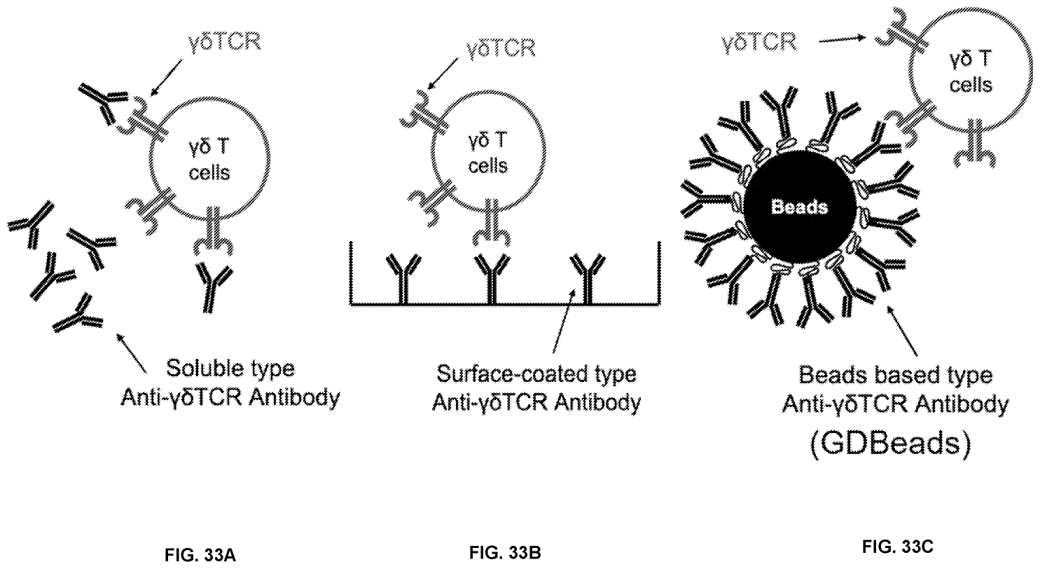

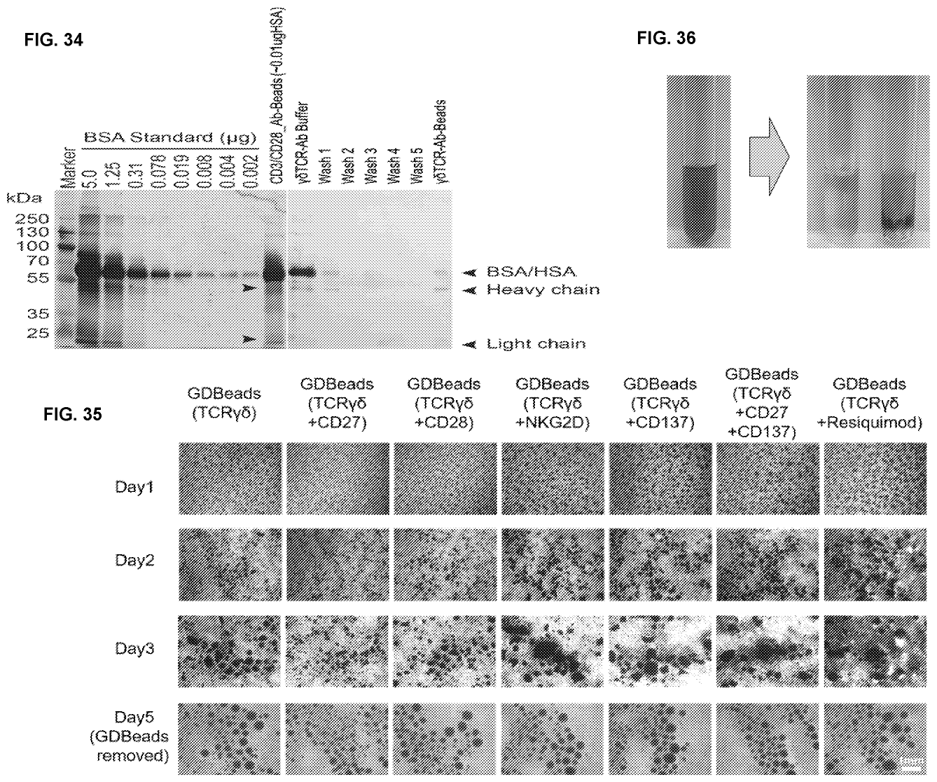

Both soluble and immobilized anti-γδTCR antibodies can promote the activation and proliferation of γδ T cells (FIGS. 33A-33B). As an alternate and improved approach, anti-γδTCR antibodies were effectively coated onto 0.45 m diameter magnetic beads, either alone or in combination with secondary co-stimulatory receptor antibodies including anti-CD27 antibodies, anti-CD28 antibodies, anti-NKG2D antibodies, anti-CD137 (4-1BB) antibodies, or anti-CD277 (BTN3A1) antibodies (FIG. 33C and FIG. 34). The antibody-coated beads are termed “GDBeads” herein. The presence of the GDBeads with PBMCs at a ratio of 1:1 in vitro culture can stimulate γδ T cell expansion clonal formation (FIG. 35). GDbeads can be removed by magnetic field at any time during the culture process to obtain cells free from antibodies and beads contamination (FIG. 36).

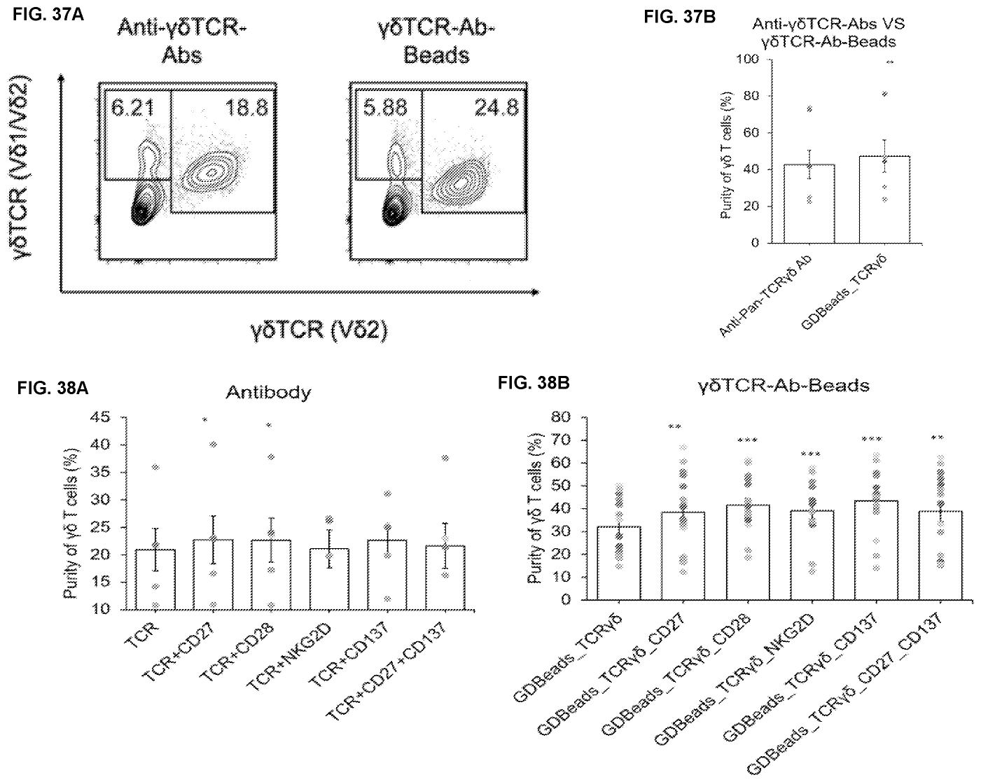

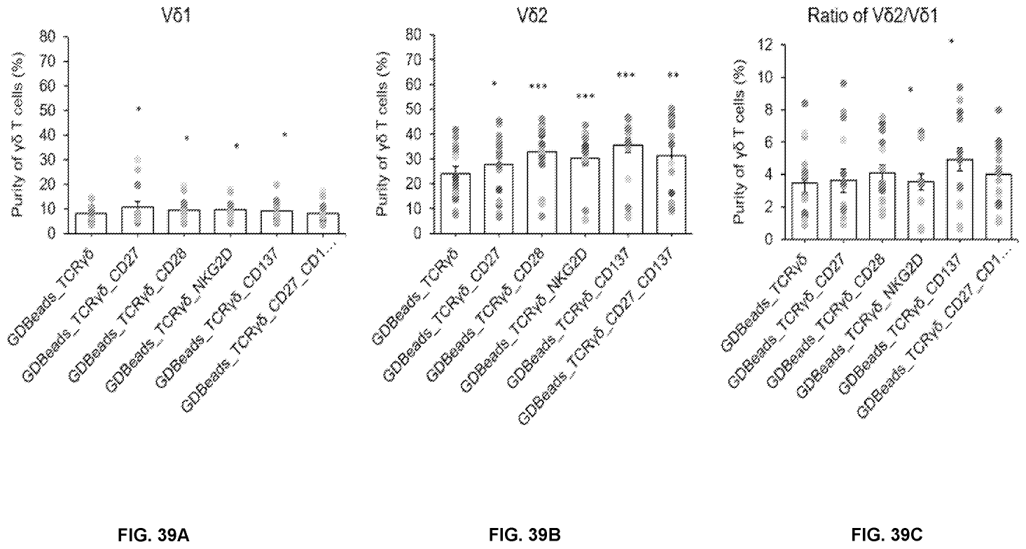

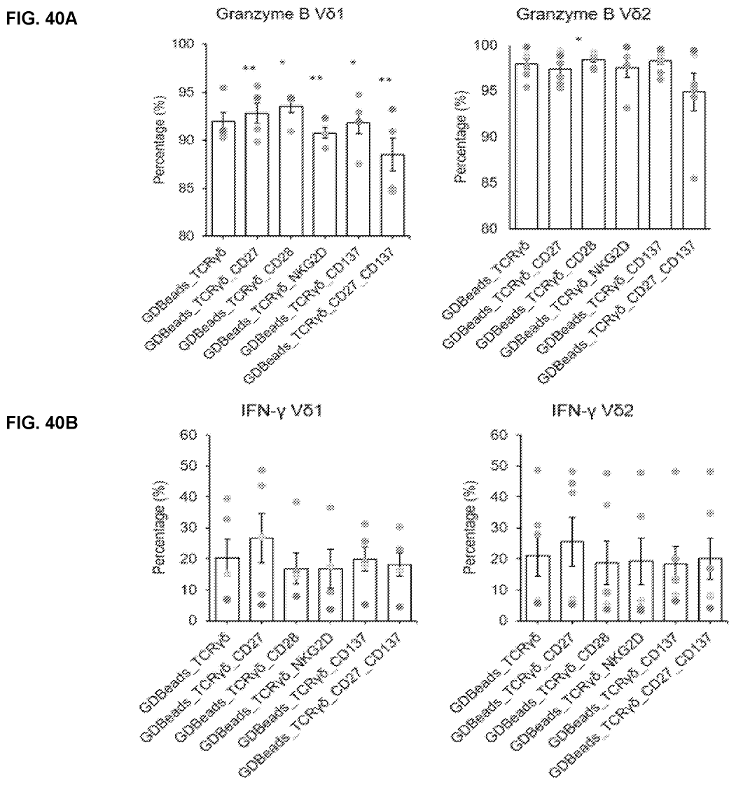

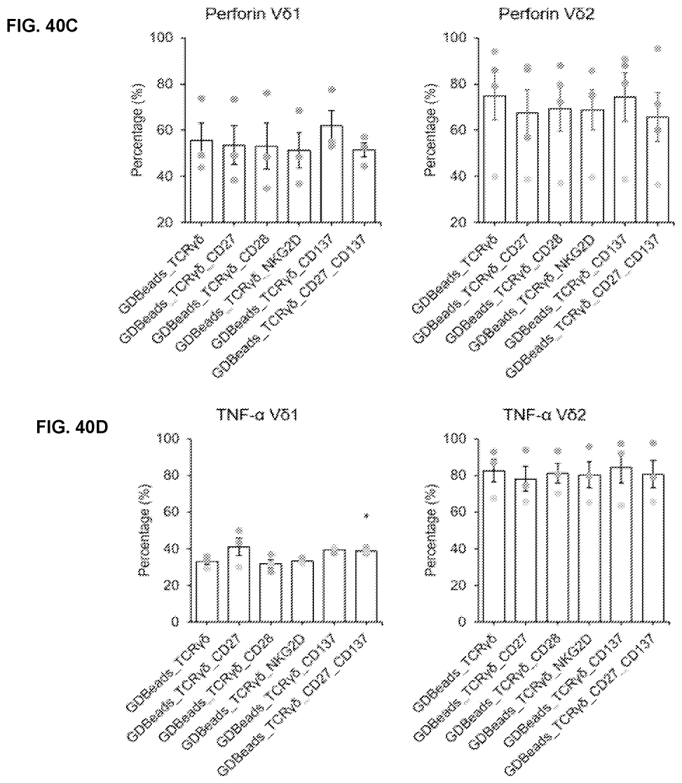

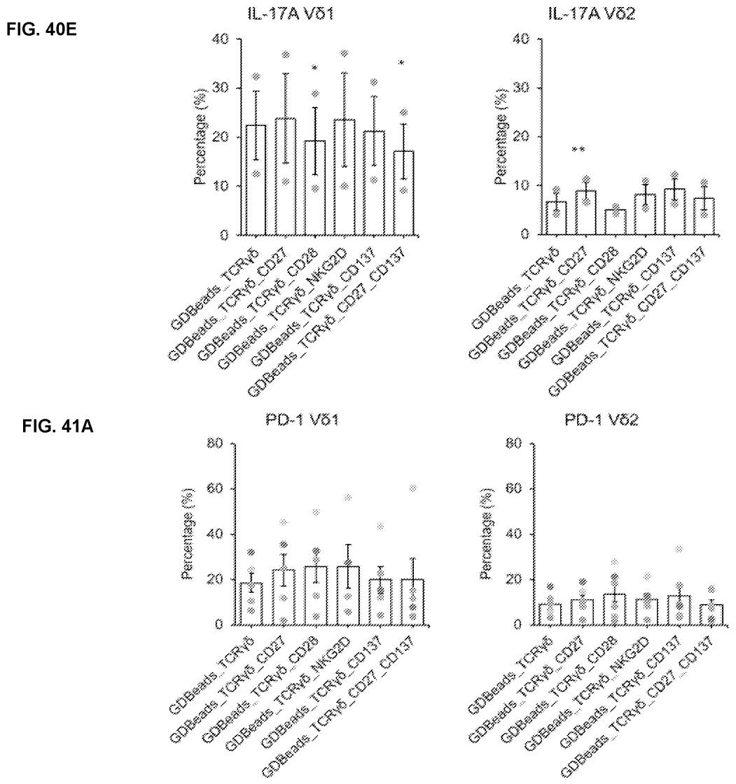

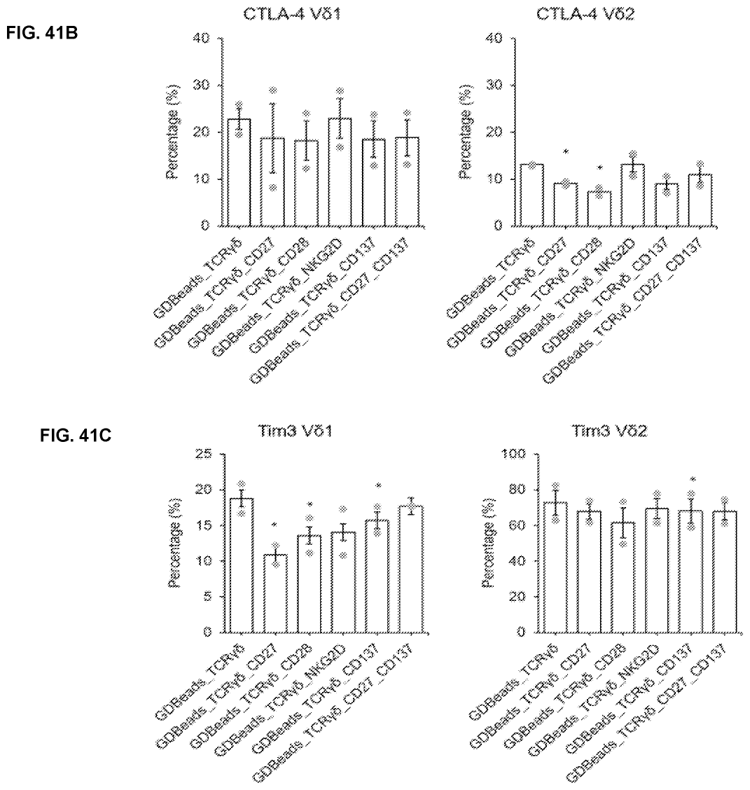

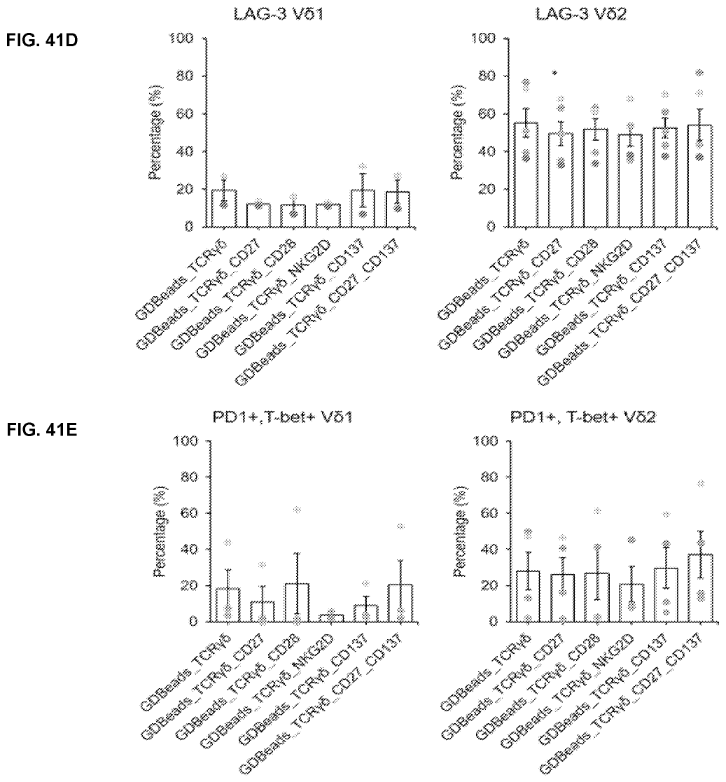

PBMCs cultured with GDBeads for 8 days were analyzed by flow cytometry, which showed that GDBeads effectively enriched and expanded γδ T cells, like soluble antibodies, including both Vδ1 T cells and Vδ2 T cells (FIGS. 37A-37B, 38A-38B, and 39A-39C). Surprisingly, GDBeads coated with both anti-TCRγδ antibody and co-stimulatory receptor antibodies significantly promoted the γδ T cells expansion compared to GDBeads coated only with anti-TCRγδ antibodies (FIGS. 38A-38B and 39A-39C). This was unexpected because soluble co-stimulatory receptor antibodies did not enhance the soluble anti-TCRγδ antibody-mediated γδ T cell expansion (FIG. 38A). Next, the change of surface markers on γδ T cells expanded by GDBeads was examined on day 8 (FIGS. 40A-40E and 41A-41E). The results showed that co-stimulatory receptor antibodies enhance the expression of cytotoxicity functional markers of Vδ1 and Vδ2 T cells, including Granzyme B (FIG. 40A), IFN-7 (FIG. 40B), Perforin (FIG. 40C), TNF-α (FIG. 40D), and IL-17A (FIG. 40E). The proportion of immune checkpoint-positive γδ T cells is significantly reduced, including reduced PD-1+ cells (FIG. 41A), CTLA-4+ cells (FIG. 41B), Tim3+ cells (FIG. 41C), LAG-3+ cells (FIG. 41D), and T-Bet+ cells (in PD1+ cells) (FIG. 41E) when cultured with co-stimulatory receptor antibodies. All of these data suggest that GDBeads stimulate the activation and function of expanded γδ T cells by changing the expression of surface markers on expanded γδ T cells.

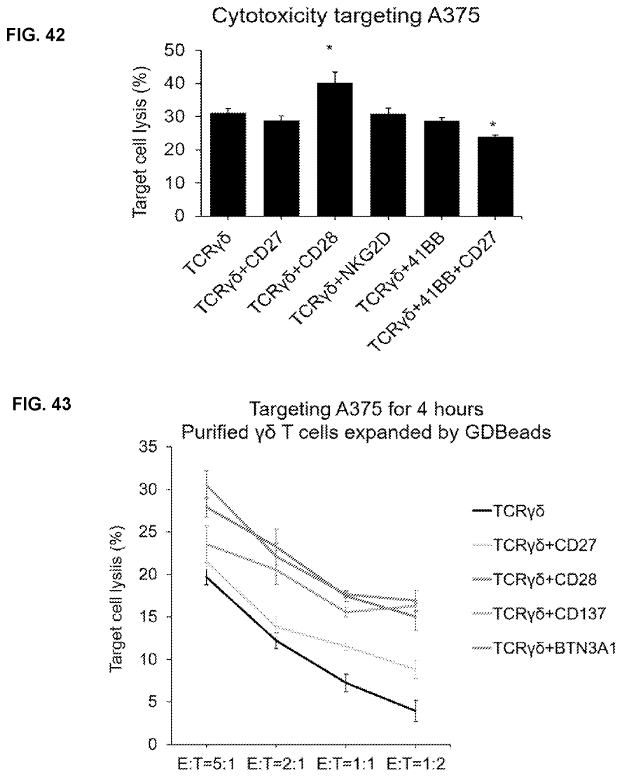

Next, the cytotoxicity of the γδ T cells expanded ex vivo with GDBeads on human melanoma cell line (A375) was examined (FIGS. 42-43). GDBeads coated with anti-γδTCR antibodies or with anti-γδTCR antibodies and co-stimulatory receptor antibodies significantly promoted the cytotoxicity of γδ T cells to melanoma cell lines at an effector/target ratio of 5 (FIG. 42) and different effector:target ratios (FIG. 43).

Thus, GDBeads coated with anti-γδTCR antibodies or with anti-γδTCR antibodies and co-stimulatory receptor antibodies, can be used alone, or with a TLR agonist, such as a TLR7/8 agonist (e.g., resiquimod), to significantly promote the expansion and anti-tumoral functions of γδ T cells.

### Example 18: Cytotoxicity Assay for Testing Anti-Tumor Effect of Anti-CD19-CAR γδT Cells

Effector cells are expanded γδ T cell using the methods indicated herein or T cells (mostly αβ T cells), transduced with indicated CAR, such as anti-CD19 CAR. Target cells are either control K562 cells that do not express CD19 (K562 cc), or K562 cells transformed with CD19 (K562.CD19), or malignant B cells isolated from CLL patients. Cytotoxicity is assessed by either a standard 51Cr-release assay, or a luciferase assay, or a LDH release assay. Effector T cells are mixed with target cells in the wells at varying ratios of effector cell:target cell (E:T). After 4 hours to 24 hours of incubation at 37° C., released 51Cr, bioluminescence, or released LDH is measured using a gamma particle counter, or a bioluminescent scanner or a microplate reader. Each condition is performed in at least triplicate, and the percentage of lysis is calculated using the corresponding calculation methods. The expanded CD19-CAR-γδ T cells or CD19-CAR-αβT are injected into mice bearing CD19+ CLL cells. Tumor volume is measured by FACS.

### Example 19: TCRγ6 Mediated gdT Cell Expansion: Adding Anti-PD1 Antibody During γδT Cell Expansion Improves Cellular Product Function

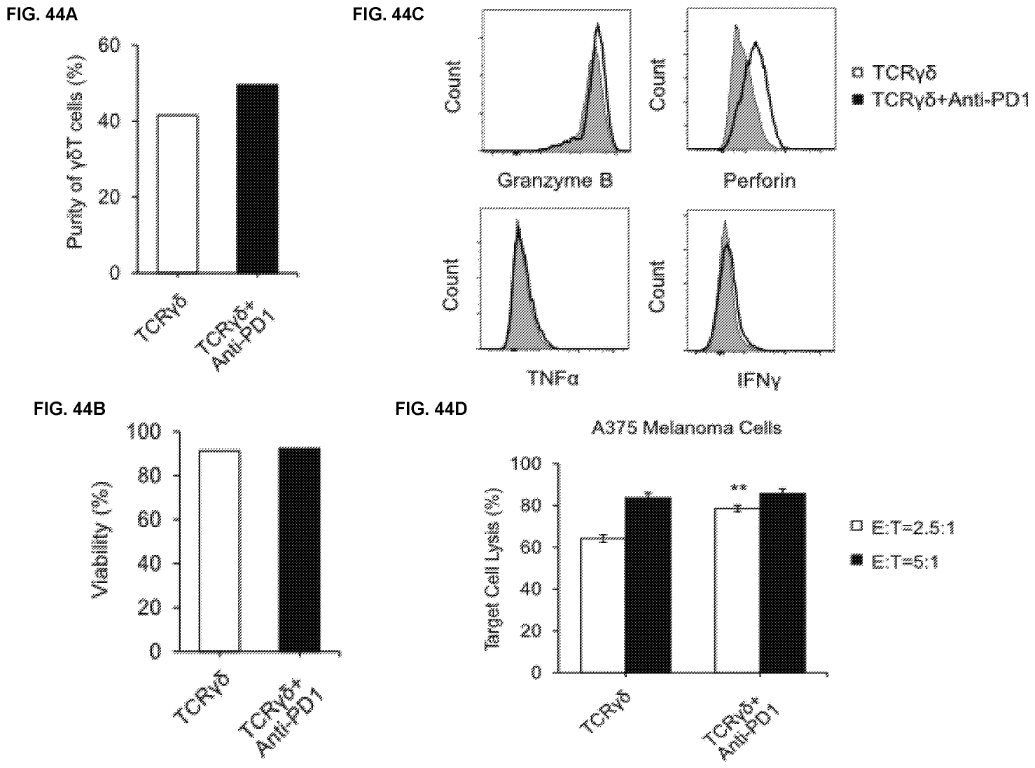

Human γδ T cells were cultured with GDBeads coated with pan-anti-human-TCRγδ antibody (TCRγδ) or pan-anti-human-TCRγδ antibody and anti-human-PD-1 antibody (TCRγδ+Anti-PD1). Flow cytometry data showing the purity of human γδ T cells on day 13 is shown in FIG. 44A. The viability of expended γδ T cells with GDBeads coated with the indicated antibodies is shown in FIG. 44B. Flow cytometry data illustrating GranzymeB, Perforin, IFN-γ and TNF-α expression in γδ T cells expanded by GDBeads coated with indicated antibodies is shown in FIG. 44C. Cytotoxicity of expanded γδ T cells to A375 melanoma cells is shown in FIG. 44D. γδ T cells were expanded from PBMC using GDBeads coated with indicated antibodies for approximately 13 days. 2×10⁴ A375 cells (Firefly Luciferase was stable transduced) were added in each well. Effector:Target (E:T) ratio was 5:1 and 2.5:1 in the coculture. Luciferase assay was performed after coculturing γδ T cells and A375 melanoma cells for 20 hours. Data shown as Mean±S.E.M. **p<0.01. These data demonstrate that adding anti-PD1 antibody during γδ T cell expansion improves the function of the cellular product.

### Enumerated Embodiments

The following enumerated embodiments are provided, the numbering of which is not to be construed as designating levels of importance.

Embodiment 1 provides a method of selecting a donor for γδ T cell-based immunotherapy. The method comprising assessing the expansion capacity of a population of γδT cells from a subject. When the expansion capacity is high, the subject is selected as a donor. When the expansion capacity is low, the subject is not selected as a donor.

Embodiment 2 provides the method of embodiment 1, the assessing comprises incubating the γδ T cells with an agent or agents that expand the γδ T cells.

Embodiment 3 provides the method of embodiment 2, wherein the agent or agents are selected from zoledronate (ZOL), an anti-γδTCR antibody, isopentenyl pyrophosphate (IPP) plus a TLR agonist, and an anti-γδTCR antibody plus a TLR.

Embodiment 4 provides the method of embodiment 1, wherein assessing the expansion capacity comprises calculating an index score.

Embodiment 5 provides the method of embodiment 4, the index score is calculated based on the baseline γδ T cell concentration in the subject's PMBC using the formula: Vδ2 Index Score=−8.23+10.25×Initial γδT %.

Embodiment 6 provides a method of selecting a donor for γδ T cell-based immunotherapy comprising measuring the basal level of Vδ2 T cells in a subject. When the basal level of Vδ2 T cells is high, the subject is selected as a donor. When the basal level of Vδ2 T cells is low, the subject is not selected as a donor.

Embodiment 7 provides the method of any of embodiments 1-6, further comprising autologously transferring the γδ T cell-based immunotherapy to the donor.

Embodiment 8 provides the method of embodiment 6, wherein when the basal level of Vδ2 T cells is between 0.5-3% of the total PBMC population, the level is considered high.

Embodiment 9 provides the method of embodiment 6, wherein when the basal level of Vδ2 T cells is at least 0.82% of the total PBMC population, the level is considered high.

Embodiment 10 provides the method of any one of embodiments 1-8, wherein the method further comprising assesses the immune phenotypes of the γδ T cells.

Embodiment 11 provides the method of embodiment 10, wherein when the subject's γδT cells display effector memory type and central memory phenotype the subject is selected as a donor.

Embodiment 12 provides the method of any one of embodiments, 1-11, wherein, the method further comprises assessing the levels of PD-1, CTLA-4, Eomes, IFN-γ, Granzyme B, and CD86 in the γδ T cells.

Embodiment 13 provides the method of embodiment 12, wherein when the levels of PD-1, CTLA-4 and Eomes are low, and the levels of IFN-γ, Granzyme B, and CD86 are high in the subject's γδ T cells compared to a reference sample, then the subject is selected as a donor.

Embodiment 14 provides the method of embodiment 13, wherein the reference sample comprises γδ T cells from a poor expansion group.

Embodiment 15 provides a composition comprising a population of γδ T cells for use in γδ T cell immunotherapy that is generated by any of the methods in embodiments 1-14.

Embodiment 16 provides a method of treating a disease or disorder in a subject in need thereof with a γδ T cell-based immunotherapy. The method comprises selecting a donor for the γδ T cell-based immunotherapy by assessing the expansion capacity of a population of γδ T cells from a healthy subject. When the expansion capacity is high, the healthy subject is selected as a donor, and a γδ T cell-based immunotherapy comprising the γδ T cells from the donor is administered to the subject in need thereof, thus treating the disease or disorder. When the expansion capacity is low, the healthy subject is not selected as a donor and an alternative treatment is administered or a different donor is selected.

Embodiment 17 provides a method of treating a disease or disorder in a subject in need thereof with a γδ T cell-based immunotherapy. The method comprises assessing the expansion capacity of a population of γδ T cells from the subject. When the expansion capacity is high, the subject is administered a γδ T cell-based immunotherapy comprising the γδ T cells from the subject, thus treating the disease or disorder. When the expansion capacity is low, the γδ T cell-based immunotherapy is not administered and an alternative treatment is administered and/or a new donor is selected.

Embodiment 18 provides the method of embodiment 17, wherein the assessing comprises incubating the γδ T cells with an agent or agents that expand the γδ T cells.

Embodiment 19 provides the method of embodiment 18, wherein the agent or agents are selected from ZOL, an anti-γδTCR antibody, IPP plus a TLR agonist, and an anti-γδTCR antibody plus a TLR agonist.

Embodiment 20 provides the method of embodiment 16 or 17, wherein assessing the expansion capacity comprises calculating an index score.

Embodiment 21 provides the method of embodiment 20, wherein the index score is calculated based on the baseline γδ T cell concentration in the subject's PMBC using the formula: Vδ2 Index Score=−8.23+10.25×Initial γδT %.

Embodiment 22 provides a method of treating a disease or disorder in a subject in need thereof with a γδ T cell-based immunotherapy. The method comprises selecting a donor for the γδ T cell-based immunotherapy by measuring the basal level of Vδ2 T cells in a healthy subject. When the basal level of Vδ2 T cells is high, the healthy subject is selected as a donor, and a γδT cell-based immunotherapy comprising the γδ T cells from the donor is administered to the subject in need thereof, thus treating the disease or disorder. When the basal level of Vδ2 T cells is low, the healthy subject is not selected as a donor, and an alternative treatment is administered or a different donor is selected.

Embodiment 23 provides the method of embodiment 22, wherein when the basal level of Vδ2 T cells is between 0.5-3% of the total PBMC population, the level is considered high.

Embodiment 24 provides the method of embodiment 22, wherein when the basal level of Vδ2 T cells is at least 0.82% of the total PBMC population, the level is considered high.

Embodiment 25 provides the method of any of embodiments 16-24, further comprising assessing the immune phenotypes of the γδ T cells.

Embodiment 26 provides the method of embodiment 25, wherein when the subject's γδT cells display effector memory type and central memory phenotype the subject is selected as a donor.

Embodiment 27 provides the method of any of embodiments 16-26, further comprising assessing the levels of PD-1, CTLA-4, Eomes, IFN-γ, Granzyme B, and CD86 in the γδ T cells.

Embodiment 28 provides the method of embodiment 27, wherein when the levels of PD-1, CTLA-4 and Eomes are low, and the levels of IFN-γ, Granzyme B, and CD86 are high in the subject's γδ T cells compared to a reference sample, then the subject is selected as a donor.

Embodiment 29 provides the method of embodiment 28, wherein the reference sample comprises γδ T cells from a poor expansion group.

Embodiment 30 provides the method of any of embodiments 16-29, wherein the disease or disorder is cancer.

Embodiment 31 provides the method of embodiment 30, wherein the cancer is selected from the group consisting of breast cancer and lung cancer.

Embodiment 32 provides the method of any of embodiments 16-29, wherein the disease or disorder is an infection.

Embodiment 33 provides the method of embodiment 32, wherein the infection is selected from the group consisting of a viral infection, Hepatitis C, Hepatitis B, HIV, EBV, HPV, and a bacterial infection (e.g. tuberculosis).

Embodiment 34 provides the method of any of embodiments 16-33, wherein the γδ T cell-based immunotherapy further comprises a chimeric antigen receptor.

Embodiment 35 provides the method of any of embodiments 16-34, wherein, the γδT cell-based immunotherapy further comprises a modified T cell receptor (TCR).

Embodiment 36 provides a method for expanding and activating a plurality of γδ T cells, the method comprising contacting a population of cells comprising a plurality of γδ T cells with: (a) at least one γδ T cell receptor (γδTCR) activator, wherein the γδTCR activator is not zoledronate and is a weaker γδTCR activator than zoledronate; and (b) a toll-like receptor (TLR) agonist.

Embodiment 37 provides the method of embodiment 36, wherein the γδTCR activator comprises: (i) a phosphoantigen; and/or (ii) at least one anti-γδTCR antibody and/or anti-γδTCR antigen-binding domain.

Embodiment 38 provides the method of any one of embodiments 36-37, wherein the TLR agonist comprises an agonist of TLR7 and/or TLR8 (TLR7/8 agonist).

Embodiment 39 provides the method of any one of embodiments 6-38, wherein the TLR7/8 agonist comprises an imidazoquinoline compound.

Embodiment 40 provides the method of any one of embodiment 39, wherein the imidazoquinoline compound is selected from resiquimod, imiquimod, and gardiquimod.

Embodiment 41 provides the method of embodiment 39, wherein the imidazoquinoline compound is resiquimod.

Embodiment 42 provides the method of embodiment 37, wherein the phosphoantigen is selected from the group consisting of isopentenyl pyrophosphate (IPP), bromohydrin pyrophosphate (BrHPP), and 2-methyl-3-butenyl-1-pyrophosphate (2M3B1PP).

Embodiment 43 provides the method of any one of embodiments 36-43, wherein the γδTCR activator is IPP and the TLR agonist is resiquimod.

Embodiment 44 provides the method of any one of the preceding embodiments, wherein the population of cells comprises peripheral blood mononuclear cells (PBMCs).

Embodiment 45 provides the method of any one of the preceding embodiments, wherein the population of cells comprises human cells.

Embodiment 46 provides the method of any one of the preceding embodiments, wherein the plurality of γδ T cells comprises human γδ T cells.

Embodiment 47 provides the method of any one of the preceding embodiments, wherein the method is performed ex vivo.

Embodiment 48 provides the method of any one of embodiments 36-47, wherein the contacting is performed in a cell culture medium.

Embodiment 49 provides the method of any one of embodiments 36-48, wherein the contacting of step (a) and step (b) are performed on the same day.

Embodiment 50 provides the method of any one of the preceding embodiments, wherein the plurality of γδ T cells is expanded at least 10-fold, at least 100-fold, at least 1,000-fold, or at least 10,000-fold.

Embodiment 51 provides the method of any one of the preceding embodiments, wherein the expanded plurality of γδ T cells comprises Vδ2 T cells.

Embodiment 52 provides the method of any one of embodiments 46-51, wherein a plurality of the at least one anti-γδTCR antibody and/or anti-γδTCR antigen-binding domain is attached to a bead to generate an antibody-conjugated bead.

Embodiment 53 provides the method of embodiment 52, wherein the bead is further attached to a plurality of at least one anti-costimulatory receptor antibody and/or anti-costimulatory receptor antigen-binding domain.

Embodiment 54 provides the method of embodiment 53, wherein the costimulatory receptor is selected from CD27, CD28, CD137 (4-1BB), CD277 (BTN3A1), CD314 (NKG2D) and PD-1 (CD279).

Embodiment 55 provides the method of any one of embodiments 52-54, wherein the bead has a diameter ranging from about 100 nm to about 10 μm.

Embodiment 56 provides the method of any one of embodiments 52-55, wherein the bead is a magnetic bead.

Embodiment 57 provides the method of any one of embodiments 52-56, wherein the plurality of γδ T cells binds to a plurality of the antibody-conjugated beads, and wherein the method further comprises purifying the plurality of γδ T cells away from other cells within the population of cells.

Embodiment 58 provides the method of embodiment 57, wherein the method comprises applying a magnetic field, thereby separating the plurality of γδ T cells bound by the plurality of antibody-conjugated beads from other cells within the population of cells.

Embodiment 59 provides the method of any one of the preceding embodiments, wherein the expanded plurality of γδ T cells is characterized by one or more of the following phenotypes compared to a plurality of γδ T cells expanded in the absence of a TLR agonist and in the presence of the γδTCR activator or a bisphosphonate: (i) increased expression of cytotoxicity surface markers (e.g., granzyme B, CD107a, and/or CD86); (ii) increased expression of proinflammatory cytokines (e.g., IFN-γ, TNF-α, and/or IL-17A); (iii) decreased expression of immune checkpoint proteins (e.g., PD-1); (iv) increased cytotoxicity toward tumor cells; (v) enhanced tumor volume reduction in vivo; (vi) enhanced PI3K-Akt-mTOR pathway; and/or (vii) ability to suppress inhibitory functions of adherent antigen presenting cells (APCs) present in the population of cells.

Embodiment 60 provides the method of any one of the preceding embodiments, wherein the plurality of γδ T cells is genetically modified.

Embodiment 61 provides the method of any one of the preceding embodiments, wherein the plurality of γδ T cells is genetically modified to express a receptor selected from a chimeric antigen receptor (CAR), a TCR, a dominant negative receptor, a switch receptor, or any combination thereof.

Embodiment 62 provides a method for expanding and activating a plurality of γδ T cells, the method comprising contacting a population of cells comprising a plurality of γδ T cells with an antibody-conjugated bead, wherein the antibody-conjugated bead comprises a bead attached to a plurality of at least one anti-γδ T cell receptor (anti-γδTCR) antibody and/or anti-γδTCR antigen-binding domain; and wherein the method does not comprise contacting the population of cells with zoledronate.

Embodiment 63 provides the method of embodiment 62, wherein the bead is further attached to a plurality of at least one anti-costimulatory receptor antibody and/or anti-costimulatory receptor antigen-binding domain.

Embodiment 64 provides the method of embodiment 63, wherein the costimulatory receptor is selected from CD27, CD28, CD137 (4-1BB), CD277 (BTN3A1), CD314 (NKG2D) and PD-1 (CD279).

Embodiment 65 provides the method of any one of embodiments 63-64, wherein the expanded plurality of γδ T cells is characterized by one or more of the following phenotypes compared to a plurality of γδ T cells expanded in the absence of an anti-costimulatory receptor antibody and/or anti-costimulatory receptor antigen binding domain and in the presence of the anti-γδTCR antibody or anti-γδTCR antigen binding domain: (i) increased expression of cytotoxicity surface markers (e.g., granzyme B, CD107a, and/or CD86); (ii) increased expression of proinflammatory cytokines (e.g., IFN-7, TNF-α, and/or IL-17A); (iii) decreased expression of immune checkpoint proteins (e.g., PD-1); (iv) increased cytotoxicity toward tumor cells; and/or (v) enhanced tumor volume reduction in vivo.

Embodiment 66 provides the method of any one of embodiments 62-65, the method further comprising contacting the population of cells comprising a plurality of γδ T cells with a toll-like receptor (TLR) agonist.

Embodiment 67 provides the method of embodiment 66, wherein the TLR agonist comprises an agonist of TLR7 and/or TLR8 (TLR7/8 agonist).

Embodiment 68 provides the method of embodiment 67, wherein the TLR7/8 agonist comprises an imidazoquinoline compound.

Embodiment 69 provides the method of embodiment 68, wherein the imidazoquinoline compound is selected from resiquimod, imiquimod, and gardiquimod.

Embodiment 70 provides the method of embodiment 69, wherein the imidazoquinoline compound is resiquimod.

Embodiment 71 provides the method of any one of embodiments 66-70, wherein contacting the population of cells with the toll-like receptor (TLR) agonist is performed on the same day as contacting the population of cells with the antibody-conjugated bead.

Embodiment 72 provides the method of any one of embodiments 62-71, wherein the population of cells comprises peripheral blood mononuclear cells (PBMCs).

Embodiment 73 provides the method of any one of embodiments 62-72, wherein the population of cells comprises human cells.

Embodiment 74 provides the method of any one of embodiments 62-73, wherein the plurality of γδ T cells comprises human γδ T cells.

Embodiment 75 provides the method of any one of embodiments 62-74, wherein the method is performed ex vivo.

Embodiment γδ provides the method of any one of embodiments 62-76, wherein the contacting is performed in a cell culture medium.

Embodiment 77 provides the method of any one of embodiments 62-76, wherein the plurality of γδ T cells is expanded at least 10-fold, at least 100-fold, at least 1,000-fold, or at least 10,000-fold.

Embodiment 78 provides the method of any one of embodiments 62-77, wherein the expanded plurality of γδ T cells comprises Vδ2 T cells.

Embodiment 79 provides the method of any one of embodiments 62-78, wherein the bead has a diameter ranging from about 100 nm to about 10 μm.

Embodiment 80 provides the method of any one of embodiments 62-79, wherein the bead is a magnetic bead.

Embodiment 81 provides the method of any one of embodiments 62-80, wherein the plurality of γδ T cells binds to a plurality of the antibody-conjugated beads, and wherein the method further comprises purifying the plurality of γδ T cells away from other cells within the population of cells.

Embodiment 82 provides the method of embodiment 81, wherein the method comprises applying a magnetic field, thereby separating the plurality of γδ T cells bound by the plurality of antibody-conjugated beads from other cells within the population of cells.

Embodiment 83 provides the method of any one of embodiments 66-81, wherein the expanded plurality of γδ T cells is characterized by one or more of the following phenotypes compared to a plurality of γδ T cells expanded in the absence of a TLR agonist and in the presence of a γδTCR activator: (i) increased expression of cytotoxicity surface markers (e.g., granzyme B, CD107a, and/or CD86); (ii) increased expression of proinflammatory cytokines (e.g., IFN-7, TNF-α, and/or IL-17A); (iii) decreased expression of immune checkpoint proteins (e.g., PD-1); (iv) increased cytotoxicity toward tumor cells; and/or (v) enhanced tumor volume reduction in vivo; (vi) enhanced PI3K-Akt-mTOR pathway; and/or (vii) ability to suppress inhibitory functions of adherent antigen presenting cells (APCs) present in the population of cells.

Embodiment 84 provides the method of any one of embodiments 62-83, wherein the plurality of γδ T cells is genetically modified.

Embodiment 85 provides the method of any one of embodiments 62-84, wherein the plurality of γδ T cells is genetically modified to express a receptor selected from a chimeric antigen receptor (CAR), a TCR, a dominant negative receptor, a switch receptor, or any combination thereof.

Embodiment 86 provides a pharmaceutical composition comprising the plurality of γδT cells expanded and activated by the method of any one of embodiments 36-86 and a pharmaceutically-acceptable carrier.

The contents of the articles, patents, and patent applications, and all other documents and electronically available information mentioned or cited herein, are hereby incorporated by reference in their entirety to the same extent as if each individual publication was specifically and individually indicated to be incorporated by reference. Applicants reserve the right to physically incorporate into this application any and all materials and information from any such articles, patents, patent applications, or other physical and electronic documents.

While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

1. A method of selecting a donor for γδ T cell-based immunotherapy, the method comprising assessing the expansion capacity of a population of γδ T cells from a subject, wherein when the expansion capacity is high, the subject is selected as a donor, and wherein when the expansion capacity is low, the subject is not selected as a donor.

2. The method of claim 1, wherein the assessing comprises incubating the γδT cells with an agent or agents that expand the γδ T cells.

3. The method of claim 2, wherein the agent or agents are selected from zoledronate (ZOL), an anti-γδTCR antibody, isopentenyl pyrophosphate (IPP) plus a TLR agonist, and an anti-γδTCR antibody plus a TLR.

4. The method of claim 1, wherein assessing the expansion capacity comprises calculating an index score.

5. The method of claim 4, wherein the index score is calculated based on the baseline γδ T cell concentration in the subject's PMBC using the formula: Vδ2 Index Score=−8.23+10.25×Initial γδT %.

6. A method of selecting a donor for γδ T cell-based immunotherapy, the method comprising measuring the basal level of Vδ2 T cells in a subject, wherein when the basal level of Vδ2 T cells is high, the subject is selected as a donor, and wherein when the basal level of Vδ2 T cells is low, the subject is not selected as a donor.

7. The method of claim 1 any of the above claim 1, further comprising autologously transferring the γδ T cell-based immunotherapy to the donor.

8. The method of claim 6, wherein when the basal level of Vδ2 T cells is between 0.5-3% of the total PBMC population, the level is considered high.

9. The method of claim 6, wherein when the basal level of Vδ2 T cells is at least 0.82% of the total PBMC population, the level is considered high.

10. The method of claim 1, further comprising assessing the immune phenotypes of the γδ T cells.

11. The method of claim 7, wherein when the subject's γδ T cells display effector memory type and central memory phenotype the subject is selected as a donor.

12. The method of claim 1, further comprising assessing the levels of PD-1, CTLA-4, Eomes, IFN-γ, Granzyme B, and CD86 in the γδ T cells.

13. The method of claim 12, wherein when the levels of PD-1, CTLA-4 and Eomes are low, and the levels of IFN-γ, Granzyme B, and CD86 are high in the subject's γδ T cells compared to a reference sample, then the subject is selected as a donor.

14. The method of claim 13, wherein the reference sample comprises γδ T cells from a poor expansion group.

15. A composition comprising a population of γδ T cells for use in γδT cell immunotherapy, wherein the population of cells is generated by the method of claim 1.

16. A method of treating a disease or disorder in a subject in need thereof with a γδ T cell-based immunotherapy, the method comprising:
selecting a donor for the γδ T cell-based immunotherapy by assessing the expansion capacity of a population of γδ T cells from a healthy subject,
wherein when the expansion capacity is high, the healthy subject is selected as a donor, and a γδ T cell-based immunotherapy comprising the γδ T cells from the donor is administered to the subject in need thereof, thus treating the disease or disorder, and
wherein when the expansion capacity is low, the healthy subject is not selected as a donor and an alternative treatment is administered or a different donor is selected.

17. A method of treating a disease or disorder in a subject in need thereof with a γδ T cell-based immunotherapy, the method comprising:
assessing the expansion capacity of a population of γδ T cells from the subject, wherein when the expansion capacity is high, the subject is administered a γδ T cell-based immunotherapy comprising the γδ T cells from the subject, thus treating the disease or disorder, and
wherein when the expansion capacity is low, the γδ T cell-based immunotherapy is not administered and an alternative treatment is administered and/or a new donor is selected.

18. The method of claim 16, wherein the assessing comprises incubating the γδ T cells with an agent or agents that expand the γδ T cells.

19. The method of claim 18, wherein the agent or agents are selected from ZOL, an anti-γδTCR antibody, IPP plus a TLR agonist, and an anti-γδTCR antibody plus a TLR agonist.

20. The method of claim 16, wherein assessing the expansion capacity comprises calculating an index score.

21. The method of claim 20, wherein the index score is calculated based on the baseline γδ T cell concentration in the subject's PMBC using the formula: Vδ2 Index Score=−8.23+10.25×Initial γδT %.

22. A method of treating a disease or disorder in a subject in need thereof with a γδ T cell-based immunotherapy, the method comprising:
selecting a donor for the γδ T cell-based immunotherapy by measuring the basal level of Vδ2 T cells in a healthy subject,
wherein when the basal level of Vδ2 T cells is high, the healthy subject is selected as a donor, and a γδ T cell-based immunotherapy comprising the γδ T cells from the donor is administered to the subject in need thereof, thus treating the disease or disorder, and
wherein when the basal level of Vδ2 T cells is low, the healthy subject is not selected as a donor, and an alternative treatment is administered or a different donor is selected.

23. The method of claim 22, wherein when the basal level of Vδ2 T cells is between 0.5-3% of the total PBMC population, the level is considered high.

24. The method of claim 22, wherein when the basal level of Vδ2 T cells is at least 0.82% of the total PBMC population, the level is considered high.

25. The method of claim 16, further comprising assessing the immune phenotypes of the γδ T cells.

26. The method of claim 25, wherein when the subject's γδ T cells display effector memory type and central memory phenotype the subject is selected as a donor.

27. The method of claim 16, further comprising assessing the levels of PD-1, CTLA-4, Eomes, IFN-γ, Granzyme B, and CD86 in the γδ T cells.

28. The method of claim 27, wherein when the levels of PD-1, CTLA-4 and Eomes are low, and the levels of IFN-γ, Granzyme B, and CD86 are high in the subject's γδ T cells compared to a reference sample, then the subject is selected as a donor.

29. The method of claim 28, wherein the reference sample comprises γδ T cells from a poor expansion group.

30. The method of claim 16, wherein the disease or disorder is cancer.

31. The method of claim 30, wherein the cancer is selected from the group consisting of breast cancer and lung cancer.

32. The method of claim 16, wherein the disease or disorder is an infection.

33. The method of claim 32, wherein the infection is selected from the group consisting of a viral infection, Hepatitis C, Hepatitis B, HIV, EBV, HPV, and a bacterial infection.

34. The method of claim 16, wherein the γδT cell-based immunotherapy further comprises a chimeric antigen receptor.

35. The method of claim 16, wherein the γδT cell-based immunotherapy further comprises a modified T cell receptor (TCR).

36. A method for expanding and activating a plurality of γδ T cells, the method comprising contacting a population of cells comprising a plurality of γδ T cells with:
(a) at least one γδ T cell receptor (γδTCR) activator, wherein the γδTCR activator is not zoledronate and is a weaker γδTCR activator than zoledronate; and
(b) a toll-like receptor (TLR) agonist.

37. The method of claim 36, wherein the γδTCR activator comprises:
(i) a phosphoantigen; and/or
(ii) at least one anti-γδTCR antibody and/or anti-γδTCR antigen-binding domain.

38. The method of claim 36, wherein the TLR agonist comprises an agonist of TLR7 and/or TLR8 (TLR7/8 agonist).

39. The method of claim 38, wherein the TLR7/8 agonist comprises an imidazoquinoline compound.

40. The method of claim 39, wherein the imidazoquinoline compound is selected from resiquimod, imiquimod, and gardiquimod.

41. The method of claim 39, wherein the imidazoquinoline compound is resiquimod.

42. The method of claim 37, wherein the phosphoantigen is selected from the group consisting of isopentenyl pyrophosphate (IPP), bromohydrin pyrophosphate (BrHPP), and 2-methyl-3-butenyl-1-pyrophosphate (2M3B1PP).

43. The method of claim 36, wherein the γδTCR activator is IPP and the TLR agonist is resiquimod.

44. The method of claim 36, wherein the population of cells comprises peripheral blood mononuclear cells (PBMCs).

45. The method of claim 36, wherein the population of cells comprises human cells.

46. The method of claim 36, wherein the plurality of γδ T cells comprises human γδ T cells.

47. The method of claim 36, wherein the method is performed ex vivo.

48. The method of claim 36, wherein the contacting is performed in a cell culture medium.

49. The method of claim 36, wherein the contacting of step (a) and step (b) are performed on the same day.

50. The method of claim 36, wherein the plurality of γδ T cells is expanded at least 10-fold, at least 100-fold, at least 1,000-fold, or at least 10,000-fold.

51. The method of claim 36, wherein the expanded plurality of γδ T cells comprises Vδ2 T cells.

52. The method of claim 36, wherein a plurality of the at least one anti-γδTCR antibody and/or anti-γδTCR antigen-binding domain is attached to a bead to generate an antibody-conjugated bead.

53. The method of claim 52, wherein the bead is further attached to a plurality of at least one anti-costimulatory receptor antibody and/or anti-costimulatory receptor antigen-binding domain.

54. The method of claim 53, wherein the costimulatory receptor is selected from CD27, CD28, CD137 (4-1BB), CD277 (BTN3A1), CD314 (NKG2D), and PD-1 (CD279).

55. The method of claim 52, wherein the bead has a diameter ranging from about 100 nm to about 10 μm.

56. The method of claim 52, wherein the bead is a magnetic bead.

57. The method of claim 17, wherein the plurality of γδ T cells binds to a plurality of the antibody-conjugated beads, and wherein the method further comprises purifying the plurality of γδ T cells away from other cells within the population of cells.

58. The method of claim 57, wherein the method comprises applying a magnetic field, thereby separating the plurality of γδ T cells bound by the plurality of antibody-conjugated beads from other cells within the population of cells.

59. The method of claim 36, wherein the expanded plurality of γδ T cells is characterized by one or more of the following phenotypes compared to a plurality of γδ T cells expanded in the absence of a TLR agonist and in the presence of the γδTCR activator or a bisphosphonate:
(i) increased expression of cytotoxicity surface markers (e.g., granzyme B, CD107a, and/or CD86);
(ii) increased expression of proinflammatory cytokines (e.g., IFN-γ, TNF-α, and/or IL-17A);
(iii) decreased expression of immune checkpoint proteins (e.g., PD-1);
(iv) increased cytotoxicity toward tumor cells;
(v) enhanced tumor volume reduction in vivo;
(vi) enhanced PI3K-Akt-mTOR pathway; and/or
(vii) ability to suppress inhibitory functions of adherent antigen presenting cells (APCs) present in the population of cells.

60. The method of claim 36, wherein the plurality of γδ T cells is genetically modified.

61. The method of claim 36, wherein the plurality of γδ T cells is genetically modified to express a receptor selected from a chimeric antigen receptor (CAR), a TCR, a dominant negative receptor, a switch receptor, or any combination thereof.

62. A method for expanding and activating a plurality of γδ T cells, the method comprising contacting a population of cells comprising a plurality of γδ T cells with an antibody-conjugated bead,
wherein the antibody-conjugated bead comprises a bead attached to a plurality of at least one anti-γδ T cell receptor (anti-γδTCR) antibody and/or anti-γδTCR antigen-binding domain; and
wherein the method does not comprise contacting the population of cells with zoledronate.

63. The method of claim 62, wherein the bead is further attached to a plurality of at least one anti-costimulatory receptor antibody and/or anti-costimulatory receptor antigen-binding domain.

64. The method of claim 63, wherein the costimulatory receptor is selected from CD27, CD28, CD137 (4-1BB), CD277 (BTN3A1), CD314 (NKG2D), and PD-1 (CD279).

65. The method of claim 62, wherein the bead is further attached to an anti-PD1 antibody.

66. The method of claim 63, wherein the expanded plurality of γδ T cells is characterized by one or more of the following phenotypes compared to a plurality of γδ T cells expanded in the absence of an anti-costimulatory receptor antibody and/or anti-costimulatory receptor antigen binding domain and in the presence of the anti-γδTCR antibody or anti-γδTCR antigen binding domain:
(i) increased expression of cytotoxicity surface markers (e.g., granzyme B, CD107a, and/or CD86);
(ii) increased expression of proinflammatory cytokines (e.g., IFN-γ, TNF-α, and/or IL-17A);
(iii) decreased expression of immune checkpoint proteins (e.g., PD-1);
(iv) increased cytotoxicity toward tumor cells; and/or
(v) enhanced tumor volume reduction in vivo.

67. The method of claim 62, further comprising contacting the population of cells comprising a plurality of γδ T cells with a toll-like receptor (TLR) agonist.

68. The method of claim 67, wherein the TLR agonist comprises an agonist of TLR7 and/or TLR8 (TLR7/8 agonist).

69. The method of claim 68, wherein the TLR7/8 agonist comprises an imidazoquinoline compound.

70. The method of claim 69, wherein the imidazoquinoline compound is selected from resiquimod, imiquimod, and gardiquimod.

71. The method of claim 70, wherein the imidazoquinoline compound is resiquimod.

72. The method of claim 67, wherein contacting the population of cells with the toll-like receptor (TLR) agonist is performed on the same day as contacting the population of cells with the antibody-conjugated bead.

73. The method of claim 62, wherein the population of cells comprises peripheral blood mononuclear cells (PBMCs).

74. The method of claim 62, wherein the population of cells comprises human cells.

75. The method of claim 62, wherein the plurality of γδ T cells comprises human γδ T cells.

76. The method of claim 62, wherein the method is performed ex vivo.

77. The method of claim 62, wherein the contacting is performed in a cell culture medium.

78. The method of claim 62, wherein the plurality of γδ T cells is expanded at least 10-fold, at least 100-fold, at least 1,000-fold, or at least 10,000-fold.

79. The method of claim 62, wherein the expanded plurality of γδ T cells comprises Vδ2 T cells.

80. The method of claim 62, wherein the bead has a diameter ranging from about 100 nm to about 10 μm.

81. The method of claim 62, wherein the bead is a magnetic bead.

82. The method of claim 62, wherein the plurality of γδ T cells binds to a plurality of the antibody-conjugated beads, and wherein the method further comprises purifying the plurality of γδ T cells away from other cells within the population of cells.

83. The method of claim 82, wherein the method comprises applying a magnetic field, thereby separating the plurality of γδ T cells bound by the plurality of antibody-conjugated beads from other cells within the population of cells.

84. The method of claim 67, wherein the expanded plurality of γδ T cells is characterized by one or more of the following phenotypes compared to a plurality of γδ T cells expanded in the absence of a TLR agonist and in the presence of a γδTCR activator:
(i) increased expression of cytotoxicity surface markers (e.g., granzyme B, CD107a, and/or CD86);
(ii) increased expression of proinflammatory cytokines (e.g., IFN-γ, TNF-α, and/or IL-17A);
(iii) decreased expression of immune checkpoint proteins (e.g., PD-1);
(iv) increased cytotoxicity toward tumor cells; and/or
(v) enhanced tumor volume reduction in vivo;
(vi) enhanced PI3K-Akt-mTOR pathway; and/or
(vii) ability to suppress inhibitory functions of adherent antigen presenting cells (APCs) present in the population of cells.

85. The method of claim 62, wherein the plurality of γδ T cells is genetically modified.

86. The method of claim 62, wherein the plurality of γδ T cells is genetically modified to express a receptor selected from a chimeric antigen receptor (CAR), a TCR, a dominant negative receptor, a switch receptor, or any combination thereof.

87. A pharmaceutical composition comprising the plurality of γδT cells expanded and activated by the method of claim 36 and a pharmaceutically-acceptable carrier.
